SEQUENCE LISTING (60 sequences)

SEQ ID NO: 1 - protein, 7 aa, synthetic construct
  source 1..7: note = 40A3GL-LO14 VH CDR 1
RNSAVWN

SEQ ID NO: 2 - protein, 18 aa, synthetic construct
  source 1..18: note = 40A3GL-LO14 VH CDR 2
RTYYRSKWYNDYAPSVKS

SEQ ID NO: 3 - protein, 13 aa, synthetic construct
  source 1..13: note = 40A3GL-LO14 VH CDR 3
GLRQNQFYYYMDV

SEQ ID NO: 4 - protein, 11 aa, synthetic construct
  source 1..11: note = 40A3GL-LO14 VL CDR 1
RASQSVASNLA

SEQ ID NO: 5 - protein, 7 aa, synthetic construct
  source 1..7: note = 40A3GL-LO14 VL CDR 2
GASTRAT

SEQ ID NO: 6 - protein, 9 aa, synthetic construct
  source 1..9: note = 40A3GL-LO14 VL CDR 3
QQYNNWPFT

SEQ ID NO: 7 - protein, 7 aa, synthetic construct
  source 1..7: note = 40A3GL-LO11 VH CDR 1
RNSAVWN

SEQ ID NO: 8 - protein, 18 aa, synthetic construct
  source 1..18: note = 40A3GL-LO11 VH CDR 2
RTYYRSKWYNDYAPSVKS

SEQ ID NO: 9 - protein, 13 aa, synthetic construct
  source 1..13: note = 40A3GL-LO11 VH CDR 3
GLLQNQFYYYMDV

SEQ ID NO: 10 - protein, 11 aa, synthetic construct
  source 1..11: note = 40A3GL-LO11 VL CDR 1
RASQSVASNLA

SEQ ID NO: 11 - protein, 7 aa, synthetic construct
  source 1..7: note = 40A3GL-LO11 VL CDR 2
GASTRAT

SEQ ID NO: 12 - protein, 9 aa, synthetic construct
  source 1..9: note = 40A3GL-LO11 VL CDR 3
QQYNNWPFT

SEQ ID NO: 13 - protein, 7 aa, synthetic construct
  source 1..7: note = STEAP2 VH CDR1
RNSAVWN

SEQ ID NO: 14 - protein, 18 aa, synthetic construct
  source 1..18: note = STEAP2 VH CDR2
RTYYRSKWYNDYAVSVKS

SEQ ID NO: 15 - protein, 13 aa, synthetic construct
  source 1..13: note = STEAP2 VH CDR3
GLLQNNFYYYMDV

SEQ ID NO: 16 - protein, 11 aa, synthetic construct
  source 1..11: note = STEAP2 VL CDR1 (40A3)
RASQSVNSNLA

SEQ ID NO: 17 - protein, 7 aa, synthetic construct
  source 1..7: note = STEAP2 VL CDR2
GASTRAT

SEQ ID NO: 18 - protein, 9 aa, synthetic construct
  source 1..9: note = STEAP2 VL CDR3
QQYNNWPFT

SEQ ID NO: 19 - protein, 7 aa, synthetic construct
  source 1..7: note = STEAP2-3 VH CDR1
RNSAVWN

SEQ ID NO: 20 - protein, 18 aa, synthetic construct
  source 1..18: note = STEAP2-3 VH CDR2
RTYYRSKWYNDYAVSVKS

SEQ ID NO: 21 - protein, 13 aa, synthetic construct
  source 1..13: note = STEAP2-3 VH CDR3
GLLQNQFYYYMDV

SEQ ID NO: 22 - protein, 11 aa, synthetic construct
  source 1..11: note = STEAP2-3 VL CDR1(40A3GL-LO7)
RASQSVSSNLA

SEQ ID NO: 23 - protein, 7 aa, synthetic construct
  source 1..7: note = STEAP2-3 VL CDR2
GASTRAT

SEQ ID NO: 24 - protein, 9 aa, synthetic construct
  source 1..9: note = STEAP2-3 VL CDR3
QQYNNWPFT

SEQ ID NO: 25 - protein, 5 aa, synthetic construct
  source 1..5: note = Humanized STEAP2-2 VH CDR1
SYGMS

SEQ ID NO: 26 - protein, 17 aa, synthetic construct
  source 1..17: note = Humanized STEAP2-2 VH CDR2
TISSGGSYTFYPDIMKG

SEQ ID NO: 27 - protein, 13 aa, synthetic construct
  source 1..13: note = Humanized STEAP2-2 VH CDR3
RGYGTIYTFSFDA

SEQ ID NO: 28 - protein, 16 aa, synthetic construct
  source 1..16: note = Humanized STEAP2-2 VL CDR1
RSSQSVVHSNANTYLE

SEQ ID NO: 29 - protein, 7 aa, synthetic construct
  source 1..7: note = Humanized STEAP2-2 VL CDR2
KVSNRFS

SEQ ID NO: 30 - protein, 9 aa, synthetic construct
  source 1..9: note = Humanized STEAP2-2 VL CDR3
FQGSHVPYT

SEQ ID NO: 31 - protein, 125 aa, synthetic construct
  source 1..125: note = 40A3GL-LO14 VH
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSS

SEQ ID NO: 32 - protein, 107 aa, synthetic construct
  source 1..107: note = 40A3GL-LO14 VL
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIK

SEQ ID NO: 33 - protein, 125 aa, synthetic construct
  source 1..125: note = 40A3GL-LO11 VH
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLLQNQFYYYMDVWGKGTTVTVSS

SEQ ID NO: 34 - protein, 107 aa, synthetic construct; identical to SEQ ID NO: 32
  source 1..107: note = 40A3GL-LO11 VL

SEQ ID NO: 35 - protein, 125 aa, synthetic construct
  source 1..125: note = STEAP2 VH
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 36 - protein, 107 aa, synthetic construct
  source 1..107: note = STEAP2 VL
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 37 - protein, 125 aa, synthetic construct
  source 1..125: note = STEAP2-3 VH
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSS

SEQ ID NO: 38 - protein, 107 aa, synthetic construct
  source 1..107: note = STEAP2-3 VL
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 39 - protein, 122 aa, synthetic construct
  source 1..122: note = Humanized STEAP2-2 VH
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSS

SEQ ID NO: 40 - protein, 112 aa, synthetic construct
  source 1..112: note = Humanized STEAP2-2 VL
DVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIK

SEQ ID NO: 41 - protein, 455 aa, synthetic construct
  source 1..455: note = 40A3GL-LO14 Heavy chain (L234F/L235E/P331S)
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, synthetic construct
  source 1..214: note = 40A3GL-LO14 Light chain
EIVMTQSPATLSVSPGERATLSCRASQSVASNLAWYQQKPGQAPRLLIYGASTRATGIPARFSGSGSGTEFTLTISSLQSEDFAVYYCQQYNNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 455 aa, synthetic construct
  source 1..455: note = 40A3GL-LO11 Heavy chain (L234F/L235E/P331S)
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLLQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 214 aa, synthetic construct; identical to SEQ ID NO: 42
  source 1..214: note = 40A3GL-LO11 Light chain

SEQ ID NO: 45 - protein, 120 aa, synthetic construct
  source 1..120: note = STEAP2-4 VH
EVQLVESGGGLVQPGGSLRLSCAASGFTFSSFAMTWVRQAPGKGLEWVSVITYSGGRTYYADSVKGRFTISRDNSKNTLYLQMNSLRAEDTAVYFCAKDRIAAVGPFDYWGQGTLVTVSS

SEQ ID NO: 46 - protein, 107 aa, synthetic construct
  source 1..107: note = STEAP2-4 VL
DIQLTQSPSFLSASVGDRVTITCRASQGISVYLAWYQQEPGKAPKLLIYAASTLQSGVPSRFSGSGSGTEFTLTISSLQPEDFATYYCQQLNSYPRTFGQGTKVEIK

SEQ ID NO: 47 - protein, 116 aa, synthetic construct
  source 1..116: note = STEAP2-5 VH
QVQLVQSGAEVKKPGASVKVSCKASGYTFTSYGISWVRQAPGQGLEWMGWISGYTGNTNYAQKLQGRVTMTADTSTSTAYMELRSLRSDDTAVYYCARGGSYFDYWGQGTLVTVSS

SEQ ID NO: 48 - protein, 108 aa, synthetic construct
  source 1..108: note = STEAP2-5 VL
DIQMTQSPSTLSASVGDRVTITCRASQSISRWLAWYQQKPGKAPKLLIYKASSLESGVPSRFSGSGSGTEFTLTISSLQPDDFATYYCQQFNSFSPITFGQGTRLEIK

SEQ ID NO: 49 - protein, 117 aa, synthetic construct
  source 1..117: note = STEAP2-6 VH
QVQLQQPGAELVKPGASVKLSCKASGYTFTSYWMEWVKQRPGQGLEWIGMIHPNSGITNYNERFKNKATLTVDKSSSTAYMQLSSLTSEDSAVYYCARDHYYILAYWGQGTLVTVSA

SEQ ID NO: 50 - protein, 112 aa, synthetic construct
  source 1..112: note = STEAP2-6 VL
DVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 51 - protein, 120 aa, synthetic construct
  source 1..120: note = STEAP2-7 VH
QVQLQQPGADLVKPGASVKMSCKASGHTFTNYWVTWVKQRPGQGLEWIGNFYPGSGIIKYNENFRSKATLTVDISSSTAYMQLSSLTSEDSAVYYCARSKLGDSFYFDYWGQGTTLTVSS

SEQ ID NO: 52 - protein, 112 aa, synthetic construct
  source 1..112: note = STEAP2-7 VL
DVVMTQTPLSLPVSLGNQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIK

SEQ ID NO: 53 - protein, 330 aa, synthetic construct
  source 1..330: note = Wild type Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, synthetic construct
  source 1..330: note = Triple Mutation (TM)Variant IgG1 Constant Region ((L234F/L235E/P331S)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 455 aa, synthetic construct
  source 1..455: note = 40A3GL-LO14 VH with Wild Type IgG1 Fc
QVQLQQSGPGLVKPSQTLSLTCAISGDSVSRNSAVWNWIRQSPSRGLEWLGRTYYRSKWYNDYAPSVKSRITINPDTSKNQFSLQLNSVTPEDTAVYYCARGLRQNQFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 6821 bp, synthetic construct
  source 1..6821: mol_type = other DNA; note = STEAP2 Nucleotide Sequence
agtcagcgttcctcgggccctcggcgccacgagctgtccgggcacgcagcccctagcggcgcgtcgctgccaagccggcctccgcgcgcctccctccttccttctcccctggctgttcgcgatccagcttgggtaggcggggaagcagctggagtgcgaccgccgcggcagccaccctgcaaccgccagtcggagagctaagggcaagtcctgaggttgggcccaggagaaagaaggcaaggagacattgtcccaggatattcttggtgatcttggaagtgtccgtatcatggaatcaatctctatgatgggaagccctaagagccttagtgaaacttttttacctaatggcataaatggtatcaaagatgcaaggaaggtcactgtaggtgtgattggaagtggagattttgccaaatccttgaccattcgacttattagatgcggctatcatgtggtcataggaagtagaaatcctaagtttgcttctgaattttttcctcatgtggtagatgtcactcatcatgaagatgctctcacaaaaacaaatataatatttgttgctatacacagagaacattatacctccctgtgggacctgagacatctgcttgtgggtaaaatcctgattgatgtgagcaataacatgaggataaaccagtacccagaatccaatgctgaatatttggcttcattattcccagattctttgattgtcaaaggatttaatgttgtctcagcttgggcacttcagttaggacctaaggatgccagccggcaggtttatatatgcagcaacaatattcaagcgcgacaacaggttattgaacttgcccgccagttgaatttcattcccattgacttgggatccttatcatcagccagagagattgaaaatttacccctacgactctttactctctggagagggccagtggtggtagctataagcttggccacattttttttcctttattcctttgtcagagatgtgattcatccatatgctagaaaccaacagagtgacttttacaaaattcctatagagattgtgaataaaaccttacctatagttgccattactttgctctccctagtatacctcgcaggtcttctggcagctgcttatcaactttattacggcaccaagtataggagatttccaccttggttggaaacctggttacagtgtagaaaacagcttggattactaagttttttcttcgctatggtccatgttgcctacagcctctgcttaccgatgagaaggtcagagagatatttgtttctcaacatggcttatcagcaggttcatgcaaatattgaaaactcttggaatgaggaagaagtttggagaattgaaatgtatatctcctttggcataatgagccttggcttactttccctcctggcagtcacttctatcccttcagtgagcaatgctttaaactggagagaattcagttttattcagtctacacttggatatgtcgctctgctcataagtactttccatgttttaatttatggatggaaacgagcttttgaggaagagtactacagattttatacaccaccaaactttgttcttgctcttgttttgccctcaattgtaattctgggtaagattattttattccttccatgtataagccgaaagctaaaacgaattaaaaaaggctgggaaaagagccaatttctggaagaaggtatgggaggaacaattcctcatgtctccccggagagggtcacagtaatgtgatgacaaatggtgttcacagctgccatataaagttctactcatgccattatttttatgacttctacgttcagttacaagtatgctgtcaaattatcgtgggttgaaacttgttaaatgagatttcaactgacttagtgatagagttttcttcaagttaattttcacaaatgtcatgtttgccaatatgaatttttctagtcaacatattattgtaatttaggtatgttttgttttgttttgcacaactgtaaccctgttgttactttatatttcataatcaggcaaaaatacttacagttaataatatagatataatgttaaaaacaatttgcaaaccagcagaattttaagcttttaaaataattcaatggatatacatttttttctgaagattaagattttaattattcaacttaaaaagtagaaatgcattattatacatttttttaagaaaggacacgttatgttagcatctaggtaaggctgcatgatagcattcctatatttctctcataaaataggatttgaaggatgaaattaattgtatgaagcaatgtgattatatgaagagacacaaattaaaaagacaaattaaacctgaaattatatttaaaatatatttgagacatgaaatacatactgataatacatacctcatgaaagattttattctttattgtgttacagagcagtttcattttcatattaatatactgatcaggaagaggattcagtaacatttggcttccaaaactgctatctctaatacggtaccaatcctaggaactgtatactagttcctacttagaacaaaagtatcaagtttgcacacaagtaatctgccagctgacctttgtcgcaccttaaccagtcaccacttgctatggtataggattatactgatgttctttgagggattctgatgtgctaggcatggttctaagtactttacttgtattatcccatttaatacttagaacaaccccgtgagataagtagttattatcctcattttacacatgagggaccgaaggatagaaaagttatttttcaaaggtcttgcagttaataaatggcagagtgagcattcaagtccaggtagtcatattccagaggccacggttttaaccactaggctctagagctcccgccgcgcccctatgcattatgttcacaatgccaatctagatgcttcctcttttgtataaagtcactgacattctttagagtgggttgggtgcatccaaaaatgtataaaaatattattataataaacttattactgcttgtagggtaattcacagttacttaccctattcttgcttggaacatgagcctggagacccatggcagtccatatgcctccctatgcagtgaagggccctagcagtgttaacaaattgctgagatcccacggagtctttcaaaaatctctgtagagttagtcttctccttttctcttcctgagaagttctcctgcctgcataaccattcattagggagtactttacaagcatgaaggatattagggtaagtggctaattataaatctactctagagacatataatcatacagattattcataaaatttttcagtgctgtccttccacatttaattgcattttgctcaaactgtagaatgccctacattccccccaccccaatttgctatttccttattaaaatagaaaattataggcaagatacaattatatgcgttcctcttcctgaaattataacatttctaaacttacccacgtaggtactactgaatccaactgccaacaataaaaagacttttatttagtagaggctacctttcccaccagtgactctttttctacaactgccttgtcagtttggtaattcacttatgattttctaatgttctcttggtgaattttattatcttgtaccctctttttttttttttttttttttaaagacagagtcttgctctgtcacccaggctggagtgcagtggcacgatctcggctcactgcaagctctgcctcccgggttcacgccattctcctgcctcagcctcccgagtagctgggactacaggtgcccgccaccatgcccggctgatttctttttgtatttttagtagagacggagtttcaccgtgttagccaggatggtctcgatctcctgacctcgtgatccgcccgccttggcctccaaagtgctgggattacaggtgtgagctaccgcgcccggcctattatcttgtactttctaactgagccctctattttctttattttaataatatttctccccacttgagaatcacttgttagttcttggtaggaattcagttgggcaatgataacttttatgggcaaaaacattctattatagtgaactaatgaaaataacagcgtattttcaatattttcttattccttaaattccactcttttaacactatgcttaaccacttaatgtgatgaaatattcctaaaagttaaatgactattaaagcatatattgttgcatgtatatattaagtagccgatactctaaataaaaataccactgttacagataaatggggcctttaaaaatatgaaaaacaaacttgtgaaaatgtataaaagatgcatctgttgtttcaaatggcactatcttcttttcagtactacaaaaacagaataattttgaagttttagaataaatgtaatatatttactataattctaaatgtttaaatgcttttctaaaaatgcaaaactatgatgtttagttgctttattttacctctatgtgattatttttcttaattgttattttttataatcattatttttctgaaccattcttctggcctcagaagtaggactgaattctactattgctaggtgtgagaaagtggtggtgagaaccttagagcagtggagatttgctacctggtctgtgttttgagaagtgccccttagaaagttaaaagaatgtagaaaagatactcagtcttaatcctatgcaaaaaaaaaaatcaagtaattgttttcctatgaggaaaataaccatgagctgtatcatgctacttagcttttatgtaaatatttcttatgtctcctctattaagagtatttaaaatcatatttaaatatgaatctattcatgctaacattatttttcaaaacatacatggaaatttagcccagattgtctacatataaggtttttatttgaattgtaaaatatttaaaagtatgaataaaatatatttataggtatttatcagagatgattattttgtgctacatacaggttggctaatgagctctagtgttaaactacctgattaatttcttataaagcagcataaccttggcttgattaaggaattctactttcaaaaattaatctgataatagtaacaaggtatattatactttcattacaatcaaattatagaaattacttgtgtaaaagggcttcaagaatatatccaatttttaaatattttaatatatctcctatctgataacttaattcttctaaattaccacttgccattaagctatttcataataaattctgtacagtttccccccaaaaaagagatttatttatgaaatatttaaagtttctaatgtggtattttaaataaagtatcataaatgtaataagtaaatatttatttaggaatactgtgaacactgaactaattattcctgtgtcagtctatgaaatccctgttttgaaatacgtaaacagcctaaaatgtgttgaaattattttgtaaatccatgacttaaaacaagatacatacatagtataacacacctcacagtgttaagatttatattgtgaaatgagacaccctaccttcaattgttcatcagtgggtaaaacaaattctgatgtacattcaggacaaatgattagccctaaatgaaactgtaataatttcagtggaaactcaatctgtttttacctttaaacagtgaattttacatgaatgaatgggttcttcactttttttttagtatgagaaaattatacagtgcttaattttcagagattctttccatatgttactaaaaaatgttttgttcagcctaacatactgagttttttttaactttctaaattattgaatttccatcatgcattcatccaaaattaaggcagactgtttggattcttccagtggccagatgagctaaattaaatcacaaaagcagatgcttttgtatgatctccaaattgccaactttaaggaaatattctcttgaaattgtctttaaagatcttttgcagctttgcagatacccagactgagctggaactggaatttgtcttcctattgactctacttctttaaaagcggctgcccattacattcctcagctgtccttgcagttaggtgtacatgtgactgagtgttggccagtgagatgaagtctcctcaaaggaaggcagcatgtgtcctttttcatcccttcatcttgctgctgggattgtggatataacaggagccctggcagctgtctccagaggatcaaagccacacccaaagagtaaggcagattagagaccagaaagaccttgactacttccctacttccactgctttttcctgcatttaagccattgtaaatctgggtgtgttacatgaagtgaaaattaattctttctgcccttcagttctttatcctgataccatttaacactgtctgaattaactagactgcaataattctttcttttgaaagcttttaaaggataatgtgcaattcacattaaaattgattttccattgtcaattagttatactcattttcctgccttgatctttcattagatattttgtatctgcttggaatatattatcttctttttaactgtgtaattggtaattactaaaactctgtaatctccaaaatattgctatcaaattacacaccatgttttctatcattctcatagatctgccttataaacatttaaataaaaagtactatttaatgatttaacttctgttttgaaatgttgtatacacgtggatttttttctcattaaataataattctagtatttga

SEQ ID NO: 57 - protein, 490 aa, synthetic construct
  source 1..490: note = STEAP2 Sequence
MESISMMGSPKSLSETFLPNGINGIKDARKVTVGVIGSGDFAKSLTIRLIRCGYHVVIGSRNPKFASEFFPHVVDVTHHEDALTKTNIIFVAIHREHYTSLWDLRHLLVGKILIDVSNNMRINQYPESNAEYLASLFPDSLIVKGFNVVSAWALQLGPKDASRQVYICSNNIQARQQVIELARQLNFIPIDLGSLSSAREIENLPLRLFTLWRGPVVVAISLATFFFLYSFVRDVIHPYARNQQSDFYKIPIEIVNKTLPIVAITLLSLVYLAGLLAAAYQLYYGTKYRRFPPWLETWLQCRKQLGLLSFFFAMVHVAYSLCLPMRRSERYLFLNMAYQQVHANIENSWNEEEVWRIEMYISFGIMSLGLLSLLAVTSIPSVSNALNWREFSFIQSTLGYVALLISTFHVLIYGWKRAFEEEYYRFYTPPNFVLALVLPSIVILGKIILFLPCISRKLKRIKKGWEKSQFLEEGMGGTIPHVSPERVTVM

SEQ ID NO: 58 - protein, 107 aa, synthetic construct [sequence omitted]

SEQ ID NO: 59 - protein, 227 aa, synthetic construct
  source 1..227: note = FC comprising a L234F/L235E/P331S triple mutation (TM)
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, synthetic construct
  source 1..227: note = Wild Type Fc of IgG1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0041435 A1
(43) Pub. Date: Feb. 6, 2025

(54) STEAP2 ANTIBODY DRUG CONJUGATES AND USES THEREOF

(71) Applicant: MedImmune, LLC, Gaithersburg, MD (US)

(72) Inventors: Asurayya WORREDE, Gaithersburg, MD (US); Frank COMER, Gaithersburg, MD (US); Jeong Min HAN, Gaithersburg, MD (US); Eric James MOORE, Gaithersburg, MD (US); Sujiet PUTHENVEETIL, Gaithersburg, MD (US); Vanessa MUNIZ-MEDINA, Gaithersburg, MD (US); Dewald van DYK, Gaithersburg, MD (US); Sao Fong CHEUNG, Gaithersburg, MD (US); Melanie EHUDIN, Gaithersburg, MD (US); Darlene MONLISH, Gaithersburg, MD (US); Akbar Husain KHAN, Waltham, MA (US); Andreas Lothar Severino MADERNA, Waltham, MA (US); Maximillian Taro William LEE, London (GB); Nicole Danielle BARTOLO, Waltham, MA (US)

(21) Appl. No.: 18/631,656

(22) Filed: Apr. 10, 2024

Related U.S. Application Data
(60) Provisional application No. 63/495,545, filed on Apr. 11, 2023.

(51) Int. Cl. A61K 47/68; A61P 35/00; C07K 16/18; G01N 33/53; G01N 33/68

(52) U.S. Cl. CPC A61K 47/68037; A61K 47/6829; A61K 47/6843; A61K 47/6889; A61P 35/00; C07K 16/18; G01N 33/53; G01N 33/6872; G01N 2333/46

(57) ABSTRACT

The present disclosure relates to binding molecules (e.g. antibodies) for the treatment of cancer, and related antibody-drug conjugates.

### CROSS-REFERENCE TO RELATED APPLICATIONS

This application claims the benefit of U.S. Provisional Patent Application No. 63/495,545, filed Apr. 11, 2023, which is incorporated herein by reference in its entirety.

### REFERENCE TO SEQUENCE LISTING SUBMITTED ELECTRONICALLY

The content of the electronically submitted sequence listing (Name: STEAP2ADC-100-US-NP_ST26.xml; Size: 76,286 bytes; and Date of Creation: Apr. 10, 2024), filed with the application, is incorporated herein by reference in its entirety.

### FIELD OF THE DISCLOSURE

The present disclosure relates to binding molecules (e.g. antibodies) for the treatment of cancer, and related antibody-drug conjugates.

### BACKGROUND

Despite years of research into and development of potential anti-cancer drugs, cancer remains one of the leading diseases globally, with one in three individuals developing some form of cancer in their lifetime.

The principal therapies for cancer remain chemotherapy and radiotherapy. However, these therapies are associated with various undesirable side effects, from fatigue through to sickness and hair loss. These issues are exacerbated by the often-lengthy courses of chemotherapy used.

Over the last couple of decades, a number of antibody therapies for cancer have been developed and marketed, leading to a reduction in the need for harsh forms of therapy (e.g. surgery and chemotherapy) for a number of cancer types. Although the availability of methodology for producing antibodies (e.g. monoclonal antibodies) has greatly improved over this time period, there are relatively few clinically available anti-cancer antibodies, and even fewer that may be used to target a broad spectrum of cancer types. Furthermore, there is a need to increase the potency of therapeutic antibodies, which is generally limited by the target antigen and subsequent effects on the cancer cell following antibody binding.

### SUMMARY OF THE DISCLOSURE

The present disclosure is directed to an antibody or antigen binding fragment thereof which binds to STEAP2, comprising:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively.

In some aspects, the antibody or antigen binding fragment thereof comprises:
i. a variable heavy (VH) chain and a variable light (VL) chain at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively;
ii. a VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 32, respectively;
iii. a VH chain and VL chain at least 90%, at least 91% at least 92%, at least 93%, at least 94%, at least 95%, at least 96%, at least 97%, at least 98%, or at least 99% identical to NO: 35 and SEQ ID NO: 32, respectively;
iv. a VH chain and VL chain at least 90%, at least 91% at least 92%, at least 93%, at least 94%, at least 95%, at least 96%, at least 97%, at least 98%, or at least 99% identical to NO: 37 and SEQ ID NO: 32, respectively;
v. a VH chain and VL chain at least 90%, at least 91% at least 92%, at least 93%, at least 94%, at least 95%, at least 96%, at least 97%, at least 98%, or at least 99% identical to NO: 39 and SEQ ID NO: 32, respectively;
vi. a VH chain and VL chain at least 90%, at least 91% at least 92%, at least 93%, at least 94%, at least 95%, at least 96%, at least 97%, at least 98%, or at least 99% identical to NO: 45 and SEQ ID NO: 32, respectively;
vii. a VH chain and VL chain at least 90%, at least 91% at least 92%, at least 93%, at least 94%, at least 95%, at least 96%, at least 97%, at least 98%, or at least 99% identical to NO: 47 and SEQ ID NO: 32, respectively;
viii. a VH chain and VL chain at least 90%, at least 91% at least 92%, at least 93%, at least 94%, at least 95%, at least 96%, at least 97%, at least 98%, or at least 99% identical to NO: 49 and SEQ ID NO: 32, respectively;
ix. a VH chain and VL chain at least 90%, at least 91% at least 92%, at least 93%, at least 94%, at least 95%, at least 96%, at least 97%, at least 98%, or at least 99% identical to NO: 51 and SEQ ID NO: 32, respectively;
x. a VH chain and VL chain at least 90%, at least 91% at least 92%, at least 93%, at least 94%, at least 95%, at least 96%, at least 97%, at least 98%, or at least 99% identical to SEQ ID NO: 31 and SEQ ID NO: 36, respectively;
xi. VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 36, respectively;
xii. a VH chain and a VL chain at least 90%, at least 91% at least 92%, at least 93%, at least 94%, at least 95%, at least 96%, at least 97%, at least 98%, or at least 99% identical to SEQ ID NO: 35 and SEQ ID NO: 36, respectively;
xiii. a VH chain and a VL chain at least 90%, at least 91% at least 92%, at least 93%, at least 94%, at least 95%, at least 96%, at least 97%, at least 98%, or at least 99% identical to SEQ ID NO: 37 and SEQ ID NO: 38, respectively;
xiv, a VH chain and a VL chain at least 90%, at least 91% at least 92%, at least 93%, at least 94%, at least 95%, at least 96%, at least 97%, at least 98%, or at least 99% identical SEQ ID NO: 39 and SEQ ID NO: 40, respectively;
xv. a VH chain and a VL chain at least 90%, at least 91% at least 92%, at least 93%, at least 94%, at least 95%, at least 96%, at least 97%, at least 98%, or at least 99% identical to SEQ ID NO: 45 and SEQ ID NO: 46, respectively;
xvi. a VH chain and a VL chain at least 90%, at least 91% at least 92%, at least 93%, at least 94%, at least 95%, at least 96%, at least 97%, at least 98%, or at least 99% identical to SEQ ID NO: 47 and SEQ ID NO: 48, respectively;
xvii. a VH chain and a VL chain at least 90%, at least 91% at least 92%, at least 93%, at least 94%, at least 95%, at least 96%, at least 97%, at least 98%, or at least 99% identical SEQ ID NO: 49 and SEQ ID NO: 50, respectively; or
xviii. a VH chain and a VL chain at least 90%, at least 91% at least 92%, at least 93%, at least 94%, at least 95%, at least 96%, at least 97%, at least 98%, or at least 99% identical to SEQ ID NO: 51 and SEQ ID NO: 52, respectively.

In some aspects, the antibody or antigen binding fragment thereof comprises:
i. the VH chain of SEQ ID NO: 31 and the VL chain of SEQ ID NO: 32;
ii. the VH chain of SEQ ID NO: 33 and the VL chain of SEQ ID NO: 32;
iii. the VH chain of SEQ ID NO: 35 and the VL chain of SEQ ID NO: 32;
iv. the VH chain of SEQ ID NO: 37 and the VL chain of SEQ ID NO: 32;
v. the VH chain of SEQ ID NO: 39 and the VL chain of SEQ ID NO: 32;
vi. the VH chain of SEQ ID NO: 45 and the VL chain of SEQ ID NO: 32;
vii. the VH chain of SEQ ID NO: 47 and the VL chain of SEQ ID NO: 32;
viii. the VH chain of SEQ ID NO: 49 and the VL chain of SEQ ID NO: 32;
ix. the VH chain of SEQ ID NO: 51 and the VL chain of SEQ ID NO: 32;
x. the VH chain of SEQ ID NO: 31 and the VL chain of SEQ ID NO: 36;
xi. the VH chain of SEQ ID NO: 33 and the VL chain of SEQ ID NO: 36;
xii. the VH chain of SEQ ID NO: 35 and the VL chain of SEQ ID NO: 36;
xiii. the VH chain of SEQ ID NO: 37 and the VL chain of SEQ ID NO: 38;
xiv. the VH chain of SEQ ID NO: 39 and the VL chain of SEQ ID NO: 40;
xv. the VH chain of SEQ ID NO: 45 and the VL chain of SEQ ID NO: 46;
xvi. the VH chain of SEQ ID NO: 47 and the VL chain of SEQ ID NO: 48;
xvii. the VH chain of SEQ ID NO: 49 and the VL chain of SEQ ID NO: 50; or
xviii. the VH chain of SEQ ID NO: 51 and the VL chain of SEQ ID NO: 52.

In some aspects the antibody or binding fragment thereof comprises an Fc region.

In some aspects, the antibody or antigen binding fragment thereof comprises a VH chain and a VL chain comprising the amino acid sequence of SEQ ID NO: 33 and SEQ ID NO: 32, respectively, or a functional variant thereof.

In some aspects, the antibody or antigen binding fragment thereof binds a cell line selected from the group consisting of LNCaP, AD293 muSTEAP3-2, C42, and 22Rv1.

In some aspects, the antibody or antigen binding fragment thereof comprises a heavy chain constant region comprising the amino acid sequence of SEQ ID NO: 54.

In some aspects, the antibody or antigen binding fragment thereof comprises a light constant region comprising the amino acid sequence of SEQ ID NO: 58.

In some aspects, the antibody or antigen binding fragment thereof comprises a heavy chain comprising the amino acid sequence of SEQ ID NO: 41; and a light chain comprising the amino acid sequence of SEQ ID NO: 42.

In some aspects, the antibody or antigen binding fragment thereof comprises a heavy chain comprising the amino acid sequence of SEQ ID NO: 43; and a light chain comprising the amino acid sequence of SEQ ID NO: 44.

In some aspects, the antibody or antigen binding fragment thereof is conjugated to a heterologous agent.

In some aspects, the antibody or antigen binding fragment thereof is conjugated to one or more heterologous agent selected from the group consisting of a topoisomerase I inhibitor, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

In some aspects, the antibody or antigen binding fragment thereof is conjugated to one or more heterologous agent selected from a topoisomerase I inhibitor, tubulysin derivative, or a combination thereof.

In some aspects, the antibody or antigen binding fragment thereof is conjugated to a topoisomerase I inhibitor.

In some aspects, the antibody or antigen binding fragment thereof is conjugated to a heterologous agent selected from the group consisting of tubulysin AZ1508, SG3932, or a combination thereof.

In some aspects, the antibody or antigen binding fragment thereof is conjugated to a SG3932 cytotoxin:

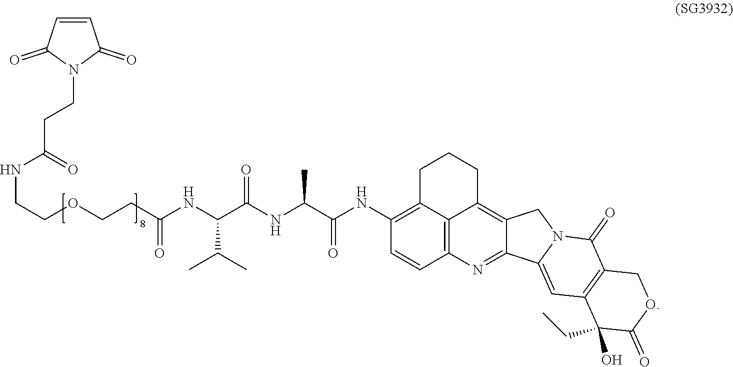

In some aspects, the antibody or antigen binding fragment thereof is conjugated to:

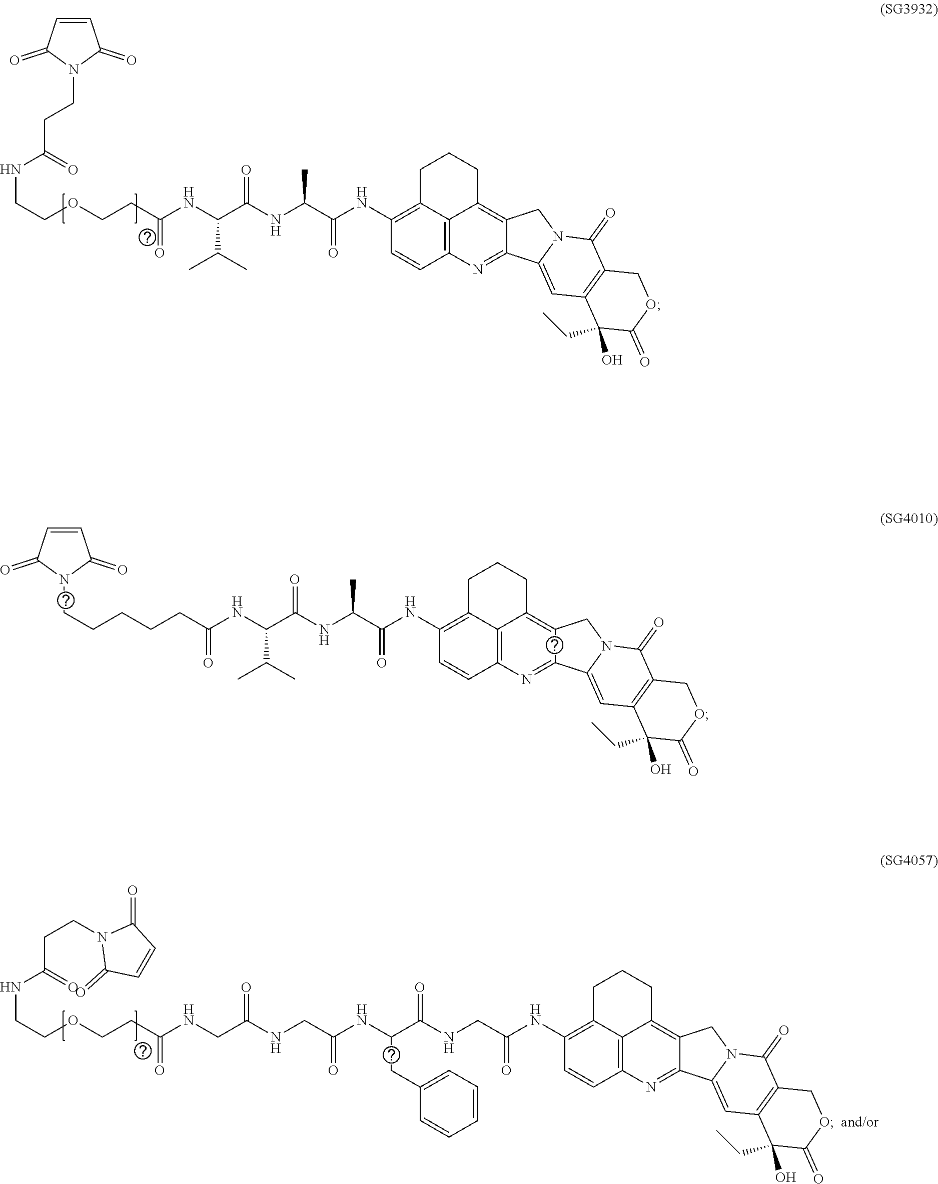

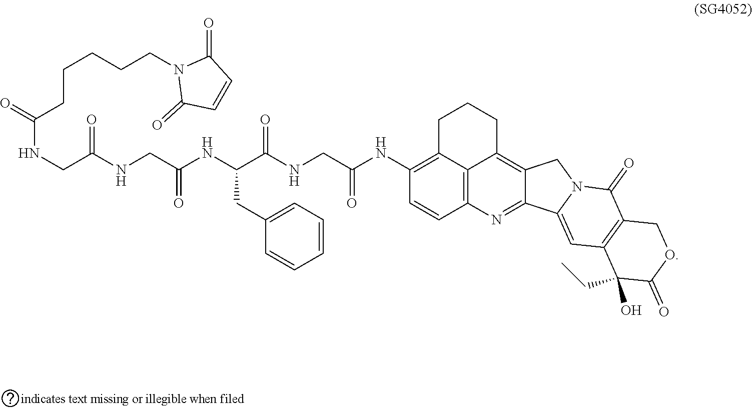 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

In some aspects, the antibody or antigen binding fragment is conjugated to a drug at a drug to antibody ratio (DAR) of about 4 or about 8.

In some aspects, the antibody or antigen binding fragment is conjugated to a drug at a drug to antibody ratio (DAR) of about 8.

In some aspects, the antibody or antigen binding fragment is conjugated to a drug at a drug to antibody ratio (DAR) of 8.

In some aspects, the antibody or antigen binding fragment thereof is a monoclonal antibody.

In some aspects, the antibody or antigen binding fragment thereof is a humanized monoclonal antibody.

In some aspects, the antibody or antigen binding fragment thereof is an IgG1, IgG2, or IgG4 or fragment thereof.

In some aspects, the antibody or antigen binding fragment thereof is an IgG1 or fragment thereof.

In some aspects, the antibody or binding fragment thereof comprises an Fc region.

In some aspects, the Fc region comprises a L234F/L235E/P331S triple mutation TM.

In some aspects, the Fc region comprises a L234F/L235E/P331S triple mutation TM according to SEQ ID NO: 59.

In some aspects, the Fc region comprising a TM has reduced antibody dependent cellular cytotoxicity (ADCC) compared to an antibody having a wild type Fc region.

In some aspects, the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

In another aspect, the disclosure is directed to a pharmaceutical composition comprising an antibody or antigen binding fragment thereof according to the description herein.

In another aspect is a polynucleotide encoding the antibody or antigen binding fragment described herein.

In another aspect, the present disclosure is directed to a host cell comprising a polynucleotide described herein.

Another aspect of the present disclosure is a method for producing an antibody or antigen binding fragment thereof that binds to STEAP2, comprising expressing a polynucleotide described herein in a host cell.

Another aspect is an antibody or antigen binding fragment thereof obtainable by the methods described herein.

In another aspect is a method of treating a cancer comprising a cancer cell which expresses STEAP2, the method comprising administering to a subject the antibody or antigen binding fragment described herein, the pharmaceutical composition described herein, or a combination thereof.

Another aspect of the present disclosure is an antibody or antigen binding fragment described herein, or the pharmaceutical composition described herein, for use in treating a cancer, wherein said cancer comprises a cancer cell which expresses STEAP2.

Another aspect of the present disclosure is a method, or antibody or antigen binding fragment thereof or pharmaceutical composition for use, where said cancer is selected from breast cancer, ovarian cancer, endometrial cancer, cholangiocarcinoma, NSCLC (squamous and/or adenocarcinoma), pancreatic cancer, gastric cancer, and prostate cancer.

In some aspects, the cancer is prostate cancer.

In another aspect, the cancer is metastatic, recurrent, or relapsed prostate cancer.

Another aspect described herein is a method for detecting the presence or absence of a STEAP2 polypeptide in a sample, comprising:
i. contacting a sample with an antibody or antigen binding fragment as described herein, or a pharmaceutical composition as described herein, to provide an antibody-antigen complex;
ii. detecting the presence or absence of said antibody-antigen complex;
iii. wherein the presence of the antibody-antigen complex confirms the presence of a STEAP2 polypeptide;
iv. wherein the absence of the antibody-antigen complex confirms the absence of STEAP2 polypeptide.

In another aspect, is a method wherein the presence of said antibody-antigen complex is indicative of the presence of a cancer cell, and wherein the absence of said antibody-antigen complex is indicative of the absence of a cancer cell.

In another aspect, the sample is an isolated sample obtainable from a subject.

In another aspect, the STEAP2 polypeptide is an integral component of a cancer cell.

The present disclosure also provides an antibody-drug conjugate (ADC) comprising:
(i) (a) an antibody or antigen binding fragment thereof which binds to a STEAP2 polypeptide comprising: a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; and a LCDR1 comprising the amino acid sequence of SEQ ID NO: 4; a LCDR2 comprising the amino acid sequence of SEQ ID NO: 5; and a LCDR3 comprising the amino acid sequence of SEQ ID NO: 6, or
   (b) an antibody or antigen binding fragment thereof which binds to a STEAP2 polypeptide comprising: a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; and a LCDR1 comprising the amino acid sequence of SEQ ID NO: 10; a LCDR2 comprising the amino acid sequence of SEQ ID NO: 11; and a LCDR3 comprising the amino acid sequence of SEQ ID NO: 12;
   (c) an antibody or antigen binding fragment thereof which binds to a STEAP2 polypeptide comprising: a HCDR1 comprising the amino acid sequence of SEQ ID NO: 19, a HCDR2 comprising the amino acid sequence of SEQ ID NO: 20, a HCDR3 comprising the amino acid sequence of SEQ ID NO: 21, a LCDR1 comprising the amino acid sequence of SEQ ID NO: 22, a LCDR2 comprising the amino acid sequence of SEQ ID NO: 23, and a LCDR3 comprising the amino acid sequence of SEQ ID NO: 24, respectively;
(ii) a cytotoxic agent wherein the cytotoxic agent is SG3932; and
(iii) wherein the ADC has a drug to antibody ratio (DAR) ranging from about 4 to 8.

In another aspect, the ADC comprises the antibody or antigen binding fragment thereof which comprises:
i. a variable heavy (VH) chain and a variable light (VL) chain at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively;
ii. a VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 32, respectively;
iii. a VH chain and VL chain at least 90%, at least 91% at least 92%, at least 93%, at least 94%, at least 95%, at least 96%, at least 97%, at least 98%, or at least 99% identical to NO: 35 and SEQ ID NO: 32, respectively;
iv. a VH chain and VL chain at least 90%, at least 91% at least 92%, at least 93%, at least 94%, at least 95%, at least 96%, at least 97%, at least 98%, or at least 99% identical to NO: 37 and SEQ ID NO: 32, respectively;
v. a VH chain and VL chain at least 90%, at least 91% at least 92%, at least 93%, at least 94%, at least 95%, at least 96%, at least 97%, at least 98%, or at least 99% identical to NO: 39 and SEQ ID NO: 32, respectively;
vi. a VH chain and VL chain at least 90%, at least 91% at least 92%, at least 93%, at least 94%, at least 95%, at least 96%, at least 97%, at least 98%, or at least 99% identical to NO: 45 and SEQ ID NO: 32, respectively;
vii. a VH chain and VL chain at least 90%, at least 91% at least 92%, at least 93%, at least 94%, at least 95%, at least 96%, at least 97%, at least 98%, or at least 99% identical to NO: 47 and SEQ ID NO: 32, respectively;
viii. a VH chain and VL chain at least 90%, at least 91% at least 92%, at least 93%, at least 94%, at least 95%, at least 96%, at least 97%, at least 98%, or at least 99% identical to NO: 49 and SEQ ID NO: 32, respectively;
ix. a VH chain and VL chain at least 90%, at least 91% at least 92%, at least 93%, at least 94%, at least 95%, at least 96%, at least 97%, at least 98%, or at least 99% identical to NO: 51 and SEQ ID NO: 32, respectively;
x. a VH chain and VL chain at least 90%, at least 91% at least 92%, at least 93%, at least 94%, at least 95%, at least 96%, at least 97%, at least 98%, or at least 99% identical to SEQ ID NO: 31 and SEQ ID NO: 36, respectively;
xi. VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 36, respectively;
xii. a VH chain and a VL chain at least 90%, at least 91% at least 92%, at least 93%, at least 94%, at least 95%, at least 96%, at least 97%, at least 98%, or at least 99% identical to SEQ ID NO: 35 and SEQ ID NO: 36, respectively;
xiii. a VH chain and a VL chain at least 90%, at least 91% at least 92%, at least 93%, at least 94%, at least 95%, at least 96%, at least 97%, at least 98%, or at least 99% identical to SEQ ID NO: 37 and SEQ ID NO: 38, respectively;
xiv, a VH chain and a VL chain at least 90%, at least 91% at least 92%, at least 93%, at least 94%, at least 95%, at least 96%, at least 97%, at least 98%, or at least 99% identical SEQ ID NO: 39 and SEQ ID NO: 40, respectively;
xv. a VH chain and a VL chain at least 90%, at least 91% at least 92%, at least 93%, at least 94%, at least 95%, at least 96%, at least 97%, at least 98%, or at least 99% identical to SEQ ID NO: 45 and SEQ ID NO: 46, respectively;
xvi. a VH chain and a VL chain at least 90%, at least 91% at least 92%, at least 93%, at least 94%, at least 95%, at least 96%, at least 97%, at least 98%, or at least 99% identical to SEQ ID NO: 47 and SEQ ID NO: 48, respectively;
xvii. a VH chain and a VL chain at least 90%, at least 91% at least 92%, at least 93%, at least 94%, at least 95%, at least 96%, at least 97%, at least 98%, or at least 99% identical SEQ ID NO: 49 and SEQ ID NO: 50, respectively; or
xviii. a VH chain and a VL chain at least 90%, at least 91% at least 92%, at least 93%, at least 94%, at least 95%, at least 96%, at least 97%, at least 98%, or at least 99% identical to SEQ ID NO: 51 and SEQ ID NO: 52, respectively.

In another aspect of the ADC, is the antibody or antigen binding fragment thereof which comprises:
i. the VH chain of SEQ ID NO: 31 and the VL chain of SEQ ID NO: 32;
ii. the VH chain of SEQ ID NO: 33 and the VL chain of SEQ ID NO: 32;
iii. the VH chain of SEQ ID NO: 35 and the VL chain of SEQ ID NO: 32;
iv. the VH chain of SEQ ID NO: 37 and the VL chain of SEQ ID NO: 32;
v. the VH chain of SEQ ID NO: 39 and the VL chain of SEQ ID NO: 32;
vi. the VH chain of SEQ ID NO: 45 and the VL chain of SEQ ID NO: 32;
vii. the VH chain of SEQ ID NO: 47 and the VL chain of SEQ ID NO: 32;
viii. the VH chain of SEQ ID NO: 49 and the VL chain of SEQ ID NO: 32;
ix. the VH chain of SEQ ID NO: 51 and the VL chain of SEQ ID NO: 32;
x. the VH chain of SEQ ID NO: 31 and the VL chain of SEQ ID NO: 36;
xi. the VH chain of SEQ ID NO: 33 and the VL chain of SEQ ID NO: 36;
xii. the VH chain of SEQ ID NO: 35 and the VL chain of SEQ ID NO: 36;
xiii. the VH chain of SEQ ID NO: 37 and the VL chain of SEQ ID NO: 38;
xiv. the VH chain of SEQ ID NO: 39 and the VL chain of SEQ ID NO: 40;
xv. the VH chain of SEQ ID NO: 45 and the VL chain of SEQ ID NO: 46;
xvi. the VH chain of SEQ ID NO: 47 and the VL chain of SEQ ID NO: 48;
xvii. the VH chain of SEQ ID NO: 49 and the VL chain of SEQ ID NO: 50; or
xviii. the VH chain of SEQ ID NO: 51 and the VL chain of SEQ ID NO: 52.

In another aspect, the heavy chain (HC) comprises the amino acid sequence of SEQ ID NO: 41, and light chain (LC) comprises the amino acid sequence of SEQ ID NO: 42.

In another aspect, the drug to antibody ratio (DAR) is about 4 or about 8.

In another aspect, the drug to antibody ratio is about 8.

In another aspect, wherein the drug to antibody ratio is 8.

In another aspect, wherein said antibody or antigen binding fragment thereof is an IgG1, IgG2 or IgG4 or fragment thereof.

In another aspect, said antibody or antigen binding fragment thereof is an IgG1 or fragment thereof.

In another aspect, the antibody or binding fragment thereof comprises an Fc region.

In another aspect, the antibody or binding fragment thereof comprises an Fc region comprising a L234F/L235E/P331S triple mutation TM.

In another aspect, the antibody or binding fragment thereof comprises an Fc region comprising a L234F/L235E/P331S triple mutation TM according to SEQ ID NO: 59.

In another aspect, the antibody or binding fragment thereof comprises an Fc region that has reduced antibody dependent cellular cytotoxicity compared to an antibody comprising a wild type Fc region according to SEQ ID NO: 60.

In another aspect, the present disclosure is directed to a pharmaceutical composition comprising the ADC as described herein.

The present disclosure also provides a method of treating a cancer expressing STEAP2, comprising administering to a subject the ADC as described herein, or the pharmaceutical composition as described herein, or a combination thereof. In another aspect, the subject is a human. In another aspect, the cancer cell has a homologous DNA repair defect.

The present disclosure also provides a method for reducing the volume of a tumor expressing STEAP2, comprising administering to a subject the antibody or antigen binding fragment described herein, the pharmaceutical composition described herein, the ADC of described herein, or a combination thereof. In another aspect, the tumor has a homologous DNA repair defect.

The present disclosure also provides a kit of parts comprising at least one of a (i) variable heavy chain, (ii) variable light chain of the (a) the antibody or antigen binding fragments described herein, (b) the antibody drug conjugate described herein, or (c) the pharmaceutical composition described herein, or a combination thereof. In another aspect, the kit further comprises instructions for use.

The present disclosure also provides an antibody-drug conjugate as in Formula (IC): Ab-(G^{A}-J^{A}-D^{C})ₖ (IC) or a pharmaceutically acceptable salt thereof, wherein Ab is an antibody or antigen-binding fragment thereof described herein, k is an integer from 1 to 10, about 4 to 8, about 4, or about 8, each G^{A} is independently a conjugation group conjugated to the antibody or antigen-binding fragment thereof, each D^{C} is

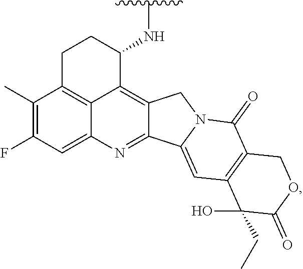 [SMILES: *N[C@H]1CCc2c(C)c(F)cc3nc4c(c1c23)Cn1c-4cc2c(c1=O)COC(=O)[C@]2(O)CC]

each J^{A} is independently a group of Formula (ICA)

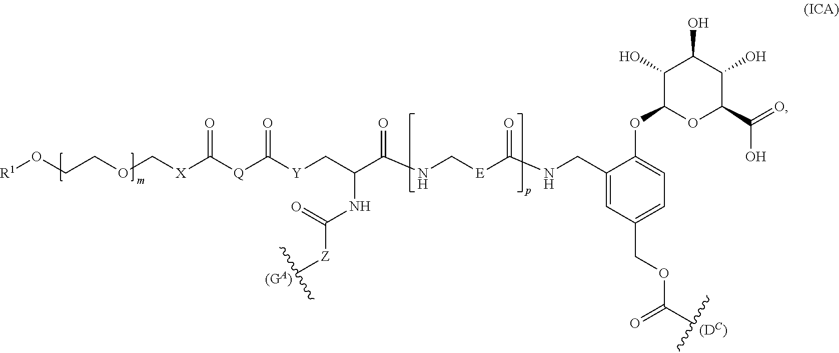

E is (CH₂)ₙ₁, wherein n1 is 0, 1, 2 or 3,
Q is

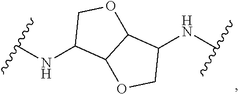 [SMILES: *NC1COC2C(N*)COC12]

R¹ is C₁₋₄ alkyl,
X is (CH₂)ₙ₂, wherein n2 is 0, 1, 2 or 3,
Y is (CH₂)ₙ₃, wherein n 3 is 0, 1, 2, 3 or 4, Z is (CH₂)ₙ₄, wherein n4 is 1, 2, 3, 4 or 5, m is an integer from 5 to 17, p is 1 or 0, (G^{A}) indicates the point of attachment to G^{A}, and (D^{C}) indicates the point of attachment to D^{C}.

In some aspects, Q is

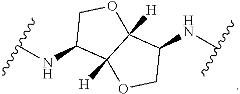 [SMILES: *N[C@H]1CO[C@H]2[C@@H]1OC[C@@H]2N*]

In some aspects m is 9, 10, 11, 12 or 13. In some aspects R¹ is CH₃. In some aspects E is CH₂. In some aspects X is CH₂. In some aspects Y is (CH₂)₂. In some aspects Z is (CH₂)₂. In some aspects p is 1. In some aspects, each J^{A} is a group of Formula (ICB)

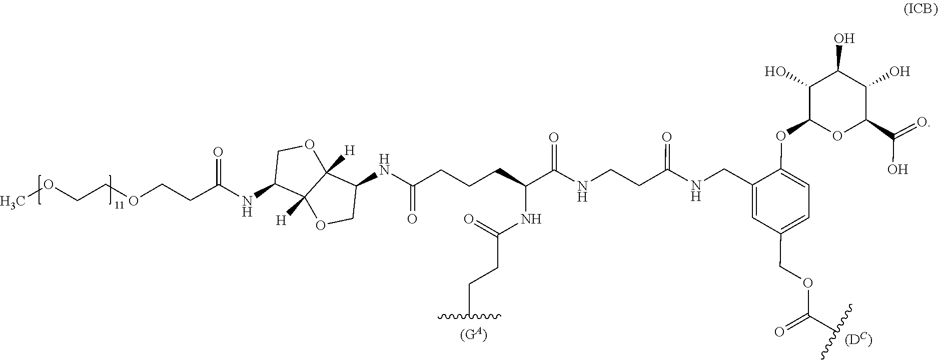

In some aspects G^{A} is selected from

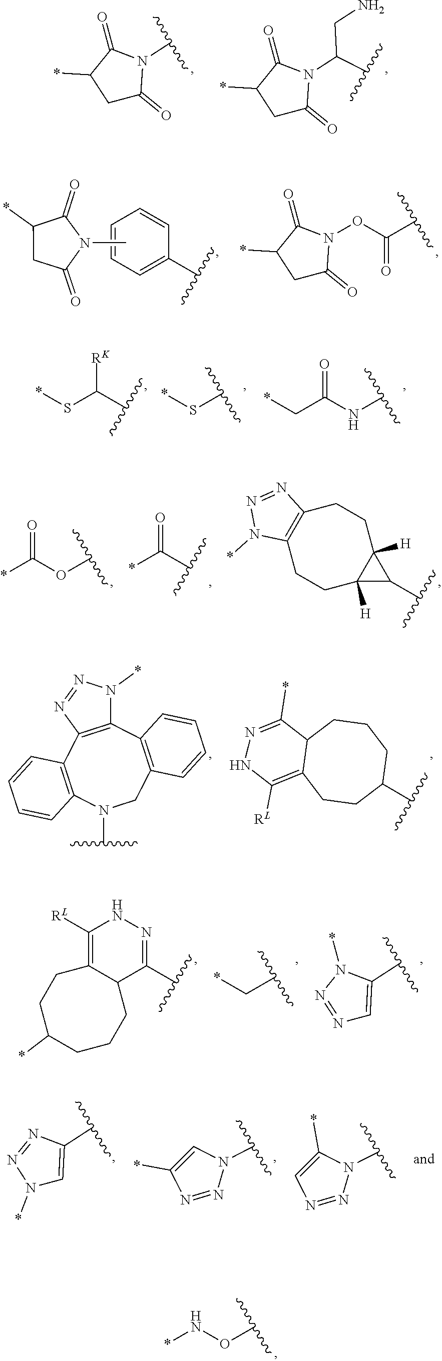

wherein R^{K} is H or CH₃, R^{L} is C₁₋₆ alkyl, and [symbol] indicates the point of attachment to the antibody or antigen-binding fragment thereof. In some aspects, G^{A} is

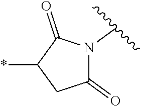 [SMILES: *C1CC(=O)N(*)C1=O]

In some aspects, G^{A} is

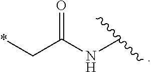 [SMILES: *CC(=O)N*]

In some aspects, k is an integer from 2 to 8. In some aspects, k is 4. In some aspects, k is 8.

In some aspects, G^{A}-J^{A}-D^{C} is LP-1:

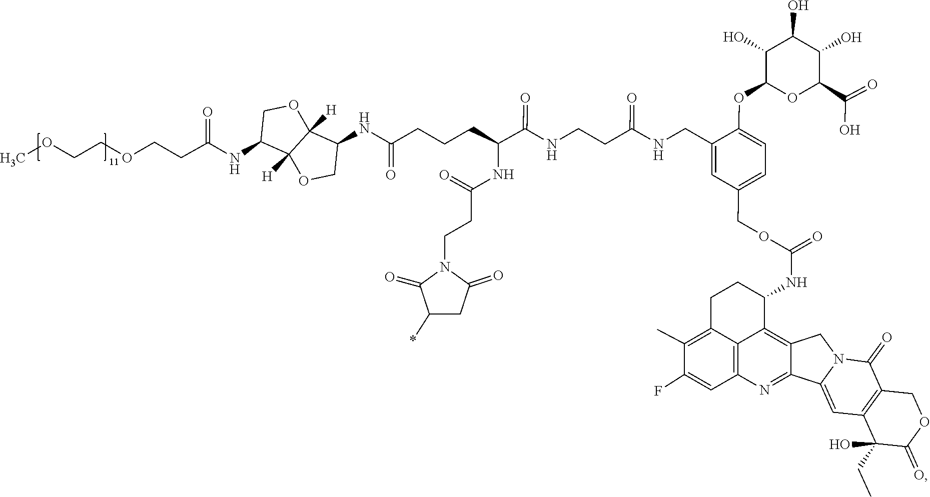

wherein [symbol] indicates the point of attachment to the antibody or antigen-binding fragment thereof. In some aspects, G^{A}-J^{A}-D^{C} is:

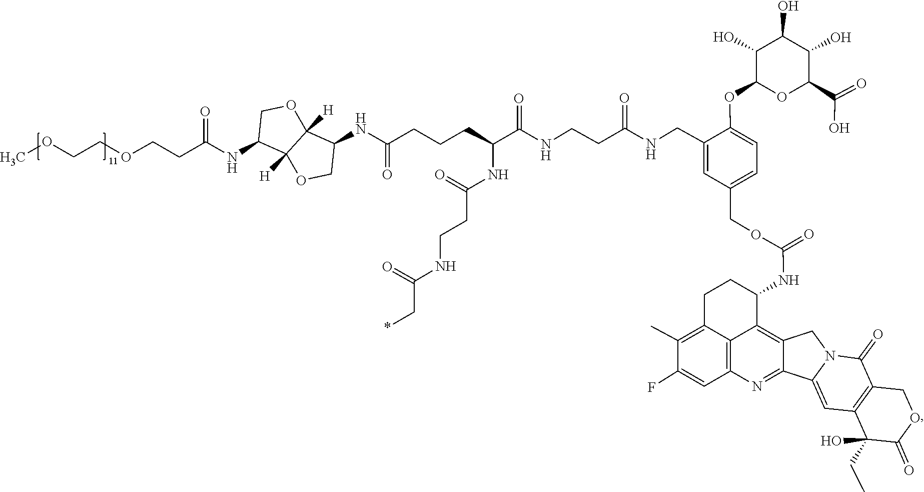

wherein [symbol] indicates the point of attachment to the antibody or antigen-binding fragment thereof.

Examples of G^{A} include, but are not limited to, the following, wherein X¹ is CH or N, h is 0 or 1, R^{K} is H or CH₃, Hal is Cl, Br or I, R^{L} is C₁₋₆ alkyl, and [symbol] indicates the point of attachment to the antibody, or antigen-binding fragment thereof.

| conjugation group conjugated to an | conjugation group for conjugation to an |
|---|---|
| antibody or antigen-binding fragment | antibody or antigen-binding fragment |
| thereof (G^{A}) | thereof |
| 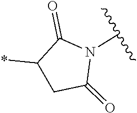 [SMILES: *C1CC(=O)N(*)C1=O] | 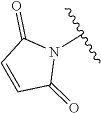 [SMILES: *N1C(=O)C=CC1=O] |
| 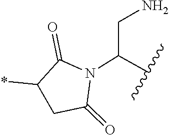 [SMILES: *C1CC(=O)N(C(*)CN)C1=O] | 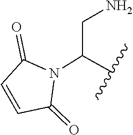 [SMILES: *C(CN)N1C(=O)C=CC1=O] |
| 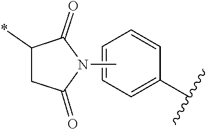 | 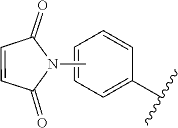 |
| 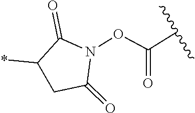 [SMILES: *C(=O)ON1C(=O)CC(*)C1=O] | 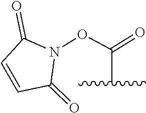 [SMILES: *C(=O)ON1C(=O)C=CC1=O] |
| 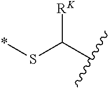 | 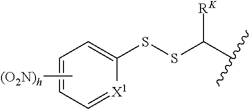 |
| 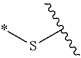 [SMILES: *S*] | 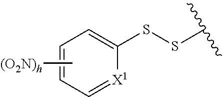 |
| 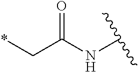 [SMILES: *CC(=O)N*] | 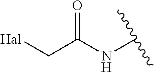 |
| 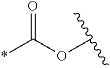 [SMILES: *OC(*)=O] | 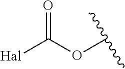 |
| 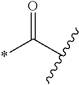 [SMILES: *C(*)=O] | 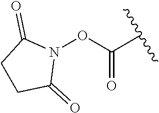 [SMILES: *C(=O)ON1C(=O)CCC1=O] |
| 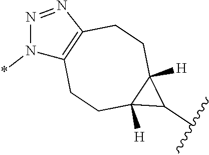 [SMILES: *C1[C@H]2CCc3nnn(*)c3CC[C@@H]12] | 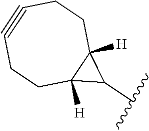 [SMILES: *C1[C@H]2CCC#CCC[C@@H]12] |
| 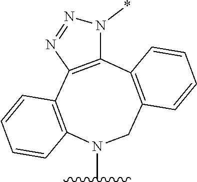 [SMILES: *N1Cc2ccccc2-c2c(nnn2*)-c2ccccc21] | 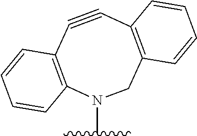 [SMILES: *N1Cc2ccccc2C#Cc2ccccc21] |
| 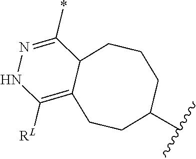 | 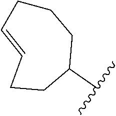 [SMILES: *C1CC/C=C/CCC1] |
| 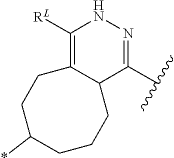 | 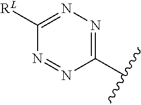 |
| 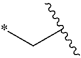 [SMILES: *C*] | 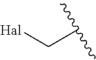 |
| 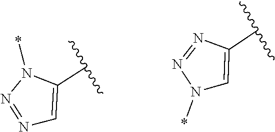 [SMILES: *c1cnnn1* ; *c1cn(*)nn1] | 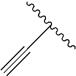 [SMILES: *C#C] |
| 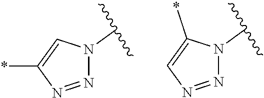 [SMILES: *c1cn(*)nn1 ; *c1cnnn1*] | 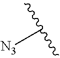 [SMILES: *N=[N+]=[N-]] |
| 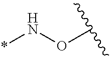 [SMILES: *NO*] | 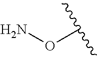 [SMILES: *ON] |
| 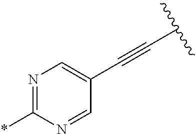 [SMILES: *C#Cc1cnc(*)nc1] | 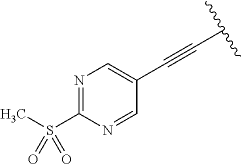 [SMILES: *C#Cc1cnc(S(C)(=O)=O)nc1] |

In some aspects, said antibody or antigen binding fragment thereof is a humanized monoclonal antibody. In some aspects, said antibody or antigen binding fragment thereof is an IgG1, IgG2, or IgG4 or fragment thereof. In some aspects, said antibody or antigen binding fragment thereof is an IgG1 or fragment thereof. In some aspects, the ADCC activity of the antibody composition is increased or decreased by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 1-fold, about 2-fold, about 3-fold, or about 4-fold, or increased or decreased by about 5% to about 400%.

The present disclosure also provides a pharmaceutical composition comprising an antibody-drug conjugate. The present disclosure also provides a method of treating a cancer comprising a cancer cell which expresses STEAP2, the method comprising administering to a subject the antibody-drug conjugate or pharmaceutical formulations herein. In some aspects, said cancer comprises a cancer cell which expresses STEAP2. In some aspects, said cancer is selected from breast cancer, ovarian cancer, endometrial cancer, cholangiocarcinoma, NSCLC (squamous and/or adenocarcinoma), pancreatic cancer, gastric cancer, and prostate cancer. In some aspects, the cancer is prostate cancer. In some aspects, the cancer is metastatic, recurrent, or relapsed prostate cancer.

The present disclosure also provides an antibody-drug conjugate (ADC) comprising: (i) (a) an antibody or antigen binding fragment thereof which binds to a STEAP2 polypeptide comprising: a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; and a LCDR1 comprising the amino acid sequence of SEQ ID NO: 4; a LCDR2 comprising the amino acid sequence of SEQ ID NO: 5; and a LCDR3 comprising the amino acid sequence of SEQ ID NO: 6, or (b) an antibody or antigen binding fragment thereof which binds to a STEAP2 polypeptide comprising: a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; and a LCDR1 comprising the amino acid sequence of SEQ ID NO: 10; a LCDR2 comprising the amino acid sequence of SEQ ID NO: 11; and a LCDR3 comprising the amino acid sequence of SEQ ID NO: 12; or (c) an antibody or antigen binding fragment thereof which binds to a STEAP2 polypeptide comprising: a HCDR1 comprising the amino acid sequence of SEQ ID NO: 19; a HCDR2 comprising the amino acid sequence of SEQ ID NO: 20; a HCDR3 comprising the amino acid sequence of SEQ ID NO: 21; and a LCDR1 comprising the amino acid sequence of SEQ ID NO: 22; a LCDR2 comprising the amino acid sequence of SEQ ID NO: 23; and a LCDR3 comprising the amino acid sequence of SEQ ID NO: 24; and (ii) a cytotoxic agent wherein the cytotoxic agent is LP-1; and (iii) wherein the ADC has a drug to antibody ratio (DAR) ranging from about 4 to about 8.

In some aspects, the antibody or antigen binding fragment thereof comprises: i. a variable heavy (VH) chain and a variable light (VL) chain at least 90%, at least 91% at least 92%, at least 93%, at least 94%, at least 95%, at least 96%, at least 97%, at least 98%, or at least 99% identical to SEQ ID NO: 31 and SEQ ID NO: 32, respectively; ii. a VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 32, respectively; iii. a VH chain and VL chain at least 90%, at least 91% at least 92%, at least 93%, at least 94%, at least 95%, at least 96%, at least 97%, at least 98%, or at least 99% identical to NO: 35 and SEQ ID NO: 32, respectively; iv. a VH chain and VL chain at least 90%, at least 91% at least 92%, at least 93%, at least 94%, at least 95%, at least 96%, at least 97%, at least 98%, or at least 99% identical to NO: 37 and SEQ ID NO: 32, respectively; v. a VH chain and VL chain at least 90%, at least 91% at least 92%, at least 93%, at least 94%, at least 95%, at least 96%, at least 97%, at least 98%, or at least 99% identical to NO: 39 and SEQ ID NO: 32, respectively; vi. a VH chain and VL chain at least 90%, at least 91% at least 92%, at least 93%, at least 94%, at least 95%, at least 96%, at least 97%, at least 98%, or at least 99% identical to NO: 45 and SEQ ID NO: 32, respectively; vii. a VH chain and VL chain at least 90%, at least 91% at least 92%, at least 93%, at least 94%, at least 95%, at least 96%, at least 97%, at least 98%, or at least 99% identical to NO: 47 and SEQ ID NO: 32, respectively; viii. a VH chain and VL chain at least 90%, at least 91% at least 92%, at least 93%, at least 94%, at least 95%, at least 96%, at least 97%, at least 98%, or at least 99% identical to NO: 49 and SEQ ID NO: 32, respectively; ix. a VH chain and VL chain at least 90%, at least 91% at least 92%, at least 93%, at least 94%, at least 95%, at least 96%, at least 97%, at least 98%, or at least 99% identical to NO: 51 and SEQ ID NO: 32, respectively; x. a VH chain and VL chain at least 90%, at least 91% at least 92%, at least 93%, at least 94%, at least 95%, at least 96%, at least 97%, at least 98%, or at least 99% identical to SEQ ID NO: 31 and SEQ ID NO: 36, respectively; xi. VH chain and VL chain at least 90%, at least 91% at least 92%, at least 93%, at least 94%, at least 95%, at least 96%, at least 97%, at least 98%, or at least 99% identical to SEQ ID NO: 33 and SEQ ID NO: 36, respectively; xii. a VH chain and a VL chain at least 90%, at least 91% at least 92%, at least 93%, at least 94%, at least 95%, at least 96%, at least 97%, at least 98%, or at least 99% identical to SEQ ID NO: 35 and SEQ ID NO: 36, respectively; xiii. a VH chain and a VL chain at least 90%, at least 91% at least 92%, at least 93%, at least 94%, at least 95%, at least 96%, at least 97%, at least 98%, or at least 99% identical to SEQ ID NO: 37 and SEQ ID NO: 38, respectively; xiv. a VH chain and a VL chain at least 90%, at least 91% at least 92%, at least 93%, at least 94%, at least 95%, at least 96%, at least 97%, at least 98%, or at least 99% identical SEQ ID NO: 39 and SEQ ID NO: 40, respectively; xv. a VH chain and a VL chain at least 90%, at least 91% at least 92%, at least 93%, at least 94%, at least 95%, at least 96%, at least 97%, at least 98%, or at least 99% identical to SEQ ID NO: 45 and SEQ ID NO: 46, respectively; xvi. a VH chain and a VL chain at least 90%, at least 91% at least 92%, at least 93%, at least 94%, at least 95%, at least 96%, at least 97%, at least 98%, or at least 99% identical to SEQ ID NO: 47 and SEQ ID NO: 48, respectively; xvii. a VH chain and a VL chain at least 90%, at least 91% at least 92%, at least 93%, at least 94%, at least 95%, at least 96%, at least 97%, at least 98%, or at least 99% identical SEQ ID NO: 49 and SEQ ID NO: 50, respectively; or xviii. a VH chain and a VL chain at least 90%, at least 91% at least 92%, at least 93%, at least 94%, at least 95%, at least 96%, at least 97%, at least 98%, or at least 99% identical to SEQ ID NO: 51 and SEQ ID NO: 52, respectively.

In some aspects, the antibody or antigen binding fragment thereof comprises:
i. the VH chain of SEQ ID NO: 31 and the VL chain of SEQ ID NO: 32;
ii. the VH chain of SEQ ID NO: 33 and the VL chain of SEQ ID NO: 32;
iii. the VH chain of SEQ ID NO: 35 and the VL chain of SEQ ID NO: 32;
iv. the VH chain of SEQ ID NO: 37 and the VL chain of SEQ ID NO: 32;
v. the VH chain of SEQ ID NO: 39 and the VL chain of SEQ ID NO: 32;
vi. the VH chain of SEQ ID NO: 45 and the VL chain of SEQ ID NO: 32;
vii. the VH chain of SEQ ID NO: 47 and the VL chain of SEQ ID NO: 32;
viii. the VH chain of SEQ ID NO: 49 and the VL chain of SEQ ID NO: 32;
ix. the VH chain of SEQ ID NO: 51 and the VL chain of SEQ ID NO: 32;
x. the VH chain of SEQ ID NO: 31 and the VL chain of SEQ ID NO: 36;
xi. the VH chain of SEQ ID NO: 33 and the VL chain of SEQ ID NO: 36;
xii. the VH chain of SEQ ID NO: 35 and the VL chain of SEQ ID NO: 36;
xiii. the VH chain of SEQ ID NO: 37 and the VL chain of SEQ ID NO: 38;
xiv. the VH chain of SEQ ID NO: 39 and the VL chain of SEQ ID NO: 40;
xv. the VH chain of SEQ ID NO: 45 and the VL chain of SEQ ID NO: 46;
xvi. the VH chain of SEQ ID NO: 47 and the VL chain of SEQ ID NO: 48;
xvii. the VH chain of SEQ ID NO: 49 and the VL chain of SEQ ID NO: 50; or
xviii. the VH chain of SEQ ID NO: 51 and the VL chain of SEQ ID NO: 52.

In some aspects, the antibody or binding fragment thereof comprises an Fc region. In some aspects, the antibody or binding fragment thereof comprises an Fc region comprising a L234F/L235E/P331S triple mutation TM. In some aspects, the antibody or binding fragment thereof comprises an Fc region comprising a L234F/L235E/P331S triple mutation TM according to SEQ ID NO: 59. In some aspects, the antibody or binding fragment thereof comprises an Fc region which has reduced antibody dependent cellular cytotoxicity.

The present disclosure also provides an ADC comprising a heavy chain (HC) comprising the amino acid sequence of SEQ ID NO: 41, and light chain (LC) comprising the amino acid sequence of SEQ ID NO: 42; or a heavy chain (HC) comprising the amino acid sequence of SEQ ID NO: 43, and light chain (LC) comprising the amino acid sequence of SEQ ID NO: 44. In some aspects, the drug to antibody ratio (DAR) is about 4 or about 8. In some aspects, the drug to antibody ratio is about 8. In some aspects, the drug to antibody ratio is 8. In some aspects, said antibody or antigen binding fragment thereof is an IgG1, IgG2 or IgG4 or fragment thereof. In some aspects, said antibody or antigen binding fragment thereof is an IgG1 or fragment thereof. In some aspects, the antibody or binding fragment thereof comprises an Fc region which has reduced antibody dependent cellular cytotoxicity compared to an antibody comprising a wild type Fc region. In some aspects, the ADC is formulated as a pharmaceutical composition.

The present disclosure also provides a method of treating a cancer expressing STEAP2, comprising administering to a subject the ADC or pharmaceutical composition described herein. In some aspects, the subject is a human. In some aspects, the cancer cell has a homologous DNA repair defect.

The present disclosure also provides a method for reducing the volume of a tumor expressing STEAP2, comprising administering to a subject an ADC or pharmaceutical composition described herein. In some aspects, the tumor has a homologous DNA repair defect. In some aspects, the present disclosure also provides a kit of parts comprising at least one of a (i) variable heavy chain, (ii) variable light chain of any ADC described herein. In some aspects, the kit further comprises instructions for use.

### BRIEF DESCRIPTION OF THE DRAWINGS

Aspects of the disclosure will now be described, by way of example only, with reference to the following Figures and Examples.

FIG. 1 shows STEAP2 RNA expression in normal tissues as compared to expression of other tumor associated antigens (TAAs) for prostate cancer, PSMA and STEAP1. Data queried from Human Protein Atlas (HPA) database.

FIG. 2 shows elevated STEAP2 expression across all stages of clinically localized prostate cancer (CaP), ranging from primary diagnosis, primary castration resistant prostate cancer (CRPC), and to lymph node and bone metastasis. Images on the right illustrate two exemplary immunohistochemistry stains (IHC) between CRPC and bone metastasis.

FIG. 3A shows the binding of 40A3-LO7 and its affinity matured variants to human STEAP2 in LNCAP cells and the lack thereof in the isogenic STEAP2 knock-out cell line.

FIG. 3B shows the binding of 40A3-LO7 and its affinity matured variants to the murine STEAP2 confirming antibody cross-reactivity to mouse. The notation STEAP3-2 refers to genetically modified cell line expressing a chimera protein with a STEAP3 transmembrane sequence and STEAP2 extracellular domain.

FIG. 4A shows the internalization kinetics of 40A3-LO7 and its affinity matured variants in human prostate cancer cells with endogenous expression of STEAP2 (C42). Representative images on the top are fluorescent and brightfield demonstrating internalization of 40A3-LO14 antibody 2 hrs after treatment. No internalization signal was observed with the Isotype control antibody.

FIG. 4B shows the internalization kinetics of 40A3-LO14 carrying the LP-1 linker-payload compared to a control LO14 antibody, an isotype control ADC with LP-1, and an isotype control antibody in human prostate cancer cells with endogenous expression of STEAP2 (C42).

FIG. 5 shows an exemplary schematic of the DAR 8 (top row) and DAR4 (bottom row) ADC structures, and the synthesis used to procure the ADC, respectively. RT=room temperature.

FIG. 6A shows the cytotoxic effect of STEAP2 SG3932 DAR8 ADCs in STEAP2 expressing human prostate cancer cell lines. STEAP2-specific killing of LO14 SG3932 DAR8 ADC was confirmed by the dose-dependent decrease in cell viability of treated LNCAP, C42, and 22Rv1 cells and the lack thereof in the isogenic STEAP2 knock-out cell line. No activity observed in the Isotype SG3932 DAR8 ADC.

FIG. 6B shows the cytotoxic effect of STEAP2 LO14 LP-1 DAR8 ADCs in a STEAP2 expressing human prostate cancer cell line. STEAP2-specific killing of LO14 LP-1 DAR8 ADC was confirmed by the dose-dependent decrease in cell viability of treated LNCAP cells.

FIG. 6C shows the cytotoxic effect of STEAP2 LO14 LP-1 DAR8 ADCs in a STEAP2 expressing human prostate cancer cell line. STEAP2-specific killing of LO14 LP-1 DAR8 ADC was confirmed by the dose-dependent decrease in cell viability of treated C42 cells.

FIG. 6D shows the cytotoxic effect of STEAP2 LO14 LP-1 DAR8 ADCs in a STEAP2 expressing human prostate cancer cell line. STEAP2-specific killing of LO14 LP-1 DAR8 ADC was confirmed by the dose-dependent decrease in cell viability of treated 22Rv1 cells.

FIG. 7A shows the plasma concentrations of either LO14 hIgG1-TM or SG3932 DAR8 ADC following a single intravenous (IV) dose of 5 milligram per kilogram (mpk). A standard 2 comportment naïve pool model was used to calculate clearance, half-life, and volume of distribution and over a 21 day duration. There is minimal difference in clearance of LO14 antibody or ADC in immunocompromised NSG mice (left). The clearance of LO14 SG3932 DAR8 ADC is similar in both immunodeficient (NSG, Athymic Nude) and immunocompetent (FcRN) mice (right).

FIG. 7B shows the plasma concentrations of either LO14 SG3932 DAR4 or DAR8 ADC following a single intravenous (IV) dose of 5 milligram per kilogram (mpk). A standard 2 comportment naïve pool model was used to calculate clearance, half-life, and volume of distribution and over a 21 day duration. There is minimal difference in clearance of LO14 DAR4 or DAR8 ADCs in immunocompromised NSG mice (left).

FIG. 7C shows the plasma concentrations of LO14 LP-1 DAR8 ADC and an LO14 IgG control following a single intravenous (IV) dose of 5 milligram per kilogram (mpk).

FIG. 8A shows the efficacy of DAR8 vs. DAR4 LO14 SG3932 ADC or Isotype control after a single IV dose ranging from 1-10 mpk in NSG mice bearing human prostate cancer cell-derived xenografts (CDX). LO14 SG3932 ADCs showed dose-dependent decreases in tumor volume in both models over a 50 day duration. Values are mean±SEM tumor volumes. Dotted line denotes the average baseline tumor volume at time of dosing.

FIG. 8B shows the efficacy of DAR8 vs. DAR4 LO14 SG3932 ADC or Isotype control after a single IV dose ranging from 1-10 mpk in NSG mice bearing human prostate cancer patient-derived xenografts (PDX). LO14 SG3932 ADCs showed dose-dependent decreases in tumor volume in both models over a 50 day duration. Values are mean±SEM tumor volumes. Dotted line denotes the average baseline tumor volume at time of dosing.

FIG. 8C shows the efficacy of DAR8 LO14 LP1 ADC or Isotype control after a single IV dose ranging from 0.5-6 mpk in NSG mice bearing human prostate cancer cell-derived xenografts (CDX). LO14 LP-1 DAR8 ADCs showed dose-dependent decreases in tumor volume in both models over a 50 day duration. Values are mean±SEM tumor volumes. Dotted line denotes the average baseline tumor volume at time of dosing.

FIG. 8D shows the efficacy of DAR8 LO14 LP-1 ADC or Isotype control after a single IV dose ranging from 0.25-5 mpk in NSG mice bearing human prostate cancer patient-derived xenografts (PDX). LO14 LP-1 DAR8 ADCs showed dose-dependent decreases in tumor volume in both models over a 50 day duration. Values are mean±SEM tumor volumes. Dotted line denotes the average baseline tumor volume at time of dosing.

FIG. 8E shows the relative tumor growth rate in NSG mice bearing CDX or PDX models (014-22Rv1, 015-C42, 017-147LuCaP, 018-73LuCaP, and 019-70LuCaP, respectively) after single dose IV administration of either DAR4 or DAR8 LO14 SG3932 ADCs.

FIG. 9A shows the median best response of 19 different human prostate PDX models following single dose intravenous (IV) administration of LO14 SG3932 DAR8 ADC at 5 milligrams per kilogram (mpk).

FIG. 9B shows the median best response of 19 different human prostate PDX models following single dose intravenous (IV) administration of LO14 SG3932 DAR8 ADC at 2.5 milligrams per kilogram (mpk).

### DETAILED DESCRIPTION

The six-transmembrane epithelial antigen of prostate 2 (STEAP2), also known as STEAP-2, metalloreductase STEAP2, is expressed in multiple cell types (e.g. breast, lung, pancreatic, and prostate cells). STEAP2 is also expressed in normal heart, brain, pancreas, ovary, skeletal muscle, mammary gland, testis, uterus, kidney, lung, trachea, colon, and liver.

However, STEAP2 is over-expressed in cancerous tissues, including prostate, bladder, cervix, lung, colon, kidney, breast, pancreatic, stomach, uterus, and ovarian tumors (Gomes, I. M. et al., 2012, Mol. Cancer Res. 10:573-587; Challita-Eid-P. M., et al., 2003, WO 03/087306; Emtage, P. C. R., 2005, WO 2005/079490). This over-expression is consistent with the role of a cancer antigen. Therefore, this disclosure includes successfully generated antibodies which show high binding to STEAP2 expressing cells. Advantageously, the antibodies can target multiple different cancer cell types expressing STEAP2, exemplifying the broad utility of the antibodies as anti-cancer therapies. Furthermore, the antibodies can advantageously be linked/conjugated to suitable drugs/cytotoxins (e.g. to provide Antibody-drug conjugates (ADC)), thus increasing the potency of the antibodies as a therapy by allowing for targeted toxin delivery to cancer cells.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower). As disclosed herein, the language “comprising” constitutes otherwise analogous aspects disclosed in the terms “consisting of” and/or “consisting essentially of”.

Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Thus, any value recited herein is inclusive of a precise value, as well as values which are about the same as the precise value. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “pharmaceutical composition” refers to a preparation that is in such form as to permit the biological activity of the active ingredient to be effective, and which contains no additional components which are unacceptably toxic to a subject to which the composition would be administered. Such composition can be sterile, and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilizing agent (e.g., human albumin), a preservative (e.g., benzyl alcohol), and absorption promoter to enhance bioavailability, and/or other conventional solubilizing or dispersing agents.

Furthermore, the antibody or antigen binding fragment thereof of the disclosure has been demonstrated to target and suppress growth of STEAP2 positive tumors in vivo. Thus, the disclosure embraces the above defined antibody or antigen binding fragment thereof and the above defined pharmaceutical composition for use in a method of treating cancer. In certain aspects, the cancer comprises a cancer cell which expresses STEAP2.

“Cancer” as used herein can encompass all types of oncogenic processes and/or cancerous growths. In this disclosure, cancer can include, but is not limited to primary tumors as well as metastatic tissues or malignantly transformed cells, tissues, or organs. Cancer can encompass histopathologies and stages, e.g., stages of invasiveness/severity, of a cancer. Cancer can include relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In one aspect there is provided an antibody or antigen binding fragment thereof for use in treating a cancer (for example, wherein said cancer comprises a cancer cell that expresses STEAP2), wherein the antibody or antigen binding fragment comprises:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof.

In other words, one aspect of the disclosure provides a method of treating a cancer (for example, wherein said cancer comprises a cancer cell that expresses STEAP2), the method comprising administering to a subject an effective amount of an antibody or antigen binding fragment comprising:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof.

In yet other words, the disclosure is directed to the use of an antibody or antigen binding fragment thereof in the manufacture of a medicament for the treatment of cancer (for example, wherein said cancer comprises a cancer cell that expresses STEAP2), said antibody or antigen binding fragment comprising:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof.

Certain definitions and aspects will now be outlined. It should be understood that the following definitions and aspects may pertain to any aspect described herein, e.g. any method, composition, and/or composition for use in therapy described herein.

The term “epitope” refers to a target protein region (e.g. polypeptide) capable of binding to (e.g. being bound by) an antibody or antigen binding fragment of the disclosure.

STEAP2 is a member of the STEAP family and encodes a multi-pass membrane protein that localizes to the Golgi complex, the plasma membrane, and the vesicular tubular structures in the cytosol. STEAP2 is understood to be expressed on the surface of antigen-presenting cells for interactions with ligands of immune cells. STEAP2 is also known as, UNQ6507/PRO23203, STMP, IPCA1, PUMPCn, STAMPI or PCANAP1, LOC261729, metalloreductase STEAP2, OTTHUMP00000067572, OTTHUMP00000067573, OTTHUMP00000196964, prostate cancer associated protein 1, prostate cancer-associated protein 1, SixTransMembrane Protein of Prostate 1, protein upregulated in metastatic prostate cancer, six transmembrane epithelial antigen of prostate 2, six-transmembrane epithelial antigen of prostate 2, and any grammatical equivalents.

Without wishing to be bound by theory, STEAP2 is understood to be expressed on cells of various cancer types suggests that this molecule is a tumor-associated antigen. As such, the ability of the claimed antibody to target (and optionally deliver a cytotoxin to) a STEAP2 expressing renders said antibody particularly suitable for use in cancer therapy. Furthermore, STEAP2 expression is not limited to a particular cancer type, such that it can represent a target antigen for treating a broad spectrum of cancer types.

The RNA, DNA, and amino acid sequences of STEAP2 are known to those skilled in the art and can be found in many databases, for example, in the databases of the National Center for Biotechnology Information (NCBI) and UniProt. Examples of these sequences found at UniProt are at Q8IUE7, Q8NFT2 for human STEAP2. The nucleotide sequence encoding for human STEAP2 may be SEQ ID NO: 56. In some aspects, the polypeptide sequence of human STEAP2 is SEQ ID NO: 57.

In one aspect, the antibody or antigen binding fragment thereof comprises a HCDR1, a HCDR2, a HCDR3, a LCDR1, LCDR2, and a LCDR3 comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof.

In one aspect, the antibody or antigen binding fragment thereof comprises a HCDR1, a HCDR2, a HCDR3, a LCDR1, LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof.

In one aspect, the antibody or antigen binding fragment thereof comprises a HCDR1, a HCDR2, a HCDR3, a LCDR1, LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof.

In one aspect, the antibody or antigen binding fragment thereof comprises a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof.

In one aspect, the antibody or antigen binding fragment thereof comprises a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof.

In other words, the antibody or antigen binding fragment thereof can comprise:
a HCDR1 comprising the amino acid sequence of SEQ ID NO: 7, or a functional variant thereof;
a HCDR2 comprising the amino acid sequence of SEQ ID NO: 8, or a functional variant thereof;
a HCDR3 comprising the amino acid sequence of SEQ ID NO: 9, or a functional variant thereof;
a LCDR1 comprising the amino acid sequence of SEQ ID NO: 10, or a functional variant thereof;
a LCDR2 comprising the amino acid sequence of SEQ ID NO: 11, or a functional variant thereof; and
a LCDR3 comprising the amino acid sequence of SEQ ID NO: 12, or a functional variant thereof.

In one aspect, the antibody or antigen binding fragment thereof comprises:
i. a HCDR1 comprising the amino acid sequence of SEQ ID NO: 1, or a functional variant thereof;
ii. a HCDR2 comprising the amino acid sequence of SEQ ID NO: 2, or a functional variant thereof;
iii. a HCDR3 comprising the amino acid sequence of SEQ ID NO: 3, or a functional variant thereof;
iv. a LCDR1 comprising the amino acid sequence of SEQ ID NO: 4, or a functional variant thereof;
v. a LCDR2 comprising the amino acid sequence of SEQ ID NO: 5, or a functional variant thereof; and
vi. a LCDR3 comprising the amino acid sequence of SEQ ID NO: 6, or a functional variant thereof.

In one aspect, the antibody or antigen binding fragment thereof comprises:
i. a HCDR1 comprising the amino acid sequence of SEQ ID NO: 13, or a functional variant thereof;
ii. a HCDR2 comprising the amino acid sequence of SEQ ID NO: 14, or a functional variant thereof;
iii. a HCDR3 comprising the amino acid sequence of SEQ ID NO: 15, or a functional variant thereof;
iv. a LCDR1 comprising the amino acid sequence of SEQ ID NO: 16, or a functional variant thereof;
v. a LCDR2 comprising the amino acid sequence of SEQ ID NO: 17, or a functional variant thereof; and
vi. a LCDR3 comprising the amino acid sequence of SEQ ID NO: 18, or a functional variant thereof.

In one aspect, the antibody or antigen binding fragment thereof comprises:
i. a HCDR1 comprising the amino acid sequence of SEQ ID NO: 19, or a functional variant thereof;
ii. a HCDR2 comprising the amino acid sequence of SEQ ID NO: 20, or a functional variant thereof;
iii. a HCDR3 comprising the amino acid sequence of SEQ ID NO: 21, or a functional variant thereof;
iv. a LCDR1 comprising the amino acid sequence of SEQ ID NO: 22, or a functional variant thereof;
v. a LCDR2 comprising the amino acid sequence of SEQ ID NO: 23, or a functional variant thereof; and
vi. a LCDR3 comprising the amino acid sequence of SEQ ID NO: 24, or a functional variant thereof.

In one aspect, the antibody or antigen binding fragment thereof comprises:
i. a HCDR1 comprising the amino acid sequence of SEQ ID NO: 25, or a functional variant thereof;
ii. a HCDR2 comprising the amino acid sequence of SEQ ID NO: 26, or a functional variant thereof;
iii. a HCDR3 comprising the amino acid sequence of SEQ ID NO: 27, or a functional variant thereof;
iv. a LCDR1 comprising the amino acid sequence of SEQ ID NO: 28, or a functional variant thereof;
v. a LCDR2 comprising the amino acid sequence of SEQ ID NO: 29, or a functional variant thereof; and
vi. a LCDR3 comprising the amino acid sequence of SEQ ID NO: 30, or a functional variant thereof.

Additionally or alternatively, an antibody or antigen binding fragment thereof described herein may be described by means of a variable heavy (VH) chain and a variable light (VL) chain thereof.

Suitable variable heavy (VH) chain sequences (which the antibody or antigen binding fragment thereof may comprise) are outlined in an individualized manner below:
SEQ ID NO: 31, or a functional variant thereof;
SEQ ID NO: 33, or a functional variant thereof
SEQ ID NO: 35, or a functional variant thereof
SEQ ID NO: 37, or a functional variant thereof
SEQ ID NO: 39, or a functional variant thereof
SEQ ID NO: 45, or a functional variant thereof
SEQ ID NO: 47, or a functional variant thereof
SEQ ID NO: 49, or a functional variant thereof
SEQ ID NO: 51, or a functional variant thereof.

Suitable variable heavy (VH) chain sequences (which the antibody or antigen binding fragment thereof may comprise) are outlined in an individualized manner below:
SEQ ID NO: 31, or a functional variant thereof
SEQ ID NO: 33, or a functional variant thereof

Suitable variable light (VL) chain sequences (which the antibody or antigen binding fragment thereof may comprise) are outlined in an individualized manner below:
SEQ ID NO: 32, or a functional variant thereof
SEQ ID NO: 34, or a functional variant thereof
SEQ ID NO: 36, or a functional variant thereof
SEQ ID NO: 38, or a functional variant thereof
SEQ ID NO: 40, or a functional variant thereof
SEQ ID NO: 46, or a functional variant thereof
SEQ ID NO: 48, or a functional variant thereof
SEQ ID NO: 50, or a functional variant thereof
SEQ ID NO: 52, or a functional variant thereof

A variable light (VL) chain sequence (which the antibody or antigen binding fragment thereof may comprise) can comprise an amino acid sequence of SEQ ID NO: 32 (or a functional variant thereof).

For example, in one aspect, the antibody or antigen binding fragment thereof comprises:
i. a variable heavy chain comprising an amino acid sequence having at least 70%, 75%, 80%, 90%, 95% or 100% sequence identity to the amino acid sequence of SEQ ID NO: 31, 33, 35, 37, 39, 45, 47, 49, or 51, or a functional variant thereof; and
ii. a variable light chain comprising an amino acid sequence having at least 70%, 75%, 80%, 90%, 95% or 100% sequence identity to the amino acid sequence of SEQ ID NO: 32, 34, 36, 38, 40, 46, 48, 50, or 52, or a functional variant thereof.

For example, in one aspect, the antibody or antigen binding fragment thereof comprises:
i. a variable heavy chain comprising an amino acid sequence having at least 70%, 75%, 80%, 90%, 95% or 100% sequence identity to the amino acid sequence of SEQ ID NO: 31, 33, 35, 37, 39, 45, 47, 49, or 51, or a functional variant thereof; and
ii. a variable light chain comprising an amino acid sequence having at least 70%, 75%, 80%, 90%, 95% or 100% sequence identity to the amino acid sequence of SEQ ID NO: 32, 34, 36, 38, 40, 46, 48, 50, or 52, or a functional variant thereof.

Suitably, the antibody or antigen binding fragment thereof may comprise:
i. a variable heavy chain comprising an amino acid sequence having at least 70%, 75%, 80%, 90%, 95% or 100% sequence identity to the amino acid sequence SEQ ID NO: 33, or a functional variant thereof; and
ii. a variable light chain comprising an amino acid sequence having at least 70%, 75%, 80%, 90%, 95% or 100% sequence identity to the amino acid sequence of SEQ ID NO: 34, or a functional variant thereof.

More suitably, the antibody or antigen binding fragment thereof may comprise:
i. a variable heavy chain comprising an amino acid sequence having at least 70%, 75%, 80%, 90%, 95% or 100% sequence identity to the amino acid sequence SEQ ID NO: 31, or a functional variant thereof; and
ii. a variable light chain comprising an amino acid sequence having at least 70%, 75%, 80%, 90%, 95% or 100% sequence identity to the amino acid sequence of SEQ ID NO: 34, or a functional variant thereof.

In one aspect, the antibody or antigen binding fragment thereof comprises:
a variable heavy (VH) chain and a variable light (VL) chain comprising the amino acid sequence of SEQ ID NO: 31, and SEQ ID NO: 32, respectively, or a functional variant thereof;
a VH chain and a VL chain comprising the amino acid sequence of SEQ ID NO: 33 and SEQ ID NO: 32, respectively, or a functional variant thereof;
a VH chain and a VL chain comprising the amino acid sequence of SEQ ID NO: 35 and SEQ ID NO: 32, respectively, or a functional variant thereof;
a VH chain and a VL chain comprising the amino acid sequence of SEQ ID NO: 37 and SEQ ID NO: 32, respectively, or a functional variant thereof;
a VH chain and a VL chain comprising the amino acid sequence of SEQ ID NO: 39 and SEQ ID NO: 32, respectively, or a functional variant thereof;
a VH chain and a VL chain comprising the amino acid sequence of SEQ ID NO: 45 and SEQ ID NO: 32, respectively, or a functional variant thereof;
a VH chain and a VL chain comprising the amino acid sequence of SEQ ID NO: 47 and SEQ ID NO: 32, respectively, or a functional variant thereof;
a VH chain and a VL chain comprising the amino acid sequence of SEQ ID NO: 49 and SEQ ID NO: 32, respectively, or a functional variant thereof; or
a VH chain and a VL chain comprising the amino acid sequence of SEQ ID NO: 51 and SEQ ID NO: 32, respectively, or a functional variant thereof.

In one aspect the antibody or antigen binding fragment thereof comprises: a variable heavy (VH) chain comprising the amino acid sequence of SEQ ID NO: 31, 33, 37, 39, 45, 47, 49 or 51 (or a functional variant thereof); and a variable light (VL) chain comprising the amino acid sequence of SEQ ID NO: 32 (or a functional variant thereof).

In one aspect, the antibody or antigen binding fragment thereof comprises: a variable heavy chain comprising the amino acid sequence of SEQ ID NO: 31, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 33, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 35, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 37, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 39, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 45, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 47, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 49, or a functional variant thereof; and a variable light chain comprising the amino acid sequence of SEQ ID NO: 32, or a functional variant thereof.

In one aspect, the antibody or antigen binding fragment thereof comprises: a variable heavy chain comprising the amino acid sequence of SEQ ID NO: 51, or a functional variant thereof; and a variable light chain comprising the amino acid sequence of SEQ ID NO: 32, or a functional variant thereof.

In one aspect, the antibody or antigen binding fragment thereof comprises a variable heavy chain comprising an amino acid sequence having at least 70%, 75%, 80%, 90%, 95% or 100% sequence identity to a reference amino acid sequence of SEQ ID NO: 33. In one aspect, the antibody or antigen binding fragment thereof comprises a variable heavy chain comprising an amino acid sequence of SEQ ID NO: 33. For example, the antibody or antigen binding fragment thereof may comprise a variable heavy chain comprising an amino acid sequence of SEQ ID NO: 33, and a variable light chain comprising an amino acid sequence of SEQ ID NO: 34.

Additionally or alternatively, an antibody or antigen binding fragment thereof described herein may be described by means of a heavy chain and/or light chain thereof.

In one aspect, the antibody or antigen binding fragment thereof comprises a light chain (e.g. comprising a VL and constant light chain) comprising an amino acid sequence having at least 70%, 75%, 80%, 90%, 95% or 100% sequence identity to the amino acid sequence of SEQ ID NO: 32. In one aspect, the antibody or antigen binding fragment thereof comprises a light chain (e.g. comprising a VL and constant light chain) comprising the amino acid sequence of SEQ ID NO: 32.

In one aspect, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 41. For example, the antibody or antigen binding fragment thereof may comprise a heavy chain (e.g. comprising a VH and constant heavy chain) comprising the amino acid sequence of SEQ ID NO: 41.

In one aspect, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 43. For example, the antibody or antigen binding fragment thereof may comprise a heavy chain (e.g. comprising a VH and constant heavy chain) comprising the amino acid sequence of SEQ ID NO: 43.

In some aspects, the antibody or antigen binding fragment thereof comprises a heavy chain (e.g. comprising a VH and constant heavy chain) comprising an amino acid sequence having at least 70%, 75%, 80%, 90%, 95% or 100% sequence identity to a reference amino acid sequence of SEQ ID NO: 55. In some aspects, the antibody or antigen binding fragment thereof comprises a heavy chain (e.g. comprising a VH and constant heavy chain) comprising the amino acid sequence of SEQ ID NO: 55.

In one aspect, the antibody or antigen binding fragment thereof comprises a light chain constant region comprising an amino acid sequence having at least 70%, 75%, 80%, 90%, 95% or 100% sequence identity to a reference amino acid sequence of SEQ ID NO: 58. In some aspects, the antibody or antigen binding fragment thereof comprises light chain constant region comprising an amino acid sequence of SEQ ID NO: 58.

In one aspect, the antibody or antigen binding fragment thereof comprises a heavy chain constant region comprising the amino acid sequence of SEQ ID NO: 55. In some aspects, the antibody or antigen binding fragment thereof comprises a heavy chain constant region comprising the amino acid sequence of SEQ ID NO: 54.

In some aspects, the antibody or antigen binding fragment thereof comprises a light chain (e.g. comprising a VL and constant light chain) comprising the amino acid sequence of SEQ ID NO: 42 and a heavy chain (e.g. comprising a VH and constant heavy chain) comprising the amino acid sequence of SEQ ID NO: 55.

Disclosed herein, is an antibody (or antigen binding fragment) thereof having affinity and specificity for a clinically relevant target and has demonstrated a unique advantage (e.g. unexpected technical effect) associated therewith.

An antibody or antigen binding fragment thereof described herein is capable of binding to STEAP2 as an integral component of a cancer cell (for example, STEAP2 as an integral component of a cell membrane of a cancer cell).

An antibody or antigen binding fragment thereof described herein may bind to an exemplary prostate cancer cell line, including but not limited to LNCaP, for example. For example, the antibody or antigen binding fragment thereof binds to a STEAP2 (e.g. a STEAP2 epitope) of a LNCaP cell line and/or any cancer cell lines (e.g. which may lack an exogenous nucleic acid encoding STEAP2). Suitably, the antibody or antigen binding fragment thereof described herein may bind to a LNCaP cell line and a CHO cell line (e.g. which may lack an exogenous nucleic acid encoding STEAP2.

The antibody binding affinity can be measured by any suitable method of measuring binding affinity described herein or known to a person of ordinary skill in the arts.

Suitably, the antibody or antigen binding fragment of the disclosure binds to STEAP2 molecule with sufficient affinity such that the antibody is useful as a therapeutic agent or a diagnostic reagent in targeting STEAP2.

In one aspect, the antibody or antigen binding fragment thereof binds to a STEAP2 (e.g., a human STEAP2) with a dissociation constant (KD) of ≤1 μM, ≤100 nM, ≤10 nM, ≤1 nM, ≤0.1 nM, ≤10 pM, ≤1 pM, or ≤0.1 pM. In one aspect, the antibody or antigen binding fragment thereof binds to a STEAP2 (e.g., a human STEAP2) with a KD of between about 0.1 nM to about 40 nM, between about 0.5 nM to about 30 nM, between about 1 nM to about 20 nM, or between about 1.5 nM to about 20 nM.

The KD measurements (binding affinity) may be carried out by any suitable assay known in the art. Suitable assays include an affinity assay performable via a KinExA system (e.g., KinExA 3100, KinExA 3200, or KinExA 4000) (Sapidyne Instruments, Idaho), or ForteBio Octet system.

In one aspect, the extent of binding of an antibody or antigen binding fragment thereof of the disclosure to an unrelated, non-STEAP2 protein is less than about 10%, 5%, 2% or 1% (e.g., less than about 10%) of the binding of the antibody (or antigen binding fragment thereof) to STEAP2 (e.g., human STEAP2). Said binding may be measured, e.g., by a radioimmunoassay (RIA), BIACORE® (using recombinant STEAP2 as the analyte and antibody as the ligand, or vice versa), KINEXA®, ForteBio Octet system, or other binding assays known in the art.

In one aspect, the STEAP2 polypeptide is comprised within a STEAP2 polypeptide sequence, or a fragment thereof.

A “STEAP2 polypeptide” may comprise the full length polypeptide sequence of STEAP2 (e.g. SEQ ID NO.: 57), or may comprise a fragment of STEAP2 of any length of the full length polypeptide sequence of STEAP2 (e.g. comprising a polypeptide sequence of 5%, 15%, 25%, 35%, 45%, 55%, 65%, 75%, 85% or 95% of the full length polypeptide sequence of STEAP2) which comprises an epitope which can bind (e.g. be bound by) an antibody or antigen binding fragment of the disclosure. The STEAP2 polypeptide may comprise a sequence having 75%, 80%, 85%, 90% or 90% sequence identity to the sequence of SEQ ID NO.: 57. The STEAP2 polypeptide can comprise the sequence of SEQ ID NO.: 57.

The antibody or antigen binding fragment has high affinity for STEAP2 both in vitro an in vivo, and thus may advantageously be used in methods for detecting a STEAP2 epitope, and associated methods of diagnosis.

As described above, an antibody or antigen binding fragment thereof of the disclosure may be comprised within a pharmaceutical composition. The pharmaceutical composition may comprise one or more pharmaceutically acceptable excipient(s). In one aspect, a pharmaceutical composition of the disclosure can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 22nd ed., Ed. Lloyd V. Allen, Jr. (2012).

In one aspect, a pharmaceutical composition of the disclosure may be comprised within one or more formulation selected from a capsule, a tablet, an aqueous suspension, a solution, a nasal aerosol, or a combination thereof.

In one aspect, the pharmaceutical composition comprises more than one type of antibody or antigen binding fragment of the disclosure. For example, a pharmaceutical composition may comprise two or more selected from an antibody, an antigen-binding fragment, an antibody or antigen binding fragment thereof conjugated to a cytotoxin, or a combination thereof.

The term “a pharmaceutically effective amount” of an antibody or antigen-binding fragment means an amount sufficient to achieve effective binding to a target and to achieve a benefit, e.g., to ameliorate symptoms of a disease or condition or to detect a substance or a cell.

In one aspect, a pharmaceutical composition may comprise a buffer (e.g., acetate, phosphate or citrate buffer), a surfactant (e.g., polysorbate), optionally a stabilizer agent (e.g., human albumin), etc.

To “treat” refers to therapeutic measures that cure, slow down, alleviate symptoms of, and/or halt progression of a diagnosed pathologic condition or disorder. Thus, those in need of treatment include those already with the disorder. In one aspect, a subject is successfully “treated” for a disease or disorder (e.g. cancer), according to the methods provided herein if the patient shows, e.g., total, partial, or transient alleviation or elimination of symptoms associated with the disease or disorder (e.g. cancer).

In one aspect, a method of the disclosure may be used to prevent the onset of a cancer comprising a cancer cell which expresses STEAP2. To “prevent” refers to prophylactic or preventative measures that prevent and/or slow the development of a targeted pathologic condition or disorder. Thus, those in need of prevention include those prone to have or susceptible to the disorder. In one aspect, a disease or disorder (e.g. cancer) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the disclosure.

The terms “subject”, “individual” and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In one aspect, the subject is a human. In methods of the disclosure, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, in one aspect, a method of the disclosure may be used to confirm the presence of cancer in a subject. For example, the subject may previously have been diagnosed with cancer by alternative means. In one aspect, the subject has been previously administered a cancer therapy.

In one aspect, methods of treatment of the disclosure comprise one or more administration step selected from oral, intravenous, intraarterial, intraperitoneal, intramuscular, subcutaneous, rectal, or vaginal, inhalation, topical, or a combination thereof. In one aspect, the administration is intravenous or intraarterial (e.g. by injection or drip), or a combination thereof.

In one aspect, the antibody or antigen binding fragment thereof is delivered directly to the site of the adverse cellular population (e.g. thereby increasing the exposure of the diseased tissue to the therapeutic agent). In one aspect, the administration is directly to the airway, e.g., by inhalation or intranasal administration.

In one aspect, a cancer referred to herein is a cancer characterized by the expression (e.g. overexpression) of a STEAP2 molecule. In other words, a cancer referred to herein may comprise a cancerous cell that expresses STEAP2. Said cancerous cell may be comprised within a tumor.

In one aspect, the cancer is one or more selected from breast cancer, ovarian cancer, endometrial cancer, prostate cancer, cholangiocarcinoma, NSCLC (squamous and adenocarcinoma), pancreatic cancer, and gastric cancer.

In one aspect, the cancer is one or more selected from colorectal cancer, HNSCC, prostate cancer, lung cancer (e.g. NSCLC or SCLC), breast cancer, ovarian cancer pancreatic cancer, gastric cancer, cholangiocarcinoma, melanoma, endometrial cancer, hematological cancer (AML, MM, DLBCL), and cancers comprising CSCs.

In one aspect, the cancer is lung cancer, breast cancer, or a combination thereof. For example, the cancer may be lung cancer. The cancer may be breast cancer. The cancer may be ovarian cancer. The cancer may be prostate cancer.

In one aspect, the cancer is one or more non-small-cell lung carcinoma (NSCLC) selected from squamous NSCLC, adenocarcinoma NSCLC, or a combination thereof.

An antibody or antigen binding fragment thereof also finds utility in detecting a cancer cell, for example as part of a diagnostic method.

In a further aspect, there is provided a method for detecting the presence or absence of a STEAP2 polypeptide (e.g. a STEAP2 polypeptide epitope) in a sample, comprising:
a. contacting a sample with an antibody or antigen binding fragment thereof, or a pharmaceutical composition comprising an antibody or antigen binding fragment thereof, to provide an antibody-antigen complex; wherein said antibody or antigen binding fragment thereof comprises:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof.
b. detecting the presence or absence of said antibody-antigen complex; and
c. wherein the presence of the antibody-antigen complex confirms the presence of a STEAP2 polypeptide (e.g. STEAP2 polypeptide epitope); or
d. wherein the absence of the antibody-antigen complex confirms the absence of STEAP2 polypeptide (e.g. STEAP2 polypeptide epitope).

In a related aspect, there is provided a method for detecting the presence or absence of a cancer cell expressing a STEAP2 polypeptide (e.g. STEAP2 polypeptide epitope) in a sample, comprising:
a. contacting a sample with an antibody or antigen binding fragment thereof, or a pharmaceutical composition comprising an antibody or antigen binding fragment thereof, to provide an antibody-antigen complex; wherein said antibody or antigen binding fragment thereof comprises:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively, or a functional variant thereof;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively, or a functional variant thereof;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively, or a functional variant thereof; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively, or a functional variant thereof;
b. detecting the presence or absence of said antibody-antigen complex; and
c. wherein the presence of the antibody-antigen complex confirms the presence of a STEAP2 polypeptide (e.g. STEAP2 polypeptide epitope); or
d. wherein the absence of the antibody-antigen complex confirms the absence of STEAP2 polypeptide (e.g. STEAP2 polypeptide epitope).

The disclosure embraces a corresponding use of the antibody or antigen binding fragment thereof of the disclosure for detecting a STEAP2 polypeptide (e.g. STEAP2 polypeptide epitope).

In one aspect, the presence of antibody-antigen complex is indicative of the presence of a cancer cell, and the absence of the antibody-antigen complex is indicative of the absence of a cancer cell. For example, the method may comprise confirming the presence of cancer where an antibody-antigen complex is detected, or not confirming the presence of cancer where an antibody-antigen complex is not detected.

In one aspect, the cancer cell is a cancer cell expressing a STEAP2 polypeptide (e.g. STEAP2 polypeptide epitope).

Thus, the present disclosure embraces corresponding use of the method steps described herein in methods of diagnosing a subject with a cancer, wherein said cancer comprises a STEAP2 expressing cancer cell.

In one aspect, a method of detection or method of diagnosis may comprise measuring the expression level of STEAP2 on a cell (or tissue) obtainable from a subject, and comparing the measured expression level with a standard STEAP2 expression in a control cell (or tissue), wherein an increase in the expression level compared to the control is indicative of the presence of cancer. Said control sample can comprise a non-cancer (e.g. normal) cell.

An “antibody-antigen complex” means a complex (e.g. macromolecular complex) comprising a STEAP2 antigen which has become bound to an antibody. The term “antibody-antigen complex” may be used synonymously with the terms “bound STEAP2-antibody complex” and “antibody bound to a STEAP2”.

An antibody-antigen complex may be detected by any means known to the skilled person. In one aspect, the antibody (or antigen binding fragment thereof) is labelled with a detectable label. Said label may be an epi-fluorescent label. In another aspect, the antibody is labelled with 800CW.

In one aspect, an antibody-antigen complex is detected by means of a secondary (e.g. detection) antibody which binds the antibody and/or antibody-antigen complex.

Suitably, said secondary antibody comprises a detection means, such as a tag/label to aid detection. Said detection means is conjugated to the secondary antibody. Examples of suitable labels include detectable labels such as radiolabels or fluorescent or colored molecules, enzymatic markers or chromogenic markers—e.g. dyes that provide a visible color change upon binding of the detection antibody to an antigen. By way of example, the label may be fluorescein-isothiocyanate (FITC), R-phycoerythrin, Alexa 532, CY3 or digoxigenin. The label may be a reporter molecule, which is detected directly, such as by detecting its fluorescent signal, or by exposure of the label to photographic or X-ray film. Alternatively, the label is not directly detectable, but may be detected, for example, in a two-phase system. An example of indirect label detection is binding of an antibody to the label.

In another aspect, said secondary antibody comprises a fluorescent tag, and an antibody-antigen complex is detected by the florescence emitted from a, antibody-antigen-secondary antibody complex. An “antibody-antigen-secondary antibody complex” means a complex comprising an antigen (e.g. STEAP2) which has become bound to an antibody, wherein said complex has further become bound by a secondary antibody which binds said antibody and/or antibody-antigen complex.

Suitably, an antibody-antigen complex is detected when the signal (e.g. fluorescence) emitted from the detection label is greater than the signal detected in a control comprising no antibody (e.g. no antibody which binds a STEAP2). Said control may alternatively comprise a STEAP2, but the sample is not applied to said control.

Suitably, a “sample” is a sample obtained from a subject (e.g. biopsy), cell line, tissue culture, or other source of cells potentially expressing STEAP2. In one aspect, a sample is a biopsy from a subject. Said biopsy may be taken from a tumor, or a site at risk of developing a tumor.

In one aspect, the sample is an isolated sample obtainable (e.g. obtained) from a subject.

In another aspect, the STEAP2 polypeptide (e.g. STEAP2 polypeptide epitope) is an integral component of a cancer cell, for example, an integral component of the cell membrane of a cancer cell.

The present disclosure encompasses the antibodies (e.g. the antibody or antigen binding fragment) defined herein having the recited CDR sequences or variable heavy and variable light chain sequences (reference antibodies), as well as functional variants thereof. A functional variant binds to the same target antigen as the reference antibody, and may exhibit the same antigen cross-reactivity as the reference antibody. In some aspects, the functional variants may have a different affinity for the target antigen when compared to the reference antibody. In another aspect, the functional variant has the same the same affinity for the target antigen when compared to the reference antibody.

The term “reference antibody” is used to conveniently refer, in comparison, to an antibody or antigen thereof of the disclosure. Thus, the term “reference antibody” refers to an antibody or antigen thereof of the disclosure. For example, the reference antibody may mean an antibody or antigen binding fragment thereof comprising a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively. The reference antibody may mean an antibody or antigen binding fragment thereof comprising a variable heavy chain comprising the amino acid sequence of SEQ ID NO: 33; and a variable light chain comprising the amino acid sequence of SEQ ID NO: 34. The reference antibody may mean an antibody or antigen binding fragment thereof comprising a variable heavy chain comprising the amino acid sequence of SEQ ID NO: 31; and a variable light chain comprising the amino acid sequence of SEQ ID NO: 32.

In one aspect functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, at most 1 amino acid differences when compared to a corresponding reference CDR sequence, and when combined with the remaining 5 CDRs (or variants thereof) enables the variant antibody to bind to the same target antigen as the reference antibody, and to exhibit the same antigen cross-reactivity as the reference antibody. A functional variant may be referred to as a “variant antibody”.

In one aspect a variant antibody (or antigen binding fragment thereof) comprises: a light chain CDR1 having at most 2 amino acid difference when compared to a corresponding reference CDR sequence; a light chain CDR2 having at most 2 amino acid difference when compared to a corresponding reference CDR sequence; a light chain CDR3 having at most 2 amino acid difference when compared to a corresponding reference CDR sequence; a heavy chain CDR1 having at most 2 amino acid difference when compared to a corresponding reference CDR sequence; a heavy chain CDR2 having at most 2 amino acid difference when compared to a corresponding reference CDR sequence; and a heavy chain CDR3 having at most 2 amino acid difference when compared to a corresponding reference CDR sequence; wherein the variant antibody binds to the same target antigen as the reference antibody, and can exhibit the same antigen cross-reactivity (or lack thereof) as the reference antibody.

A variant antibody (or antigen binding fragment thereof) can comprise: a light chain CDR1 having at most 1 amino acid difference when compared to a corresponding reference CDR sequence; a light chain CDR2 having at most 1 amino acid difference when compared to a corresponding reference CDR sequence; a light chain CDR3 having at most 1 amino acid difference when compared to a corresponding reference CDR sequence; a heavy chain CDR1 having at most 1 amino acid difference when compared to a corresponding reference CDR sequence; a heavy chain CDR2 having at most 1 amino acid difference when compared to a corresponding reference CDR sequence; and a heavy chain CDR3 having at most 1 amino acid difference when compared to a corresponding reference CDR sequence; wherein the variant antibody binds to the same target antigen as the reference antibody, and can exhibit the same antigen cross-reactivity (or lack thereof) as the reference antibody.

For example, a variant of the antibody or antigen binding fragment may comprise: a heavy chain CDR1 having at most 2 amino acid difference when compared to SEQ ID NO: 7; a heavy chain CDR2 having at most 2 amino acid difference when compared to SEQ ID NO: 8; and a heavy chain CDR3 having at most 2 amino acid difference when compared to SEQ ID NO: 9; a light chain CDR1 having at most 2 amino acid difference when compared to SEQ ID NO: 10; a light chain CDR2 having at most 2 amino acid difference when compared to SEQ ID NO: 11; a light chain CDR3 having at most 2 amino acid difference when compared to SEQ ID NO: 12; wherein the variant antibody binds to a STEAP2 polypeptide (e.g. STEAP2 polypeptide epitope), and can exhibit the same antigen cross-reactivity (or lack thereof) as the reference antibody or antigen binding fragment.

For example, a variant of the antibody or antigen binding fragment can comprise: a heavy chain CDR1 having at most 1 amino acid difference when compared to SEQ ID NO: 7; a heavy chain CDR2 having at most 1 amino acid difference when compared to SEQ ID NO: 8; and a heavy chain CDR3 having at most 1 amino acid difference when compared to SEQ ID NO: 9; a light chain CDR1 having at most 1 amino acid difference when compared to SEQ ID NO: 10; a light chain CDR2 having at most 1 amino acid difference when compared to SEQ ID NO: 11; a light chain CDR3 having at most 1 amino acid difference when compared to SEQ ID NO: 12; wherein the variant antibody binds to a STEAP2 polypeptide (e.g. STEAP2 polypeptide epitope), and exhibits the same antigen cross-reactivity (or lack thereof) as the reference antibody or antigen binding fragment.

The foregoing can be applied analogously to variants of the other antibodies described herein, wherein the amino acid differences are defined relative to the CDR sequences thereof, and wherein the variant antibody binds to the same target antigen as said antibodies, and exhibits the same antigen cross-reactivity.

In one aspect, a variant antibody may have at most 5, 4 or 3 amino acid differences total in the CDRs thereof when compared to a corresponding reference antibody, with the proviso that there is at most 2 (sometimes at most 1) amino acid differences per CDR. A variant antibody has at most 2 (sometimes at most 1) amino acid differences total in the CDRs thereof when compared to a corresponding reference antibody, with the proviso that there is at most 2 amino acid differences per CDR. Sometimes a variant antibody has at most 2 (sometimes at most 1) amino acid differences total in the CDRs thereof when compared to a corresponding reference antibody, with the proviso that there is at most 1 amino acid difference per CDR.

The amino acid difference may be an amino acid substitution, insertion or deletion. In one aspect the amino acid difference is a conservative amino acid substitution as described herein.

In one aspect a variant antibody has the same framework sequences as the exemplary antibodies described herein. In another aspect the variant antibody may comprise a framework region having at most 2, sometimes at most 1 amino acid difference (when compared to a corresponding reference framework sequence). Thus, each framework region may have at most 2, sometimes at most 1 amino acid difference (when compared to a corresponding reference framework sequence).

In one aspect a variant antibody may have at most 5, 4 or 3 amino acid differences total in the framework regions thereof when compared to a corresponding reference antibody, with the proviso that there is at most 2 (sometimes at most 1) amino acid differences per framework region. In some aspects, a variant antibody has at most 2 (sometimes at most 1) amino acid differences total in the framework regions thereof when compared to a corresponding reference antibody, with the proviso that there is at most 2 amino acid differences per framework region. Sometimes a variant antibody has at most 2 (sometimes at most 1) amino acid differences total in the framework regions thereof when compared to a corresponding reference antibody, with the proviso that there is at most 1 amino acid difference per framework region.

Thus, a variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference antibody, and exhibits the same antigen cross-reactivity (or lack thereof) as the reference antibody.

Said variant heavy or light chains may be referred to as “functional equivalents” of the reference heavy or light chains.

In one aspect a variant antibody may comprise a variable heavy chain and a variable light chain as described herein, wherein:
the heavy chain has at most 7 amino acid differences (at most 1 amino acid difference in each CDR and at most 1 amino acid difference in each framework region) when compared to a heavy chain sequence herein; and
the light chain has at most 7 amino acid differences (at most 1 amino acid difference in each CDR and at most 1 amino acid difference in each framework region) when compared to a light chain sequence herein;
wherein the variant antibody binds to the same target antigen as the reference antibody, and sometimes exhibits the same antigen cross-reactivity (or lack thereof) as the reference antibody.

### Antibody-Drug Conjugates (ADCs)

Advantageously, an antibody or antigen binding fragment thereof of the disclosure may comprise a heterologous agent. In one aspect, an antibody or antigen binding fragment of the disclosure is linked to a heterologous agent. In another aspect, the antibody or antigen binding fragment is conjugated to a heterologous agent. Suitably, “conjugated” means linked via a covalent or ionic bond. In some aspects, said heterologous agent is a cytotoxin.

The heterologous agent may simply be referred to as an “agent” or “active agent”. For example, in alternative language, an antibody or antigen binding fragment thereof of the disclosure may comprise an active agent. In one aspect, an antibody or antigen binding fragment of the disclosure is linked to an active agent. In another aspect, the antibody or antigen binding fragment is conjugated to an active agent.

The heterologous/active agent can be a drug. In some aspects, the heterologous/active is a cytotoxin.

In some aspects, that an antibody or antigen binding fragment thereof of the disclosure is linked (e.g. conjugated) to a heterologous/active agent in methods of treatment, as described below.

An agent and/or cytotoxin of the disclosure may be conjugated to the antibody or antigen binding fragment thereof by means of a spacer (e.g. at least one spacer). In one aspect, the spacer is a peptide spacer. In one aspect, the spacer is a non-peptide (e.g. chemical) spacer.

The cytotoxic agent or cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. These include, but are not limited to, topoisomerase I inhibitors, amanitins, auristatins, daunomycins, doxorubicins, duocarmycins, dolastatins, enediynes, lexitropsins, taxanes, puromycins, maytansinoids, vinca alkaloids, and tubulysins. Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof. Additional disclosure regarding cytotoxins suitable for use in ADCs can be found, for example, in International Patent Application Publication Nos. WO 2015/155345 and WO 2015/157592, incorporated by reference herein in their entirety.

For example, the antibody or antigen binding fragment may be conjugated to such heterologous agent or cytotoxic agent to provide an “antibody-drug conjugate” (ADC). In some aspects the heterologous agent or cytotoxic agent is SG3932. In some aspects the heterologous agent or cytotoxic agent is LP-1.

“SG3932” as used herein, refers to the following structure:

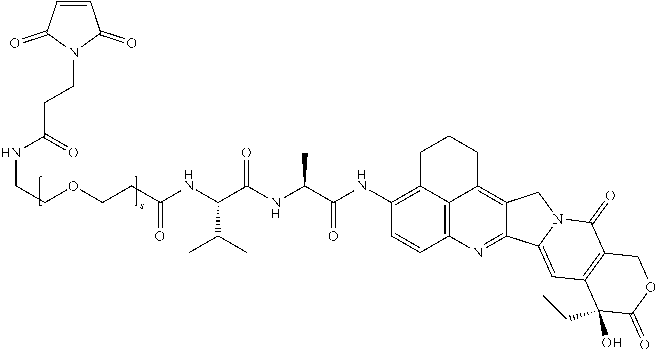

“LP-1” or “LP1” as used herein refers to the following conjugated structure:

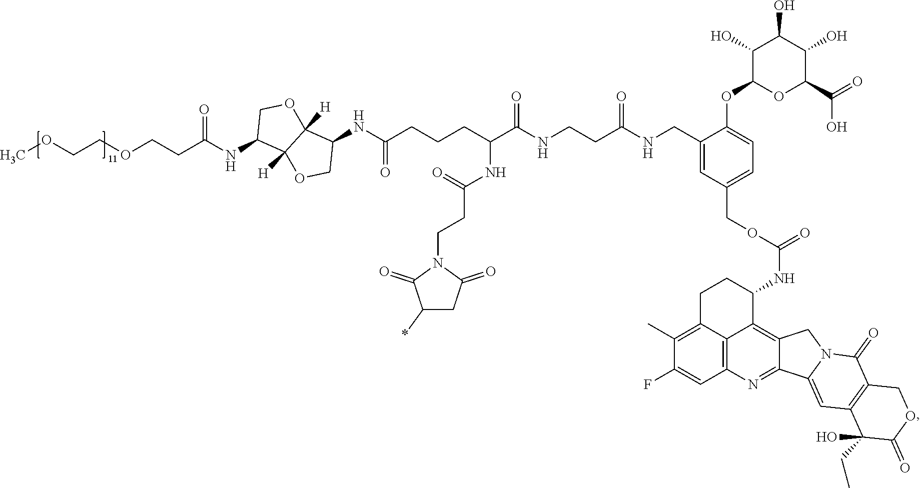

wherein [symbol] indicates the point of attachment to the antibody or antigen-binding fragment thereof. Alternatively, “unconjugated LP-1” or “unconjugated LP1” refers to the following structure:

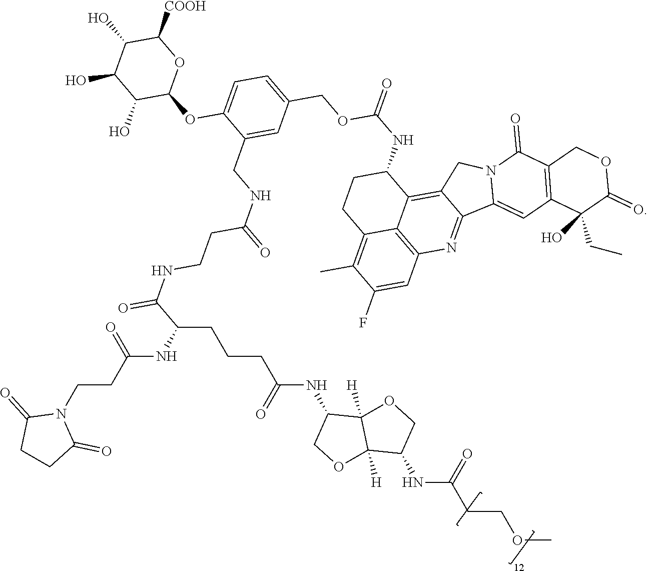

The agent is typically linked to, or “loaded onto” the antibody or antigen-binding fragment. The agent loading (p) is the average number of agent(s) per antibody or antigen-binding fragment (e.g. the Ligand unit).

The average number of agents per antibody (or antigen-binding fragment) in preparations of ADCs from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). In some instances, separation, purification, and characterization of homogeneous ADC, where p is a certain value from ADC with other drug loadings, may be achieved by means such as reverse phase HPLC or electrophoresis. Such techniques are also applicable to other types of conjugates.

Cysteine amino acids may be engineered at reactive sites in an antibody (or antigen-binding fragment thereof) and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with a linker within an agent (e.g. of formula I below) which may have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody or antigen binding fragment thereof reacts with an agent, then the resulting product may be a mixture of ADC compounds with a distribution of agent units attached to an antibody, e.g. 1, 2, 3, etc. Liquid chromatography methods such as polymeric reverse phase (PLRP) and hydrophobic interaction (HIC) may separate compounds in the mixture by agent loading value. Preparations of ADC with a single agent loading value (p) may be isolated.

Thus, the antibody-drug conjugate compositions of the disclosure may include mixtures of antibody-drug conjugates where the antibody or antigen binding fragment thereof has one or more agent moieties and where the agent moieties may be attached to the antibody or antigen binding fragment thereof at various amino acid residues.

In one aspect, the average number of agents per antibody (or antigen-binding fragment thereof) is in the range 1 to 20. In some aspects the range is selected from 1 to 10, 2 to 10, 2 to 8, 2 to 6, and 4 to 10. In some aspects, there is one agent per antibody (or antigen-binding fragment thereof). In some aspects, the number of agents per antibody (or antigen-binding fragment thereof) can be expressed as a ratio of agent (i.e., drug) to antibody. This ratio is referred to as the Drug to Antibody Ratio (DAR).” The DAR is the average number of drugs (i.e., agents) linked to each antibody. In one aspect of the present disclosure, the DAR is in the range 1 to 20. In some aspects the range of DAR is selected from 1 to 10, 2 to 10, 2 to 8, 2 to 6, and 4 to 10. In a particular aspect of the present disclosure, the DAR is about 8. In a particular aspect of the present disclosure, the DAR is 8. In a particular aspect of the present disclosure, the DAR is about 4. In a particular aspect of the present disclosure, the DAR is 4.

In one aspect, the antibody or antigen-binding fragment is conjugated to one or more heterologous agent selected from the group consisting of a topoisomerase I inhibitor, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

In one aspect, the antibody antigen binding fragment is conjugated to one or more cytotoxin selected from a topoisomerase I inhibitor, tubulysin derivative, or a combination thereof. For example, the antibody or antigen binding fragment thereof is conjugated to one or more cytotoxin selected from the group consisting of topoisomerase I inhibitor SG3932, SG4010, SG4057 or SG4052 (the structures of which are provided below); tubulysin AZ1508, or a combination thereof. In some aspects, the topoisomerase I inhibitor is SG3932.

The antibody or antigen binding fragment thereof may be conjugated to a topoisomerase I inhibitor. Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle.

A general example of a suitable topoisomerase I inhibitor is represented by the following compound:

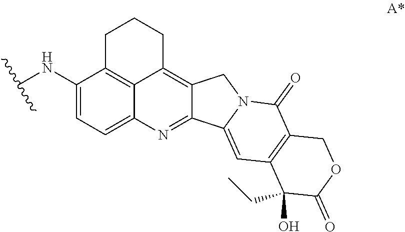 [SMILES: *Nc1ccc2nc3c(c4c2c1CCC4)Cn1c-3cc2c(c1=O)COC(=O)[C@]2(O)CC]

Said compound is denoted as A*, and may be referred to as a “Drug Unit” herein.

The compound (e.g. A*) is provided with a linker for connecting (conjugating) to an antibody or antigen binding fragment described herein (which may be referred to as a “Ligand Unit”). Suitably, the linker is attached (e.g. conjugated) in a cleavable manner to an amino residue, for example, an amino acid of an antibody or antigen binding fragment described herein.

An example of a suitable topoisomerase I inhibitor is represented by the following compound, with the formula “I”:

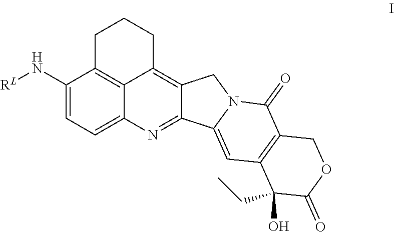

and salts and solvates thereof, wherein R^{L} is a linker for connection to an antibody or antigen binding fragment thereof described herein (e.g. the Ligand Unit), wherein said linker is selected from:

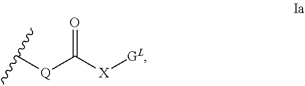

wherein
Q is:

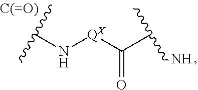

where Q^{X} is such that Q is an amino-acid residue, a dipeptide residue, a tripeptide residue or a tetrapeptide residue;
X is:

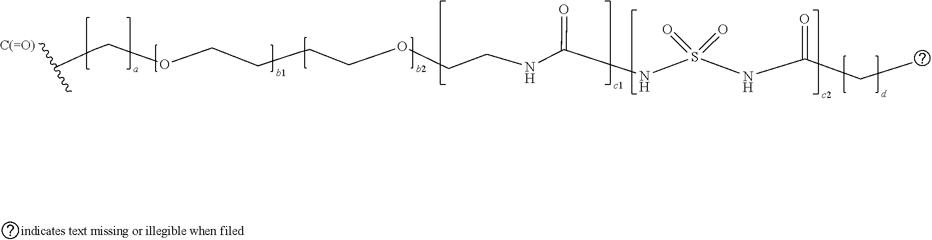

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);
G^{L} is a linker for connecting to an antibody or antigen binding fragment thereof described herein (e.g. the Ligand Unit); or
   (ib):

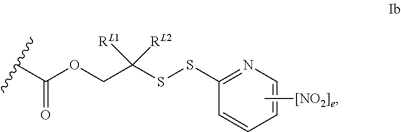

where R^{L1} and R^{L2} are independently selected from H and methyl, or together with the carbon atom to which they are bound form a cyclopropylene or cyclobutylene group; and
e is 0 or 1.

It will be understood by the person skilled in the art that more than one of said agent(s) (e.g. topoisomerase I inhibitor) may be conjugated to the antibody or antigen binding fragment thereof.

For example, a conjugate (e.g. antibody-drug conjugate) of the disclosure may be of the general formula IV:

L-(D^{L})ₚ (IV)

or a pharmaceutically acceptable salt or solvate thereof, wherein L is an antibody or antigen binding fragment thereof described herein (e.g. the Ligand Unit), D^{L} is a topoisomerase I inhibitor having a linker (e.g. Drug Linker unit) that is of formula III:

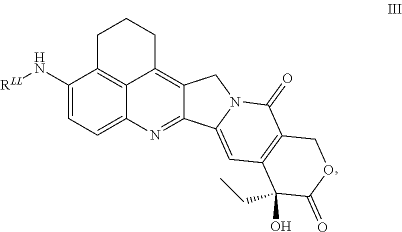

R^{LL} is a linker connected to an antibody or antigen binding fragment thereof described herein (e.g. the Ligand Unit), wherein the linker is selected from
   (ia′):

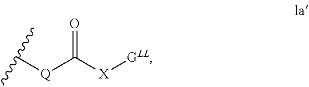

where Q and X are as defined above and G^{LL} is a linker connected to an antibody or antigen binding fragment thereof described herein (e.g. the Ligand Unit); and
(ib′):

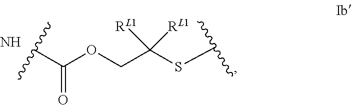

where R^{L1} and R^{L2} are as defined above; and
p is an integer of from 1 to 20.

The drug loading is represented by p, the number of topoisomerase I inhibitor(s) (e.g. Drug units) per antibody or antigen binding fragment thereof (e.g. Ligand Unit). Drug loading may range from 1 to 20 Drug units (D) per Ligand unit. For compositions, p represents the average drug loading of the conjugates in the composition, and p ranges from 1 to 20.

Accordingly, disclosed within is a conjugate comprising an antibody or antigen binding fragment thereof described herein (e.g. the Ligand Unit) covalently linked to at least one topoisomerase I inhibitor (e.g. Drug unit, such as A* illustrated above). Said inhibitor is linked to the antibody or antigen binding fragment thereof by a linker (e.g. Linker unit), such as a linker described above as R^{L} and/or R^{LL}. In other words, the disclosure embraces an antibody or antigen binding fragment thereof described herein (e.g. the Ligand Unit) with one or more topoisomerase I inhibitors attached, via a linker (e.g. Drug-Linker units). The antibody or antigen binding fragment thereof (representing a Ligand unit), described more fully above, is a targeting agent that binds to a target moiety. This Ligand unit can, for example, specifically bind to a STEAP2 on a target cell, to which the Drug unit is thus delivered. Accordingly, the present disclosure also provides methods for the treatment of, for example, various cancers and other disorders with an ADC (e.g. cancers/disorders which are associated with the presence of cells, such as cancerous cells, which express STEAP2).

### Further Aspects

Certain features of the topoisomerase I inhibitors are described above and may be defined in more detail as set out below. By way of example, an aspect of feature Q^{X} (e.g. within the linker of 1a described above) will be outlined.

The following may apply to all aspects of the disclosure as described above, may relate to a single aspect, or may be combined together in any combination.

Various definitions which pertain to certain terms in this section are provided under the heading “Definitions” provided below.

### Q^{X}

In one aspect, Q is an amino acid residue. The amino acid may be a natural amino acid or a non-natural amino acid. For example, Q may be selected from: Phe, Lys, Val, Ala, Cit, Leu, Ile, Arg, and Trp, where Cit is citrulline.

In one aspect, Q comprises a dipeptide residue. The amino acids in the dipeptide may be any combination of natural amino acids and non-natural amino acids. In some aspects, the dipeptide comprises natural amino acids. Where the linker is a cathepsin labile linker, the dipeptide is the site of action for cathepsin-mediated cleavage. The dipeptide then is a recognition site for cathepsin.

In one aspect, Q is selected from:
^{NH}-Phe-Lys-^{C═O},
^{NH}-Val-Ala-^{C═O},
^{NH}-Val-Lys-^{C═O},
^{NH}-Ala-Lys-^{C═O},
^{NH}-Val-Cit-^{C═O},
^{NH}-Phe-Cit-^{C═O},
^{NH}-Leu-Cit-^{C═O},
^{NH}-Ile-Cit-^{C═O}
^{NH}-Phe-Arg-^{C═O},
^{NH}-Trp-Cit-^{C═O}, and
^{NH}-Gly-Val-^{C═O}.
where Cit is citrulline.

In another aspect, Q is selected from:
^{NH}-Phe-Lys-^{C═O},
^{NH}-Val-Ala-^{C═O},
^{NH}-Val-Lys-^{C═O},
^{NH}-Ala-Lys-^{C═O}, and
NH Val-Cit-^{C═O}.

In another aspect, Q is selected from ^{NH}-Phe-Lys-^{C═O}, ^{NH}-Val-Cit-^{C═O} or ^{NH}-Val-Ala-^{C═O}.

Other suitable dipeptide combinations include:
^{NH}-Gly-Gly-^{C═O},
^{NH}-Gly-Val-^{C═O}
^{NH}-Pro-Pro-^{C═O} and
^{NH}-Val-Glu-^{C═O}.

Other dipeptide combinations may be used, including those described by Dubowchik et al., Bioconjugate Chemistry, 2002, 13, 855-869, which is incorporated herein by reference.

In some aspects, Q is a tripeptide residue. The amino acids in the tripeptide may be any combination of natural amino acids and non-natural amino acids. In some aspects, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-^{C═O}
^{NH}-Glu-Val-Cit-^{C═O}
^{NH}-αGlu-Val-Ala-^{C═O}
^{NH}-αGlu-Val-Cit-^{C═O}

In some aspects, Q is a tetrapeptide residue. The amino acids in the tetrapeptide may be any combination of natural amino acids and non-natural amino acids. In some aspects, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:
^{NH}-Gly-Gly-Phe-Gly^{C═O}; and
^{NH}-Gly-Phe-Gly-Gly^{C═O}.

In some aspects, the tetrapeptide is:
^{NH}-Gly-Gly-Phe-Gly^{C═O}.

In the above representations of peptide residues, ^{NH}-represents the N-terminus, and -^{C═O} represents the C-terminus of the residue. The C-terminus binds to the NH of A*.

Glu represents the residue of glutamic acid, i.e.:

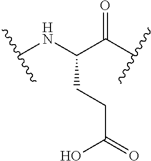 [SMILES: *N[C@@H](CCC(=O)O)C(*)=O]

αGlu represents the residue of glutamic acid when bound via the α-chain, i.e.:

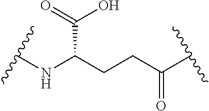 [SMILES: *N[C@@H](CCC(*)=O)C(=O)O]

In one aspect, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

### G^{L}

G^{L} may be selected from:

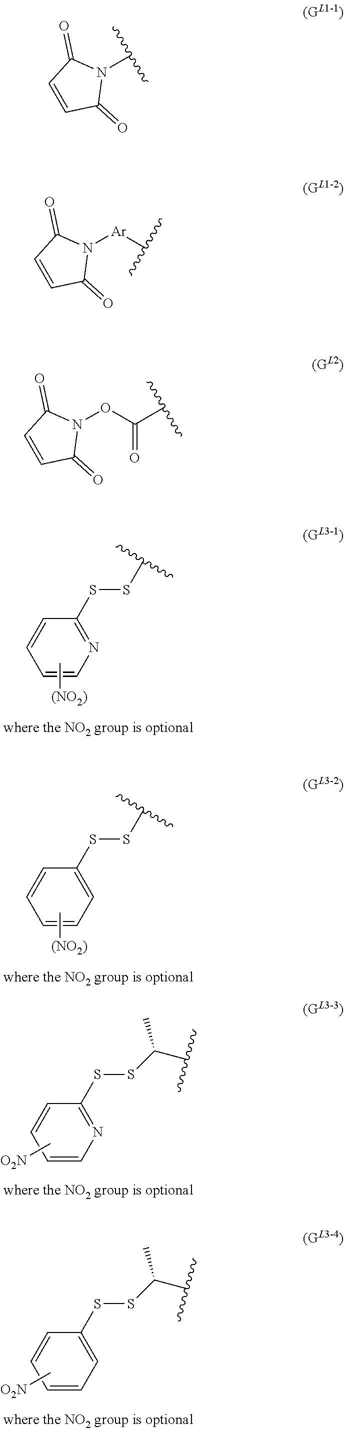

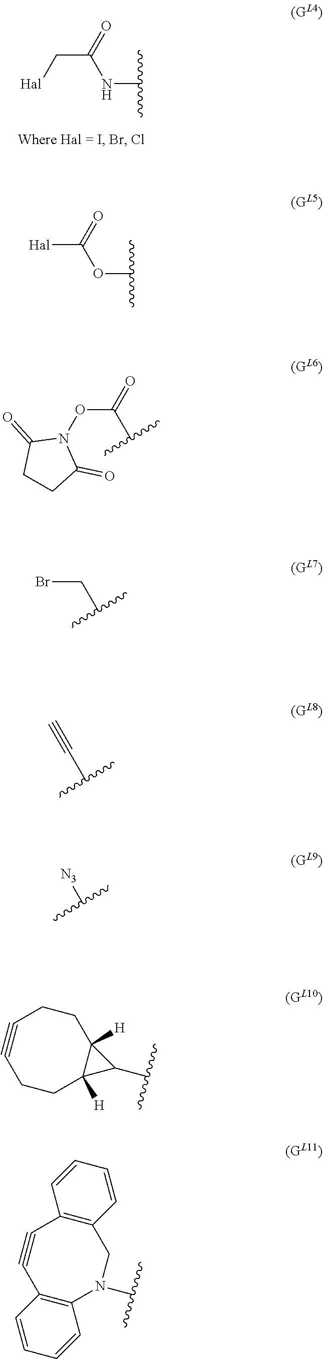

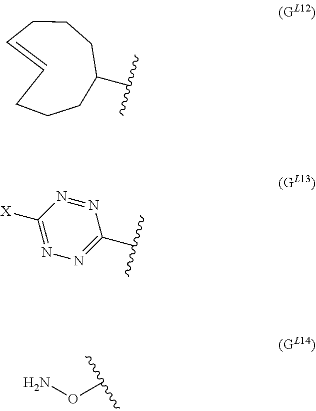

where Ar represents a C₅₋₆ arylene group, e.g. phenylene, and X represents C₁₋₄ alkyl.

In some aspects, G^{L} is selected from G^{L1-1} and G^{L1-2}. In some of these aspects, G^{L} is G^{L1-1}.

### G^{LL}

G^{LL} may be selected from:

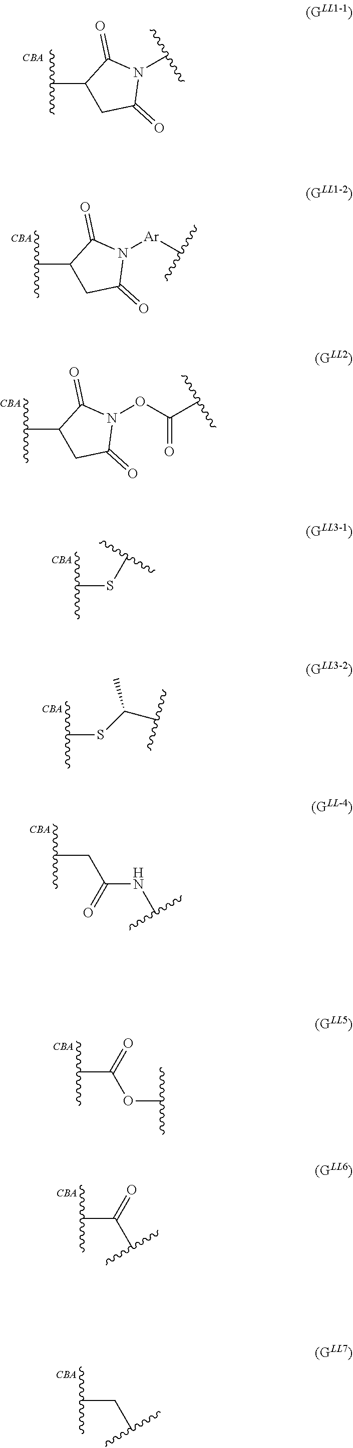

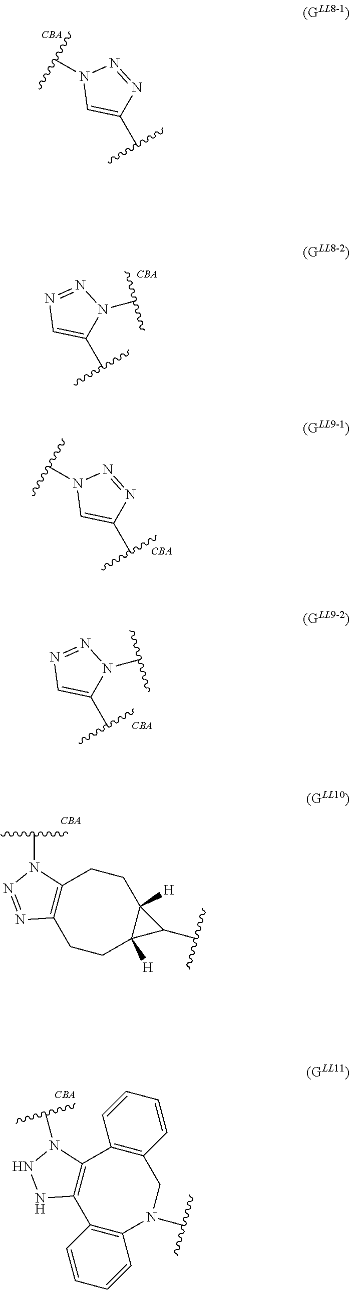

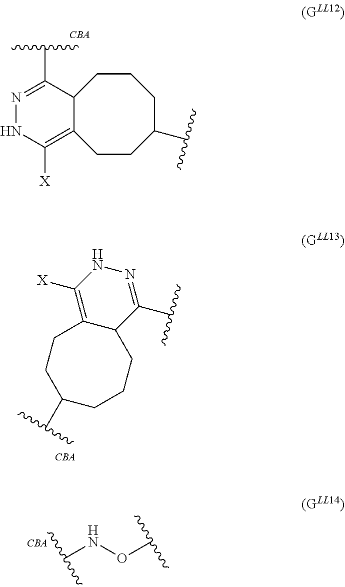

where Ar represents a C₅₋₆ arylene group, e.g. phenylene and X represents C₁₋₄ alkyl.

In some aspects, G^{LL} is selected from G^{LL1-1} and G^{LL1-2}. In some of these aspects, G^{LL} is G^{LL1-1}.

### X

In one aspect, X is:

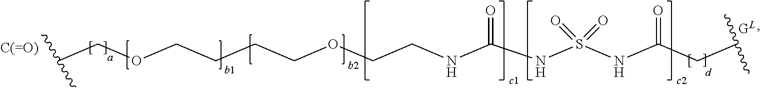

where a=0 to 5, b1=0 to 16, b2=0 to 16, c=0 or 1, d=0 to 5, wherein at least b1 or b2=0 and at least c1 or c2=0.
a may be 0, 1, 2, 3, 4 or 5. In some aspects, a is 0 to 3. In some of these aspects, a is 0 or 1.
In further aspects, a is 0.
b1 may be 0, 1, 2, 3, 4, 5, 6, 7, 8, 9, 10, 11, 12, 13, 14, 15 or 16. In some aspects, b1 is 0 to 12.
In some of these aspects, b1 is 0 to 8, and may be 0, 2, 3, 4, 5 or 8.
b2 may be 0, 1, 2, 3, 4, 5, 6, 7, 8, 9, 10, 11, 12, 13, 14, 15 or 16. In some aspects, b2 is 0 to 12.
In some of these aspects, b2 is 0 to 8, and may be 0, 2, 3, 4, 5 or 8. Sometimes, only one of b1 and b2 may not be 0.
c1 may be 0 or 1. c2 may be 0 or 1. Sometimes, only one of c1 and c2 may not be 0.
d may be 0, 1, 2, 3, 4 or 5. In some aspects, d is 0 to 3. In some of these aspects, d is 1 or 2.
In further aspects, d is 2. In further aspects, d is 5.

In some aspects of X, a is 0, b1 is 0, c1 is 1, c2 is 0 and d is 2, and b2 may be from 0 to 8. In some of these aspects, b2 is 0, 2, 3, 4, 5 or 8. In some aspects of X, a is 1, b2 is 0, c1 is 0, c2 is 0 and d is 0, and b1 may be from 0 to 8. In some of these aspects, b1 is 0, 2, 3, 4, 5 or 8. In some aspects of X, a is 0, b1 is 0, c1 is 0, c2 is 0 and d is 1, and b2 may be from 0 to 8. In some of these aspects, b2 is 0, 2, 3, 4, 5 or 8. In some aspects of X, b1 is 0, b2 is 0, c1 is 0, c2 is 0 and one of a and d is 0. The other of a and d is from 1 to 5. In some of these aspects, the other of a and d is 1. In other of these aspects, the other of a and d is 5. In some aspects of X, a is 1, b2 is 0, c1 is 0, c2 is 1, d is 2, and b1 may be from 0 to 8. In some of these aspects, b2 is 0, 2, 3, 4, 5 or 8.

In some aspects, R^{L} is of formula Ib. In some aspects, R^{LL} is formula Ib′.

R^{L1} and R^{L2} may be independently selected from H and methyl, or together with the carbon atom to which they are bound form a cyclopropylene or cyclobutylene group.

In some aspects, both R^{L1} and R^{L2} are H. In some aspects, R^{L1} is H and R^{L2} is methyl. In some aspects, both R^{L1} and R^{L2} are methyl.

In some aspects, R^{L1} and R^{L2} together with the carbon atom to which they are bound form a cyclopropylene group. In some aspects, R^{L1} and R^{L2} together with the carbon atom to which they are bound form a cyclobutylene group.

In the group Ib, in some aspects, e is 0. In other aspects, e is 1 and the nitro group may be in any available position of the ring. In some of these aspects, it is in the ortho position. In others of these aspects, it is in the para position.

In some aspects where compounds described herein are provided in a single enantiomer or in an enantiomerically enriched form, the enantiomerically enriched form has an enantiomeric ratio greater than 60:40, 70:30; 80:20 or 90:10. In further aspects, the enantiomeric ratio is greater than 95:5, 97:3 or 99:1.

In some aspects, R^{L} is selected from:

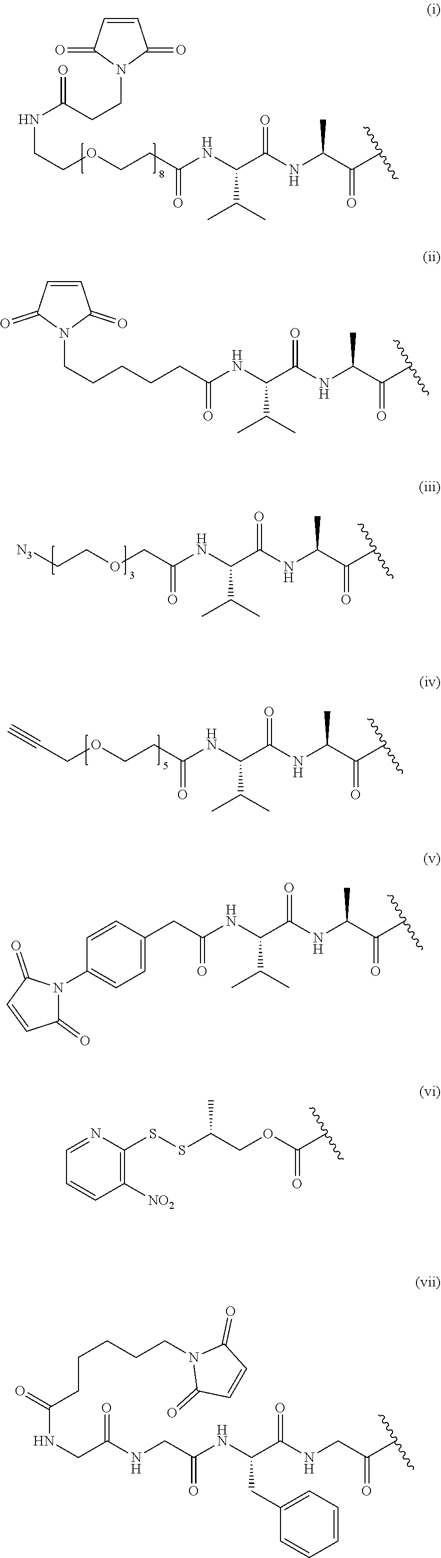

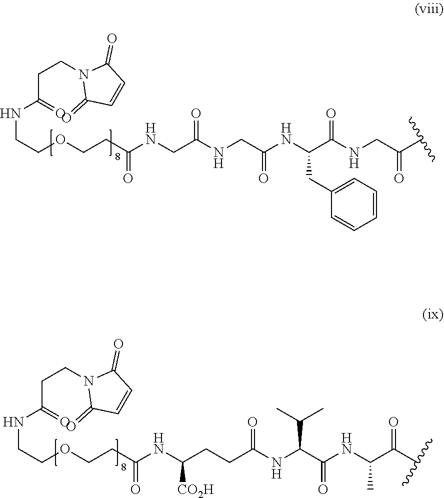

In some aspects, R^{LL} is a group derived from the R^{L} groups above.

Having outlined said details above, certain topoisomerase I-linker (e.g. Drug Linker unit) formulas are now described.

In some aspects, the compound of formula I is of the formula I^{P}:

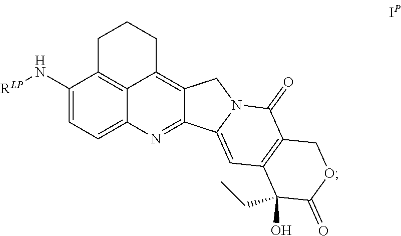

and salts and solvates thereof, wherein R^{LP} is a linker for connection to an antibody or antigen binding fragment thereof described herein, wherein said linker is selected from:
(ia):

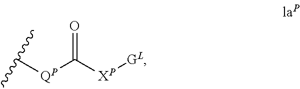

wherein

### Q^{P} is:

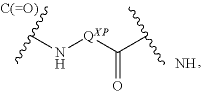

where Q^{XP} is such that Q^{P} is an amino-acid residue, a dipeptide residue or a tripeptide residue;

### X^{P} is:

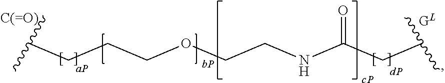

where aP=0 to 5, bP=0 to 16, cP=0 or 1, dP=0 to 5;
G^{L} is a linker for connecting to an antibody or antigen binding fragment thereof described herein (e.g. Ligand Unit);
   (ib):

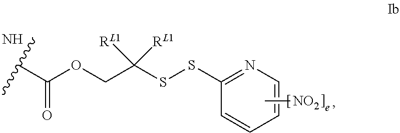

where R^{L1} and R^{L2} are independently selected from H and methyl, or together with the carbon atom to which they are bound form a cyclopropylene or cyclobutylene group; and
e is 0 or 1.
   aP may be 0, 1, 2, 3, 4 or 5. In some aspects, aP is 0 to 3. In some of these aspects, aP is 0 or 1. In further aspects, aP is 0.
   bP may be 0, 1, 2, 3, 4, 5, 6, 7, 8, 9, 10, 11, 12, 13, 14, 15 or 16. In some aspects, b is 0 to 12.
   In some of these aspects, bP is 0 to 8, and may be 0, 2, 4 or 8. cP may be 0 or 1.
   dP may be 0, 1, 2, 3, 4 or 5. In some aspects, dP is 0 to 3. In some of these aspects, dP is 1 or 2. In further aspects, dP is 2.

In some aspects of X^{P}, aP is 0, cP is 1 and dP is 2, and bP may be from 0 to 8. In some of these aspects, bP is 0, 4 or 8.

The preferences for Q^{X} above for compounds of Formula I may apply to Q^{XP} (for example, where appropriate).

The preferences for G^{L}, R^{L1}, R^{L2} and e above for compounds of Formula I may apply to compounds of Formula I^{P}.

In some aspects, the conjugate of formula IV is of the formula IV^{P}:

L-(D^{LP})ₚ (IV^{P})

or a pharmaceutically acceptable salt or solvate thereof, wherein L is an antibody or antigen binding fragment thereof described herein (e.g. Ligand Unit), D^{LP} is a topoisomerase I inhibitor (e.g. Drug Linker unit) that is of formula III^{P}:

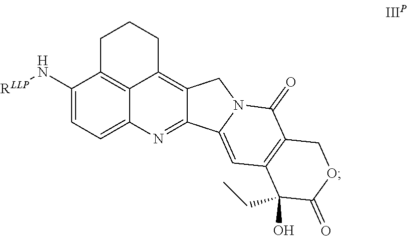

R^{LLP} is a linker connected to the antibody or antigen binding fragment thereof (e.g. Ligand unit), wherein said linker is selected from
   (ia′):

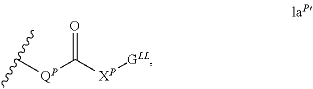

where Q^{P} and X^{P} are as defined above and G^{LL} is a linker connected to an antibody or antigen binding fragment thereof described herein (e.g. Ligand Unit); and
(ib′):

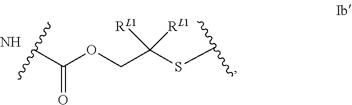

where R^{L1} and R^{L2} are as defined above; and
p is an integer of from 1 to 20.

In some aspects, the compound of formula I is of the formula I^{P2}:

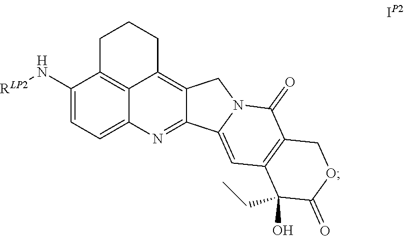

and salts and solvates thereof, wherein R^{LP2} is a linker for connection to an antibody or antigen binding fragment thereof described herein, wherein said linker is selected from:
(ia):

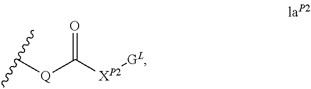

wherein
Q is:

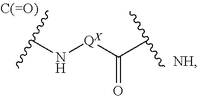

where Q^{X} is such that Q is an amino-acid residue, a dipeptide residue, a tripeptide residue or a tetrapeptide residue;
X^{P2} is:

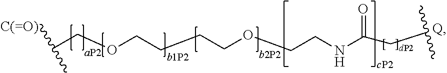

where aP2=0 to 5, b1P2=0 to 16, b2P2=0 to 16, cP2=0 or 1, dP2=0 to 5, wherein at least b1P2 or b2P2=0 (i.e. only one of b1 and b2 may not be 0);
G^{L} is a linker for connecting to an antibody or antigen binding fragment thereof described herein (e.g. Ligand Unit);
   (ib):

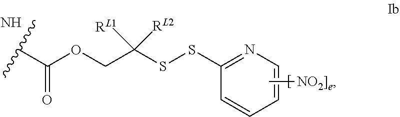

where R^{L1} and R^{L2} are independently selected from H and methyl, or together with the carbon atom to which they are bound form a cyclopropylene or cyclobutylene group; and
e is 0 or 1.

aP2 may be 0, 1, 2, 3, 4 or 5. In some aspects, aP2 is 0 to 3. In some of these aspects, aP2 is 0 or 1. In further aspects, aP2 is 0.

b1P2 may be 0, 1, 2, 3, 4, 5, 6, 7, 8, 9, 10, 11, 12, 13, 14, 15 or 16. In some aspects, b1P2 is 0 to 12. In some of these aspects, b1P2 is 0 to 8, and may be 0, 2, 3, 4, 5 or 8.

b2P2 may be 0, 1, 2, 3, 4, 5, 6, 7, 8, 9, 10, 11, 12, 13, 14, 15 or 16. In some aspects, b2P2 is 0 to 12. In some of these aspects, b2P2 is 0 to 8, and may be 0, 2, 3, 4, 5 or 8.

Sometimes, only one of b1P2 and b2P2 may not be 0. cP2 may be 0 or 1.

dP2 may be 0, 1, 2, 3, 4 or 5. In some aspects, dP2 is 0 to 3. In some of these aspects, dP2 is 1 or 2. In further aspects, dP2 is 2. In further aspects, dP2 is 5.

In some aspects of X¹², aP2 is 0, b1P2 is 0, cP2 is 1 and dP2 is 2, and b2P2 may be from 0 to 8. In some of these aspects, b2P2 is 0, 2, 3, 4, 5 or 8. In some aspects of X¹², aP2 is 1, b2P2 is 0, cP2 is 0 and dP2 is 0, and b1P2 may be from 0 to 8. In some of these aspects, b1P2 is 0, 2, 3, 4, 5 or 8. In some aspects of X¹², aP2 is 0, b1P2 is 0, cP2 is 0 and dP2 is 1, and b2P2 may be from 0 to 8. In some of these aspects, b2P2 is 0, 2, 3, 4, 5 or 8. In some aspects of X¹², b1P2 is 0, b2P2 is 0, cP2 is 0 and one of aP2 and dP2 is 0. The other of aP2 and d is from 1 to 5. In some of these aspects, the other of aP2 and d is 1. In other of these aspects, the other of aP2 and dP2 is 5.

The preferences for Q^{X} above for compounds of Formula I may apply to Q^{X} in Formula Ia^{P2} (e.g. where appropriate).

The preferences for G^{L}, R^{L1}, R^{L2} and e above for compounds of Formula I may apply to compounds of Formula I^{P2}.

In some aspects, the conjugate of formula IV is of the formula IV^{P2}:

L-(D^{LP2})ₚ (IV^{P2})

or a pharmaceutically acceptable salt or solvate thereof, wherein L is an antibody or antigen binding fragment thereof described herein (e.g. Ligand unit), D^{LP2} is a topoisomerase I inhibitor (e.g. Drug Linker unit) that is of formula III^{P2}:

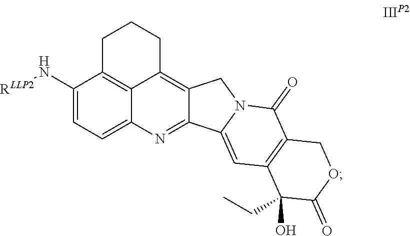

R^{LLP2} is a linker connected to the antibody or antigen binding fragment thereof (e.g. Ligand unit), wherein said linker is selected from
   (ia′):

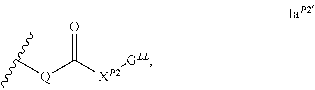

where Q and X^{P2} are as defined above and G^{LL} is a linker connected to the antibody or antigen binding fragment thereof; and
(ib′):

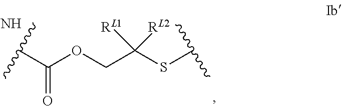

where R^{L1} and R^{L2} are as defined above; and
p is an integer of from 1 to 20.

Suitable topoisomerase I inhibitors include those having the following formulas:

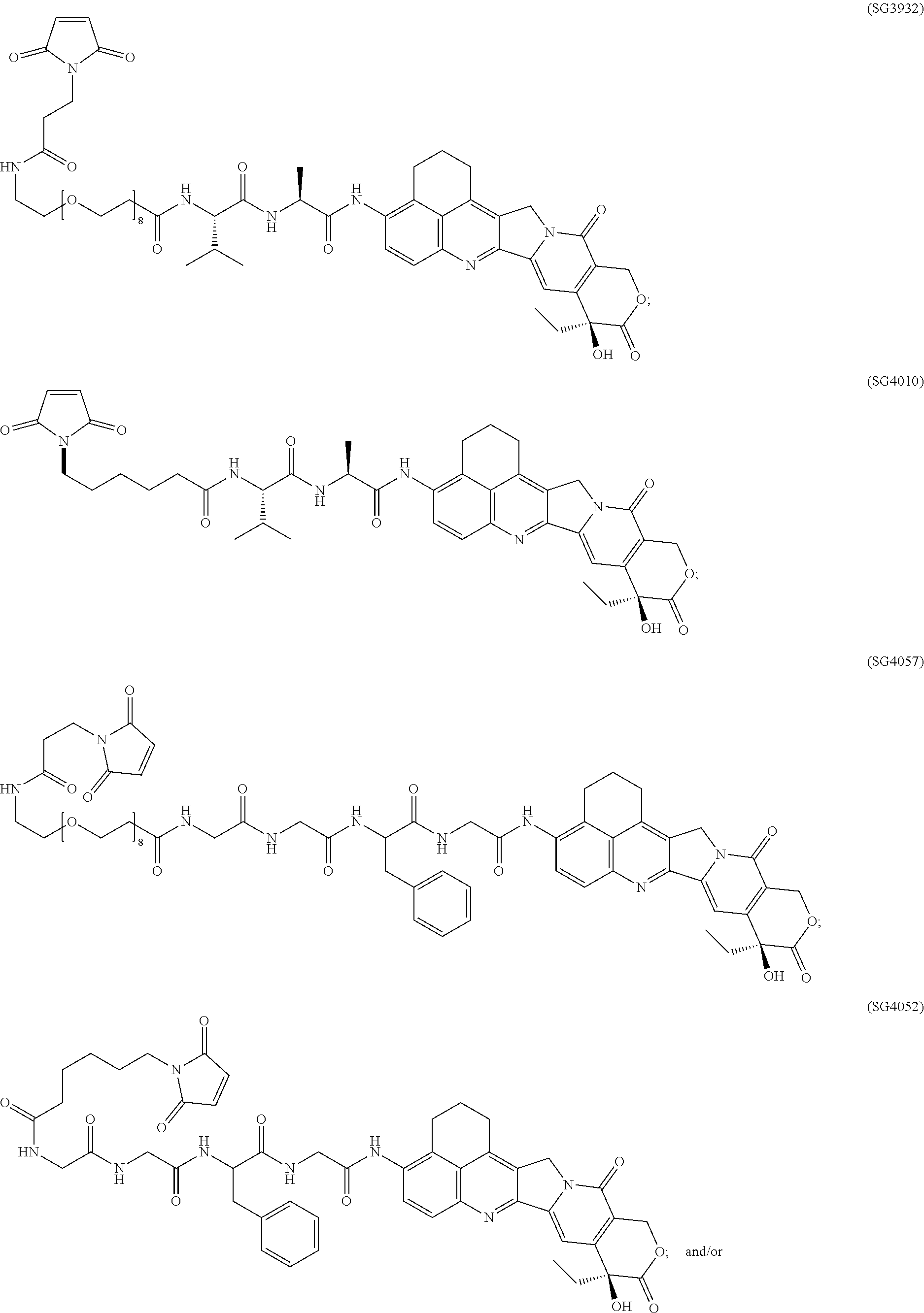

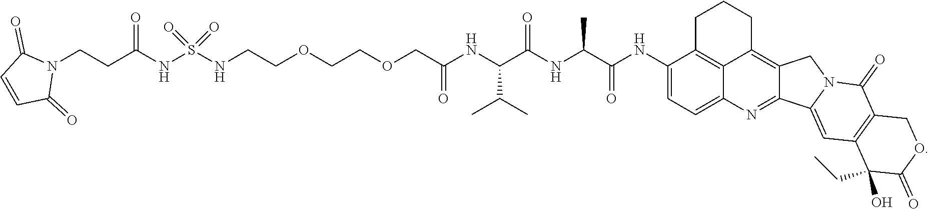 [SMILES: CC[C@@]1(O)C(=O)OCc2c1cc1n(c2=O)Cc2c-1nc1ccc(NC(=O)[C@H](C)NC(=O)[C@@H](NC(=O)COCCOCCNS(=O)(=O)NC(=O)CCN3C(=O)C=CC3=O)C(C)C)c3c1c2CCC3]

In some aspects, SG3932 is utilized. Thus, in some aspects, an antibody or antigen binding fragment thereof described herein is conjugated to a topoisomerase I inhibitor having the following formula (e.g. SG3932):

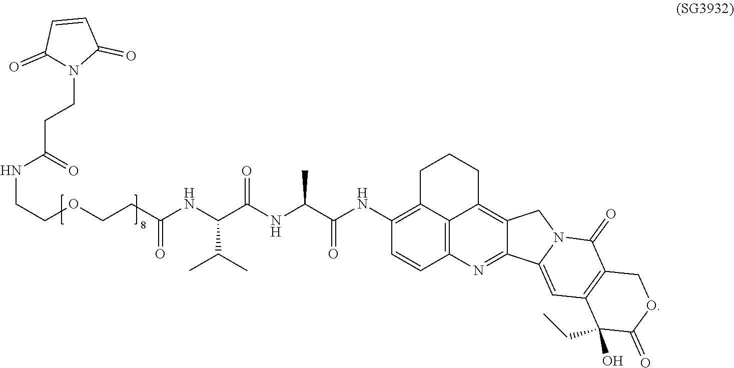

For the avoidance of doubt, the numeral ‘8’ specifies that the structure within boxed parentheses is repeated eight times. Thus, another representation of SG3932 is:

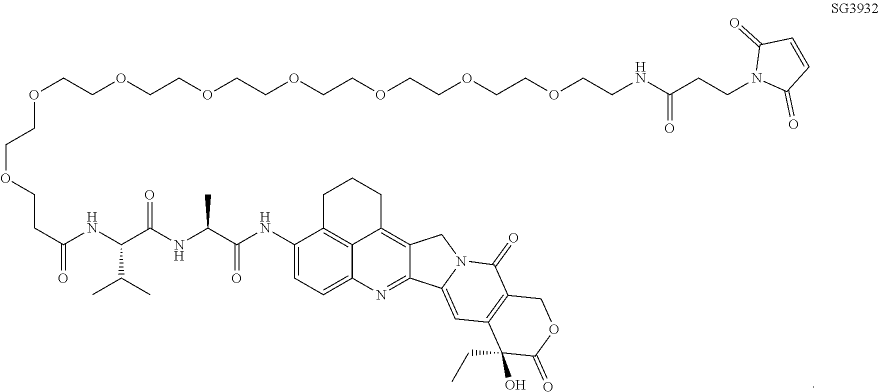 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

Another representation of SG4010 is:

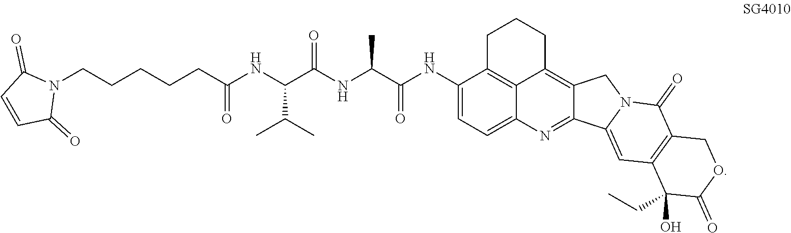 [SMILES: CC[C@@]1(O)C(=O)OCc2c1cc1n(c2=O)Cc2c-1nc1ccc(NC(=O)[C@H](C)NC(=O)[C@@H](NC(=O)CCCCCN3C(=O)C=CC3=O)C(C)C)c3c1c2CCC3]

Another representation of SG4057 is:

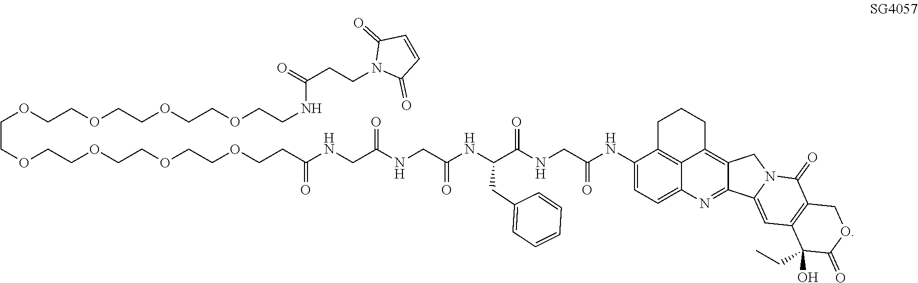 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCOCCOCCOCCOCCOCCOCCOCCOCCNC(=O)CCN3C(=O)C=CC3=O)c3c1c2CCC3]

Another representation of SG4052 is:

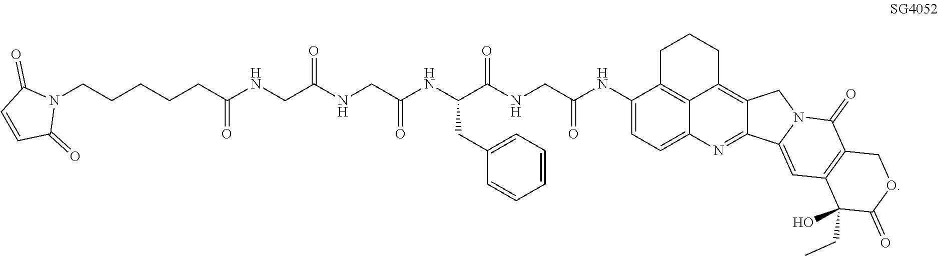 [SMILES: CC[C@@]1(O)C(=O)OCc2c1cc1n(c2=O)Cc2c-1nc1ccc(NC(=O)CNC(=O)[C@H](Cc3ccccc3)NC(=O)CNC(=O)CNC(=O)CCCCCN3C(=O)C=CC3=O)c3c1c2CCC3]

Any antibody or antigen binding fragment thereof described herein may be conjugated to one or more of said topoisomerase I inhibitor(s).

In one aspect, provided is an antibody or antigen binding fragment thereof which binds to a STEAP2 polypeptide (e.g. STEAP2 polypeptide epitope), comprising:
i. a HCDR1 comprising the amino acid sequence of SEQ ID NO: 1, or a functional variant thereof;
ii. a HCDR2 comprising the amino acid sequence of SEQ ID NO: 2, or a functional variant thereof;
iii. a HCDR3 comprising the amino acid sequence of SEQ ID NO: 3, or a functional variant thereof;
iv. a LCDR1 comprising the amino acid sequence of SEQ ID NO: 4, or a functional variant thereof;
v. a LCDR2 comprising the amino acid sequence of SEQ ID NO: 5, or a functional variant thereof; and
vi. a LCDR3 comprising the amino acid sequence of SEQ ID NO: 6, or a functional variant thereof;
vii. a HCDR1 comprising the amino acid sequence of SEQ ID NO: 7, or a functional variant thereof;
viii. a HCDR2 comprising the amino acid sequence of SEQ ID NO: 8, or a functional variant thereof;
iix. a HCDR3 comprising the amino acid sequence of SEQ ID NO: 9, or a functional variant thereof;
ix. a LCDR1 comprising the amino acid sequence of SEQ ID NO: 10, or a functional variant thereof;
x. a LCDR2 comprising the amino acid sequence of SEQ ID NO: 11, or a functional variant thereof; and
xi. a LCDR3 comprising the amino acid sequence of SEQ ID NO: 12, or a functional variant thereof;
wherein the antibody or antigen binding fragment thereof is conjugated to a SG3932:

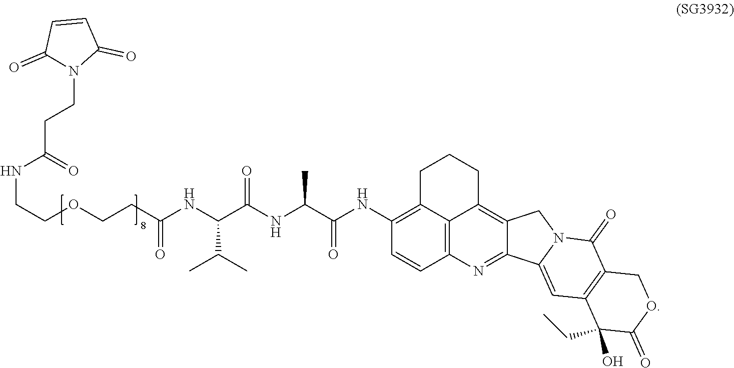

Another aspect provides an antibody or antigen binding fragment thereof comprising: a variable heavy chain comprising the amino acid sequence of SEQ ID NO: 31, or a functional variant thereof; and a variable light chain comprising the amino acid sequence of SEQ ID NO: 32, or a functional variant thereof;

wherein the antibody or antigen binding fragment thereof is conjugated to a SG3932:

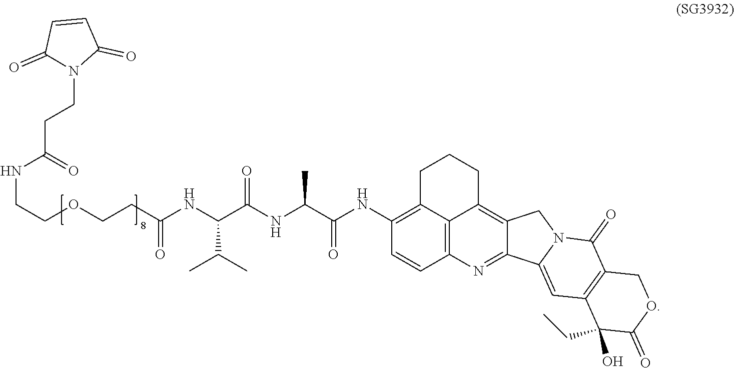

Another aspect provides an antibody or antigen binding fragment thereof comprising: a variable heavy chain comprising the amino acid sequence of SEQ ID NO: 31, or a functional variant thereof; and a variable light chain comprising the amino acid sequence of SEQ ID NO: 32, or a functional variant thereof;

wherein the antibody or antigen binding fragment thereof is conjugated to a SG3932:

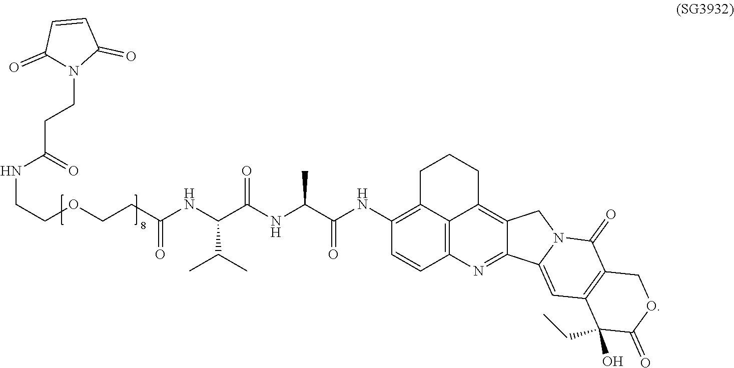

Another aspect provides and antibody or antigen binding fragment thereof comprising: a heavy chain comprising the amino acid sequence of SEQ ID NO: 33, or a functional variant thereof; and a light chain comprising the amino acid sequence of SEQ ID NO: 34, or a functional variant thereof;

wherein the antibody or antigen binding fragment thereof is conjugated to a SG3932:

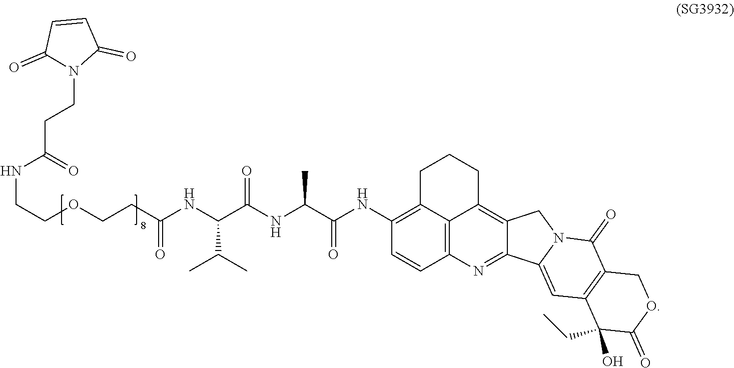

### Synthesis of Topoisomerase I Inhibitors

Illustrative examples of the synthesis of Topoisomerase I inhibitors and key intermediates are well-known in the art, and disclosed, for example, in WO 2020/200880, incorporated herein by reference.

### Amine Protecting Groups:

Amine protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

### Further ADCs

Although topoisomerase I inhibitors are as outlined above, it should be noted that any suitable agent (e.g. drug/cytotoxin) may be linked to an antibody or antigen binding fragment thereof of the disclosure. Examples of other suitable agents are outlined below.

In one aspect, the cytotoxin is a tubulysin or tubulysin derivative. In one aspect, the cytotoxin is Tubulysin A, having the following chemical structure:

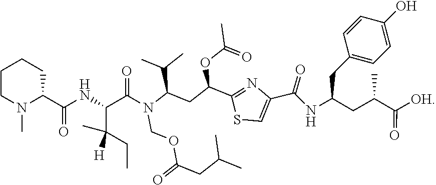 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

Tubulysins are members of a class of natural products isolated from myxobacterial species. As cytoskeleton-interacting agents, tubulysins are mitotic poisons that inhibit tubulin polymerization and lead to cell cycle arrest and apoptosis. As used herein, the term “tubulysin” refers both collectively and individually to the naturally occurring tubulysins and analogs and derivatives of tubulysins. Illustrative examples of tubulysins are disclosed, for example, in WO2004005326A2, WO2012019123A1, WO2009134279A1, WO2009055562A1, WO2004005327A1, U.S. Pat. Nos. 7,776,841, 7,754,885, US20100240701, U.S. Pat. No. 7,816,377, US20110021568, and US20110263650, incorporated herein by reference. It is to be understood that such derivatives include, for example, tubulysin prodrugs or tubulysins that include one or more protection or protecting groups, one or more linking moieties.

In one aspect, the cytotoxin is tubulysin 1508, also referred to herein as “AZ1508” and described in more detail in WO 2015157594, incorporated herein by reference, having the following structure:

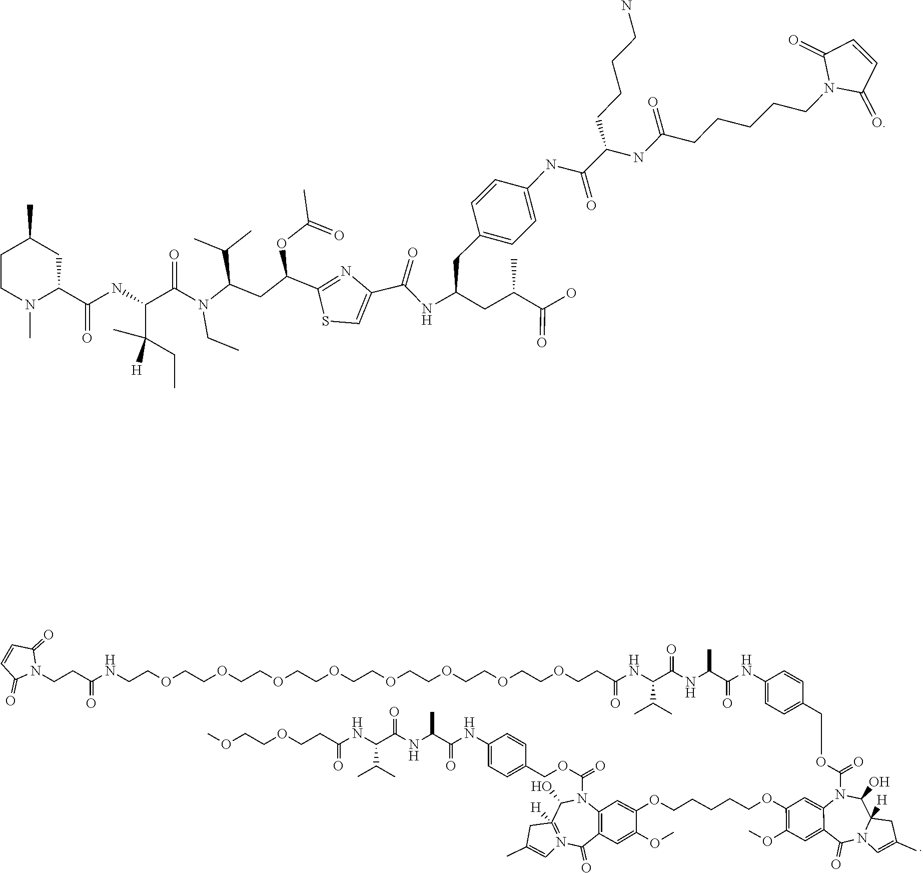

The antibody or antigen fragment thereof of the disclosure may be conjugated to heterologous agents (such as a cytotoxin) using site-specific or non-site specific methods of conjugation. In one aspect, the antibodies and antigen fragment thereof comprise one, two, three, four or more therapeutic moieties. In one aspect, all therapeutic moieties are the same.

Conventional conjugation strategies for antibodies or antigen-binding fragments thereof rely on randomly conjugating the payload to the antibody or fragment through lysines or cysteines. In one aspect, the antibody or antigen-binding fragment thereof is randomly conjugated to a heterologous agent (such as a cytotoxin), for example, by partial reduction of the antibody or fragment, followed by reaction with a desired agent, with or without a linker moiety attached. The antibody or fragment may be reduced using DTT or similar reducing agent. The agent with or without a linker moiety attached can then be added at a molar excess to the reduced antibody or fragment in the presence of DMSO. After conjugation, excess free cysteine may be added to quench unreacted agent. The reaction mixture may then be purified and buffer-exchanged into PBS.

Through transcriptomic and proteomic profiling of various solid tumour cell lines, β-glucuronidase expression has been identified as being upregulated and typically localised to the lysosome. WO2007011968 and WO2015182984 disclose certain antibody drug conjugates comprising β-glucuronidase-cleavable linkers. There remains a need for conjugates having β-glucuronidase-cleavable linkers that can selectively deliver a drug to a biological target and that have favourable physicochemical properties, including solubility and lipophilicity. The conjugates of the disclosure may be used for the treatment of diseases such as cancer.

In other aspects there is provided a conjugate of Formula (IC):

Ab-(G^{A}-J^{A}-D^{C})ₖ (IC),

or a pharmaceutically acceptable salt thereof, wherein Ab is an antibody or antigen-binding fragment thereof, k is an integer from 1 to 10, each G^{A} is independently a conjugation group conjugated to the antibody or antigen-binding fragment thereof, each D^{C} is

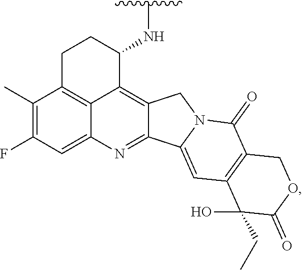 [SMILES: *N[C@H]1CCc2c(C)c(F)cc3nc4c(c1c23)Cn1c-4cc2c(c1=O)COC(=O)[C@]2(O)CC]

each J^{A} is independently a group of Formula (ICA)

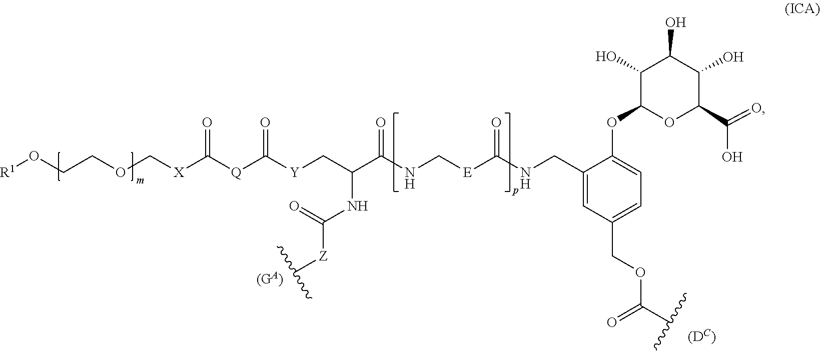

E is (CH₂)ₙ₁, wherein n1 is 0, 1, 2 or 3,
Q is

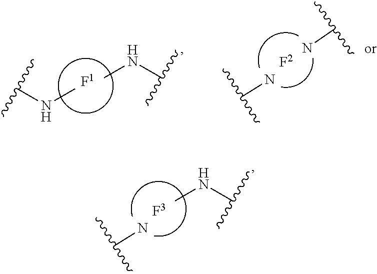

wherein Ring F¹ is a saturated bicyclic ring having 6, 7, or 8 carbon atoms and optionally 1 or 2 oxygen atoms, Ring F² is a saturated bicyclic ring having the 2 nitrogen atoms shown, 4, 5, 6, 7 or 8 carbon atoms and optionally 1 oxygen atom, and Ring F³ is a saturated bicyclic ring having the 1 nitrogen atom shown, 5, 6, 7 or 8 carbon atoms and optionally 1 oxygen atom,
R¹ is C₁₋₄ alkyl,
X is (CH₂)ₙ₂, wherein n2 is 0, 1, 2 or 3,
Y is (CH₂)ₙ₃, wherein n3 is 0, 1, 2, 3 or 4,
Z is (CH₂)ₙ₄, wherein n4 is 1, 2, 3, 4 or 5,
m is an integer from 5 to 17,
p is 1 or 0,
(G^{A}) indicates the point of attachment to G^{A}, and
(D^{C}) indicates the point of attachment to D^{C}.

In a further aspect there is provided a pharmaceutical composition comprising a conjugate of Formula (IC), or a pharmaceutically acceptable salt thereof and a pharmaceutically acceptable excipient.

In a further aspect there is provided a conjugate of Formula (IC), or a pharmaceutically acceptable salt thereof, for use in therapy.

In a further aspect there is provided a conjugate of Formula (IC), or a pharmaceutically acceptable salt thereof, for use in the treatment of cancer.

In a further aspect there is provided the use of a conjugate of Formula (IC), or a pharmaceutically acceptable salt thereof, in the manufacture of a medicament.

In a further aspect there is provided the use of a conjugate of Formula (IC), or a pharmaceutically acceptable salt thereof, in the manufacture of a medicament for the treatment of cancer.

In a further aspect there is provided a method of treating cancer in a patient comprising administering to the patient an effective amount of a conjugate of Formula (IC), or a pharmaceutically acceptable salt thereof.

A conjugate of Formula (IC), or a pharmaceutically acceptable salt thereof, may undergo enzymatic cleavage to release a free drug (exatecan). Conjugates of Formula (IC) may exhibit improved efficacy and/or advantageous physical properties (for example, higher stability, lower lipophilicity, higher aqueous solubility, higher permeability and/or lower plasma protein binding), and/or favourable toxicity profiles (for example reduced off target toxicity), and/or favourable metabolic or pharmacokinetic profiles, in comparison with other conjugates. As such, conjugates of Formula (IC) may especially be suitable for use in therapy, such as the treatment of cancer.

So that the present specification may be more readily understood, certain terms are explicitly defined below. In addition, definitions are set forth as appropriate throughout the detailed description. Where examples are provided for a definition, they are not limiting.

The prefix C_{x-y}, where x and y are integers, indicates the numerical range of carbon atoms that are present in a group.

As used herein the term “alkyl” refers to a saturated, linear or branched hydrocarbon radical having the specified number of carbon atoms. Examples of C₁₋₄ alkyl groups include methyl (Me), ethyl (Et), n-propyl (ⁿPr), i-propyl (ⁱPr), n-butyl (ⁿBu), i-butyl (ⁱBu), s-butyl (^{s}Bu), and t-butyl (^{t}Bu). Examples of C₁₋₆ alkyl groups include methyl, ethyl, n-propyl, i-propyl, n-butyl, i-butyl, s-butyl, t-butyl, n-pentyl and n-hexyl.

As used herein the term “bicyclic ring” refers to a fused, bridged or spirocyclic bicyclic ring.

As used herein the term “conjugation group for conjugation to an antibody, or antigen-binding fragment thereof” refers to an atom or group of atoms capable of forming a covalent bond to an antibody, or antigen-binding fragment thereof, through a chemical reaction.

The use of “ [symbol] ” in formulas of this specification indicates the point of attachment to the antibody or antigen-binding fragment thereof. By way of illustration

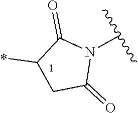 [SMILES: *C1CC(=O)N(*)C1=O]

indicates that that there is a covalent bond connecting the antibody, or antigen-binding fragment thereof, to the carbon atom marked 1.

For the avoidance of doubt, the use of “ [symbol] ” in formulas of this specification denotes the point of covalent attachment to a group, where the group is other than the antibody or antigen-binding fragment thereof.

Certain embodiments of this specification include a group which is said to be “optionally substituted”. In further embodiments said group is unsubstituted.

As used herein

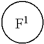

is ring F¹,

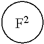

is ring F², and

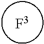

is ring F³.

Units, prefixes, and symbols are denoted in their International System of Units (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

In one aspect, this specification provides a conjugate of Formula (IC), or a pharmaceutically acceptable salt thereof, as defined above.

In aspects there is provided a conjugate of Formula (IC), or a pharmaceutically acceptable salt thereof, wherein k is 1, 2, 3, 4, 5, 6, 7, 8, 9 or 10. In further aspects k is an integer from 2 to 10. In further aspects k is an integer from 2 to 8. In further aspects k is about 4. In further aspects k is about 8. In further aspects k is 4. In further aspects k is 8.

In aspects there is provided a conjugate of Formula (IC), or a pharmaceutically acceptable salt thereof, wherein G^{A} is selected from

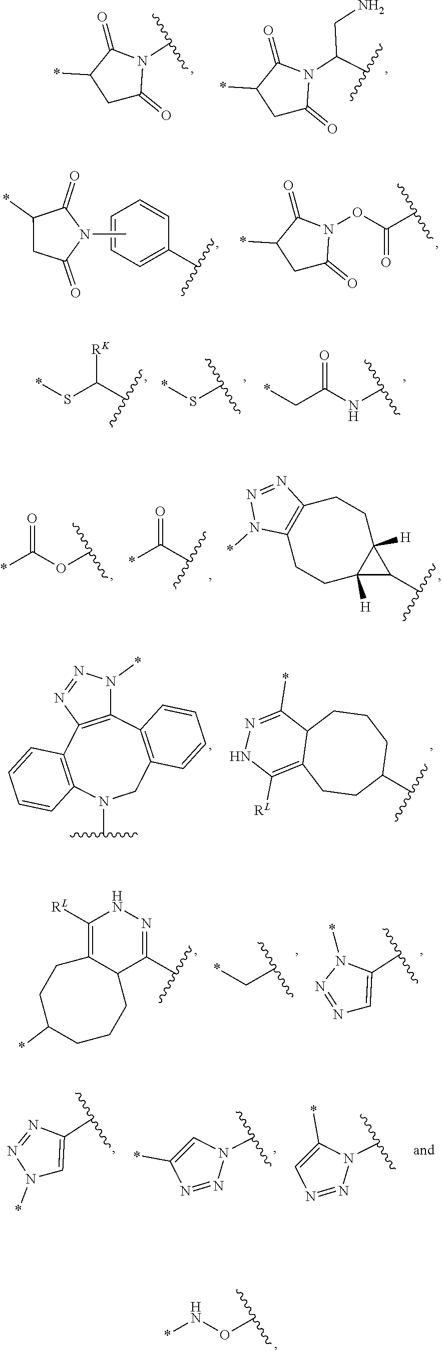

wherein R^{K} is H or CH₃, R^{L} is C₁₋₆ alkyl, and [symbol] indicates the point of attachment to the antibody, or antigen-binding fragment thereof.

In aspects there is provided a conjugate of Formula (IC), or a pharmaceutically acceptable salt thereof, wherein G^{A} is selected from

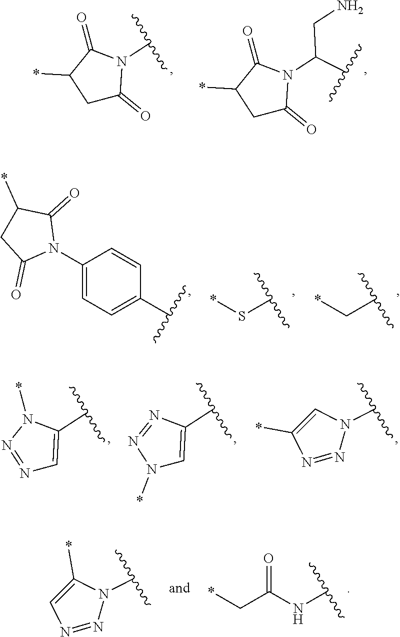

In aspects there is provided a conjugate of Formula (IC), or a pharmaceutically acceptable salt thereof, wherein G^{A} is

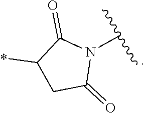 [SMILES: *C1CC(=O)N(*)C1=O]

In aspects there is provided a conjugate of Formula (IC), or a pharmaceutically acceptable salt thereof, wherein G^{A} is

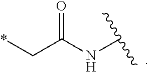 [SMILES: *CC(=O)N*]

In aspects there is provided a conjugate of Formula (IC), or a pharmaceutically acceptable salt thereof, wherein Q is

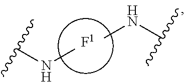

and wherein ring F¹ is a saturated bicyclic ring having 6, 7 or 8 carbon atoms and optionally 1 or 2 oxygen atoms. In further aspects the bicyclic ring is a fused bicyclic ring.

In aspects there is provided a conjugate of Formula (IC), or a pharmaceutically acceptable salt thereof, wherein Q is

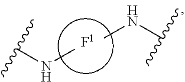

and ring F¹ is a saturated bicyclic ring having 6, 7 or 8 carbon atoms and 1 or 2 oxygen atoms. In further aspects the bicyclic ring is a fused bicyclic ring.

In aspects there is provided a conjugate of Formula (IC), or a pharmaceutically acceptable salt thereof, wherein Q is

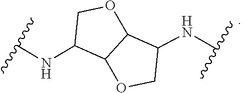 [SMILES: *NC1COC2C(N*)COC12]

In aspects there is provided a conjugate of Formula (IC), or a pharmaceutically acceptable salt thereof, wherein Q is

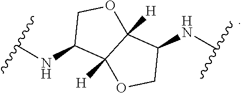 [SMILES: *N[C@H]1CO[C@H]2[C@@H]1OC[C@@H]2N*]

In aspects there is provided a conjugate of Formula (IC), or a pharmaceutically acceptable salt thereof, wherein Q is

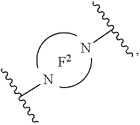

and wherein Ring F² is a saturated bicyclic ring having the 2 nitrogen atoms shown, 4, 5, 6, 7 or 8 carbon atoms and optionally 1 oxygen atom. In further aspects the bicyclic ring is a spirocyclic bicyclic ring. In further aspects the bicyclic ring is a bridged bicyclic ring.

In aspects there is provided a conjugate of Formula (IC), or a pharmaceutically acceptable salt thereof, wherein Q is

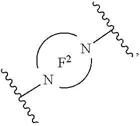

and wherein Ring F² is a saturated bicyclic ring having the 2 nitrogen atoms shown and 4, 5, 6, 7 or 8 carbon atoms. In further aspects the bicyclic ring is a spirocyclic bicyclic ring. In further aspects the bicyclic ring is a bridged bicyclic ring.

In aspects there is provided a conjugate of Formula (IC), or a pharmaceutically acceptable salt thereof, wherein Q is

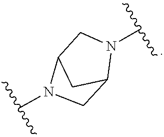 [SMILES: *N1CC2CC1CN2*]

In aspects there is provided a conjugate of Formula (IC), or a pharmaceutically acceptable salt thereof, wherein Q is

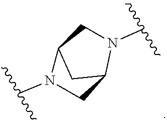 [SMILES: *N1C[C@H]2C[C@@H]1CN2*]

In aspects there is provided a conjugate of Formula (IC), or a pharmaceutically acceptable salt thereof, wherein Q is

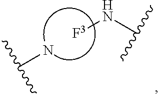

wherein Ring F³ is a saturated bicyclic ring having the 1 nitrogen atom shown, 5, 6, 7 or 8 carbon atoms and optionally 1 oxygen atom. In further aspects the bicyclic ring is a spirocyclic bicyclic ring. In further aspects the bicyclic ring is a bridged bicyclic ring.

In aspects there is provided a conjugate of Formula (IC), or a pharmaceutically acceptable salt thereof, wherein Q is

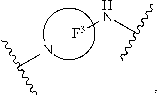

wherein Ring F³ is a saturated bicyclic ring having the 1 nitrogen atom shown and 5, 6, 7 or 8 carbon atoms. In further aspects the bicyclic ring is a spirocyclic bicyclic ring. In further aspects the bicyclic ring is a bridged bicyclic ring.

In aspects there is provided a conjugate of Formula (IC), or a pharmaceutically acceptable salt thereof, wherein E is (CH₂)ₙ₁, wherein n1 is 0, 1, 2 or 3. In further aspects E is a covalent bond. In further aspects E is CH₂. In further aspects E is (CH₂)₂. In further aspects E is (CH₂)₃.

In aspects there is provided a conjugate of Formula (IC), or a pharmaceutically acceptable salt thereof, wherein X is (CH₂)ₙ₂, wherein n2 is 0, 1, 2 or 3. In further aspects X is a covalent bond. In further aspects X is CH₂. In further aspects X is (CH₂)₂. In further aspects X is (CH₂)₃.

In aspects there is provided a conjugate of Formula (IC), or a pharmaceutically acceptable salt thereof, wherein Y is (CH₂)ₙ₃, wherein n3 is 0, 1, 2, 3 or 4. In further aspects Y is a covalent bond. In further aspects Y is CH₂. In further aspects Y is (CH₂)₂. In further aspects Y is (CH₂)₃. In further aspects Y is (CH₂)₄.

In aspects there is provided a conjugate of Formula (IC), or a pharmaceutically acceptable salt thereof, wherein Z is (CH₂)ₙ₄, wherein n4 is 1, 2, 3, 4 or 5. In further aspects Z is CH₂. In further aspects Z is (CH₂)₂. In further aspects Z is (CH₂)₃. In further aspects Z is (CH₂)₄. In further aspects Z is (CH₂)₅.

In aspects there is provided a conjugate of Formula (IC), or a pharmaceutically acceptable salt thereof, wherein m is 5, 6, 7, 8, 9, 10, 11, 12, 13, 14, 15, 16 or 17. In further aspects m is an integer from 6 to 16. In further aspects m is an integer from 7 to 15. In further aspects m is an integer from 8 to 14. In further aspects m is an integer from 9 to 13. In further aspects m is an integer from 10 to 12. In further aspects m is 11.

In aspects there is provided a conjugate of Formula (IC), or a pharmaceutically acceptable salt thereof, wherein R¹ is C₁₋₄ alkyl. In further aspects R¹ is CH₃.

In aspects there is provided a conjugate of Formula (IC), or a pharmaceutically acceptable salt thereof, wherein p is 1.

In aspects there is provided a conjugate of Formula (IC), or a pharmaceutically acceptable salt thereof, wherein each J^{A} is a group of Formula (ICB)

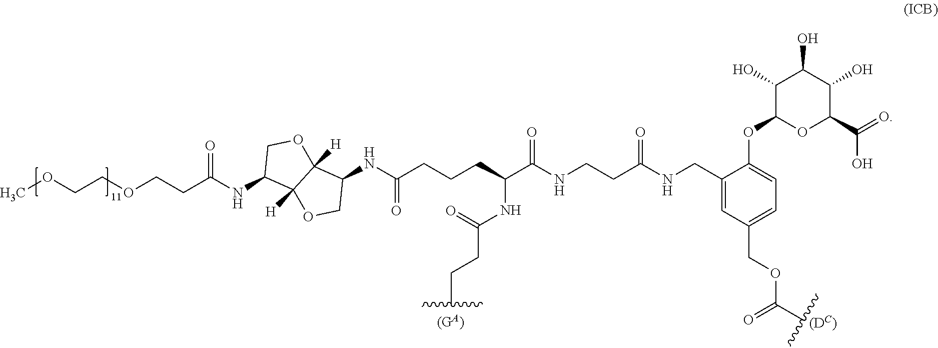

In aspects there is provided a conjugate of Formula (IC), or a pharmaceutically acceptable salt thereof, wherein each J^{A} is a group of Formula (ICB′)

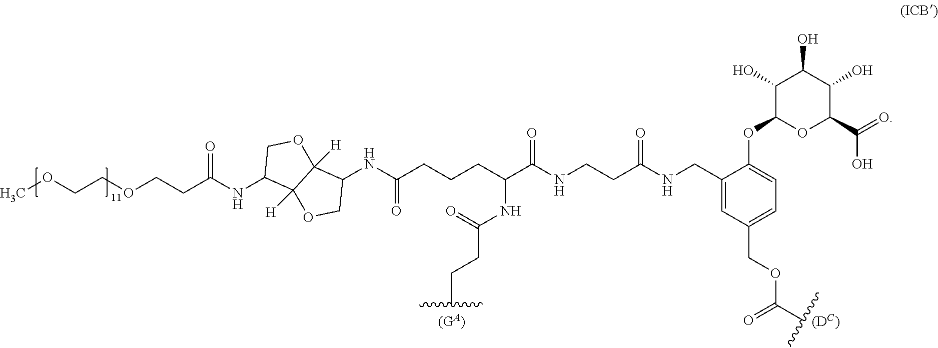

In aspects there is provided a conjugate of Formula (IC), or a pharmaceutically acceptable salt thereof, wherein each J^{A} is a group of Formula (ICB2)

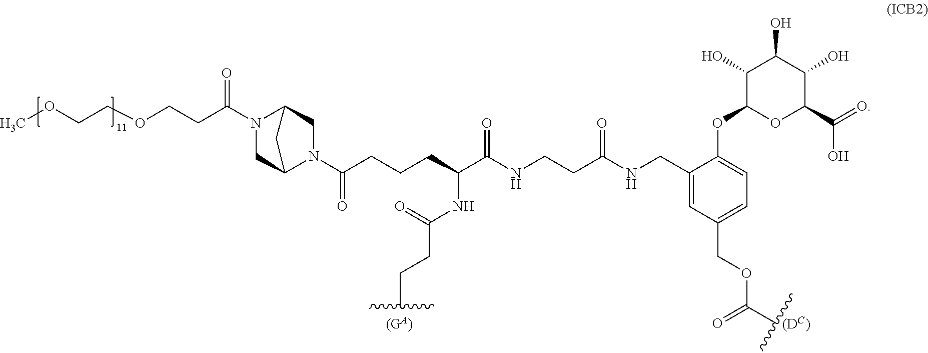

The present specification is intended to include all isotopes of atoms occurring in the present compounds and conjugates. Isotopes will be understood to include those atoms having the same atomic number but different mass numbers. For example, isotopes of hydrogen include tritium and deuterium. Isotopes of carbon include ¹³C and ¹⁴C. Isotopes of nitrogen include ¹⁵N.

The compounds disclosed herein may contain one or more chiral centers. Accordingly, if desired, such compounds can be prepared or isolated as pure stereoisomers, i.e. as individual enantiomers, diastereoisomers, or as a stereoisomerically enriched mixture. All such stereoisomer (and enriched) mixtures are included within the scope of the aspects, unless otherwise stated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless stereochemistry is explicitly indicated in a chemical structure or chemical name, the chemical structure or chemical name is intended to embrace all possible stereoisomers, diastereoisomers, conformers, rotamers and tautomers of the compound depicted. For example, a compound containing a chiral carbon atom is intended to embrace both the (R) enantiomer and the (S) enantiomer, as well as mixtures of the enantiomers, including racemic mixtures; and a compound containing two chiral carbons is intended to embrace all enantiomers and diastereoisomers including (R,R), (S,S), (R,S) and (S,R).

In one aspect, an agent (e.g. cytotoxin) is conjugated to an antibody or antigen binding fragment thereof by site-specific conjugation. In one aspect, site-specific conjugation of therapeutic moieties to antibodies using reactive amino acid residues at specific positions yields homogeneous ADC preparations with uniform stoichiometry.

The site specific conjugation can be through a cysteine, residue or a non-natural amino acid. In one aspect, the heterologous agent (such as a cytotoxin) is conjugated to the antibody or antigen binding fragment thereof through at least one cysteine residue.

In one aspect, the heterologous agent (such as a cytotoxin) is chemically conjugated to the side chain of an amino acid (e.g. at a specific Kabat position in the Fc region). In one aspect, the agent (e.g. the cytotoxic or imaging agent) is conjugated to the antibody or antigen binding fragment thereof through a cysteine substitution of at least one of positions 239, 248, 254, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 440, 441, 442, 443 and 446, wherein the numbering corresponds to the EU index in Kabat. In one aspect, the specific Kabat positions are 239, 442, or both. In one aspect, the specific positions are Kabat position 442, an amino acid insertion between Kabat positions 239 and 240, or both. In one aspect, the heterologous agent (such as a cytotoxin) is conjugated to the antibody or antigen binding fragment thereof through a thiol-maleimide linkage. In some aspects, the amino acid side chain is a sulfhydryl side chain.

Reference herein to an antibody or antigen-binding fragment conjugated to a cytotoxin is synonymous with the term “antibody drug conjugate (ADC)”, or “anti-STEAP2 ADC”.

In one aspect, the antibody or antigen binding fragment thereof (e.g., anti-STEAP2 ADC) delivers a cytotoxic payload to a cell (such as a STEAP2-expressing cell) and inhibits or suppresses proliferation (e.g. of a tumor) by at least 10%, or at least 20%, or at least 30%, or at least 40%, or at least 50%, or at least 60%, or at least 70%, or at least 80%, or at least 90% or about 100% (at least 40%) relative to a level of inhibition or suppression in the absence of the antibody or antigen binding fragment thereof (e.g., anti-STEAP2 ADC). Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

In one aspect, the antibody or antigen fragment thereof (e.g., anti-STEAP2 ADC) of the disclosure binds to STEAP2 on the surface of a cell, and is internalized into the cell. In one aspect, the antigen or antibody fragment thereof is internalized into a cell (such as a STEAP2-expressing cell) with an IC50 at 10 minutes of about 100 ng/ml to about 1 μg/ml, about 100 ng/ml to about 500 ng/ml, about 100 ng/ml to about 250 ng/ml, about 250 ng/ml to about 500 ng/ml, about 350 ng/ml to about 450 ng/ml, about 500 ng/ml to about 1 μg/ml, about 500 ng/ml to about 750 ng/ml, about 750 ng/ml to about 850 ng/ml, or about 900 ng/ml to about 1 μg/ml.

In one aspect, the antibody or antigen fragment thereof (e.g., anti-STEAP2 ADC) is internalized into a cell (such as a STEAP2-expressing cell) with an IC50 at 30 minutes of about 100 ng/ml to about 1 μg/ml, about 100 ng/ml to about 500 ng/ml, about 100 ng/ml to about 250 ng/ml, about 250 ng/ml to about 500 ng/ml, about 250 ng/ml to about 350 ng/ml, about 350 ng/ml to about 450 ng/ml, about 500 ng/ml to about 1 μg/ml, about 500 ng/ml to about 750 ng/ml, about 750 ng/ml to about 850 ng/ml, or about 900 ng/ml to about 1 μg/ml.

In one aspect, the antibody or antigen fragment thereof (e.g., anti-STEAP2 ADC) is internalized into a cell (such as a STEAP2-expressing cell) with an IC50 at 120 minutes of about 50 ng/ml to about 500 ng/ml, about 50 ng/ml to about 100 ng/ml, about 100 ng/ml to about 200 ng/ml, about 200 ng/ml to about 300 ng/ml, about 300 ng/ml to about 400 ng/ml, or about 400 ng/ml to about 500 ng/ml.

In one aspect, the antibody or antigen fragment thereof (e.g., anti-STEAP2 ADC) is internalized into a cell (such as a STEAP2-expressing cell) with an IC50 at 8 hours of about 5 ng/ml to about 250 ng/ml, about 10 ng/ml to about 25 ng/ml, about 25 ng/ml to about 50 ng/ml, about 50 ng/ml to about 100 ng/ml, about 100 ng/ml to about 150 ng/ml, about 150 ng/ml to about 200 ng/ml, or about 200 ng/ml to about 250 ng/ml.

For the avoidance of doubt, reference to a “conjugate” herein means an antibody or antigen binding fragment conjugated to a heterologous agent (such as a cytotoxin) including any such agent described above.

In addition to the therapeutic applications of an antibody or antigen binding fragment of the disclosure described above, the “conjugates” of the present disclosure may be also used in a method of therapy. Thus, also provided is a method of treatment, comprising administering to a subject in need of treatment a therapeutically effective amount of a conjugate described herein (e.g. conjugate of formula IV). The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g. drugs); surgery; and radiation therapy.

Pharmaceutical compositions according to the present disclosure, and for use in accordance with the present disclosure, may comprise, in addition to the active ingredient, i.e. a conjugate/ADC of the disclosure (e.g. formula IV), a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

In some aspects, the conjugates can be used to treat proliferative disease. The term “proliferative disease” pertains to an unwanted or uncontrolled cellular proliferation of excessive or abnormal cells which is undesired, such as, neoplastic or hyperplastic growth, whether in vitro or in vivo. The term “proliferative disease” may alternatively be referred to as “cancer”.

A suitable proliferative disease (e.g. cancer) can be characterized by the presence cancerous cells that express STEAP2.

Examples of proliferative conditions include, but are not limited to, benign, pre-malignant, and malignant cellular proliferation, including but not limited to, neoplasms and tumors (e.g., histocytoma, glioma, astrocyoma, osteoma), cancers (e.g. lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreatic cancer, brain cancer, sarcoma, osteosarcoma, Kaposi's sarcoma, melanoma), leukemias, psoriasis, bone diseases, fibroproliferative disorders (e.g. of connective tissues), and atherosclerosis. Other cancers of interest include, but are not limited to, haematological; malignancies such as leukemias and lymphomas, such as non-Hodgkin lymphoma, and subtypes such as DLBCL, marginal zone, mantle zone, and follicular, Hodgkin lymphoma, AML, and other cancers of B or T cell origin. Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

The antibody-drug conjugate may be labelled, for example to aid detection of cell binding (in vitro or in vivo). The label may be a biotin label. In another aspect, the label may be a radioisotope.

In another aspect, there is provided a polynucleotide comprising a nucleic acid sequence encoding an antibody or antigen binding fragment thereof of the disclosure.

In one aspect, the polynucleotide may be an isolated polynucleotide.

The sequence(s) (e.g. polynucleotide sequence(s)) of the present disclosure include sequences that have been removed from their naturally occurring environment, recombinant or cloned (e.g. DNA) isolates, and chemically synthesized analogues or analogues biologically synthesized by heterologous systems.

The sequence(s) (e.g. polynucleotide sequence(s)) of the present disclosure may be prepared by any means known in the art. For example, large amounts of the sequence(s) may be produced by replication and/or expression in a suitable host cell. The natural or synthetic DNA fragments coding for a desired fragment will typically be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured bacterial, insect, mammalian, plant or other eukaryotic cell lines.

The sequence(s) (e.g. polynucleotide sequence(s)) of the present disclosure may also be produced by chemical synthesis, e.g. a polynucleotide by the phosphoramidite method or the tri-ester method and may be performed on commercial automated oligonucleotide synthesizers. A double-stranded (e.g. DNA) fragment may be obtained from the single stranded product of chemical synthesis either by synthesizing the complementary strand and annealing the strand together under appropriate conditions or by adding the complementary strand using DNA polymerase with an appropriate primer sequence.

When applied to a sequence (e.g. polynucleotide sequence) of the disclosure, the term “isolated” denotes that the sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

Another aspect provided herein is a host cell comprising a polynucleotide, said polynucleotide comprising a nucleic acid sequence encoding an antibody or antigen binding fragment thereof of the disclosure.

In one aspect, the polynucleotide encodes a VH chain of an antibody or antigen binding fragment thereof. In one aspect, a polynucleotide of the disclosure may encode a VL chain of an antibody or antigen binding fragment thereof. In one aspect, the polynucleotide may encode a VH and a VL chain of an antibody or antigen binding fragment thereof. In one aspect, the polynucleotide may further encode a leader sequence (e.g. which functions as a secretory sequence for controlling transport of a polypeptide from the cell).

In another aspect there is provided a vector (e.g. plasmid) comprising the polynucleotide of the disclosure.

Variants of a polynucleotide described above are embraced by the disclosure. Polynucleotide variants can contain alterations in the coding regions, non-coding regions, or both. In one aspect, a polynucleotide variant comprises an alteration that produces silent substitutions, additions, or deletions, but do not alter the properties or activities of the encoded polypeptide. In one aspect, a polynucleotide variant is produced by a silent substitution due to the degeneracy of the genetic code. A polynucleotide variant can be produced for a variety of reasons, e.g., to optimize codon expression for a particular host (change codons in the human mRNA to those preferred by a bacterial host such as E. coli). Vectors and cells comprising said polynucleotide variant are also provided.

The present disclosure embraces methods for producing an antibody or antigen binding fragment thereof that binds to a STEAP2 polypeptide (e.g. STEAP2 polypeptide epitope), comprising expressing a polynucleotide in a host cell, said polynucleotide comprising a nucleic acid sequence encoding an antibody or antigen binding fragment thereof of the disclosure.

The present disclosure further embraces an antibody or antigen binding fragment thereof obtainable by said methods for producing an antibody or antigen binding fragment thereof that binds to a STEAP2 polypeptide (e.g. STEAP2 polypeptide epitope).

In one aspect, the method for producing an antibody or antigen binding fragment thereof comprises (a) culturing the host cell and (b) isolating the antibody or antigen binding fragment thereof expressed from the cell.

Suitable host cells for expression of an antibody or antigen binding fragment thereof of the disclosure include a prokaryote, yeast, insect, or higher eukaryotic cells (e.g. wherein the polynucleotide is under the control of appropriate promoters). Prokaryotes include gram negative or gram-positive organisms, for example E. coli or bacilli. Higher eukaryotic cells include established cell lines of mammalian origin as described herein. Cell-free translation systems can also be employed.

In one aspect, there is provided a kit comprising an antigen or antibody binding fragment described herein. There is further embraced use of said kit in the methods of the present disclosure.

In one aspect, a kit comprises an isolated (e.g. purified) antigen or antibody binding fragment of the disclosure. In one aspect, a kit comprises an isolated (e.g. purified) antigen or antibody binding fragment of the disclosure, wherein the antigen or antibody binding fragment comprises an agent (e.g. conjugated cytotoxin) described herein. In one aspect, the kit comprises one or more container. The kit may provide the antigen or antibody binding fragment and the agent individually (e.g. the agent is not conjugated to the antigen or antibody binding fragment, but is in a form suitable for conjugation thereto); optionally wherein the kit is further provided with instructions and/or reagents for conjugating the agent to the antigen or antibody binding fragment. In one aspect, the kit comprises all of the components necessary and/or sufficient to perform a detection assay, including all controls, directions for performing assays, and any necessary software for analysis and presentation of results.

An antibody or antigen binding fragment thereof of the disclosure can be used in assays for immunospecific binding by any method known in the art. The immunoassays that can be used include, but are not limited to, competitive and non-competitive assay systems using techniques such as Western blot, RIA, ELISA, ELISPOT, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

An antibody or antigen binding fragment thereof of the disclosure can be employed histologically, as in immunofluorescence, immunoelectron microscopy, or non-immunological assays, for example, for in situ detection of STEAP2 or conserved variants or peptide fragments thereof. In situ detection can be accomplished by removing a histological specimen from a patient, and applying thereto a labelled an antibody or antigen binding fragment thereof of the disclosure, e.g., applied by overlaying the labelled antibody or antigen binding fragment thereof onto a biological sample. Through the use of such a procedure, it is possible to determine not only the presence of STEAP2, or conserved variants or peptide fragments, but also its distribution in the examined tissue. Using the present disclosure, those of ordinary skill will readily perceive that any of a wide variety of histological methods (such as staining procedures) can be modified in order to achieve such in situ detection.

### Antibodies

The term “antibody” covers monoclonal antibodies and fragments thereof (e.g. exhibiting the desired biological activity). In one aspect, an antibody of the present disclosure is a monoclonal antibody. In another aspect, the antibody is a fully human monoclonal antibody. In one aspect, methods of the disclosure may employ polyclonal antibodies.

An antibody is a protein including at least one or two, heavy (H) chain variable regions (abbreviated herein as VHC), and at least one or two light (L) chain variable regions (abbreviated herein as VLC). The VHC and VLC regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. Sequences of Proteins of Immunological Interest, Fifth Edition, U.S. Department of Health and Human Services, NIH Publication No. 91-3242, 1991, and Chothia, C. et al, J. MoI. Biol. 196:901-917, 1987). Each VHC and VLC is composed of three CDRs and four FRs, arranged from amino-terminus to carboxy-terminus in the following order: FR1, CDR1, FR2, DR2, FR3, CDR3, FR4. The VHC or VLC chain of the antibody can further include all or part of a heavy or light chain constant region. In one aspect, the antibody is a tetramer of two heavy immunoglobulin chains and two light immunoglobulin chains, wherein the heavy and light immunoglobulin chains are interconnected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The term “antibody” includes intact immunoglobulins of types IgA, IgG, IgE, IgD, IgM (as well as subtypes thereof), wherein the light chains of the immunoglobulin may be of types kappa or lambda. The term antibody, as used herein, also refers to a portion of an antibody that binds to one of the above-mentioned markers, e.g., a molecule in which one or more immunoglobulin chains is not full length, but which binds to a marker. Examples of binding portions encompassed within the term antibody include (i) a Fab fragment, a monovalent fragment consisting of the VLC, VHC, CL and CH1 domains; (ii) a F(ab′)₂ fragment, a bivalent fragment comprising two Fab fragments linked by a disulfide bridge at the hinge region; (iii) a Fc fragment consisting of the VHC and CH1 domains; (iv) a Fv fragment consisting of the VLC and VHC domains of a single arm of an antibody, (v) a dAb fragment (Ward et al, Nature 341:544-546, 1989), which consists of a VHC domain; and (vi) an isolated complementarity determining region (CDR) having sufficient framework to bind, e.g. an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. (1988) Science 1Al-ATi-Alβ; and Huston et al. (1988) Proc. Natl. Acad. ScL USA 85:5879-5883). Such single chain antibodies are also encompassed within the term antibody. These may be obtained using conventional techniques known to those skilled in the art, and the portions are screened for utility in the same manner as are intact antibodies.

In one aspect, the antibody or antigen binding fragment is one or more selected from a murine antibody, a humanized antibody, a chimeric antibody, a monoclonal antibody, a polyclonal antibody, a recombinant antibody, a multispecific antibody, or a combination thereof.

In one aspect, the antigen-binding fragment is one or more selected from a Fv fragment, a Fab fragment, an F(ab′)2 fragment, a Fab′ fragment, a dsFv fragment, an scFv fragment, an sc(Fv)2 fragment, or a combination thereof.

In one aspect, the antibody or antigen binding fragment thereof is a monoclonal antibody (mAb).

In one aspect, the antibody or antigen binding fragment thereof (e.g. mAb) of the disclosure is a scFV.

In one aspect, the antibody or antigen binding fragment thereof can bind to STEAP2 molecules across species, e.g., the antibody or fragment can bind to mouse STEAP2, rat STEAP2, rabbit, human STEAP2 and/or cynomolgus monkey STEAP2. In one aspect, the antibody or fragment can bind to human STEAP2 and cynomolgus monkey STEAP2. In one aspect, the antibody or antigen binding fragment can also bind to mouse STEAP2.

In one aspect, the antibody or antigen binding fragment thereof can specifically bind to STEAP2, e.g., human STEAP2 and cynomolgus monkey STEAP2.

In one aspect, the antibody or antigen-binding fragment thereof can include, in addition to a VH and a VL, a heavy chain constant region or fragment thereof. In one aspect, the heavy chain constant region is a human heavy chain constant region, e.g., a human IgG constant region, e.g., a human IgG1, IgG2, or IgG4 constant region. In one aspect (wherein the antibody or antigen-binding fragment thereof is conjugated to an agent, such as a cytotoxic agent), a cysteine residue is inserted between amino acid S239 and V240 in the CH2 region of IgG1. This cysteine is referred to as “a 239 insertion” or “239i.”

In one aspect, the antibody or antigen binding fragment thereof may comprise a heavy chain constant region comprising the amino acid sequence of SEQ ID NO: 60. In another aspect, the antibody or antigen binding fragment thereof may comprise a heavy chain constant region comprising the amino acid sequence of SEQ ID NO: 54.

In one aspect, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 234-257, 285-290, 308-331, 385-389, and 428-436, wherein the amino acid position numbering is according to the EU index as set forth in Kabat. In one aspect the IgG constant domain can contain one or more of a substitution of the amino acid at Kabat position 234 with Phenylalanine (F), a substitution of the amino acid at Kabat position 235 with Glutamic Acid (E), a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 331 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. In one aspect, the IgG constant domain can contain amino acid substitutions relative to a wild-type human IgG constant domain including as substitution of the amino acid at Kabat position 252 with Tyrosine (Y), a substitution of the amino acid at Kabat position 254 with Threonine (T), and a substitution of the amino acid at Kabat position 256 with Glutamic acid (E). In one aspect, the antibody or antigen-binding fragment thereof comprises a heavy chain, wherein the heavy chain is a human IgG1 YTE mutant.

In one aspect, the antibody or antigen-binding fragment thereof can include, in addition to a VH and a VL, and optionally a heavy chain constant region or fragment thereof, a light chain constant region or fragment thereof. In one aspect, the light chain constant region is a kappa lambda light chain constant region, e.g., a human kappa constant region or a human lambda constant region.

In one aspect, the antibody or antigen binding fragment thereof comprises a light chain constant region comprising the amino acid sequence of SEQ ID NO: 42.

In one aspect, a VH and/or VL amino acid sequence can have 85%, 90%, 95%, 96%, 97%, 98% or 99% similarity to a sequence set forth herein. In one aspect, a VH and/or VL amino acid sequence may comprise 1, 2, 3, 4, 5 or more substitutions, e.g., conservative substitutions relative to a sequence set forth herein. A STEAP2 antibody having VH and VL regions having a certain percent similarity to a VH region or VL region, or having one or more substitutions, e.g., conservative substitutions can be obtained by mutagenesis (e.g., site-directed or PCR-mediated mutagenesis) of nucleic acid molecules encoding VH and/or VL regions described herein, followed by testing of the encoded altered antibody for binding to STEAP2 and optionally testing for retained function using the functional assays described herein.

The affinity or avidity of an antibody or antigen binding fragment thereof for an antigen can be determined experimentally using any suitable method well known in the art, e.g., flow cytometry, enzyme-linked immunosorbent assay (ELISA), or radioimmunoassay (RIA), or kinetics (e.g., KINEXA® or BIACORE™ analysis). Direct binding assays as well as competitive binding assay formats can be readily employed. (See, e.g., Berzofsky et al., Antibody-Antigen Interactions, In Fundamental Immunology, Paul, W. E., Ed., Raven Press: New York, N.Y. (1984); Kuby, Immunology, W. H. Freeman and Company: New York, N.Y. (1992); and methods described herein.) The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art.

In one aspect, the antibody or antigen-binding fragment thereof, can bind to STEAP2-expressing cells with an IC50 lower than about 500 nM, lower than about 350 nM, lower than about 250 nM, lower than about 150 nM, lower than about 100 nM, lower than about 75 nM, lower than about 60 nM, lower than about 50 nM, lower than about 40 nM, lower than about 30 nM, lower than about 20 nM, lower than about 15 nM, lower than about 10 nM, lower than about 5 nM, lower than about 1 nM, lower than about 500 pM, lower than about 350 pM, lower than about 250 pM, lower than about 150 pM, lower than about 100 pM, lower than about 75 pM, lower than about 60 pM, lower than about 50 pM, lower than about 40 pM, lower than about 30 pM, lower than about 20 pM, lower than about 15 pM, lower than about 10 pM, or lower than about 5 pM. In one aspect, said IC50 is measured by flow cytometry.

A “monoclonal antibody” (mAb) refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals.

In another aspect, the antibody or antigen binding fragment thereof (e.g. mAb) of the disclosure is a humanized antibody or antigen binding fragment thereof. Suitably, said humanized the antibody or antigen binding fragment thereof is an IgG.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FW) connected by three complementarity-determining regions (CDRs), also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs.

The “Kabat numbering system” is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The table below lists the positions of the amino acids comprising the variable regions of the antibodies in each system.

| Region | Kabat | AbM | Chothia |
|---|---|---|---|
| LCDR1 | L24-L34 | L24-L34 | L24-L34 |
| LCDR2 | L50-L56 | L50-L56 | L50-L56 |
| LCDR3 | L89-L97 | L89-L97 | L89-L97 |
| HCDR11 | H31-H35B | H26-H35B | H26-H32 . . . 34 |
| HCDR12 | H31-H35 | H26-H35 | H26-H32 |
| HCDR2 | H50-H65 | H50-H58 | H52-H56 |
| HCDR3 | H95-H102 | H95-H102 | H95-H102 |

¹Kabat Numbering
²Chothia Numbering

ImMunoGeneTics (IMGT) also provides a numbering system for the immunoglobulin variable regions, including the CDRs. See, e.g., Lefranc, M. P. et al., Dev. Comp. Immunol. 27: 55-77(2003). The IMGT numbering system is based on an alignment of more than 5,000 sequences, structural data, and characterization of hypervariable loops and allows for easy comparison of the variable and CDR regions for all species. According to the IMGT numbering schema, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is a positions 50-65, and VH-CDR3 is at positions 95-102. VL-CDR1, VL-CDR2 and VL-CDR3 also correspond to classical Kabat numbering locations, namely positions 24-34, 50-56 and 89-97, respectively.

In one aspect, an antibody of the disclosure a human antibody.

The term “human antibody” means an antibody produced in a human or an antibody having an amino acid sequence corresponding to an antibody produced in a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

In one aspect, an antibody of the disclosure a chimeric antibody.

The term “chimeric antibodies” refers to antibodies in which the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006); Robbie et al., (2013) Antimicrob. Agents Chemother. 57, 6147-6153). See also U.S. Pat. No. 7,083,784, which is hereby incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

Potency of the antibody or antigen binding fragment thereof is normally expressed as an IC50 value, in ng/ml unless otherwise stated. IC50 is the median inhibitory concentration of an antibody molecule. In functional assays, IC50 is the concentration that reduces a biological response by 50% of its maximum. In ligand-binding studies, IC50 is the concentration that reduces receptor binding by 50% of maximal specific binding level. IC50 can be calculated by any number of means known in the art.

The fold improvement in potency for the antibody or antigen binding fragment thereof of the disclosure as compared to a reference antibody can be at least about 2-fold, at least about 4-fold, at least about 6-fold, at least about 8-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more.

Binding potency of an antibody is normally expressed as an EC50 value, in nM or pM unless otherwise stated. EC50 is the concentration of a drug that induces a median response between baseline and maximum after a specified exposure time. EC50 can be calculated by any number of means known in the art.

### Antibody Preparation

The antibodies of the present disclosure can be obtained using conventional techniques known to persons skilled in the art and their utility confirmed by conventional binding studies. By way of example, a simple binding assay is to incubate the cell expressing an antigen with the antibody. If the antibody is tagged with a fluorophore, the binding of the antibody to the antigen can be detected by FACS analysis.

Antibodies of the present disclosure can be raised in various animals including mice, rats, rabbits, goats, sheep, monkeys, or horses. Antibodies may be raised following immunization with individual capsular polysaccharides, or with a plurality of capsular polysaccharides. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g. Making and Using Antibodies: A Practical Handbook. GC Howard. CRC Books. 2006. ISBN 0849335280). Polyclonal and monoclonal antibodies are often purified using Protein A/G or antigen-affinity chromatography.

The antibody or antigen binding fragment thereof of the disclosure may be prepared as a monoclonal anti-STEAP2 antibody, which can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or an in vitro binding assay, e.g., radioimmunoassay (RIA) or enzyme-linked immunosorbent assay (ELISA), can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid using known methods.

Alternatively, the antibody or antigen binding fragment thereof (e.g. as monoclonal antibodies) can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

The polynucleotide(s) encoding an antibody or an antigen-binding fragment thereof of the disclosure can further be modified in a number of different manners using recombinant DNA technology to generate alternative antibodies. In some aspects, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In one aspect, the antibody or antigen-binding fragment thereof is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Pat. No. 5,750,373.

In one aspect, the antibody or antigen-binding fragment thereof can be selected from a phage library, where that phage library expresses human antibodies, as described, for example, in Vaughan et al., Nat. Biotech. 14:309-314 (1996); Sheets et al., Proc. Natl. Acad. Sci. USA, 95:6157-6162 (1998); Hoogenboom and Winter, J. Mol. Biol. 227:381 (1991); and Marks et al., J. Mol. Biol. 222:581 (1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Molec. Biol. 376:1182-1200 (2008), each of which is incorporated by reference in its entirety.

Affinity maturation strategies and chain shuffling strategies are known in the art and can be employed to generate high affinity human antibodies or antigen-binding fragments thereof. See Marks et al., BioTechnology 10:779-783 (1992), incorporated by reference in its entirety.

In one aspect, the antibody or antigen binding fragment thereof (e.g. a monoclonal antibody) can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate, or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. Suitably, the CDR residues may be directly and most substantially involved in influencing STEAP2 binding. Accordingly, part or all of the non-human or human CDR sequences can be maintained while the non-human sequences of the variable and constant regions can be replaced with human or other amino acids.

Antibodies can also optionally be humanized, resurfaced, engineered or human antibodies engineered with retention of high affinity for the antigen STEAP2 and other favorable biological properties. To achieve this goal, humanized (or human) or engineered anti-STEAP2 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as STEAP2. In this way, FW residues can be selected and combined from the consensus and import sequences so that the desired antibody characteristic, such as increased affinity for the target antigen(s), is achieved.

Humanization, resurfacing or engineering of anti-STEAP2 antibodies or antigen-binding fragments thereof of the present disclosure can be performed using any known method, such as but not limited to those described in, Jones et al., Nature 321:522 (1986); Riechmann et al., Nature 332:323 (1988); Verhoeyen et al., Science 239:1534 (1988); Sims et al., J. Immunol. 151: 2296 (1993); Chothia and Lesk, J. Mol. Biol. 196:901 (1987); Carter et al., Proc. Natl. Acad. Sci. USA 89:4285 (1992); Presta et al., J. Immunol. 151:2623 (1993); U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246; each of which is entirely incorporated herein by reference, including the references cited therein.

Anti-STEAP2 humanized antibodies and antigen-binding fragments thereof can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In one aspect, a fragment (e.g. antibody fragment) of the antibody (e.g. anti-STEAP2 antibody) is provided. Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In one aspect, anti-STEAP2 antibody fragments are produced recombinantly. Fab, Fv, and scFv antibody fragments can all be expressed in and secreted from E. coli or other host cells, thus allowing the production of large amounts of these fragments. Such anti-STEAP2 antibody fragments can also be isolated from the antibody phage libraries discussed above. The anti-STEAP2 antibody fragments can also be linear antibodies as described in U.S. Pat. No. 5,641,870. Other techniques for the production of antibody fragments will be apparent to the skilled practitioner.

According to the present disclosure, techniques can be adapted for the production of single-chain antibodies specific to STEAP2. See, e.g., U.S. Pat. No. 4,946,778). In addition, methods can be adapted for the construction of Fab expression libraries to allow rapid and effective identification of monoclonal Fab fragments with the desired specificity for STEAP2, or derivatives, fragments, analogs or homologs thereof. See, e.g., Huse et al., Science 246:1275-1281 (1989). Antibody fragments can be produced by techniques known in the art including, but not limited to: F(ab′)2 fragment produced by pepsin digestion of an antibody molecule; Fab fragment generated by reducing the disulfide bridges of an F(ab′)2 fragment; Fab fragment generated by the treatment of the antibody molecule with papain and a reducing agent; or Fv fragments.

In one aspect, an antibody or antigen-binding fragment thereof of the disclosure can be modified in order to increase its serum half-life. This can be achieved, for example, by incorporation of a salvage receptor binding epitope into the antibody or antibody fragment, by mutation of the appropriate region in the antibody or antibody fragment or by incorporating the epitope into a peptide tag that is then fused to the antibody or antibody fragment at either end or in the middle (e.g., by DNA or peptide synthesis), or by YTE mutation. Other methods to increase the serum half-life of an antibody or antigen-binding fragment thereof, e.g., conjugation to a heterologous molecule, such as PEG, are known in the art.

A modified antibody or antigen-binding fragment thereof as provided herein can comprise any type of variable region that provides for the association of the antibody or polypeptide with STEAP2. In this regard, the variable region can comprise or be derived from any type of mammal that can be induced to mount a humoral response and generate immunoglobulins against the desired antigen. As such, the variable region of an anti-STEAP2 antibody or antigen-binding fragment thereof can be, for example, of human, murine, non-human primate (e.g., cynomolgus monkeys, macaques, etc.) or lupine origin. In one aspect, both the variable and constant regions of the modified antibody or antigen-binding fragment thereof are human. In one aspect, the variable regions of a compatible antibody (usually derived from a non-human source) can be engineered or specifically tailored to improve the binding properties or reduce the immunogenicity of the molecule. In this respect, variable regions useful in the present disclosure can be humanized or otherwise altered through the inclusion of imported amino acid sequences.

In one aspect, the variable domains in both the heavy and light chains of an antibody or antigen-binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain aspects from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art to carry out routine experimentation to obtain a functional antibody with reduced immunogenicity.

Alterations to the variable region notwithstanding, those skilled in the art will appreciate that a modified antibody or antigen-binding fragment thereof of this disclosure will comprise an antibody (e.g., full-length antibody or antigen-binding fragment thereof) in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In one aspect, the constant region of the modified antibody will comprise a human constant region. Modifications to the constant region compatible with this disclosure comprise additions, deletions or substitutions of one or more amino acids in one or more domains. That is, the modified antibody disclosed herein can comprise alterations or modifications to one or more of the three heavy chain constant domains (CH1, CH2 or CH3) and/or to the light chain constant domain (CL). In one aspect, a modified constant region wherein one or more domains are partially or entirely deleted are contemplated. In one aspect, a modified antibody will comprise domain deleted constructs or variants wherein the entire CH2 domain has been removed (ΔCH2 constructs). In one aspect, the omitted constant region domain can be replaced by a short amino acid spacer (e.g., 10 residues) that provides some of the molecular flexibility typically imparted by the absent constant region.

Besides their configuration, it is known in the art that the constant region mediates several effector functions. For example, antibodies bind to cells via the Fc region, with an Fc receptor site on the antibody Fc region binding to an Fc receptor (FcR) on a cell. There are a number of Fc receptors that are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In one aspect, an antibody or an antigen-binding fragment thereof provides for altered effector functions that, in turn, affect the biological profile of the administered antibody or antigen-binding fragment thereof. For example, the deletion or inactivation (through point mutations or other means) of a constant region domain can reduce Fc receptor binding of the circulating modified antibody. In other cases it can be that constant region modifications, consistent with this disclosure, moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this disclosure can easily be made using well-known biochemical or molecular engineering techniques well within the purview of the skilled artisan.

In one aspect, the antibody or antigen-binding fragment thereof does not have one or more effector functions. For instance, in one aspect, the antibody or antigen-binding fragment thereof has no antibody-dependent cellular cytotoxicity (ADCC) activity and/or no complement-dependent cytotoxicity (CDC) activity. In one aspect, the antibody or antigen-binding fragment thereof does not bind to an Fc receptor and/or complement factors. In one aspect, the antibody or antigen-binding fragment thereof has no effector function. In one aspect, the antibody or antigen-binding fragment comprises an Fc region comprising a triple mutation (TM) which has reduced antibody dependent cellular cytotoxicity (ADCC) compared to an antibody having a wild type Fc region. In one aspect, the antibody or antigen binding fragment thereof has an Fc region which comprises a L234F/L235E/P331S triple mutation (TM). In one aspect, the antibody or antigen binding fragment thereof has an Fc region which comprises a L234F/L235E/P331S triple mutation (TM) according to SEQ ID NO: 59.

In one aspect, the antibody or antigen-binding fragment thereof can be engineered to fuse the CH3 domain directly to the hinge region of the respective modified antibodies or fragments thereof. In other constructs a peptide spacer can be inserted between the hinge region and the modified CH2 and/or CH3 domains. For example, compatible constructs can be expressed in which the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. In one aspect, any spacer added to the construct can be relatively non-immunogenic, or even omitted altogether, so as to maintain the desired biochemical qualities of the modified antibodies.

Besides the deletion of whole constant region domains, an antibody or antigen-binding fragment thereof provided herein can be modified by the partial deletion or substitution of a few or even a single amino acid in a constant region. For example, the mutation of a single amino acid in selected areas of the CH2 domain can be enough to substantially reduce Fc binding and thereby increase tumor localization. Similarly one or more constant region domains that control the effector function (e.g., complement C1Q binding) can be fully or partially deleted. Such partial deletions of the constant regions can improve selected characteristics of the antibody or antigen-binding fragment thereof (e.g., serum half-life) while leaving other desirable functions associated with the subject constant region domain intact. Moreover, the constant regions of the antibody and antigen-binding fragment thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. In one aspect, there may be an addition of one or more amino acids to the constant region to enhance desirable characteristics such as decreasing or increasing effector function or provide for more cytotoxin or carbohydrate attachment. In one aspect, it can be desirable to insert or replicate specific sequences derived from selected constant region domains.

The present disclosure further embraces variants and equivalents that are substantially homologous an antibody or antigen binding fragment of the disclosure (e.g. murine, chimeric, humanized or human antibody, or antigen-binding fragments thereof). These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In one aspect, the antibody or antigen-binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### Definitions

The following definitions pertain to the description of topoisomerase I inhibitors above.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃) and butyl (C₄).

Examples of saturated linear alkyl groups include, but are not limited to, methyl (C₁), ethyl (C₂), n-propyl (C₃) and n-butyl (C₄).

Examples of saturated branched alkyl groups include iso-propyl (C₃), iso-butyl (C₄), sec-butyl (C₄) and tert-butyl (C₄).

C₂₋₄ Alkenyl: The term “C2-4 alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂) and butenyl (C₄).

C₂₋₄ alkynyl: The term “C₂₋₄ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₄ cycloalkyl: The term “C₃₋₄ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
cyclopropane (C₃) and cyclobutane (C₄); and
unsaturated monocyclic hydrocarbon compounds:
cyclopropene (C₃) and cyclobutene (C₄).

Connection labels: In the formula

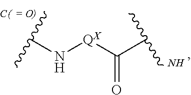

the superscripted labels ^{C(═O)} and ^{NH} indicate the group to which the atoms are bound. For example, the NH group is shown as being bound to a carbonyl (which is not part of the moiety illustrated), and the carbonyl is shown as being bound to a NH group (which is not part of the moiety illustrated).

### Salts

It may be convenient or desirable to prepare, purify, and/or handle a corresponding salt of the active compound/agent, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

### Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc. Isomers

Certain compounds/agents of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Enantiomerically enriched form” refers to a sample of a chiral substance whose enantiomeric ratio is greater than 50:50 but less than 100:0.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/enediamine, nitroso/oxime, thioketone/enethiol, N-nitroso/hyroxyazo, and nitro/aci-nitro.

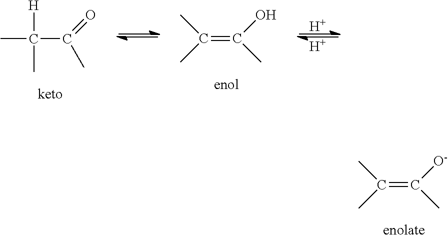

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, chlorine and iodine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallization and chromatographic means) of such isomeric forms are either known in the art or are readily obtained by adapting the methods taught herein, or known methods, in a known manner.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. MoI. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the number of \\ gaps introduced into the longer sequence in order \\ to align the two sequences ]\end{matrix}} \times 100$$

Substantially homologous polypeptides are characterized as having one or more amino acid substitutions, deletions or additions. These changes are of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine; lysine; histidine
Acidic: glutamic acid; aspartic acid
Polar: glutamine; asparagine
Hydrophobic: leucine; isoleucine; valine
Aromatic: phenylalanine; tryptophan; tyrosine
Small: glycine; alanine; serine; threonine; methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present disclosure. A limited number of non-conservative amino acids, amino acids that are not encoded by the genetic code, and unnatural amino acids may be substituted for polypeptide amino acid residues. The polypeptides of the present disclosure can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present disclosure.

Essential amino acids in the polypeptides of the present disclosure can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present disclosure.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of aspects of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or aspects of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

Other definitions of terms may appear throughout the specification. Before the exemplary aspects are described in more detail, it is to be understood that this disclosure is not limited to particular aspects described, and as such may vary. It is also to be understood that the terminology used herein is for the purpose of describing particular aspects only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an agent” includes a plurality of such agents and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

All publications mention in the above specification are herein incorporated by reference. Various modifications and variations of the described methods and system of the present disclosure will be apparent to those skilled in the art without departing from the scope and spirit of the present disclosure. Although the present disclosure has been described in connection with specific aspects, it should be understood that the disclosure as claimed should not be unduly limited to such specific aspects. Indeed, various modifications of the described modes for carrying out the disclosure which are obvious to those skilled in biochemistry and biotechnology or related fields are intended to be within the scope of the following claims.

### EXAMPLES

### Example 1

### STEAP2 is Over-Expressed in Prostate Cancer

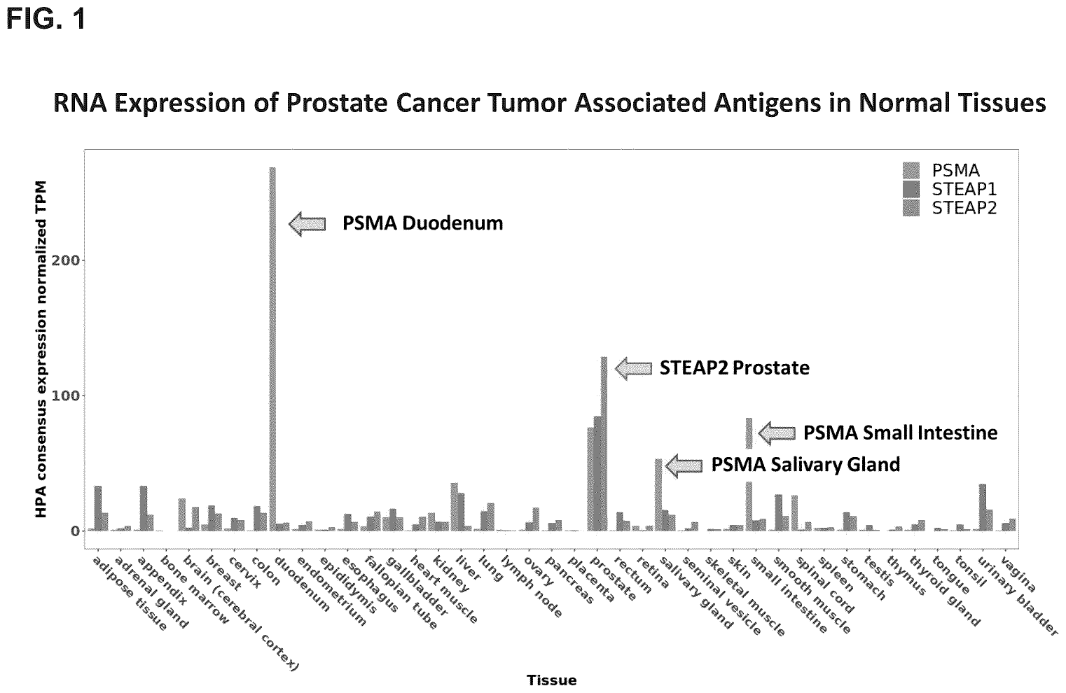

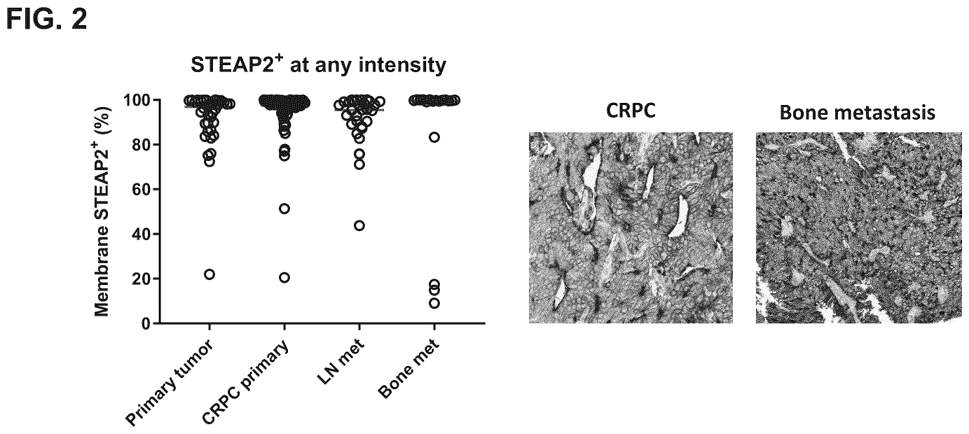

STEAP2 is a metalloreductase that reduces iron and copper to facilitate cellular uptake, metabolism, and proliferation, which is predominantly expressed in prostate cancer, with little expression in healthy tissue outside the prostate (FIG. 1). A query with human protein atlas confirms that shows less RNA expression in vital organs than PSMA & STEAP1. STEAP2 has high, homogeneous cell surface expression across all disease stages of prostate cancer, including metastases and castration-resistant prostate cancer (CRPC) (FIG. 2).

The expression profile of STEAP2 was assessed using a validated IHC protocol to demonstrate STEAP2 expression in human tissues and human tumor tissues (FIG. 2). Immunohistochemistry was carried out from a number of sections of tumors taken acquired from human subjects with primary (n=36), CRPC (n=78), lymph node metastases (n=30), or bone metastases (n=18). Expression across the collection of human tumors was similarly high.

### Example 2

### Generation of Anti-STEAP2 Antibodies

### STEAP2 Variable Domain Discovery

STEAP2 is a member of the STEAP family of metalloreductases, which reduce copper and iron molecules to forms that can be used by cells for metabolic purposes. STEAP2 has been shown to be widely expressed in prostate tumors throughout all stages of disease while displaying limited normal tissue expression.

40A3 is a human IgG1 κ antibody that binds to the extra-cellular domains (ECDs) of STEAP2, discovered using Del-1 humanized transgenic mouse technology. mAb 40A3, was further optimized, germ-lined and affinity matured to yield 40A3GL-LO14. The parental anti-STEAP2 mAb, 40A3, was isolated from a hybridoma campaign following immunization of transgenic Del-1 mice with STEAP2 expressing cells. A chimeric cell line was created by grafting the STEAP2 extracellular loops onto the backbone of the STEAP3 protein to exploit the cell surface localization of STEAP3.

Four to six week old transgenic female Del-1 mice (C57BL/6 background) were immunized with Ad293 cells overexpressing STEAP3-2. Three days post pre-fusion boost, splenocytes and lymph node cells were harvested. B-cells were isolated using a pan B-cell enrichment kit from Miltenyi. Isolated B-cells were further enriched by panning on irradiated STEAP2 knock out cell line. The antigen enriched B-cells were then fused to P3X63Ag8.653 (CRL-1580-ATCC) and seeded into 96 well plates in HAT selection medium. Supernatants from 96 well plates were screened using high through put flow cytometry. Hybridomas specific to Ad293 OE STEAP2 were also tested for binding to primary cancer cell lines (LNCaP, LNCaP-STEAP2-KO) and additional STEAP family members. STEAP2-specific hybridomas were moved into limited dilution cloning. V-genes were rescued from all clones that retained specific binding to LNCaP. Recombinant antibodies were generated and used for downstream characterization.

Clone 40A3 was selected as a lead for further development based on its cell-binding affinity, STEAP family member selectivity, and human/murine cross-reactivity. The parental mAb was then optimized with the introduction of a germline leucine residue in framework three (FW3) of the VH domain and two potential deamidation liabilities were removed from CDRs L1 and H3, to render mAb 40A3. In order to ensure that binding was not compromised as a result of the germ-lining and de-risking mutation modifications, 40A3-LO7 binding was assessed on STEAP2 expressing LNCaP prostate cancer cells. The flow cytometry experiment yielded an on-cell binding EC50 value of 43.33 nM.

To improve the affinity of 40A3-LO7, the clone was subjected to site saturation mutagenesis and cell-based screening. Three affinity matured variants with limited background binding were subsequently identified, 40A3-LO11 (CDRL1_S30A CDRH2_V61P), 40A3-LO12 (CDRL1_S30A CDRH3_L97R) and 40A3-LO14 (CDRL1_S30A CDRH2_V61P CDRH3_L97R). The combined CDRH2_V61P CDRH3_L97R substitution mutations in 40A3-LO14 exhibited improved binding by 26-fold relative to the starting antibody, 40A3-LO7.

### In-Vitro Characterization

The binding affinities for parental 40A3-LO7 and its affinity matured derivatives were assessed on LNCaP cells. 40A3-LO14 demonstrated the strongest binding with an EC50 of 1.67 nM. Variants 40A3-LO11 and LO12 had slightly poorer EC50 values of 2.38 and 1.72 nM, respectively. None of the variants tested showed binding to LNCaP STEAP2 KO cells. Our data demonstrated a clear correlation between the binding of 40A3 variants to human and murine chimeric STEAP3-2 proteins, with 40A3-LO14 being the strongest with EC50s of 1.67 and 0.97 to human and murine chimeras, respectively.

### FACS Binding Assay in Human Cell Lines

Seven point 3-fold serial dilutions of 5× antibodies (mAbs) were prepared in FACS Buffer (5% Heat Inactivated Fetal Bovine Serum (GibCo Ref #10082-147; Lot #2370845P) in PBS pH 7.4 (Sigma cat #806552-500ML); Sterile filtered (Thermo Scientific cat #09-740-64B)). Then, 10 ul of mAb serial dilutions, in duplicates, were added into designated wells in a 96-well U-bottom clear plate (Costar cat #07-200-95). 10 ul of FACS Buffer was added to 2 wells per cell line as an “untreated” control. One plate was prepared per cell line. The plates were placed on ice until needed. The tested mAbs 5× and final assay starting concentrations were as follows:

ug/ml

| mAbs | 5X | 1X |
|---|---|---|
| 40A3-LO7 | 315 | 63 |
| 40A3-LO11 | 105 | 21 |
| 40A3-LO12 | 105 | 21 |
| 40A3-LO14 | 35 | 7 |

High Steap2 expressing LNCap and isogenic non-expressing LNCAP STEAP2 CRSPR 2X KO Prostate Cancer (CaP) cells were cultured in T175 flasks (Greiner 660160), pre-treated with Poly-L-Lysine (P4707-50ML; SIGMA-ALDRICH) as per vendors. Both lines were maintained in RPMI 10% FBS 1× Glutamax (ATCC Modified RPMI R7388-500ML Sigma; Heat Inactivated FBS from GibCo Ref #10082-147; Lot #2370845P; GlutaMax from GibCo Ref #35050-061)

The human CaP cells were harvested for FACS by washing them once with PBS pH 7.4, adding cold 0.25% Trypsin-EDTA, and left at room temperature (RT) for approximately 5 min. After the RT incubation, the flasks were gently rocked side-to-side to ensure cells were disassociated from the plastic. 10 ml of maintenance media was added into the flask to collect the cells and transferred into a 50 ml conical tube by filtering through a 40-um cell strainer (Falcon cat #352340). The cells were centrifuged at 1,200 RPMs for 3 minutes at 4 C on an Allegra X-15R centrifuge (Beckman Culture), the supernatant was aspirated, and the cell pellets were resuspended in 10 ml ice-cold FACS Buffer. A small aliquot (200 ul) was used to count the cells and assess the percent (%) viability in a Cell Viability Analyzer (V-Cell BLU). Cell density was adjusted to 2e6 cells/ml in 12 ml of ice-cold FACS Buffer.

Forty microliters of LNCap and LNCAP STEAP2 CRSPR 2X KO cell lines were seeded, on their respective plates, on top of the previously chilled 96-well U-bottom plates containing the mAbs. The cells were gently mixed in wells containing antibodies to ensure distribution (used 200 uL multichannel set to 30 uL). After covering the plates with lids and aluminum paper, the plates were incubated on ice for 30 mins in the dark.

After incubation, the cells were washed twice with ice-cold FACS Buffer by centrifuging the plates at 1,200 RPMs for 2 min, at 4 C, flicked on a biohazard container with absorbent pads, and then resuspended in 100 ul/well with a solution containing 1:400 secondary antibody (Goat anti-Human AF647, 2 mg/mL, Invitrogen #A21445) and 1:1,000 DAPI (1000× DAPI, Cell Signaling Technology cat #4083S) in ice-cold FACS buffer. The plates were covered with a lid and aluminum paper and incubated on ice for 30 min in the dark. After the secondary and nuclei staining incubation, the cells were washed as previously described and resuspended in 80 ul of ice-cold FACS Buffer. Plates were kept on ice and covered till processed by the FACS instrument.

### Example 3

### In Vitro Cell-Based Binding of Anti-STEAP2 Antibodies

The cell-binding assays were designed to assess the binding affinities for parental 40A3-LO7 and its affinity matured derivatives to both human and murine STEAP2. The binding of the anti-STEAP2 antibodies were detected using a fluorescently labelled anti-human secondary antibody and standard methods of flow cytometry.

Briefly, a seven point 3-fold serial dilutions of 5× antibodies (mAbs) was prepared in sterile filtered FACS Buffer (5% Heat Inactivated Fetal Bovine Serum in PBS pH 7.4). Ten microliters of the antibody dilutions were added into designated wells in a 96-well U-bottom clear plate. The evaluated cells lines were harvested using cold 0.25% Trypsin-EDTA, resuspended in maintenance media, pelleted, and counted. Cell density was adjusted to 2×10⁶ cells/ml and forty microliters of cells were seeded on top of the antibody dilutions and incubated for 30 minutes. Cells were washed and a solution containing a 1:400 dilution of anti-human secondary antibody and a 1:1000 dilution of DAPI. After another 30 minute incubation, cells were washed, and then transferred to the FACS instrument for analysis.

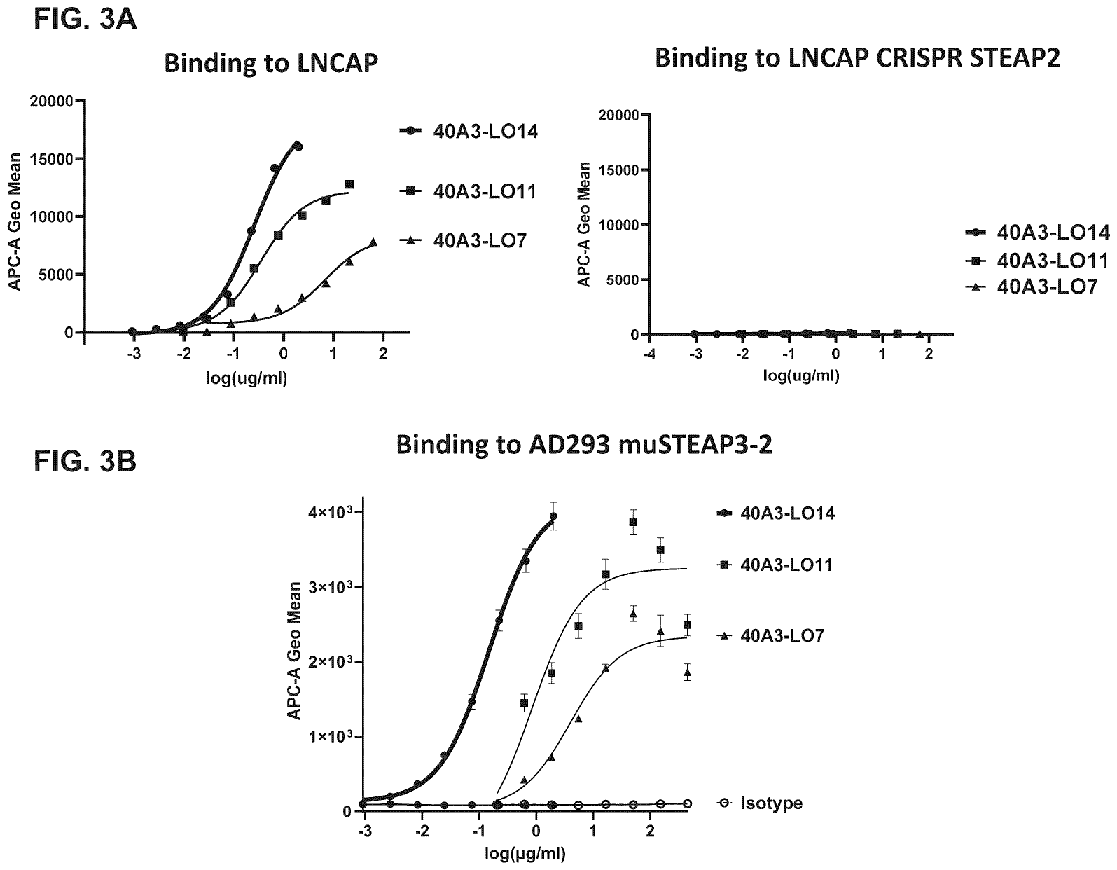

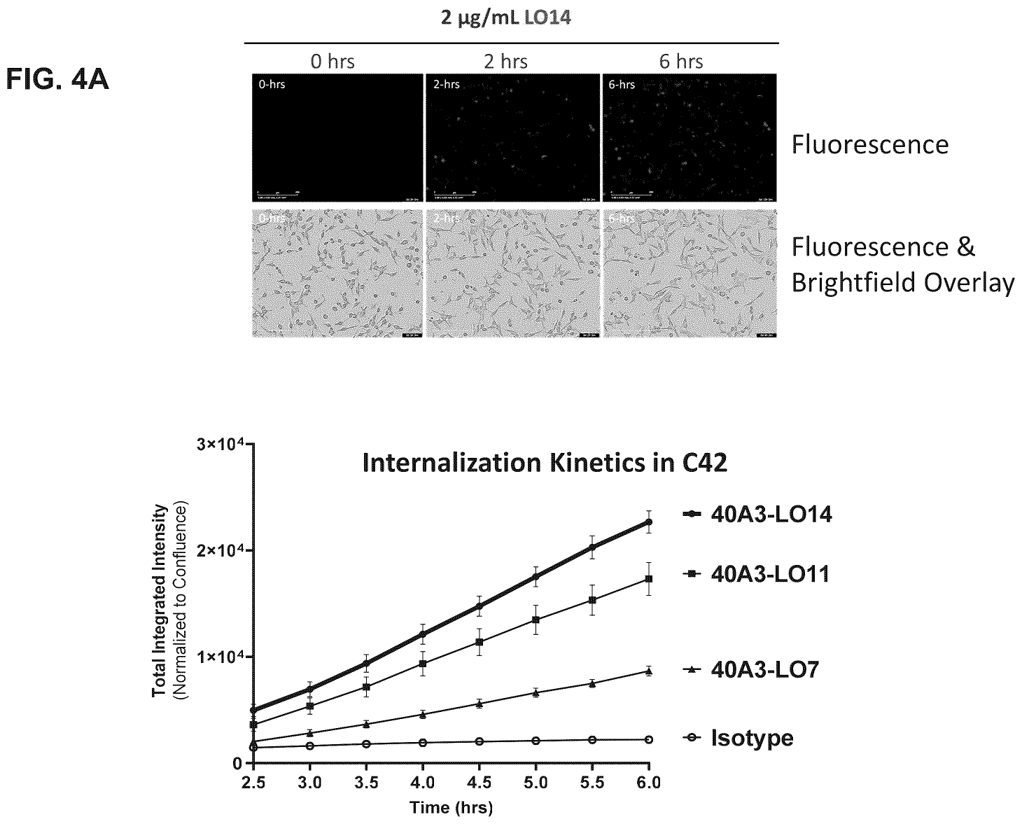

Anti-STEAP2 antibody 40A3-LO14 also showed the strongest binding affinity to LNCaP cells, with an EC50 of 1.67 nM (FIG. 3A, left). Variant 40A3-LO11 exhibited a slightly poorer EC50 value of 2.38 nM. None of the variants tested showed binding to LNCaP STEAP2 CRISPR KO cells, signifying that cell binding was specific to the surface expression of STEAP2 (FIG. 3A, right). 40A3-LO14 also demonstrated the strongest binding to AD293 muSTEAP3-2 cells, thus confirming the cross-reactivity of LO14 mAb to murine STEAP (FIG. 3B). Binding affinity, cross-reactivity, and developability characteristics for each of LO11 and LO14 mAbs are summarized in Table 1 below:

TABLE 1

| | | Mu X- | | |
|---|---|---|---|---|
| | Binding | reactivity | Heat Stress | Photo stress |
| Clone ID | Affinity | (AD293 | (% aggregate | (% aggregate |
| (hIgG1-TM) | (LNCaP) | STEAP3-2) | increase) | increase) |
| 40A3-LO14 | 1.67 nM | 0.97 nM | 0.27% | 1.75% |
| 40A3-LO11 | 2.38 nM | 5.78 nM | 0.24% | 0.8% |

### Example 4

### Internalization Evaluation of Anti-STEAP2 Antibodies

The internalization assays were designed to determine the rate at which parental 40A3-LO7 and its affinity matured derivatives could enter the cell after target engagement. The assay relies on labeling the Fc-containing test antibodies with a Fab fragment-conjugated pH-sensitive fluorophore (Sartorius/Essen BioScience) and the inherent ability of STEAP2 receptor to internalize when bound to antibody. The internalization of the anti-STEAP2 antibodies was detected with standard methods of live cell imaging using the Incucyte SX5.

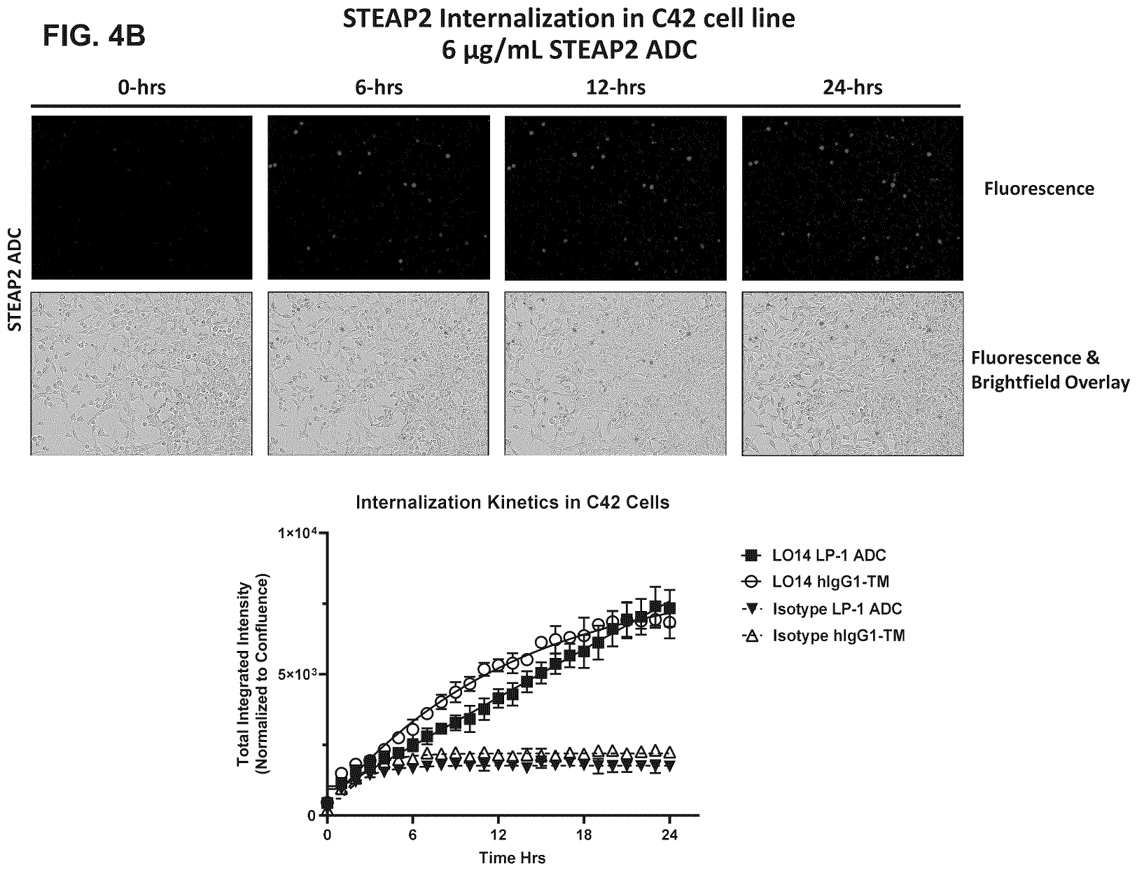

Briefly, 24 hrs prior to starting the assay, the cells lines being evaluated were harvested using cold 0.25% Trypsin-EDTA, resuspended in maintenance media, pelleted, and counted. Cell density was adjusted to 0.15 e6 cells/ml in RPMI1640 media without phenol red. One hundred microliters of cells were added to each well and incubated overnight at 37 C, 5% CO2. On the day of the assay, a 0.5 mg/mL stock solution of Red Fab-Fluor was combined with either anti-STEAP2 antibodies or isotype control antibody at a molar ratio of 1:3 test mAb to Fab (3× labeling solution). After a 20 min incubation, 50 microliters of 3× labeling solution was added to the cells. Imaging began immediately after the addition of labeling solution and continued every 30 minutes for 6 hours using a 20× objective, imaging channels “Phase/Birghtfield” and “Orange”, and the standard scan settings. The data was normalized analyzed by dividing the “Total Integrated Intensity (TII=RCU×μm²/Image)” by the “% Confluence,” then multiplying by an appropriate scaling factor. The normalized data plotted as a function of time, and the slope was calculated to determine the internalization rate (A intensity/min). The earliest timepoint at which fluorescent signal was detected above background was at 2 hours (FIG. 4A-4B, top), so the internalization kinetics were calculated between 2.5-6 hours.

C42 cells are human prostate cancer cells that endogenously express high levels of wild-type STEAP2. When bound to these cells, 40A3-LO14 showed the fastest internalization rate, with an increase of 85 TII/min (FIG. 4A-4B, bottom). Variant 40A3-LO11 had a slightly slower internalization rate with an increase of 66 TII/min. Fluorescent signal was not detected in the isotype control group suggesting that antibody internalization on these cells is a function of STEAP2 receptor binding.

### Example 5

### Potency of STEAP2 TOP1i and LP-1 ADCs on Human Prostate Cancer Cell Lines

The affinity matured anti-STEAP2 human IgG1κ monoclonal antibodies 40A3-LO14 (known herein as LO14) and 40A3-LO11 (known herein as LO11) were conjugated via a cleavable maleimide-PEG8-valine-alanine linker (cleavable mal-PEG8-val-ala linker) to a TOP1i warhead.

| | | | | Con- | | | ADC | | | |
|---|---|---|---|---|---|---|---|---|---|---|
| | | | | centration | Volume | Starting | recovered | % | % | Endotoxin |
| Entry | Antibody | Payload | DAR | (mg/ml) | (mL) | mAb (g) | (g) | Yield | Monomer | (EU/mg) |
| 1 | L014 | TOPO1i | 8 | 11.74 | 59 | 1 | 0.693 | 69.3 | 99.3 | 0.135 |
| | | (SG3932) | | | | | | | | |
| 2 | L014 | TOPO1i | 8 | 10.43 | 800 | 10 | 8.3 | 83 | 99.3 | 0.459 |
| | | (SG3932) | | | | | | | | |

The TOP1i warhead released from SG3932 referred to herein can be:

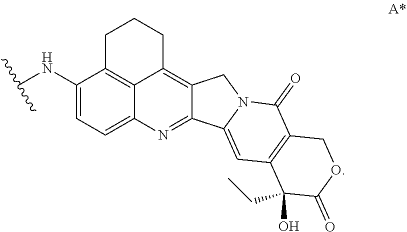 [SMILES: *Nc1ccc2nc3c(c4c2c1CCC4)Cn1c-3cc2c(c1=O)COC(=O)[C@]2(O)CC]

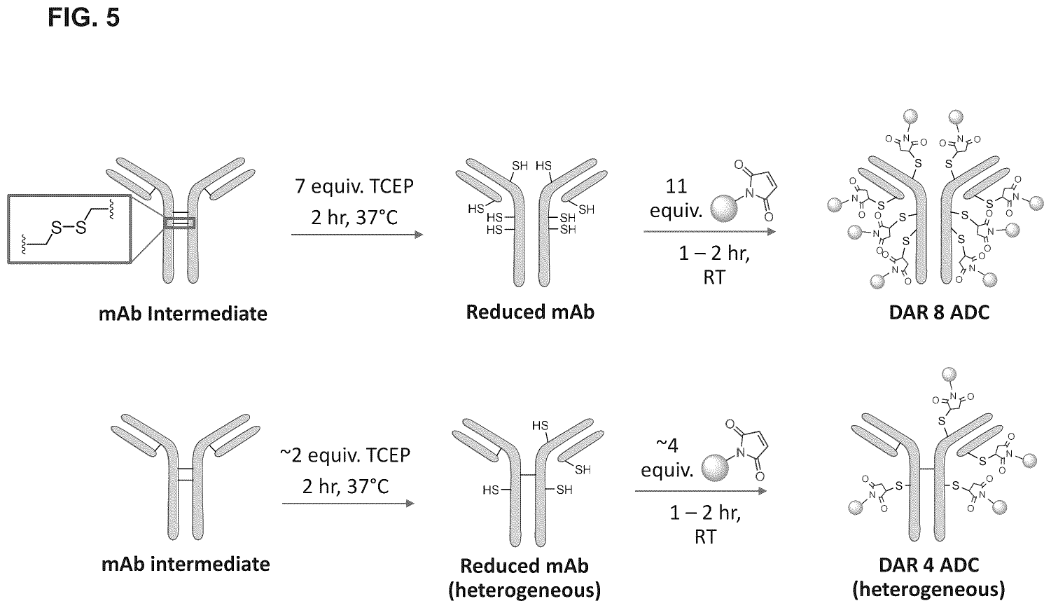

The TOP1i drug is covalently bound to native cysteines in the antibody through a thiosuccinimide linkage, with about 4 to 8 drugs bound per antibody (i.e., DAR of about 4 to about 8). A schematic of DAR8 and DAR4 versions of the STEAP2 ADCs is shown in FIG. 5.

The cytotoxicity assays were designed to determine whether LO11 and LO14 TOP1i ADCs could reduce the cell viability of STEAP2 expressing prostate cancer cell lines. The assay relies on the principle that dying cells will produce less adenosine triphosphate (ATP). By using a reagent that exhibits bioluminescence in the presence of ATP (CellTiter-Glo 2.0), dose-dependent decreases in cell viability are reflected in decreased bioluminescent signal. The bioluminescent signal was detected using standard plate-reading methods (SpectraMax M5).

Briefly, 24 hours prior to starting the assay, human prostate cancer cells were harvested and counted described above. Cell density was adjusted to 0.05 e6 cells/ml for 22Rv1, LNCAP, and LNCAP STEAP2 CRSPR 2X KO, and 0.03 e6 cells/ml for C42 in cell the appropriate culture media. One hundred ul of cells were added to each well and cells were incubated overnight at 37 C, 5% CO2. The following day, a 9-point, 4-fold serial dilution of 3× stock ADCs (STEAP2 or Isotype control TOP1i DAR8), was prepared in cell maintenance media. Fifty microliters of each dilution was added to the designated wells and cells were incubated for 5. After removing the treatment media, 50 microliters of RPMI1640 media without Phenol Red and 50 microliters of CTG2.0 (Promega) was added to each well. Plates were incubated for 20 minutes and then transferred to the SpectraMax M5 for luminescence detection.

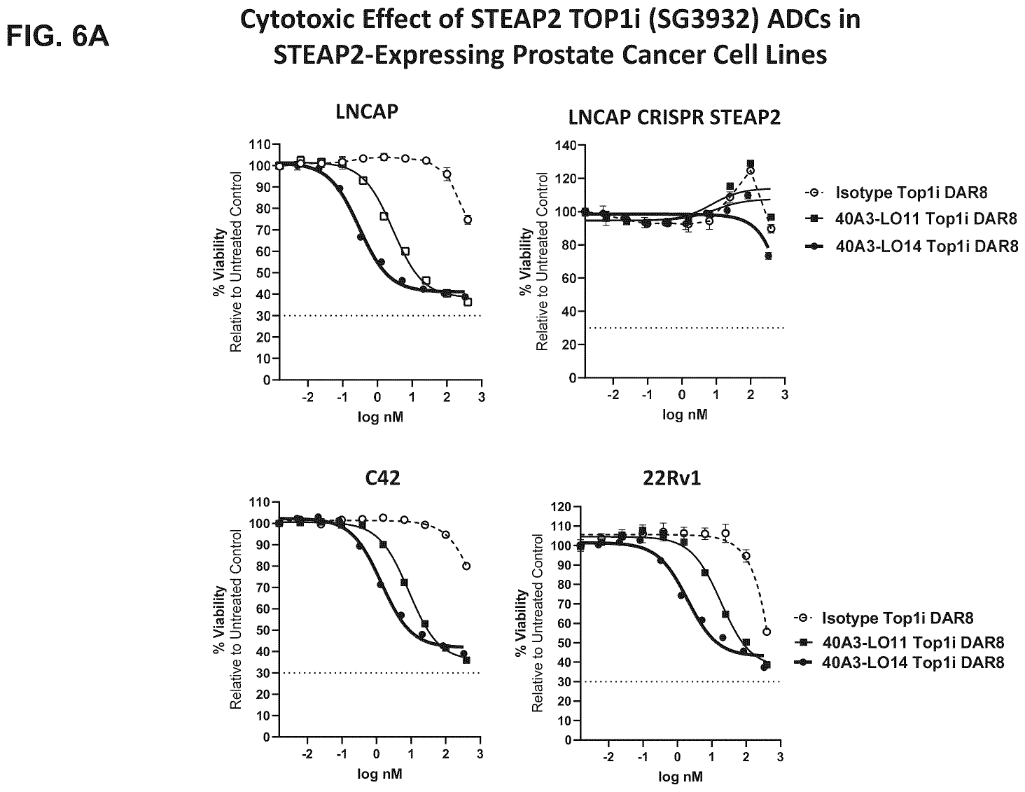

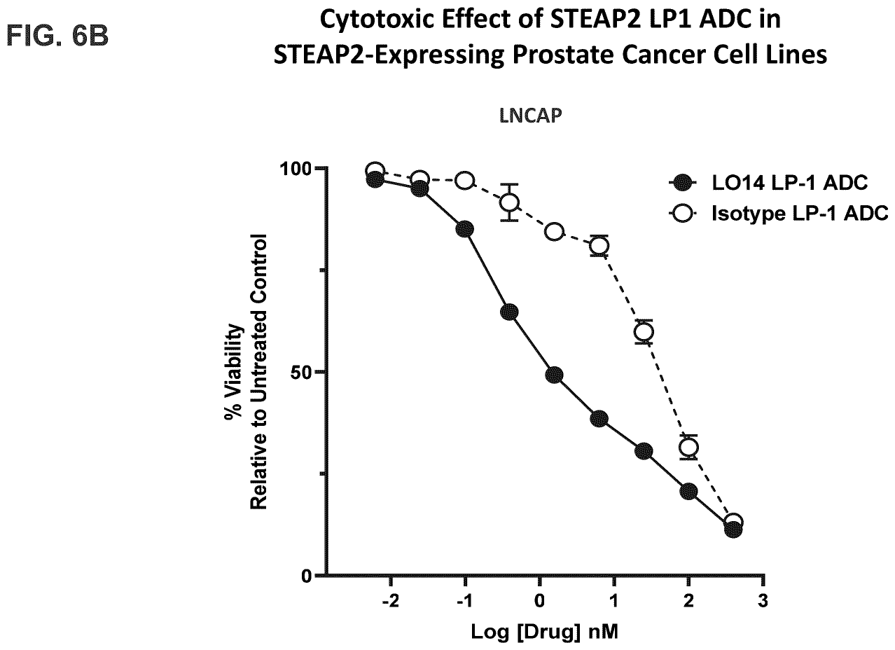

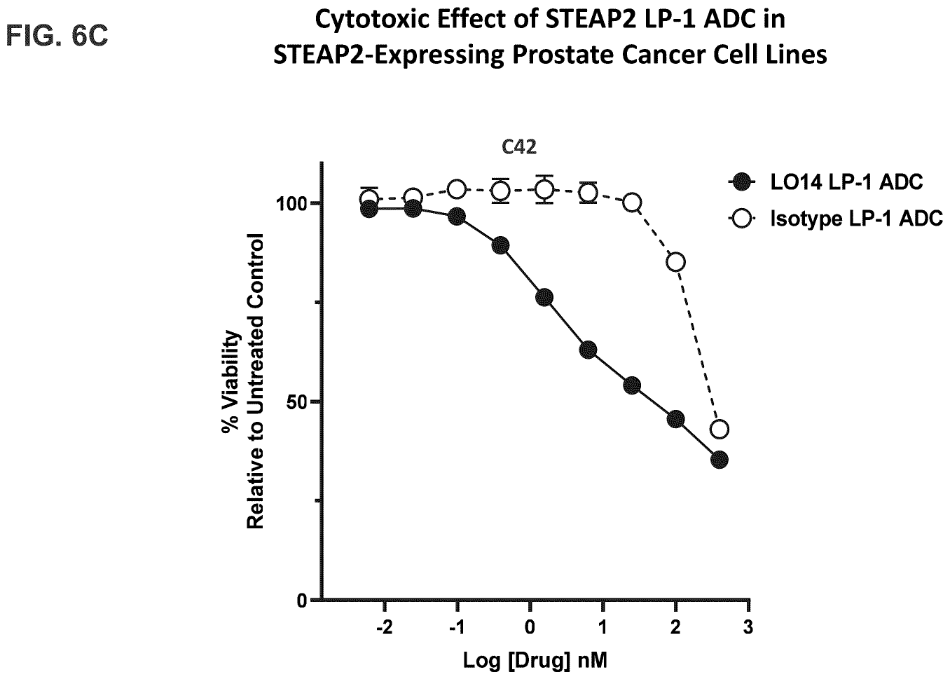

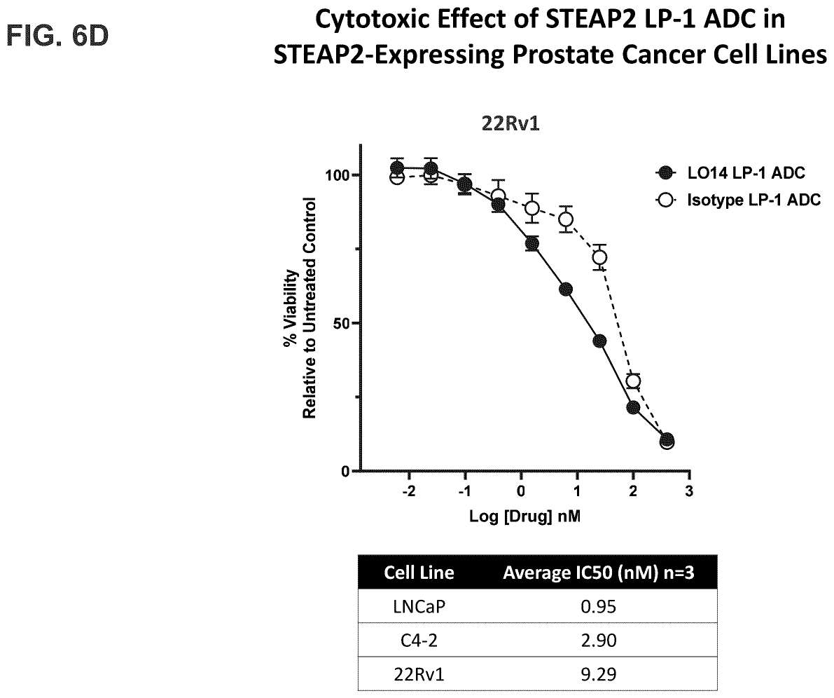

The results from the potency screening of LO14 and LO11 TOP1i and LP1 DAR8 ADCs are shown in FIG. 6A-6D. Treatment of LNCaP cells with either LO14 or LO11 ADCs showed a dose-dependent decrease in the cell viability (IC50 0.30 and 2.78 nm respectively) that was not observed when treated with the Isotype TOP1i ADC. The lack of cytotoxic effect of LO14 and LO11 ADCs on the isogenic cell line KO for STEAP2 confirms that the cell killing activity is STEAP2-specific. Although C42 and 22Rv1 cells have varying levels of STEAP2 expression, both LO14 and LO11 ADCs were able to reduce the viability of these cell lines. expression. In all cell lines, LO14 TOP1i ADC showed a more potent effect than LO11 TOP1i ADC. IC50s for both ADCs are shown in Table 2 below:

TABLE 2

| Clone ID | In Vitro Potency | In Vitro Potency | In Vitro Potency |
|---|---|---|---|
| (IgG1-TM) | (LNCaP) | (C4-2) | (22Rv1) |
| 40A3-LO14 | 0.22 nM | 2.23 nM | 4.53 nM |
| 40A3-LO11 | 1.70 nM | 16.53 nM | 32.08 nM |

### Example 6

### Pharmacokinetic Evaluation of LO14 TOP1i ADC

A study was designed to characterize the pharmacokinetics (PK) properties of LO14 unconjugated antibody, DAR4, and DAR8 TOP1i ADCs in mice. Four to six week old male immunodeficient (athymic nude, NOD Scid Gamma) or humanized mice (FcRn) were administered a single dose 5 milligrams per kilogram (mpk) intravenous injection of test reagent. Animals were monitored and 100-200 microliters of blood was collected at each timepoint (n=9 timepoints between 0 to 21 days post injection). The mice for each test group (n=9) were distributed among three cohorts to enable 6 survival bleeds and 3 terminal bleeds over the course of the study. Standard ELISA and Mass Spectrometry methods were used to quantify plasma concentration (ug/mL) of each test article over time. A standard 2 compartment model was fit to the pooled data from all the mice on study. All the individual datapoints are utilized but assuming that there is no inter-animal variability in any of the parameters (naive pooled approach). The 2 compartment model was used to calculate drug clearance (C1), the apparent volume of distribution at steady state (Vₛₛ), and half-life (T_{1/2}).

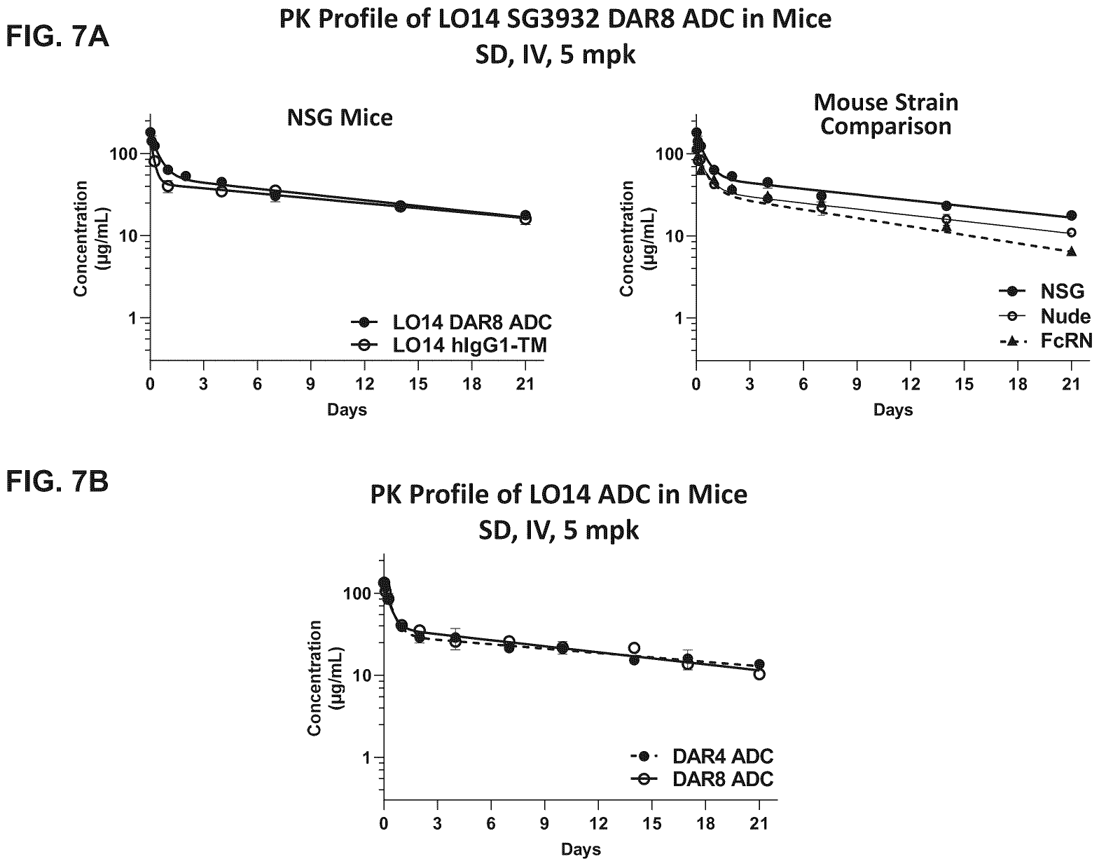

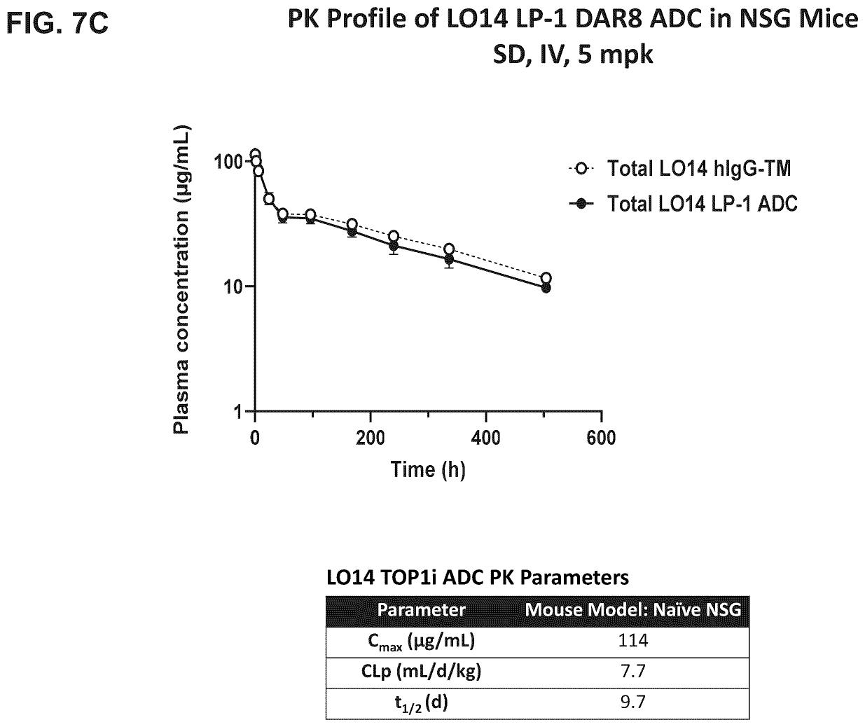

The results of each PK study are shown in FIGS. 7A-7B. To confirm that ADC synthesis did not impact PK properties of LO14, animals were injected with either unconjugated LO14 hIgG1-TM antibody (LO14 mAb) or LO14 DAR8 ADC. In NSG mice, the clearance rate of LO14 mAb and DAR8 ADC were similar at 5.2 and 6 mL·day⁻¹·kg⁻¹ respectively (FIG. 7A, left). The FcRn humanized mouse model is used to confirm that PK parameters are retained in an immune competent host. The clearance rate of LO14 TOP1i DAR8 ADC was slightly higher in FcRn mice (10.7 mL·day⁻¹·kg⁻¹) than in immunocompromised NSG and Athymic Nude mice (6 and 7.6 mL·day⁻¹·kg⁻¹ respectively) resulting in a shorter half-life of the molecule in this animal model (8.8 days) (FIG. 7A, right). Additionally, in NSG mice there was minimal difference between the clearance of LO14 TOP1i DAR 4 as compared to LO14 TOP1i DAR8 (6.4 and 6 mL·day⁻¹·kg⁻¹ respectively) (FIG. 7B). PK results are summarized in Table 3 below:

TABLE 3

| | Animal Strain | | | | | |
|---|---|---|---|---|---|---|
| | NSG | NSG | NSG* | Athymic Nude | FcRn | |
| | LO14 | LO14 | LO14 | | | |
| | hIgG1 | TOP1i | TOP1i | | | |
| | TM | DAR4 | DAR8 | | | |
| Cl | 5.2 | 6.4 | 6 | 7.6 | 10.7 | mL.day⁻¹.kg⁻¹ |
| Vₛₛ | 110 | 146 | 104 | 126 | 126 | mL.kg⁻¹ |
| T_{1/2} | 15 | 16.8 | 12.6 | 12.2 | 8.8 | days |

*LO14 TOPli DAR8 PK parameters in NSG mice averaged from n = 2 independent experiments

### Example 7

### In Vivo Efficacy of LO14 TOP1i and LP-1 ADCs

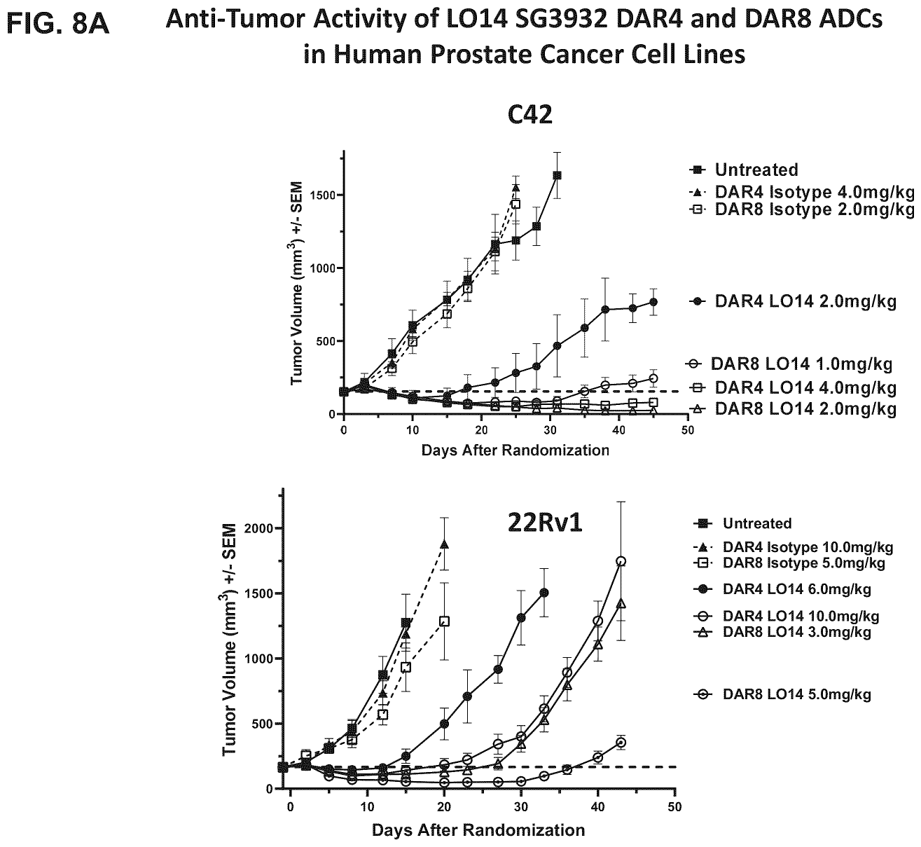

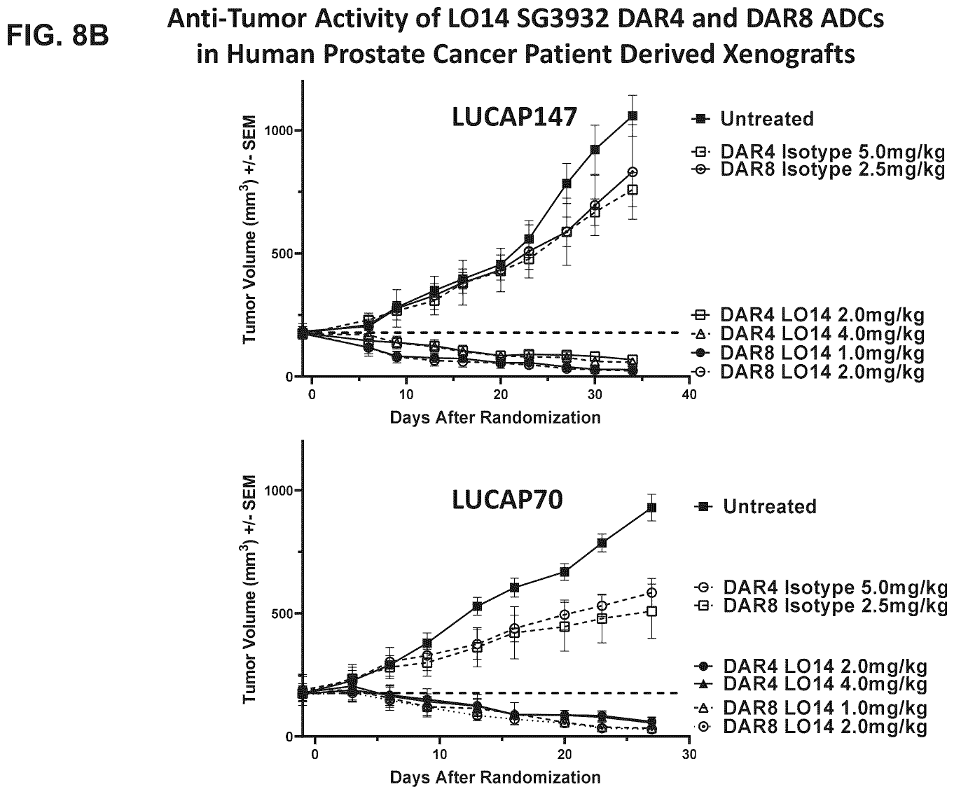

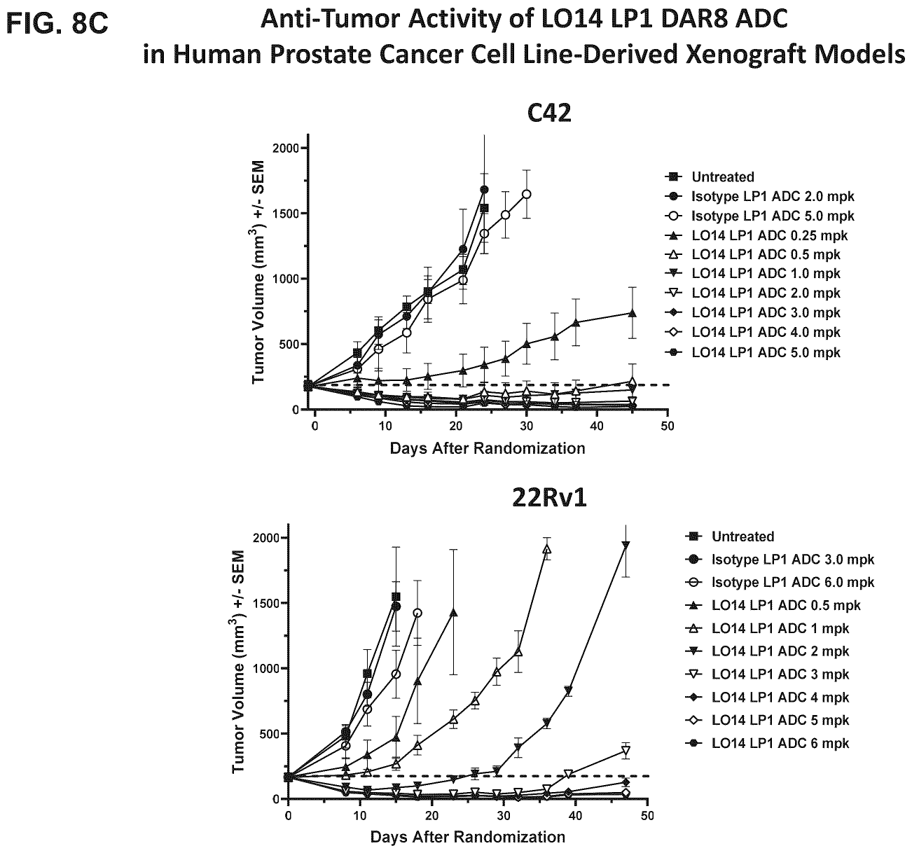

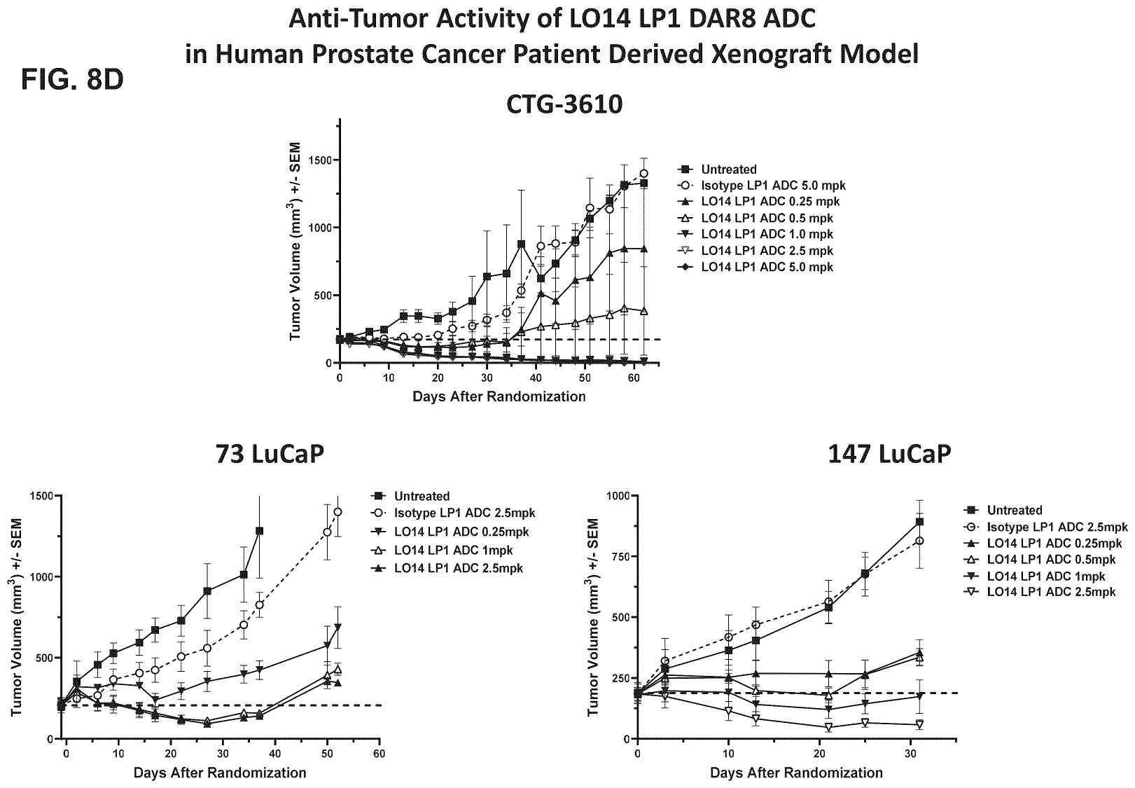

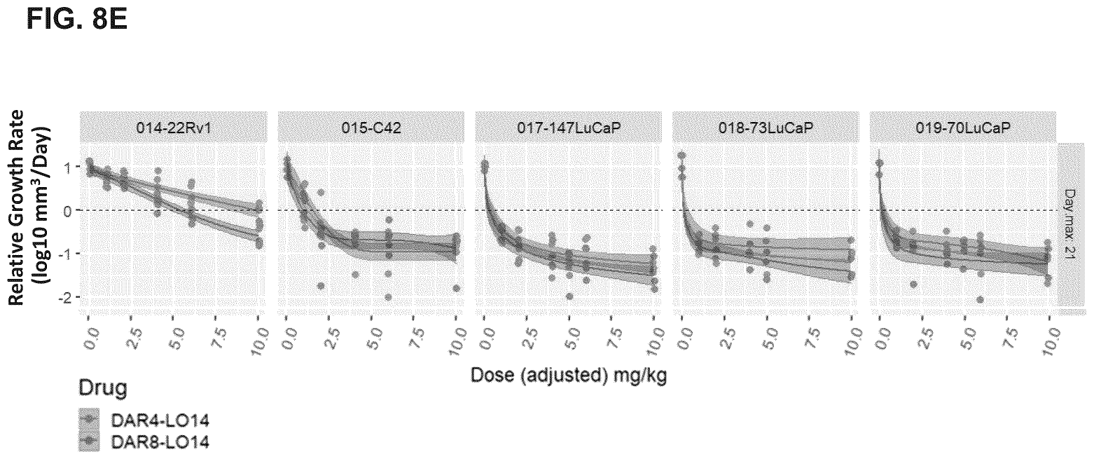

To determine whether LO14 TOP1i DAR4 and DAR8 ADCs showed dose-dependent anti-tumor effects in vivo, dose levels ranging from 1.0 mg/kg to 4 mg/kg were evaluated in human prostate cancer tumor models. Cell lines or tumor tissue fragments were implanted subcutaneously into male NSG mice between 6 to 8 weeks of age. When tumors reached the appropriate tumor volume range (150-300 mm³), animals were randomized into treatment and control groups (n=5 animals/group) and dosing was initiated. A single dose of test article was administered to the tumor-bearing mice via intravenous injection. Animals were observed daily, and tumor dimensions and body weight were measured and recorded twice weekly. Tumor volumes were measured by digital caliper and the volumes of tumors were calculated using the following formula: tumor volume=[length (mm)×width (mm)²×0.52, where the length and width are the longest and shortest diameters of the tumor, respectively. Results shown in FIGS. 8A-8B demonstrate that treatment with either LO14 TOP1i DAR4 or DAR8 results in dose-dependent growth inhibition of prostate tumor xenographs.

Anti-tumor activity of LO14 TOP1i ADCs was demonstrated in prostate cancer cell lines (C42 and 22Rv1) and patient derived xenographs (LUCAP147 and LUCAP70). In all models DAR4 had slightly reduced potency compared to DAR8 at the payload matched dose. Isotype control TOP1i DAR4 and DAR8 ADCs did not cause tumor regression in any of the models tested.

In the C42 model, significant tumor regression was observed at doses as low as 4 mpk and 1 mpk of LO14 TOP1i DAR4 and DAR8 respectively (FIG. 8A, top). In the 22Rv1 model, which has lower STEAP2 expression, higher doses were required of both DAR4 and DAR8 to achieve significant tumor regression (10 and 3 mpk respectively, FIG. 8A, bottom). Although LUCAP147 and LUCAP70 PDX models also have low-endogenous STEAP2 expression, these models were highly sensitive to treatment with STEAP2-targeting ADCs. In both models, significant tumor regressions were observed at doses as low as 2 mpk and 1 mpk of LO14 TOP1i DAR4 and DAR8 respectively (FIG. 8B).

In the C42 model, significant tumor regression was observed at doses as low as 0.25 mpk and 0.5 mpk of LO14 LP-1 DAR8 (FIG. 8C, top). In the 22Rv1 model, which has lower STEAP2 expression, significant tumor regression was achieved at doses of 3-6 mpk (FIG. 8C, bottom). Although LUCAP147 and LUCAP70 PDX models also have low-endogenous STEAP2 expression, these models were highly sensitive to treatment with STEAP2-targeting ADCs. In both models, significant tumor regressions were observed at doses as low as 0.25 mpk and 1 mpk of LO14 LP-1 DAR8 (FIG. 8D).

Monotonic (always decreasing) generalized additive model (GAM) spline curves were fit to the dose response data to illustrate the statistical difference between the effects of LO14 TOP1i DAR4 and DAR8 ADCs across the tested CDX and PDX models (FIG. 8E). The results confirm that 22Rv1, C42, and LUCAP147 tumor treated with LO14 TOP1i DAR8 ADC had a significantly slower growth rate than the same tumor models treated with LO14 TOP1i DAR4 ADC. Additional data is shown in Table 4 below:

TABLE 4

| | Day | | | | | |
|---|---|---|---|---|---|---|
| Study* | Range | DAR4.Ix50 | DAR8.Ix50 | Diff | SE | Pval |
| 014-22Rv1 | 0-21 | 9.83 | 5.34 | −4.49 | 0.88 | 0.000 |
| 015-C42 | 0-40 | 1.49 | 0.93 | −0.56 | 0.17 | 0.000 |
| 017-147LuCaP | | 0.32 | 0.27 | −0.05 | 0.14 | 0.002 |
| 018-73LuCaP | | 0.17 | 0.12 | −0.05 | 0.07 | 0.086 |
| 019-70LuCaP | | 0.19 | 0.15 | −0.05 | 0.09 | 0.736 |

*Study refers to the CDX and PDX models; potency comparison for 22Rvl could only be performed out to 21 days as data was not available for all groups at 40 day timepoint

DAR4.Ix50/DAR8.Ix50 refers to the best fit value of the lambda, wherein best is based on the r-squared value.
Diff is the difference in relative growth rate between DAR4 and DAR8.
SE=standard error

### Example 8

### Therapeutic Activity of LO14 TOP1i DAR8 ADC

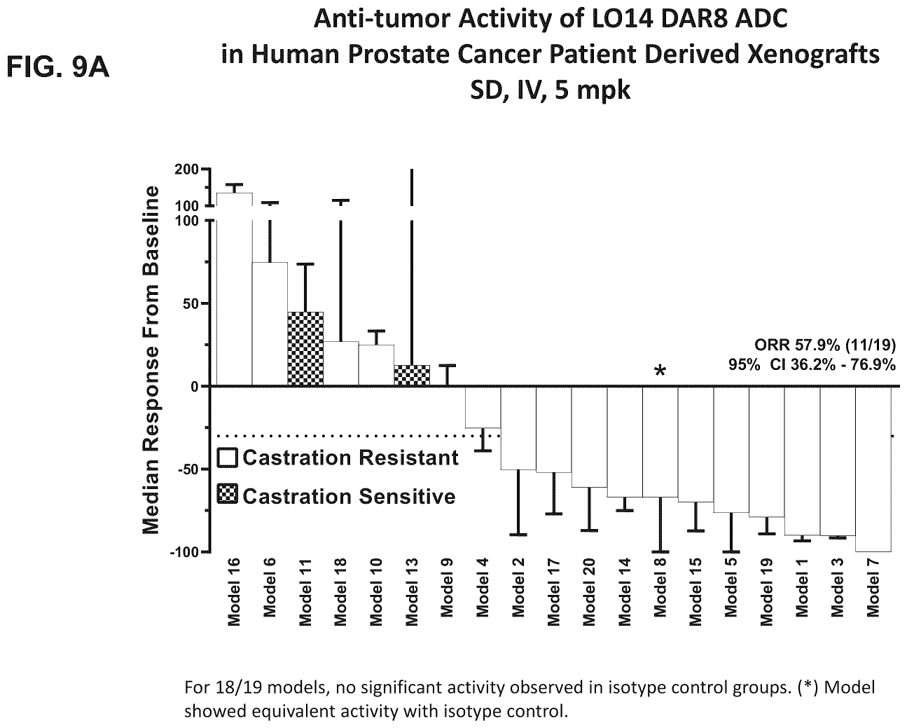

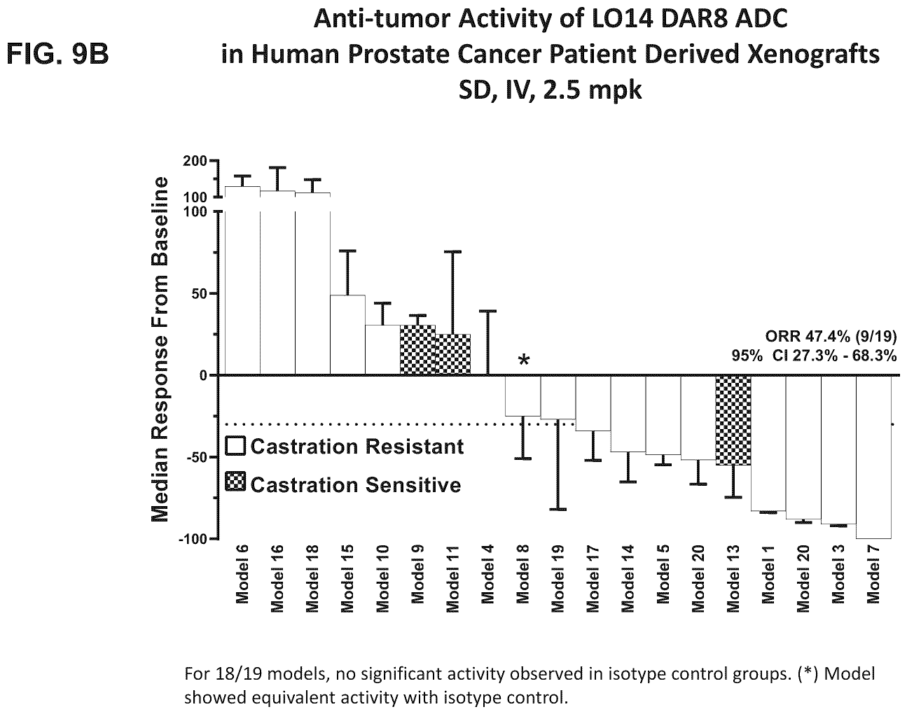

To determine whether LO14 TOP1i DAR8 ADCs demonstrates a therapeutic potential in prostate cancer, 19 patient derived xenografts were treated with either 2.5 or 5 mpk of ADC. Tumor tissue fragments were implanted subcutaneously into male NSG mice between 6 to 8 weeks of age. When tumors reached the appropriate tumor volume range, tumors were resected and expanded into a cohort of study animals. When the tumors in the study animals reached the appropriate tumor volume (150-300 mm³), animals were randomized into treatment and control groups (n=3 animals/group) and dosing was initiated. A single dose of test article was administered and animal body weights and tumor volumes were recorded as described above. The results shown in FIGS. 9A-9B are sorted in descending order of median response from baseline.

The PDX models evaluated in FIGS. 9A-9B represent tumor material from 19 distinctly different patients. Overall response rate (ORR) is defined as the percent of tumor models which showed a median response from baseline of −30% or lower (also described as significant 130% tumor growth inhibition). When administered at 5 mpk, LO14 TOP1i DAR8 ADC has an ORR of 57.9% (11/19) in comparison to the 5% (1/19) ORR with Isotype ADC administered at an equivalent dose (FIG. 9A). When administered at 2.5 mpk, LO14 TOP1i DAR8 ADC has an ORR of 47.4% (9/19) in comparison to the 5% (1/19) ORR with Isotype ADC administered at an equivalent dose (FIG. 9B). Overall, these results demonstrate that treatment with LO14 TOP1i DAR8 ADC is broadly efficacious in human prostate cancer.

### Example 9

### LP-1 Synthesis and ADC Conjugation

### General Information

Flash chromatography was performed using a BIOTAGE ISOLERA and fractions checked for purity using thin-layer chromatography (TLC). TLC was performed using MERCK KIESELGEL 60 F254 silica gel, with fluorescent indicator on aluminium plates. Visualisation of TLC was achieved with UV light.

Extraction and chromatography solvents were bought and used without further purification from VWR U.K.

All fine chemicals were purchased from SIGMA-ALDRICH unless otherwise stated.

Pegylated reagents were obtained from QUANTA BIODESIGN US via STRATECH UK.

### LC/MS Conditions

Positive mode electrospray mass spectrometry was performed using a WATERS ACQUITY H-CLASS SQD2 using one of the following methods.

(a) The HPLC (WATERS ALLIANCE 2695) was run using a mobile phase of water (A) (formic acid 0.1%) and acetonitrile (B) (formic acid 0.1%).

LCMS 3 min: Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Wavelength detection range: 190 to 800 nm. Columns: WATERS ACQUITY UPLC BEH SHIELD RP18 1.7 μm 2.1×50 mm at 50° C. fitted with WATERS ACQUITY UPLC BEH SHIELD RP18 VANGUARD Pre-column, 130A, 1.7 μm, 2.1 mm×5 mm.

LCMS 15 min: initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: WATERS ACQUITY UPLC CSH C18 1.7 μm 2.1×100 mm fitted with WATERS ACQUITY UPLC CSH C18 VANGUARD Pre-column, 1.7 μm, 2.1 mm×5 mm.

(b) The HPLC (Agilent 1290) was run using a mobile phase of water (A) (TFA 0.03%) and acetonitrile (B) (0.03% TFA), or water (A) (TFA 0.05%) and acetonitrile (B) (0.05% TFA). Initial composition was (a) 100% A held for 2-4 minutes then increased to 90% B over 2-5 minutes, or (b) 5%-20% B increased to 90%-98% B over 3-17 minutes. Flow rate was 0.3-1.5 mL/minute. Column was (1) ATLANTIS T3 3 μm 4.6*150 mm at 40° C. (Detector ELSD or Wavelength detection range: 210 nm), (2) ACQUITY UPLC BEH C18 2.1*100 mm 1.7 μm at 40° C. (Wavelength detection range: 210 nm or 220 nm), (3) UPLC BEH C18 1.7 μm, 2.1*100 mm at 40° C. (Wavelength detection range: 223 nm), (4) XBRIDGE C18 (4.6*150, 3.5 μm) at 40° C., (5) ACQUITY UPLC HSS PFP 2.1*150 mm 1.8 μm at 40° C. (Wavelength detection range: 220 nm), (6) UPLC BEH Phenyl 1.7 μm, 2.1*150 mm at 40° C. (Wavelength detection range: 210 nm), (7) EC-C18 2.7 μm, 3.0*50 mm at 40° C. (Wavelength detection range: 210 nm), or (8) YMC-Triart C18 50*3.0 mm S-3 um, 12 nm at 45° C. (Detector ELSD). Injection volume 2 μL.

### HPLC Conditions

Reverse-phase ultra-fast high-performance liquid chromatography (UFLC) was carried out on a SHIMADZU PROMINENCE machine using a PHENOMENEX GEMINI NX 5μ C18 column (at 50° C.) dimensions: 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 30% B over a 3 minutes period, then increased to 45% B over 8 minutes and again to 100% over 6 minutes before returning to 13% over 2 min and hold for 1 min. The total duration of the gradient run was 20.0 minutes. Flow rate was 20.0 mL/minute and detection was at 254 and 223 nm.

### NMR Method

Proton NMR chemical shift values were measured on the delta scale at 400 MHz using a BRUKER AV400. The following abbreviations have been used: s, singlet; d, doublet; t, triplet; q, quartet; quin, quintet; m, multiplet; br, broad. Coupling constants are reported in Hz.

### Abbreviations

TLC Thin layer chromatography
UV Ultra violet
LCMS Liquid chromatography mass spectrometry
CSH Charged surface hybrid
UPLC Ultra-performance liquid chromatography
RP Reverse phase
NMR Nuclear magnetic resonance
DBU 1,8-Diazabicyclo[5.4.0]undec-7-ene
DMF Dimethyl formamide
DCM Dichloromethane
TBS Tert-butyl dimethyl silane
MS Molecular sieves
TFA Trifluoro acetic acid
DMSO Dimethyl sulfoxide
ESI Electrospray ionisation
DIPEA Di isopropyl ethylamine
THF Tetra hydro furan
HATU Hexafluorophosphate azabenzotriazole tetramethyl uronium
TEA Triethylamine
HOPO 1-Hydroxy-2-pyridone
RT Retention time
ADC Antibody-drug conjugate
UHPLC Ultra-high performance liquid chromatography
mAb Monoclonal antibody
SEC Size exclusion chromatography
DAR Drug to antibody ratio
RPMI Roswell Park Memorial Institute
ND Not detectable
IC₅₀ Inhibitory concentration 50%
Fmoc fluorenylmethoxycarbonyl

### Intermediate 1

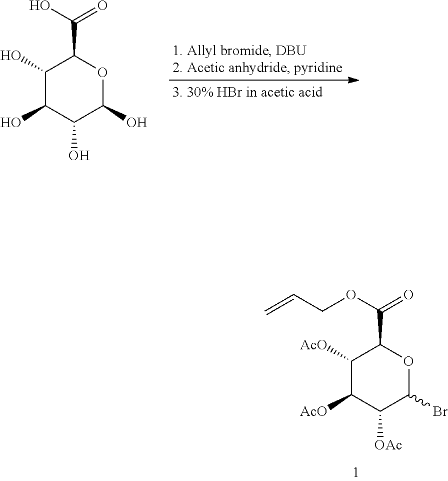 [SMILES: O=C(O)[C@H]1O[C@@H](O)[C@H](O)[C@@H](O)[C@@H]1O ; C=CCOC(=O)[C@H]1OC(Br)[C@H](OC(C)=O)[C@@H](OC(C)=O)[C@@H]1OC(C)=O]

2,3,4,6,7,8,9,10-octahydropyrimido[1,2-a]azepine (26.5 ml, 177.35 mmol) was added dropwise to a 1-L round bottom flask containing (2S,3S,4S,5R,6R)-3,4,5,6-tetrahydroxytetrahydro-2H-pyran-2-carboxylic acid (31.3 g, 161.22 mmol) in DMF (100 ml) at 21° C. Next, 3-bromoprop-1-ene (16.72 ml, 193.47 mmol) was added to the reaction mixture dropwise over 10 minutes and the reaction was stirred at 21° C. for 24 hours. Reaction mixture was cooled to 0° C. and treated with pyridine (104 mL, 1289.60 mmol). Acetic anhydride (244 mL, 2579.20 mmol) was next added to the reaction mixture. The reaction was warmed up to room temperature and run for 2 hours at 21° C. Reaction mixture concentrated under reduced vacuum and the remaining pyridine was azeotropically removed with toluene (1×100 mL). Crude material was diluted with DCM (65 mL) and cooled to 0° C. 30% Hydrobromic acid in acetic acid (175 mL, 3226.03 mmol) was next added to the reaction mixture at 0° C. The reaction was warmed up to room temperature and run for 2 hours 30 minutes at 21° C. Solvent was evaporated then the compound was purified by normal phase flash column chromatography to afford (2S,3S,4S,5R,6R)-2-((allyloxy)carbonyl)-6-bromotetrahydro-2H-pyran-3,4,5-triyl triacetate Intermediate 1 (33 g, 48% yield) as a beige translucent material. ¹H NMR (500 MHz, CDCl₃) δ 6.67 (d, J=4.0 Hz, 1H), 5.92 (ddt, J=16.6, 10.3, 6.0 Hz, 1H), 5.64 (t, J=9.7 Hz, 1H), 5.42-5.23 (m, 3H), 4.88 (dd, J=10.0, 4.0 Hz, 1H), 4.71-4.58 (m, 3H), 2.12 (s, 3H), 2.07 (s, 3H), 2.05 (s, 3H); LCMS (ESI) m/z 445.0 (M+Na)+.

### Alternative Synthesis of Intermediate 1

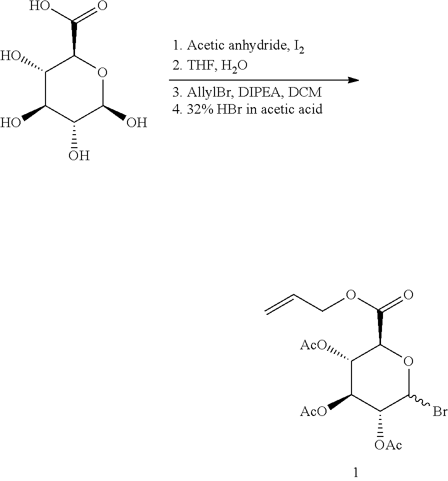 [SMILES: O=C(O)[C@H]1O[C@@H](O)[C@H](O)[C@@H](O)[C@@H]1O ; C=CCOC(=O)[C@H]1OC(Br)[C@H](OC(C)=O)[C@@H](OC(C)=O)[C@@H]1OC(C)=O]

Iodine (1.19 kg, 4.69 mol) was added to acetic anhydride (3500 mL) stirred at 0-10° C. under nitrogen. The resulting mixture was adjusted to 20-30° C. and Glucuronic acid (7 kg, 36.06 mol) was added portion wise, maintaining the temperature at 25-30° C. The reaction was stirred at this temperature for 1 hour under nitrogen and then cooled to 0° C. A solution of sodium thiosulfate pentahydrate (2.33 kg) in water (35.2 L) was added to the stirred mixture at 0-10° C., and then stirred to 2 hours at 20-30° C. Water (35.2 L) was added to the stirred mixture, extracted with isopropyl acetate (3×35.2 L), and the organic layer was concentrated to dryness to give crude 1,2,3,4-tetra-O-acetyl-β-D-glucuronic acid (16.08 kg, 61% w/w assay, 75%). LCMS m/z (ES+), [M+Na]⁺=384.6

Crude 1,2,3,4-tetra-O-acetyl-β-D-glucuronic acid, 61% w/w (16 kg; 27.05 mol) was dissolved in isopropyl acetate (42.83 kg) and stirred at 20-30° C. N,N-diispropylethylamine (12.25 kg, 94.68 mol) was added to the reaction at 20-30° C. over 11 minutes followed by 3-bromopropene (9.8 kg, 81.15 mol) added dropwise at 20-30° C. over 5 minutes. The resulting mixture was stirred for 48 hours at 20-30° C. Isopropyl acetate (42.83 kg) and water (49 kg) were added to the stirred mixture. The organic layer separated and adjusted to pH 4-5 at 20-30° C. by addition of aqueous hydrochloric acid (0.6N, 43.71 kg). The separated organic layer was washed with brine (25% aqueous solution, 49 L), and concentrated to dryness to give crude 1,2,3,4-tetra-O-acetyl-β-D-glucuronic acid allyl ester as a brown solid (12.0 kg, 87.5% w/w assay, 86.6%). LCMS m/z (ES+), [M+Na]⁺=424.833% hydrobromic acid in acetic acid (534 mL, 2.982 mol) was added dropwise to a stirred mixture of crude 1,2,3,4-tetra-O-acetyl-β-D-glucuronic acid allyl ester (200 g, 0.497 mol) in isopropyl acetate (500 mL) at 0° C. The reaction was adjusted to 20-30° C. and stirred for 8 hours. The reaction mixture was extracted with isopropyl acetate (2400 mL), the extract washed with brine (25% aqueous, 3×2000 mL) and concentrated to dryness to give crude product as a black oil (231.3 g, 80.5%). LCMS (ES+), [M+Na]⁺=445.2 & 447, ¹H NMR (300 MHz, CDCl₃) δ 6.65 (d, J=4.2 Hz, 1H), 5.59-5.84 (m, 1H), 5.62 (t, J=9.6 Hz, 1H), 5.40-5.23 (m, 3H), 4.87 (dd, J=9.9, 3.9 Hz, 1H), 4.66-4.59 (m, 3H), 2.21-2.03 (m, 9H)

### Intermediate 2

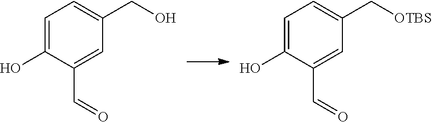 [SMILES: O=Cc1cc(CO)ccc1O ; CC(C)(C)[Si](C)(C)OCc1ccc(O)c(C=O)c1]

TBS-Cl (20.80 g, 138.02 mmol) in DCM (25 mL) was added dropwise to 1H-imidazole (17.90 g, 262.90 mmol) and 2-hydroxy-5-(hydroxymethyl)benzaldehyde (20 g, 131.45 mmol) in DCM (500 mL) at 0° C. over a period of 2 hours under nitrogen. The resulting mixture was stirred at 0° C. for 2 hours. The reaction mixture was quenched with water (500 mL), extracted with DCM (2×300 mL), the organic layer was dried over Na2SO4, filtered and evaporated to afford 5-(((tert-butyldimethylsilyl)oxy)methyl)-2-hydroxybenzaldehyde Intermediate 2 (35.0 g, 100%) as a colourless material. m/z (ES+), [M+Na]⁺=289; NH₄HCO₃, HPLC tR=1.505 min

### Intermediate 3

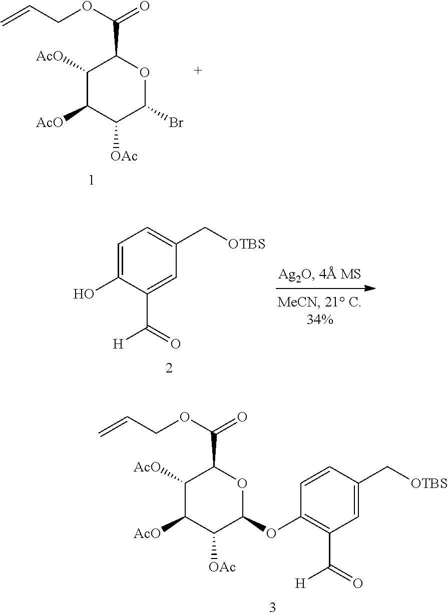 [SMILES: C=CCOC(=O)[C@H]1O[C@H](Br)[C@H](OC(C)=O)[C@@H](OC(C)=O)[C@@H]1OC(C)=O ; CC(C)(C)[Si](C)(C)OCc1ccc(O)c(C=O)c1 ; C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O]

To a vacuum-dried 500 mL round-bottom flask was added molecular sieves (4 Å beads, 5.0 g), silver oxide (29.2 g, 125.8 mmol) and acetonitrile (150 mL), producing a black slurry. To this slurry was added a solution of Intermediate 1 (10.7 g, 25.2 mmol) in acetonitrile (50 mL) over 20 min followed by the addition of 5-(((tert-butyldimethylsilyl)oxy)methyl)-2-hydroxybenzaldehyde (Intermediate 2, 13.6 g, 51.1 mmol) in acetonitrile (50 mL) in one portion. The resulting mixture was stirred vigorously at 20° C. for 16 h. After 16 h, the reaction mixture was filtered through a 5-cm pad of Celite and rinsed with dichloromethane (3×25 mL). Solvent was evaporated then the compound was purified by normal phase flash column chromatography to afford (2S,3S,4S,5R,6S)-2-((allyloxy)carbonyl)-6-(4-(((tert-butyldimethylsilyl)oxy)methyl)-2-formylphenoxy)tetrahydro-2H-pyran-3,4,5-triyl triacetate as a white material Intermediate 3 (5.2 g, 34% yield). ¹H NMR (400 MHz, CDCl₃) δ 10.34 (s, 1H), 7.77 (d, J=1.8 Hz, 1H), 7.58 (dd, J=8.6, 2.1 Hz, 1H), 7.14 (d, J=8.6 Hz, 1H), 5.81-5.92 (m, 1H), 5.39-5.35 (m, 4H), 5.28-5.22 (m, 2H), 4.71 (s, 2H), 4.58-4.67 (m, 2H), 4.20-4.28 (m, 1H), 2.073 (s, 3H), 2.069 (s, 3H), 2.04 (s, 3H), 0.94 (s, 9H), 0.11 (s, 6H); LCMS (ESI) m/z 626.3 (M+NH₄)+.

### Intermediate 4

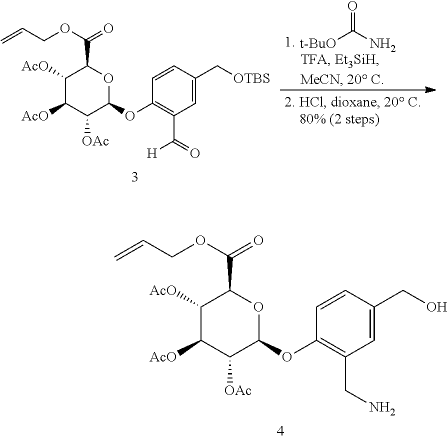 [SMILES: C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O ; CC(C)(C)OC(N)=O ; C=CCOC(=O)[C@H]1O[C@@H](Oc2ccc(CO)cc2CN)[C@H](OC(C)=O)[C@@H](OC(C)=O)[C@@H]1OC(C)=O]

To a solution of Intermediate 3 (5.2 g, 8.6 mmol) in acetonitrile (40 mL) was added tert-butyl carbamate (3.8 g, 32.3 mmol), trifluoroacetic acid (2.0 mL, 25.9 mmol), and triethylsilane (4.1 mL, 25.8 mmol). Stirred for 2 h at 20° C. then solvent was evaporated. To the resulting colorless oil was added 1,4-dioxane (8 mL) and HCl (4.0 M in 1,4-dioxane, 50 mL, 200 mmol). The mixture was stirred at 20° C. for 30 min the solvent was evaporated. The resulting white powder was dissolved in DMSO (3 mL) then passed through cation-exchange resin pre-treated with methanol (WATERS PORAPAK CX). The desired compound was eluted off the resin with methanol to afford (2S,3S,4S,5R,6S)-2-((allyloxy)carbonyl)-6-(2-(aminomethyl)-4-(hydroxymethyl)phenoxy)tetrahydro-2H-pyran-3,4,5-triyl triacetate as a white material Intermediate 4 (2.5 g, 80% over 2 steps). ¹H NMR (500 MHz, CDCl₃) δ 7.26 (d, J=2.2 Hz, 1H), 7.21 (dd, J=8.3, 2.2 Hz, 1H), 7.01 (d, J=8.3 Hz, 1H), 5.90-5.82 (m, 1H), 5.42-5.24 (m, 6H), 5.16 (d, J=7.1 Hz, 1H), 4.64-4.55 (m, 4H), 4.19 (d, J=9.3 Hz, 1H), 3.84 (d, J=14.0 Hz, 1H), 3.67 (d, J=14.0 Hz, 1H), 2.32 (s, 3H), 2.09 (s, 3H), 2.07 (s, 3H), 2.03 (s, 3H). LCMS (ESI) m/z 496.5 (M+H)+.

### Intermediate 5

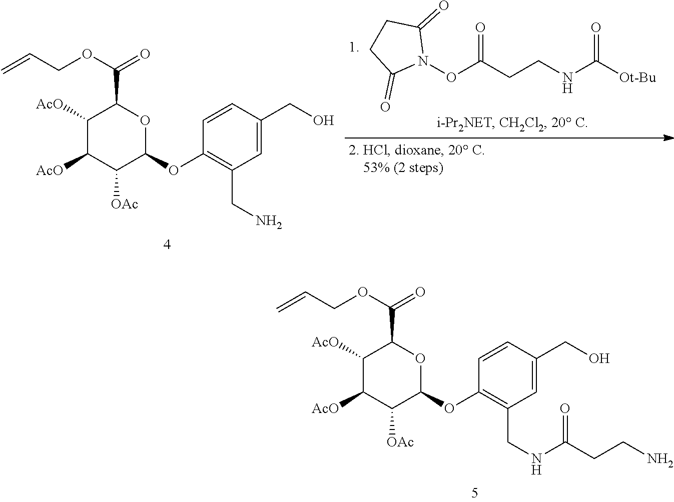 [SMILES: C=CCOC(=O)[C@H]1O[C@@H](Oc2ccc(CO)cc2CN)[C@H](OC(C)=O)[C@@H](OC(C)=O)[C@@H]1OC(C)=O ; CC(C)(C)OC(=O)NCCC(=O)ON1C(=O)CCC1=O ; C=CCOC(=O)[C@H]1O[C@@H](Oc2ccc(CO)cc2CNC(=O)CCN)[C@H](OC(C)=O)[C@@H](OC(C)=O)[C@@H]1OC(C)=O]

To a suspension of Intermediate 4 (2.5 g, 5.0 mmol) in dichloromethane (20 mL) was added N-ethyl-N-isopropylpropan-2-amine (1.8 mL, 10.1 mmol) and 2,5-dioxopyrrolidin-1-yl 3-((tert-butoxycarbonyl)amino)propanoate (1.3 g, 4.4 mmol). Stirred at 20° C. for 10 minutes then water (50 mL) was added. Organic layer was separated then the aqueous layer was extracted with dichloromethane (3×30 mL). Combined organic layers were dried over Na₂SO₄ the solvent was evaporated. To a solution of (2S,3S,4S,5R,6S)-2-((allyloxy)carbonyl)-6-(2-((3-((tert-butoxycarbonyl)amino)propanamido)methyl)-4-(hydroxymethyl)phenoxy)tetrahydro-2H-pyran-3,4,5-triyl triacetate (2.8 g, 4.2 mmol) in dichloromethane (20 mL) was added hydrochloric acid (4.0 M in 1,4-dioxane, 2.6 mL, 83.9 mmol). Stirred at 20° C. for 2 h then solvent was evaporated. The compound was purified by reverse phase flash column chromatography to afford (2S,3S,4S,5R,6S)-2-((allyloxy)carbonyl)-6-(2-((3-aminopropanamido)methyl)-4-(hydroxymethyl)phenoxy)tetrahydro-2H-pyran-3,4,5-triyl triacetate as a colorless material Intermediate 5 (1.3 g, 53% over 2 steps). ¹H NMR (500 MHz, D₂O) δ 7.22 (d, J=2.2 Hz, 1H), 7.16 (d, J=8.2 Hz, 1H), 7.09 (d, J=8.4 Hz, 1H), 5.84 (ddt, J=16.6, 10.5, 6.0 Hz, 1H), 5.44 (t, J=9.2 Hz, 1H), 5.38 (dd, J=7.6, 3.3 Hz, 1H), 5.35-5.23 (m, 4H), 4.62 (d, J=9.8 Hz, 1H), 4.56 (d, J=6.0 Hz, 2H), 4.51 (s, 2H), 4.26 (q, J=15.3 Hz, 2H), 3.23 (t, J=6.8 Hz, 2H), 2.68 (td, J=6.8, 1.9 Hz, 2H), 2.06 (d, J=10.4 Hz, 9H). LCMS (ESI) m/z 567.2 (M+H)+.

### Alternative Synthesis of Intermediate 5

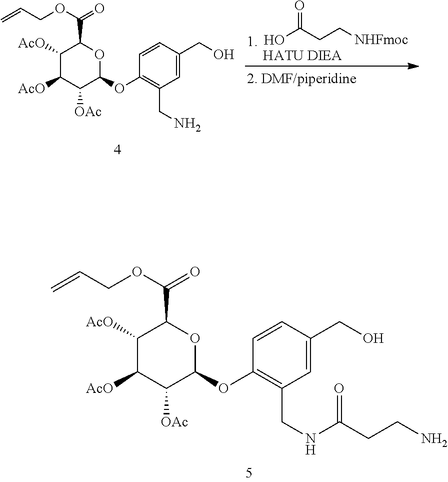

To a stirred reactor containing Intermediate 4 (2.1 kg, 90.5% w/w, 3.57 mol) and acetonitrile (19 L) was added Fmoc-β-alanine (1.11 kg, 3.57 mol). The stirred mixture was cooled to 0° C. To this was added hexafluorophosphate azabenzotriazole tetramethyl uronium (1.36 kg, 3.57 mol) and N,N-diispropylethylamine (0.92 kg, 7.14 mol), and stirred for 4 hours, maintaining the temperature at 0° C. Water (19 L) and ethyl acetate (19 L) was added to the stirred mixture. The organic phase was separated and concentrated to ˜19 L under vacuum. Ethyl acetate (28.5 L) was added to the concentrated solution and stirred at 20-25° C. for 18 hours. The resulting suspension was filtered, the cake washed with ethyl acetate (3.87 L), and dried under vacuum to (2S,3R,4S,5S,6S)-2-(2-((3-((((9H-fluoren-9-yl)methoxy)carbonyl)amino)propanamido)methyl)-4-(hydroxymethyl)phenoxy)-6-((allyloxy)carbonyl)tetrahydro-2H-pyran-3,4,5-triyl triacetate (1.6 kg, 99% w/w, 56%). LCMS m/z 789 [M+H]⁺

To a stirred reactor containing (2S,3R,4S,5S,6S)-2-(2-((3-((((9H-fluoren-9-yl)methoxy)carbonyl)amino)propanamido)methyl)-4-(hydroxymethyl)phenoxy)-6-((allyloxy)carbonyl)tetrahydro-2H-pyran-3,4,5-triyl triacetate (1 kg, 1.27 mol) and tetrahydrofuran (10 L) at −45° C. under nitrogen was added 2,3,4,6,7,8,9,10-octahydropyrimido[1,2-a]azepine (385.98 g, 2.54 mol). The mixture was stirred at −45° C. for four hours then diluted with acetonitrile (5 L) and quenched by the addition of hydrogen chloride in tert-butyl methyl ether solution (2.54 L, 2.0 M, 5.07 mol). The mixture was concentrated to ˜5 L under vacuum, and diluted with n-heptane (5 L). The acetonitrile layer was collected containing Intermediate 8 (3.88 kg of MeCN solution, 89.97% area, assumed 100%). LCMS m/z 566.6 [M+H]⁺

### Intermediate 7

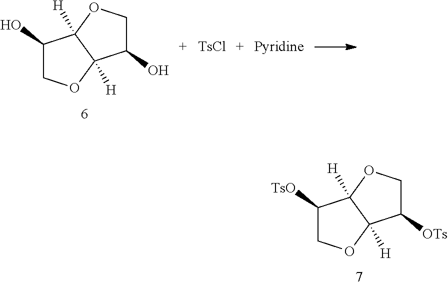 [SMILES: O[C@@H]1CO[C@H]2[C@@H]1OC[C@H]2O ; "TsCl" ; Cc1ccc(S(=O)(=O)O[C@@H]2CO[C@H]3[C@@H]2OC[C@H]3OS(=O)(=O)c2ccc(C)cc2)cc1]

To a 250 mL round bottom flask was added Intermediate 6 (5.0 g, 34.21 mmol)) in dry DCM (100 mL) under nitrogen gas. To the solution was added pyridine (13.84 mL, 171.07 mmol) followed by tosyl-Cl (16.31 g, 85.53 mmol). The reaction mixture was stirred at 20° C. for 16 h. LC-MS analysis showed formation of desired product and completion of reaction. The reaction mixture was diluted with dichloromethane (200). Organic layer was separated, and compound was extracted in 200 mL dichloromethane. Combined organic layer was washed with HCl solution (1M-300 mL), brine (200 mL) and dried over magnesium sulfate. Solvent was removed under reduced pressure to get crude products. The compound was purified via silica gel column to give (3R,3aS,6R,6aS)-hexahydrofuro[3,2-b]furan-3,6-diyl bis(4-methylbenzenesulfonate) Intermediate 7 (14.90 g, 96%). 1H NMR (500 MHz, CDCl3) δ 7.90-7.76 (m, 4H), 7.45-7.33 (m, 4H), 4.94-4.80 (m, 2H), 4.55-4.44 (m, 2H), 3.94 (dd, J=9.6, 6.7 Hz, 2H), 3.75 (dd, J=9.6, 7.6 Hz, 2H), 2.48 (s, 6H). LCMS (ESI) m/z 455.21 (M+H)⁺.

### Intermediate 8

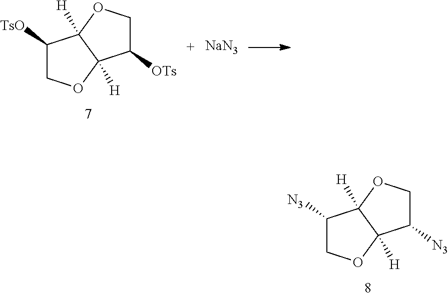 [SMILES: Cc1ccc(S(=O)(=O)O[C@@H]2CO[C@H]3[C@@H]2OC[C@H]3OS(=O)(=O)c2ccc(C)cc2)cc1 ; [N-]=[N+]=N[C@H]1CO[C@H]2[C@@H]1OC[C@@H]2N=[N+]=[N-] ; "NaN3"]

To a 50 mL round bottom flask was added Intermediate 7 (6.0 g, 13.20 mmol) in dry DMF (15 mL) under nitrogen gas. To the solution was added sodium azide (2.146 g, 33.00 mmol). The reaction mixture was at 140° C. for 3 hrs. LC-MS analysis showed formation of desired product and completion of reaction. The reaction mixture was diluted with dichloromethane (200×2 mL), and organic layer was separated, washed with water (200 mL), brine (200 mL) and dried over magnesium sulfate. Solvent was removed under reduced pressure to get (3S,3aR,6S,6aR)-3,6-diazidohexahydrofuro[3,2-b]furan Intermediate 8 (2.050 g, 79%). ¹H NMR (500 MHz, CDCl₃) δ 4.61 (d, J=1.9 Hz, 2H), 4.05 (d, J=4.0 Hz, 2H), 3.97-3.82 (m, 4H). LCMS (ESI) m/z 197.1 (M+H)⁺.

### Intermediate 9

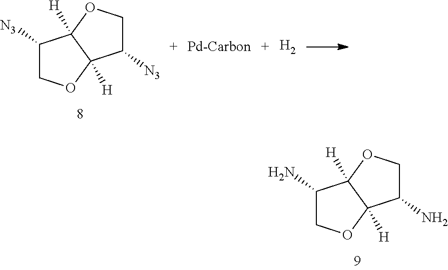 [SMILES: [N-]=[N+]=N[C@H]1CO[C@H]2[C@@H]1OC[C@@H]2N=[N+]=[N-] ; "H2" ; N[C@H]1CO[C@H]2[C@@H]1OC[C@@H]2N]

To a 250 mL round bottom flask was added Intermediate 8 (1 g, 5.10 mmol) in dry THF (20 mL) under nitrogen gas. To the solution was added barium palladium(II) carbonate (0.618 g, 0.51 mmol). The reaction mixture was flushed with hydrogen (1.028 g, 509.76 mmol) gas and stirred at 23° C. for 3 hrs. under H₂ gas. LC-MS analysis showed formation of desired product and completion of reaction. The reaction mixture was diluted with methanol (20 mL) filtered through celite pad. Celite pad was washed with methanol (50 mL). Filtrate was dried over magnesium sulfate. Solvent was removed under reduced pressure to get (3S,3aR,6S,6aR)-hexahydrofuro[3,2-b]furan-3,6-diamine Intermediate 9 (0.590 g, 80%). ¹H NMR (500 MHz, DMSO) δ 4.23 (s, 2H), 3.68 (dd, J=8.7, 4.5 Hz, 2H), 3.41 (dd, J=8.7, 1.9 Hz, 2H), 3.23 (dd, J=4.5, 1.9 Hz, 2H), 1.54 (s, 4H). LCMS (ESI) m/z 145.2 (M+H)⁺.

### Alternative Synthesis of Intermediate 9

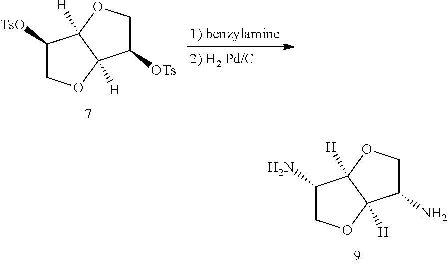 [SMILES: Cc1ccc(S(=O)(=O)O[C@@H]2CO[C@H]3[C@@H]2OC[C@H]3OS(=O)(=O)c2ccc(C)cc2)cc1 ; N[C@H]1CO[C@H]2[C@@H]1OC[C@@H]2N]

To reactor was added intermediate 6 (4 kg, 8.8 mol) and benzylamine (12 L) under nitrogen. The stirred mixture was heated to 160° C. for 24 hours then cooled to 20-25° C., diluted with tert-butyl methyl ether (80 L) and cooled further to 10° C. To this was added para-toluene sulfonic acid (12.11 kg, 70.44 mol), the mixture stirred for 2.5 hours at 20-25° C. and then filtered. The cake was washed with tert-butyl methyl ether (8 L) and the combined filtrates washed with saturated aqueous sodium hydrogen carbonate solution (20 L). The organic phase was evaporated to dryness, dissolved in ethanol (20 L) and evaporated to dryness to give crude intermediate 7 (3.05 kg, 80.5% w/w, 85.9%) LCMS m/z (ES+), [M+H]⁺=325.1

To a reactor was added Intermediate 7 (1.5 kg, 3.08 mol) and ethanol (12.12 L) under a dry under nitrogen atmosphere. To the solution was added 10% wt palladium on carbon (120.8, 10% w/w). The reaction mixture was flushed with hydrogen gas and stirred at 80° C. for 16 hours under hydrogen. Mixture cooled to 20-25° C. and filtered through cellulose (2.42 kg). The cake was washed with ethanol (2.44 L) and combined filtrates were concentrated to dryness. The residue was dissolved in acetonitrile (6.04 L) and concentrated to dryness to give Intermediate 9 (509 g, 84% w/w, 80.2%). LCMS m/z (ES+), [M+H]⁺=145

### Intermediate 11

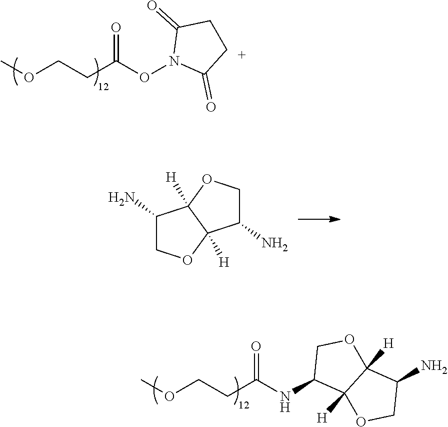

To a 250 mL round bottom flask was added Intermediate 9 (1.50 g, 10.40 mmol) in dry THF (25 mL) under nitrogen gas. To the solution was added sodium hydrogen carbonate (1.748 μg, 20.81 mmol) and 2,5-dioxopyrrolidin-1-yl 2,5,8,11,14,17,20,23,26,29,32,35-dodecaoxaoctatriacontan-38-oate Intermediate 10 (7.13 g, 10.40 mmol) in portions under nitrogen gas and stirred at 20° C. for 6 hrs. LC-MS analysis showed formation of desired product and completion of reaction. The reaction mixture was quenched by addition of methanol (10 mL). The reaction mixture was diluted with methanol (20 mL) filtered through celite pad. Celite pad was washed with methanol (50 mL). Filtrate was dried over magnesium sulfate. Solvent was removed under reduced pressure to get crude product. the crud product was purified via silica gel column to give N-((3S,3aR,6S,6aR)-6-aminohexahydrofuro[3,2-b]furan-3-yl)-2,5,8,11,14,17,20,23,26,29,32,35-dodecaoxaoctatriacontan-38-amide Intermediate 11 (4.00 g, 53.8%). ¹H NMR (500 MHz, MeOD) δ 4.64-4.59 (m, 1H), 4.45 (dd, J=4.1, 1.3 Hz, 1H), 4.29 (dt, J=4.1, 1.9 Hz, 1H), 3.96 (ddd, J=10.2, 9.3, 4.9 Hz, 2H), 3.81-3.74 (m, 3H), 3.73-3.62 (m, 45H), 3.61-3.56 (m, 2H), 3.45 (dt, J=3.8, 1.8 Hz, 1H), 3.40 (s, 3H), 2.52-2.47 (m, 2H). LCMS (ESI) m/z 715.6 (M+H)⁺.

### Intermediate 13

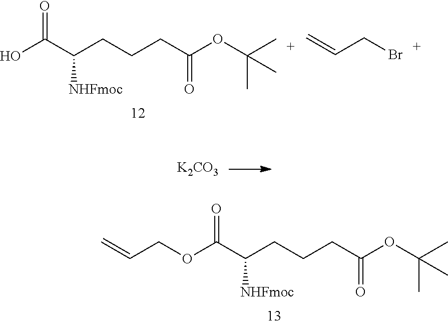

To a 250 mL round bottom flask was added (S)-2-((((9H-fluoren-9-yl)methoxy)carbonyl)amino)-6-(tert-butoxy)-6-oxohexanoic acid Intermediate 12 (5 g, 11.38 mmol) in dry DMF (20 mL) under nitrogen gas. To the solution was added potassium carbonate (3.14 g, 22.75 mmol) and 3-bromoprop-1-ene (1.485 mL, 17.06 mmol) in portions under nitrogen gas and stirred at 20° C. for 16 hrs. LC-MS analysis showed formation of desired product and completion of reaction. The reaction mixture was diluted with water (500 mL) and organic layer was extracted with ethyl acetate (2×300 mL), washed with water (300 mL), brine (200 mL) and dried over sodium sulfate (20 g). The solvent was removed to get crude product. The crude product was purified via silica gel column to give 1-allyl 6-(tert-butyl) (S)-2-((((9H-fluoren-9-yl)methoxy)carbonyl)amino)hexanedioate Intermediate 13 (5.10 g, 93%). ¹H NMR (500 MHz, CDCl₃) δ 7.79-7.73 (m, 2H), 7.61 (q, J=3.9 Hz, 2H), 7.40 (t, J=7.5 Hz, 2H), 7.32 (tt, J=7.4, 1.2 Hz, 2H), 5.91 (ddt, J=16.5, 10.9, 5.8 Hz, 1H), 5.42-5.23 (m, 3H), 4.66 (d, J=5.8 Hz, 2H), 4.40 (q, J=4.8 Hz, 3H), 4.23 (t, J=7.1 Hz, 1H), 2.26 (t, J=7.2 Hz, 2H), 1.96-1.82 (m, 1H), 1.72 (dq, J=13.5, 6.1 Hz, 3H), 1.60-1.47 (m, 1H), 1.45 (s, 9H). LCMS (ESI) m/z 480.2 (M+H)⁺.

### Intermediate 14

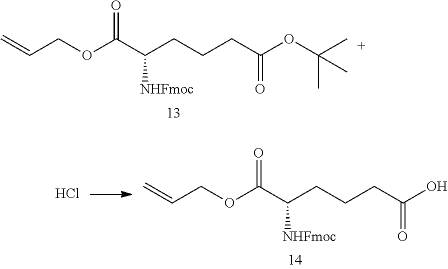

To a 100 mL round bottom flask was added Intermediate 13 (5 g, 10.43 mmol) in dry THF (20 mL) under nitrogen gas. To the solution was added HCl (13.03 mL, 52.13 mmol), 4 molar in dioxane under nitrogen gas and stirred at 20° C. for 6 hrs. LC-MS analysis showed formation of desired product and completion of reaction. The reaction mixture was diluted with water (200 mL) and organic layer was extracted with dichloromethane (2×300 mL), washed with brine (200 mL) and dried over sodium sulfate (20 g). The solvent was removed to get (S)-5-((((9H-fluoren-9-yl)methoxy)carbonyl)amino)-6-(allyloxy)-6-oxohexanoic acid Intermediate 14 (4.20 g, 95%). ¹H NMR (500 MHz, CDCl₃) δ 7.75 (dq, J=7.6, 1.0 Hz, 2H), 7.62-7.52 (m, 2H), 7.42-7.35 (m, 2H), 7.30 (tt, J=7.4, 1.2 Hz, 2H), 5.90 (ddt, J=16.4, 10.8, 5.8 Hz, 1H), 5.48 (d, J=8.4 Hz, 1H), 5.37-5.20 (m, 2H), 4.64 (d, J=5.8 Hz, 2H), 4.40 (d, J=7.2 Hz, 3H), 4.22 (t, J=7.0 Hz, 1H), 2.45-2.24 (m, 2H), 1.93 (p, J=5.6 Hz, 1H), 1.72 (td, J=13.9, 6.8 Hz, 3H). (ESI) m/z 424.5 (M−H)⁻.

### Intermediate 15

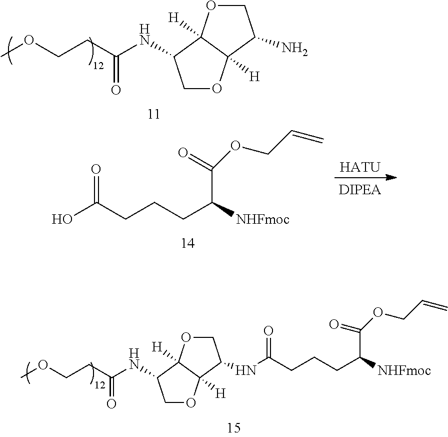

To a 100 mL round bottom flask was added Intermediate 14 (1.925 g, 4.55 mmol) under nitrogen gas. To the solution was added HATU (1.862 g, 4.90 mmol) followed by DIPEA (1.222 mL, 6.99 mmol). The reaction mixture was stirred at room temperature for 15 min then Intermediate 11 (2.5 g, 3.50 mmol). was added and reaction mixture was stirred at 23° C. for 3 hrs. LC-MS analysis showed formation of desired product and completion of reaction. The reaction mixture was diluted with DCM (300 mL), washed with water (200 mL), organic layer was extracted (2×100 mL), washed with Brine (50 mL), dried over sodium sulfate (5 g). Solvent was removed under reduced pressure to get crude product. The crude product was purified via silica gel column to give allyl (S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-((((9H-fluoren-9-yl)methoxy)carbonyl)amino)-6-oxohexanoate Intermediate 15 (3.40 g, 87%) ¹H NMR (500 MHz, MeOD) δ 7.86 (dd, J=7.6, 1.2 Hz, 2H), 7.74 (t, J=7.8 Hz, 2H), 7.46 (td, J=7.5, 1.4 Hz, 2H), 7.38 (tt, J=7.5, 1.3 Hz, 2H), 6.05-5.93 (m, 1H), 5.39 (dq, J=17.2, 1.6 Hz, 1H), 5.28 (dq, J=10.5, 1.4 Hz, 1H), 4.73-4.65 (m, 2H), 4.58 (qd, J=4.1, 1.0 Hz, 2H), 4.46 (dd, J=10.6, 7.0 Hz, 1H), 4.40 (dd, J=10.6, 7.0 Hz, 1H), 4.36-4.31 (m, 2H), 4.31-4.24 (m, 2H), 4.01 (ddd, J=9.6, 5.0, 1.1 Hz, 2H), 3.84-3.73 (m, 5H), 3.72-3.60 (m, 44H), 3.60-3.56 (m, 2H), 3.41 (s, 3H), 2.49 (td, J=6.0, 1.9 Hz, 2H), 2.31 (hept, J=7.2 Hz, 2H), 1.96-1.85 (m, 1H), 1.85-1.68 (m, 3H). LCMS (ESI) m/z 1121.3 (M+H)⁺.

### Intermediate 16

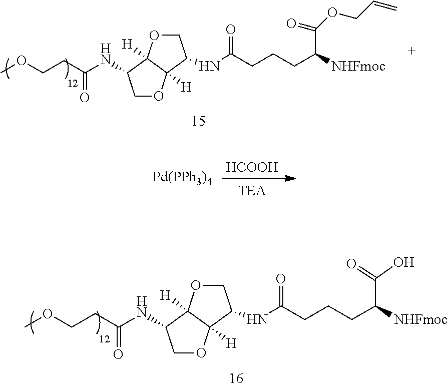

To a 50 mL round bottom flask was added Intermediate 15 (4.3 g, 3.84 mmol) in dry DCM (10 mL) under nitrogen gas. To the solution was added triethylamine (0.535 mL, 3.84 mmol) followed by triphenylphosphine (0.101 g, 0.38 mmol). To the reaction mixture was added Pd(PPh₃)₄ (0.444 g, 0.38 mmol) then formic acid (0.147 mL, 3.84 mmol) was added and reaction mixture was stirred at 23° C. for 6 hrs. LC-MS analysis showed formation of desired product and completion of reaction. Solvent was removed under reduced pressure to get crude products. The crude product was purified via silica gel column to give (S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-((((9H-fluoren-9-yl)methoxy)carbonyl)amino)-6-oxohexanoic acid Intermediate 16 (3.50 g, 84%). ¹H NMR (500 MHz, DMSO) δ 8.12 (dd, J=10.2, 7.0 Hz, 2H), 7.90 (d, J=7.6 Hz, 2H), 7.74 (d, J=7.5 Hz, 2H), 7.63 (d, J=8.1 Hz, 1H), 7.46-7.38 (m, 2H), 7.34 (td, J=7.4, 1.2 Hz, 2H), 4.38 (s, 2H), 4.32-4.20 (m, 3H), 4.11 (ddt, J=7.2, 4.8, 2.1 Hz, 2H), 3.93 (td, J=8.4, 4.6 Hz, 1H), 3.85 (dd, J=9.3, 5.1 Hz, 2H), 3.63-3.57 (m, 4H), 3.54-3.45 (m, 42H), 3.45-3.40 (m, 2H), 3.24 (s, 3H), 2.33 (t, J=6.5 Hz, 2H), 2.09 (s, 3H), 1.68 (d, J=9.1 Hz, 1H), 1.64-1.48 (m, 3H). LCMS (ESI) m/z 1080.6 (M+H)⁺.

### Intermediate 17

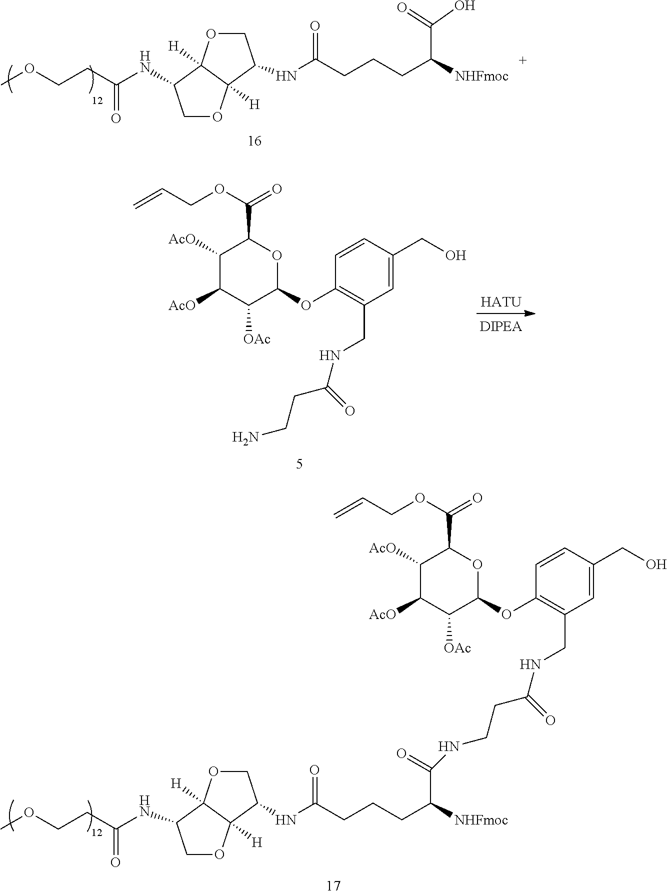

To a 100 mL round bottom flask was added Intermediate 16 (0.8 g, 0.74 mmol) under nitrogen gas. To the solution was added HATU (0.366 g, 0.96 mmol) followed by DIPEA (0.388 mL, 2.22 mmol). The reaction mixture was stirred at room temperature for 15 min then Intermediate 5 (0.670 g, 1.11 mmol). was added and reaction mixture was stirred at 23° C. for 3 hrs. LC-MS analysis showed formation of desired product and completion of reaction. The reaction mixture was diluted with DCM (100 mL), washed with water (100 mL), organic layer was extracted (2×100 mL), washed with Brine (100 mL), dried over sodium sulfate (15 g). Solvent was removed under reduced pressure and purified via silica gel column to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-4-oxobutyl)-1-(9H-fluoren-9-yl)-3,6,10-trioxo-2-oxa-4,7,11-triazadodecan-12-yl)-4-(hydroxymethyl)phenoxy)-6-((allyloxy)carbonyl)tetrahydro-2H-pyran-3,4,5-triyl triacetate Intermediate 17 (0.640 g, 53.1%). 1H NMR (500 MHz, DMSO) δ 8.21 (t, J=6.0 Hz, 1H), 8.11 (dd, J=19.5, 6.9 Hz, 2H), 7.94 (t, J=5.8 Hz, 1H), 7.89 (d, J=7.5 Hz, 2H), 7.73 (t, J=7.0 Hz, 2H), 7.49-7.39 (m, 3H), 7.33 (td, J=7.5, 1.2 Hz, 2H), 7.17 (dd, J=8.4, 2.2 Hz, 1H), 7.13 (s, 1H), 7.01 (d, J=8.4 Hz, 1H), 5.89 (ddt, J=17.3, 10.5, 5.7 Hz, 1H), 5.56 (d, J=7.9 Hz, 1H), 5.48 (t, J=9.6 Hz, 1H), 5.33 (dq, J=17.2, 1.6 Hz, 1H), 5.26 (dq, J=10.5, 1.4 Hz, 1H), 5.19-5.07 (m, 3H), 4.76 (d, J=10.0 Hz, 1H), 4.62 (ddt, J=13.3, 5.6, 1.4 Hz, 1H), 4.54 (ddt, J=13.3, 5.8, 1.4 Hz, 1H), 4.44-4.36 (m, 4H), 4.31-4.18 (m, 4H), 4.11 (d, J=5.9 Hz, 3H), 3.93 (d, J=5.9 Hz, 1H), 3.88-3.81 (m, 2H), 3.66-3.56 (m, 5H), 3.56-3.45 (m, 42H), 3.45-3.39 (m, 3H), 3.29-3.27 (m, 1H), 3.24 (s, 3H), 3.18 (d, J=5.0 Hz, 1H), 2.33 (ddt, J=14.6, 10.0, 7.6 Hz, 4H), 2.10-2.05 (m, 2H), 2.04 (s, 3H), 1.99 (d, J=4.6 Hz, 6H), 1.54 (dt, J=43.7, 8.9 Hz, 4H). LCMS (ESI) m/z 1629.8 (M+H)⁺.

### Intermediate 18

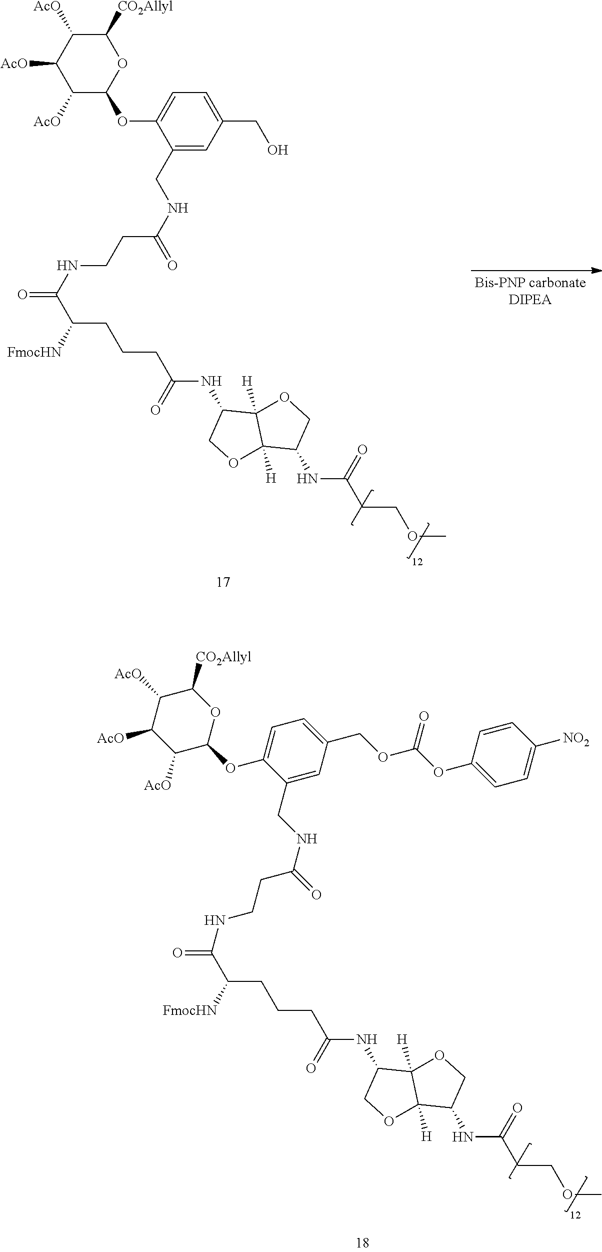

To a solution of Intermediate 17 (25.00 mg, 0.02 mmol, 1.0 eq) in DMF was added bis(4-nitrophenyl) carbonate (28.0 mg, 0.09 mmol, 4.5 eq) and DIPEA (0.013 mL, 0.08 mmol, 4.0 eq). The mixture was stirred at 23° C. for 2 hours. Following this time the mixture was concentrated in vacuo and the residue was sonicated in DCM (200 μL) and diethyl ether (2 mL). The resulting suspension was dried on vacuum filter and the process repeated. The residue was dried to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-4-oxobutyl)-1-(9H-fluoren-9-yl)-3,6,10-trioxo-2-oxa-4,7,11-triazadodecan-12-yl)-4-((((4-nitrophenoxy)carbonyl)oxy)methyl)phenoxy)-6-((allyloxy)carbonyl)tetrahydro-2H-pyran-3,4,5-triyl triacetate Intermediate 18 (32 mg, 0.02 mmol, 91%) as a yellow material. RT 7.74 min. LCMS (ESI) m/z 1794.7 [M+H]+.

### Intermediate 19

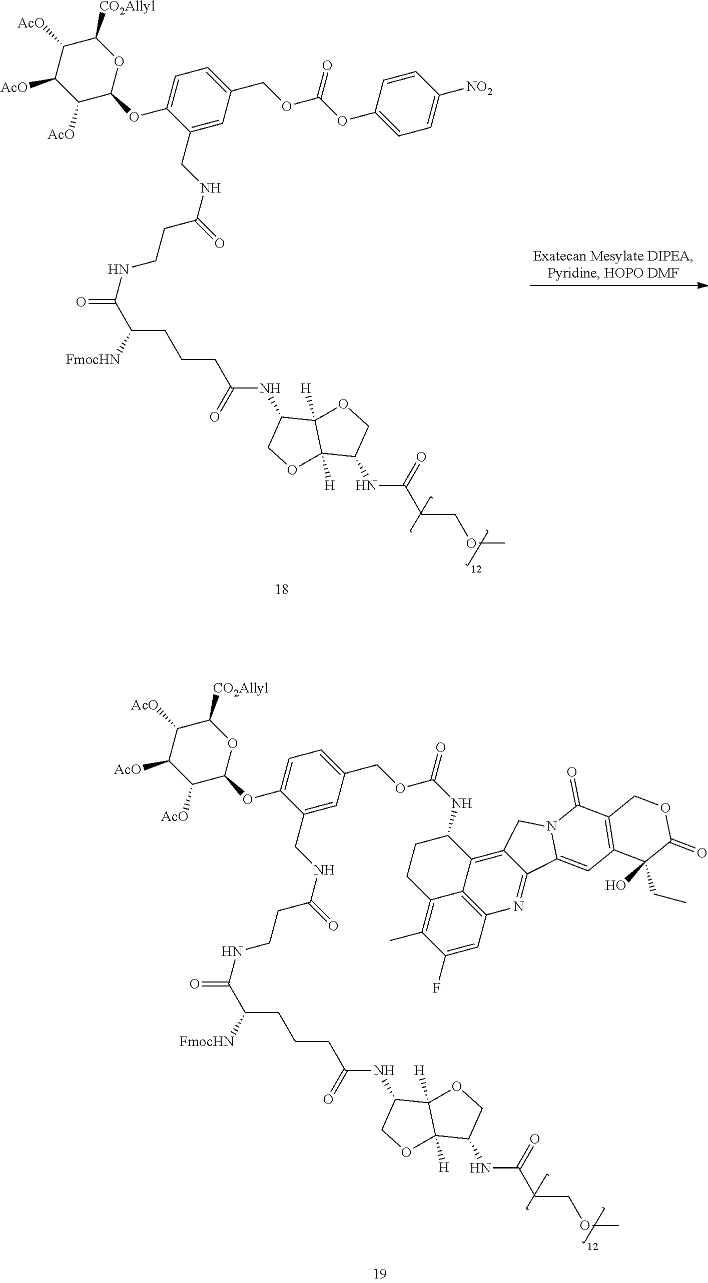

To a solution of exatecan mesylate (7.41 mg, 0.01 mmol, 1.0 eq) in DCM (1 mL) and DMF (1.000 mL) was added DIPEA (7.28 μl, 0.04 mmol, 4.0 eq), Intermediate 18 (25.00 mg, 0.01 mmol, 1.0 eq), and HOPO (1.703 mg, 0.02 mmol, 2.0 eq) and the resultant mixture was stirred at 23° C. for 18 hours. Following this time the mixture was concentrated in vacuo and the residue was purified by reverse phase flash column chromatography (C18 BIOTAGE prepacked column, 40-60% MeCN [0.1% formic acid]/water [0.1% formic acid]) to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-4-oxobutyl)-1-(9H-fluoren-9-yl)-3,6,10-trioxo-2-oxa-4,7,11-triazadodecan-12-yl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-6-((allyloxy)carbonyl)tetrahydro-2H-pyran-3,4,5-triyl triacetate Intermediate 19 (13 mg, 0.01 mmol, 63%) as a yellow material. RT 7.66 min. LCMS (ESI) 2090.3 [M+H]+.

### Intermediate 20

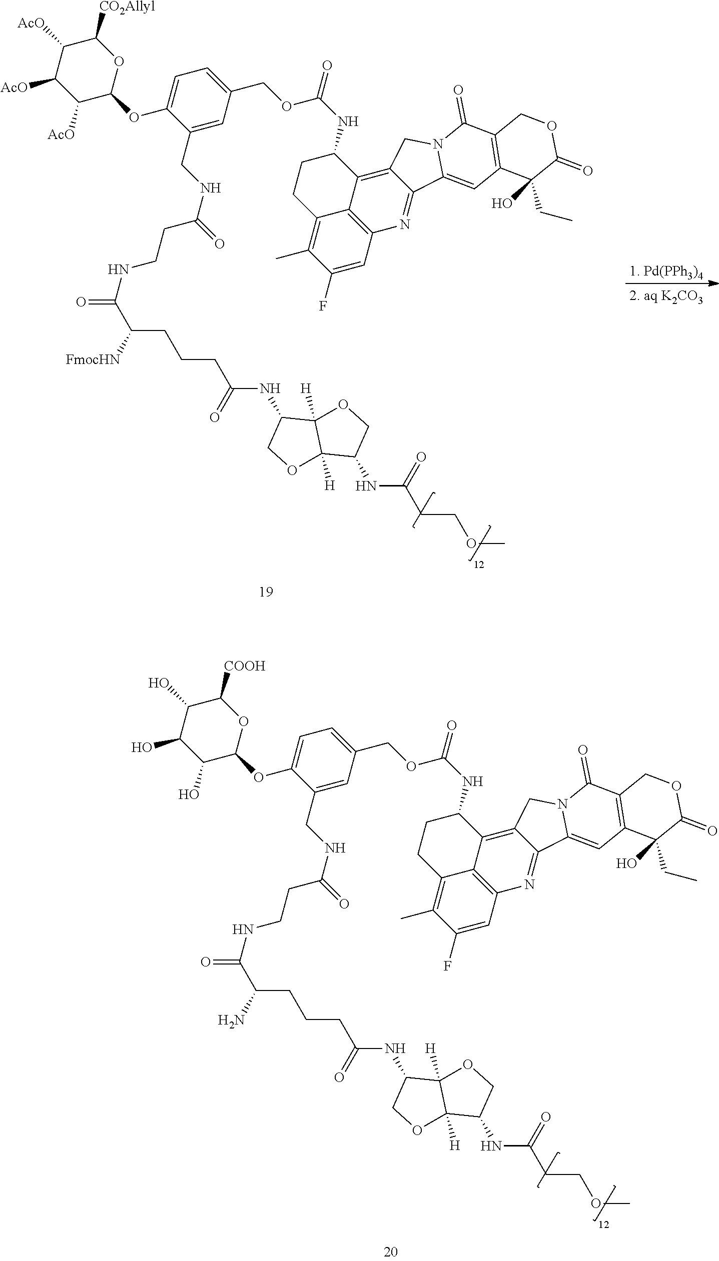

To a solution of Intermediate 19 (15.00 mg, 7.18 μmol, 1.0 eq) in DCM (1 mL) was added triethylamine (2.00 μl, 0.01 mmol, 1.4 eq), Pd(PPh₃)₄ (1.00 mg, 0.87 μmol, 12 mol %) and formic acid (0.541 μl, 0.01 mmol, 1.4 eq) and the mixture stirred at 23° C. for 18 hours the mixture. Following this time the reaction mixture was concentrated and the crude residue was dissolved in methanol (0.25 mL) and THF (0.25 mL). To this solution was added potassium carbonate (9.44 mg, 0.07 mmol, 10 eq) in water (0.5 mL) and the mixture was stirred at 23° C. for 3 hours. Following this time the mixture was concentrated in vacuo to remove organics. The remaining aqueous solution was acidified with citric acid (1 N) until pH 4 was reached and the mixture was stirred for 1 hour at 23° C. Following this time the mixture was filtered and the residue was purified by reverse phase flash column chromatography (C18 BIOTAGE prepacked column, 20-40% MeCN [0.1% formic acid]/water [0.1% formic acid]) to give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-amino-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid Intermediate 20 (8.6 mg, 5.03 μmol, 70%) as a yellow material. RT 5.08 min. LCMS (ESI) 1702.6 [M+H]+.

### Linker-Payload LP-1

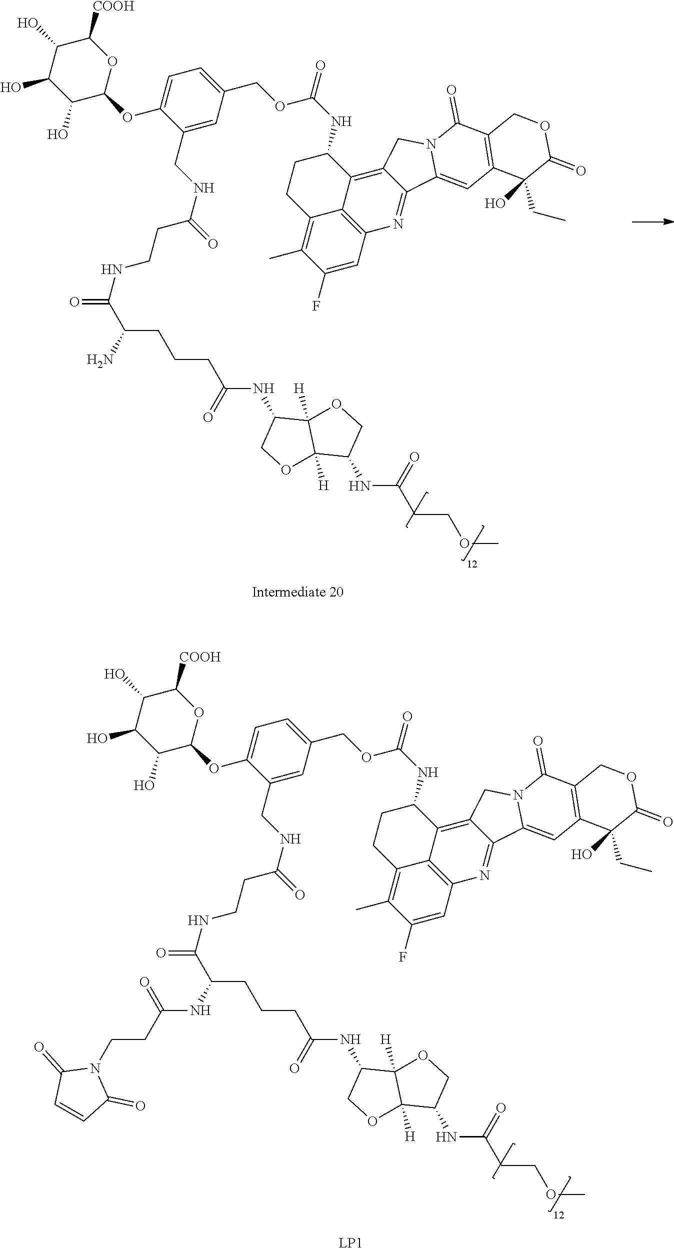

To a solution of Intermediate 20 (6.20 mg, 3.64 μmol, 1.0 eq) in DMF (0.5 mL) was added pyridine (0.7 μl, 5.5 μmol, 1.5 eq) and 2,5-dioxopyrrolidin-1-yl 3-(2,5-dioxo-2,5-dihydro-1H-pyrrol-1-yl)propanoate (0.970 mg, 3.64 μmol, 1.0 eq). The mixture was stirred at 23° C. for 3 hours. Following this time the reaction mixture was concentrated in vacuo and the residue purified by reverse phase HPLC (CSH 35% MeCN [0.1% formic acid]/water [0.1% formic acid] over 7 min) to give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid LP-1 (2.7 mg, 1.46 μmol, 40%) as a white material. RT 5.87 min. LCMS (ESI) 1853.5 [M+H]+.

### Alternative Synthesis of LP-1

### Intermediate A

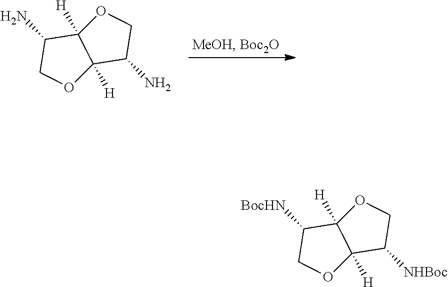 [SMILES: N[C@H]1CO[C@H]2[C@@H]1OC[C@@H]2N ; CC(C)(C)OC(=O)N[C@H]1CO[C@H]2[C@@H]1OC[C@@H]2NC(=O)OC(C)(C)C]

To intermediate 9 (22.0 g, 152.6 mmol) in methanol (440 mL) was added boc anhydride (83.3 g, 381.5 mmol, 2.5 eq) and the reaction was stirred for 2 hours at 20° C. ELSD analysis showed formation of the desired product and completion of the reaction. Solvent was removed under reduced pressure to get crude product. The crude product was slurried with MTBE (200 mL), filtered, washed with MTBE (40 mL) and dried to give tert-butyl N—[(3S,3aR,6S,6aR)-6-(tert-butoxycarbonylamino)-2,3,3a,5,6,6a-hexahydrofuro[3,2-b]furan-3-yl]carbamate Intermediate A (44.0 g, 84.3%). LCMS m/z 366.8 [M+Na]⁺

### Intermediate B

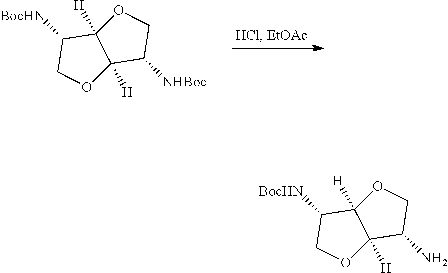 [SMILES: CC(C)(C)OC(=O)N[C@H]1CO[C@H]2[C@@H]1OC[C@@H]2NC(=O)OC(C)(C)C ; CC(C)(C)OC(=O)N[C@H]1CO[C@H]2[C@@H]1OC[C@@H]2N]

To intermediate A (2.0 g, 5.81 mmol, 1.0 eq) in Ethyl acetate (40 mL) was added HCl in ethyl acetate (4M, 8.0 mL) and the reaction was stirred for 6 hours at 20° C. ELSD analysis showed formation of the desired product and completion of the reaction. The reaction was quenched with potassium phosphate solution (2M, 20 mL), the layers were separated and the organic layer retained. The aqueous layer was extracted with ethyl acetate (10 mL) and the organic layers combined. Solvent was removed under reduced pressure to give tert-butyl N-[(3S,3aR,6S,6aR)-3-amino-2,3,3a,5,6,6a-hexahydrofuro[3,2-b]furan-6-yl]carbamate Intermediate B (800 mg, 57.1%). ¹H NMR (400 MHz, DMSO-d6) δ 4.32 (m, 1H), 3.93 (m, 1H), 3.85 (m, 2H), 3.6 (m, 2H), 3.28 (m, 1H), 1.47 (s, 9H). LCMS m/z 145 [M+H-Boc]⁺

### Intermediate C

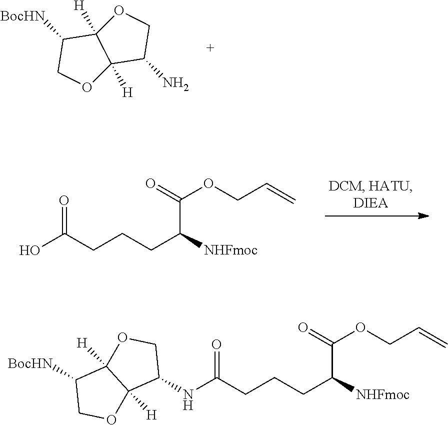

To a solution of Intermediate 14 (17.16 g, 40.5 mmol, 1.1 eq) in DCM (180 mL) at 25° C., was added HATU (16.8 g, 44.2 mmol, 1.2 eq) and DIPEA (10.9 ml, 62.6 mmol, 1.7 eq). The reaction mixture was stirred for 15 mins. To the reaction solution was added Intermediate B (9.0 g, 36.8 mmol, 1.0 eq) in DCM 90 mL)). The reaction mixture was stirred for 2 hours. LC-MS analysis showed formation of the desired product and completion of the reaction. The reaction mixture was washed twice with NaCl (15% aq, 90 mL) and the aqueous layers were discarded. Solvent was removed under reduced pressure to give allyl (2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoate Intermediate C (9.0 g, 65.2%). LCMS m/z 671.6 [M+Na]⁺

### Intermediate D

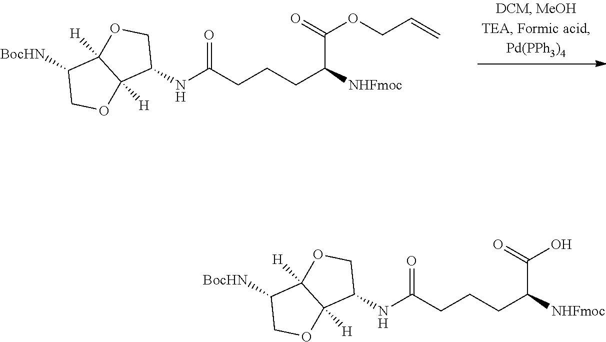

To a solution of Intermediate C (9.0 g, 13.9 mmol, 1.0 eq) in DCM/MeOH (20:1, 128.6 ml:6.4 mL) was added formic acid (1.05 mL, 27.7 mmol, 2.0 eq), triethylamine (5.79 mL, 41.6 mmol, 3.0 eq) and Pd(PPh₃)₄ (1.6 g, 1.39 mmol, 0.1 eq). The reaction mixture was stirred for 3 hours at 20-25° C. LC-MS analysis showed formation of the desired product and completion of the reaction. Solvent was removed under reduced pressure to get crude product. The crude product was purified via silica gel column (DCM to DCM:MeOH 4:1) to give (2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoic acid Intermediate D (7.0 g, 82.9%). LCMS m/z 610.1 [M+H]⁺

### Intermediate E

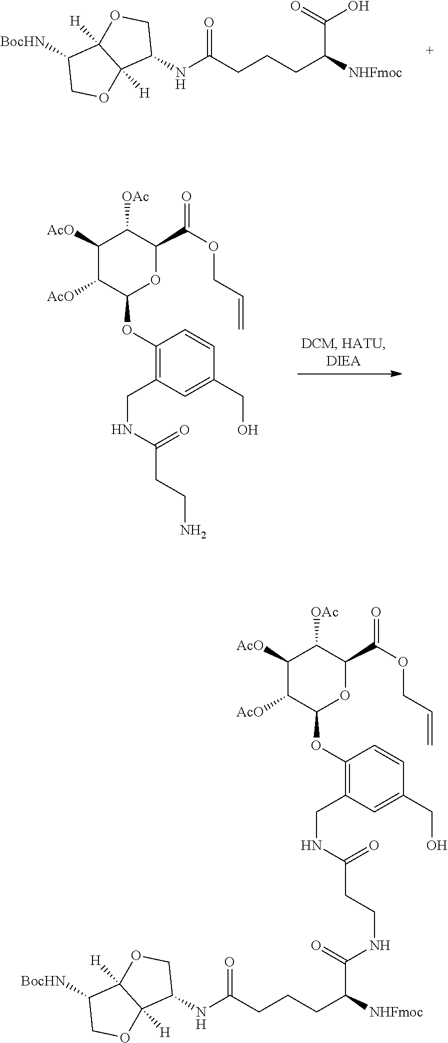

To a solution of Intermediate D (4.0 g, 6.56 mmol, 1.0 eq) in DMF (80 mL) at 0-5° C. was added Intermediate 5 (11.2 g, 19.7 mmol, 3.0 eq), HATU (3.74 g, 9.84 mmol, 1.5 eq), and DIPEA (3.43 mL, 19.7 mmol, 3.0 eq). The reaction mixture was stirred for 1 hours at 0-5° C. LC-MS analysis showed formation of the desired product and completion of the reaction. The reaction mixture was diluted with ethyl acetate (80 mL) and washed twice with NaCl (15% aq, 40 mL). The aqueous layers were discarded and the solvent was removed from the organic layer under reduced pressure to get crude product. The crude product was purified via silica gel column (EtOAC to 20% MeOH) to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-(hydroxymethyl)phenoxy]-3,4,5-triacetoxy-tetrahydropyran-2-carboxylate Intermediate E (3.6 g, 47.4%) LCMS m/z 1158.2 [M+H]⁺

### Intermediate F

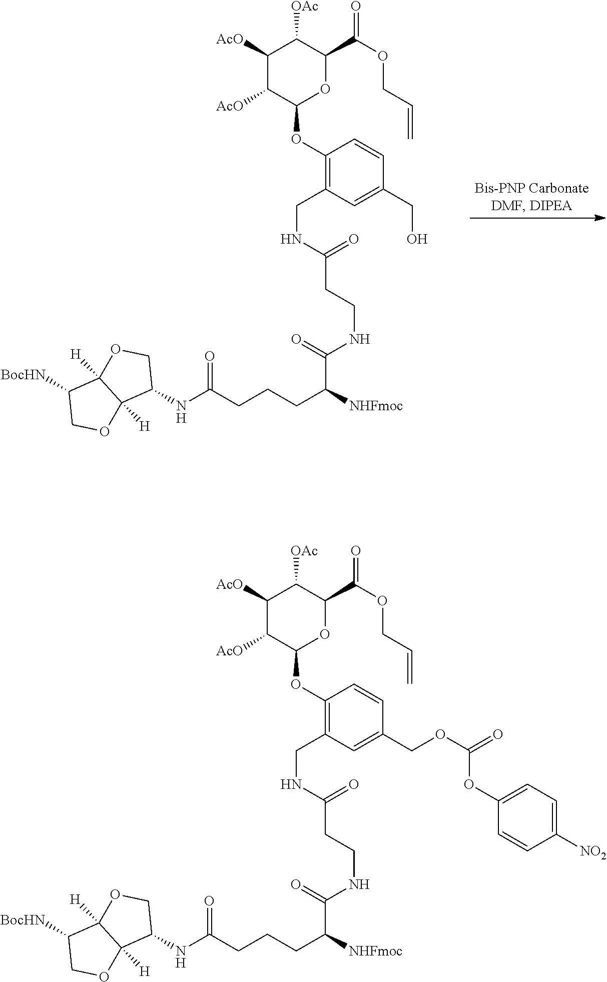

To a solution of Intermediate E (800 mg, 691 μmol, 1.0 eq) in DMF (8 mL) was added bis(4-nitrophenyl) carbonate (840 mg, 2.76 mmol, 4.0 eq) and DIPEA (481 μL, 2.76 mmol, 4.0 eq). The mixture was stirred at 20-25° C. for 2 hours. LC-MS analysis showed formation of the desired product and completion of the reaction. Solvent was removed under reduced pressure to get crude product. The crude product was purified via silica gel column (5%-50% MeCN in water, 0.1% formic acid) to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-[(4-nitrophenoxy)carbonyloxymethyl]phenoxy]-3,4,5-triacetoxy-tetrahydropyran-2-carboxylate Intermediate F (400 mg, 43.8%) 1H NMR (300 MHz, DMSO-d6) δ ppm 1.14-1.29 (m, 3H) 1.38 (s, 9H) 1.53 (br dd, J=18.52, 8.25 Hz, 4H) 1.98-2.09 (m, 12H) 2.13 (d, J=2.93 Hz, 1H) 2.32-2.45 (m, 2H) 3.35 (s, 11H) 3.58 (br d, J=6.42 Hz, 2H) 3.71 (s, 12H) 3.78-3.88 (m, 3H) 3.90-4.17 (m, 4H) 4.18-4.42 (m, 6H) 4.50-4.67 (m, 2H) 4.80 (d, J=10.09 Hz, 1H) 5.09-5.38 (m, 6H) 5.47-5.55 (m, 1H) 5.65 (d, J=7.89 Hz, 1H) 5.82-5.96 (m, 1H) 6.10 (s, 4H) 7.11 (d, J=8.44 Hz, 1H) 7.21 (br d, J=5.50 Hz, 1H) 7.28-7.37 (m, 4H) 7.37-7.44 (m, 3H) 7.46-7.61 (m, 3H) 7.65-7.76 (m, 2H) 7.89 (d, J=7.34 Hz, 2H) 7.99 (br t, J=5.50 Hz, 1H) 8.09 (d, J=6.97 Hz, 1H) 8.27-8.34 (m, 3H) LCMS m/z 1323.1 [M+H]⁺

### Intermediate G

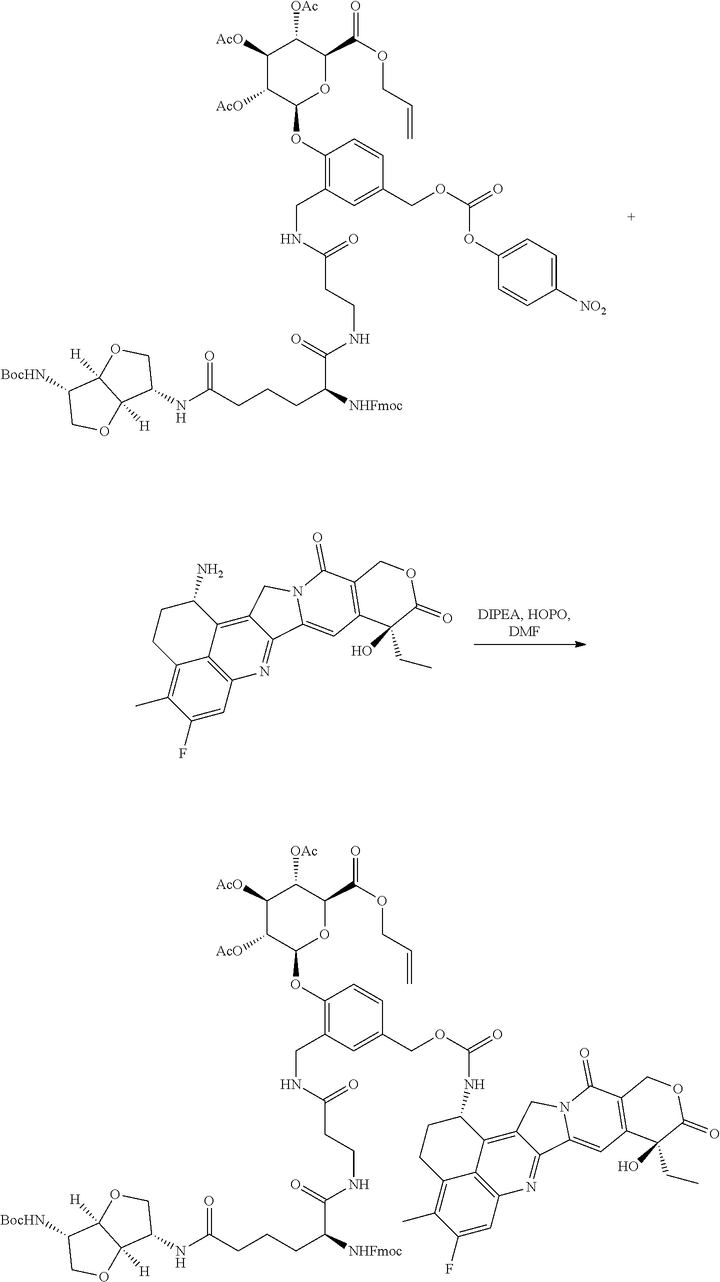

To a solution of exatecan mesylate (1.12 g, 2.12 mmol, 4.0 eq) in DMF (7 mL) was added DIPEA (369 μl, 2.12 mmol, 4.0 eq), Intermediate F (700 mg, 529 μmol, 1.0 eq), and HOPO (117.5 mg, 1.06 mmol, 2.0 eq) and the resultant mixture was stirred at 20-25° C. for 1 hour. LC-MS analysis showed formation of the desired product and completion of the reaction. The reaction mixture was diluted with ethyl acetate (9 mL) and washed with NaCl (15% aq, 12 mL). The solvent was removed under reduced pressure to get crude product. The crude product was slurried with ethyl acetate/MTBE (1:1, 5 mL), filtered and dried to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-[[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-triacetoxy-tetrahydropyran-2-carboxylate Intermediate G (360 mg, 73.5%). 1H NMR (400 MHz, DMSO-d6) δ ppm 0.81-0.94 (m, 2H) 1.11-1.26 (m, 3H) 1.37 (s, 6H) 1.42-1.59 (m, 3H) 1.81-1.92 (m, 1H) 1.95-2.15 (m, 9H) 2.29-2.44 (m, 3H) 2.73 (s, 1H) 2.89 (s, 1H) 3.49-3.61 (m, 2H) 3.71 (s, 16H) 3.81 (br d, J=6.36 Hz, 2H) 3.85-3.95 (m, 1H) 3.99-4.12 (m, 2H) 4.12-4.28 (m, 2H) 4.33 (br d, J=3.91 Hz, 1H) 4.39 (br d, J=4.16 Hz, 1H) 4.48-4.64 (m, 1H) 4.78 (br d, J=10.27 Hz, 1H) 5.06-5.34 (m, 5H) 5.41-5.53 (m, 2H) 5.60 (br d, J=7.58 Hz, 1H) 5.81-5.93 (m, 1H) 6.09 (s, 5H) 6.92 (d, J=8.26 Hz, 2H) 7.09 (br d, J=8.56 Hz, 1H) 7.17-7.42 (m, 5H) 7.64-7.79 (m, 2H) 7.80-7.90 (m, 1H) 7.93-7.99 (m, 1H) 8.00-8.14 (m, 3H) LCMS m/z 810.3 [M+2H]²⁺

### Intermediate H

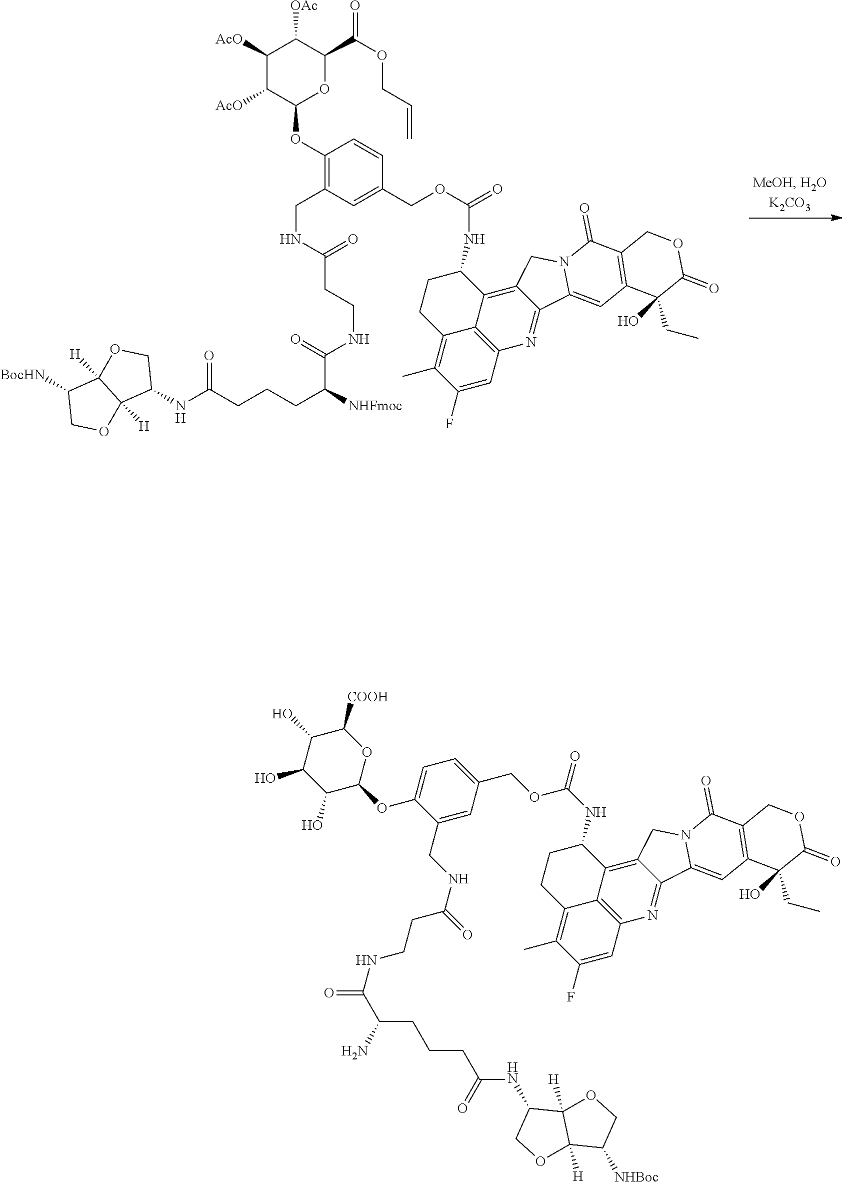

To a solution of Intermediate G (1.5 g 80%, 741 μmol, 1.0 eq) in MeOH (2.6 mL) was added K₂CO₃ (1.02 g, 7.41 mmol, 10.0 eq) and water (260 μL). The resultant mixture was stirred at 20-25° C. for 1 hour. LC-MS analysis showed formation of the desired product and completion of the reaction. Solvent was removed under reduced pressure to get crude product. The crude product was purified via silica gel column (5%-50% MeCN in water, 0.1% formic acid) to give (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-amino-6-oxo-hexanoyl]amino]propanoylamino]methyl]-4-[[[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-trihydroxy-tetrahydropyran-2-carboxylic acid Intermediate H (450 mg, 67%) 1H NMR (500 MHz, DMSO-d6) δ ppm 0.84-0.92 (m, 3H) 1.24 (br s, 4H) 1.29 (br d, J=7.32 Hz, 1H) 1.38 (s, 8H) 1.43-1.53 (m, 2H) 1.60 (br s, 2H) 1.81-2.11 (m, 5H) 2.14-2.24 (m, 2H) 2.29-2.36 (m, 1H) 2.39 (s, 3H) 2.40-2.44 (m, 1H) 3.13-3.23 (m, 7H) 3.23-3.32 (m, 14H) 3.35 (br s, 12H) 3.50-3.60 (m, 8H) 3.77-3.85 (m, 3H) 4.05 (br s, 1H) 4.17 (br dd, J=13.43, 3.97 Hz, 1H) 4.34 (br d, J=3.36 Hz, 1H) 4.39 (d, J=3.97 Hz, 1H) 4.49 (br dd, J=13.28, 7.78 Hz, 1H) 4.60 (br d, J=6.71 Hz, 1H) 5.03 (br d, J=12.21 Hz, 1H) 5.10 (br d, J=12.21 Hz, 1H) 5.28 (br s, 3H) 5.45 (s, 2H) 6.52 (br s, 1H) 7.12 (d, J=8.24 Hz, 1H) 7.20 (br s, 1H) 7.32 (s, 1H) 7.32-7.34 (m, 1H) 7.40 (s, 1H) 7.78 (d, J=10.68 Hz, 1H) 8.03-8.15 (m, 1H) 8.17 (s, 2H) 8.24 (br d, J=6.10 Hz, 1H) 8.47 (br s, 1H) 9.30 (br s, 1H). LCMS m/z 616.2 [M+2H]²⁺

### Intermediate I

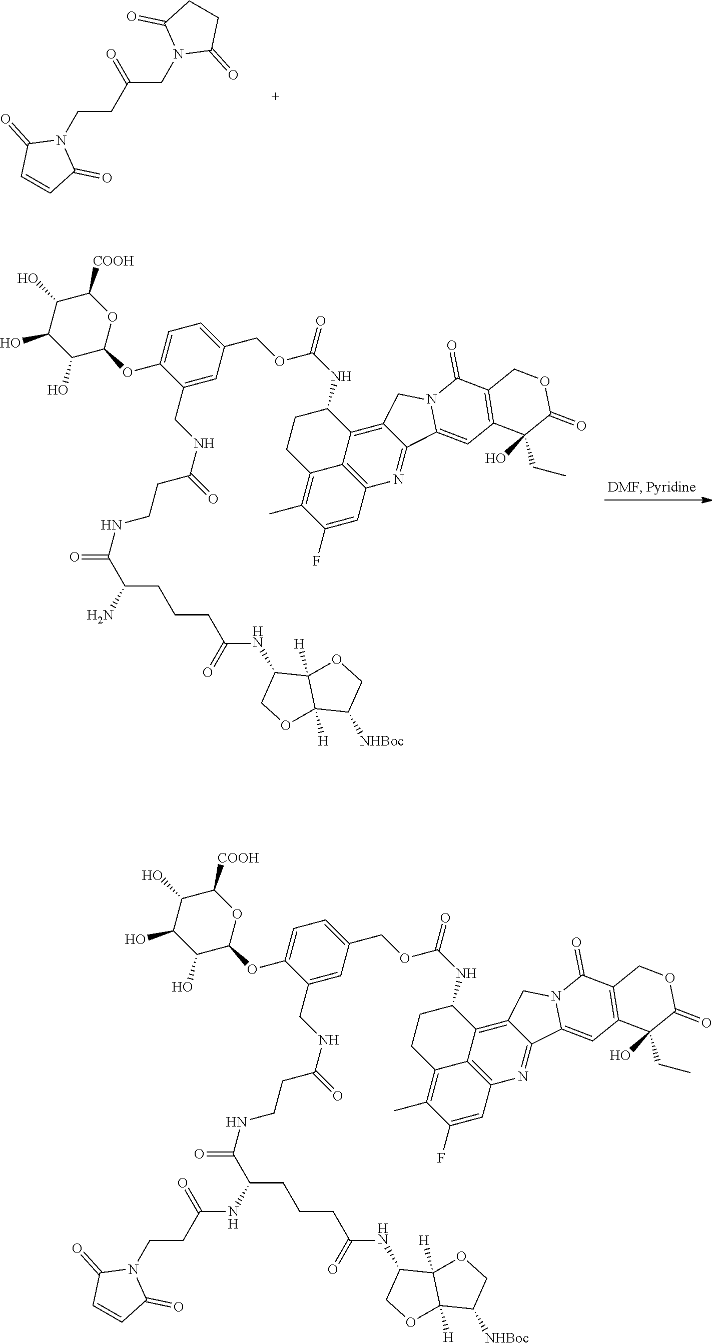 [SMILES: O=C(CCN1C(=O)C=CC1=O)CN1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@@H](N)CCCC(=O)N[C@H]2CO[C@H]4[C@@H]2OC[C@@H]4NC(=O)OC(C)(C)C)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4NC(=O)OC(C)(C)C)NC(=O)CCN2C(=O)C=CC2=O)c1)CC3]

To a solution of Intermediate H (450 mg, 365 μmol, 1.0 eq) in DMF (4.5 mL) at 20-25° C. was added pyridine (225 μL) and N-succinimidyl 3-maleimidiopropionate (195 mg, 731 μmol, 2.0 eq) and the reaction mixture was stirred for 1 hour. LC-MS analysis showed formation of the desired product and completion of the reaction. Solvent was removed under reduced pressure to get crude product. The crude product was purified via silica gel column (5%-50% MeCN in water, 0.1% formic acid) to give (2S,3S,4S,5R,6S)-6-(2-((3-((S)-6-(((3S,3aR,6S,6aR)-6-((tert-butoxycarbonyl)amino)hexahydrofuro[3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid Intermediate I (230 mg, 52.4%) 1H NMR (500 MHz, DMSO-d6) δ ppm 0.84-0.91 (m, 2H) 1.23 (br d, J=9.77 Hz, 5H) 1.38 (s, 6H) 1.81-1.94 (m, 1H) 1.96-2.07 (m, 2H) 2.13-2.24 (m, 1H) 2.25-2.36 (m, 1H) 2.50-2.52 (m, 7H) 3.05-3.18 (m, 1H) 3.24 (br d, J=6.10 Hz, 2H) 3.29 (br s, 3H) 3.34 (br s, 24H) 3.51-3.63 (m, 4H) 3.71 (s, 9H) 3.75-3.88 (m, 3H) 4.01-4.15 (m, 2H) 4.21 (br d, J=9.46 Hz, 1H) 4.34 (d, J=3.66 Hz, 1H) 4.36-4.46 (m, 1H) 4.72 (br s, 1H) 5.06-5.15 (m, 1H) 5.27 (br s, 2H) 5.45 (s, 2H) 6.09 (s, 3H) 6.51 (s, 1H) 6.97 (s, 1H) 7.10 (d, J=8.24 Hz, 1H) 7.19 (br d, J=5.80 Hz, 1H) 7.31 (s, 1H) 7.33 (s, 1H) 7.77 (d, J=10.99 Hz, 1H) 7.94 (br t, J=5.34 Hz, 1H) 8.06 (br d, J=8.55 Hz, 1H) 8.12 (br d, J=6.71 Hz, 1H) 8.20 (br d, J=8.24 Hz, 1H) LCMS m/z 1382.1 [M+H]⁺

### Intermediate J

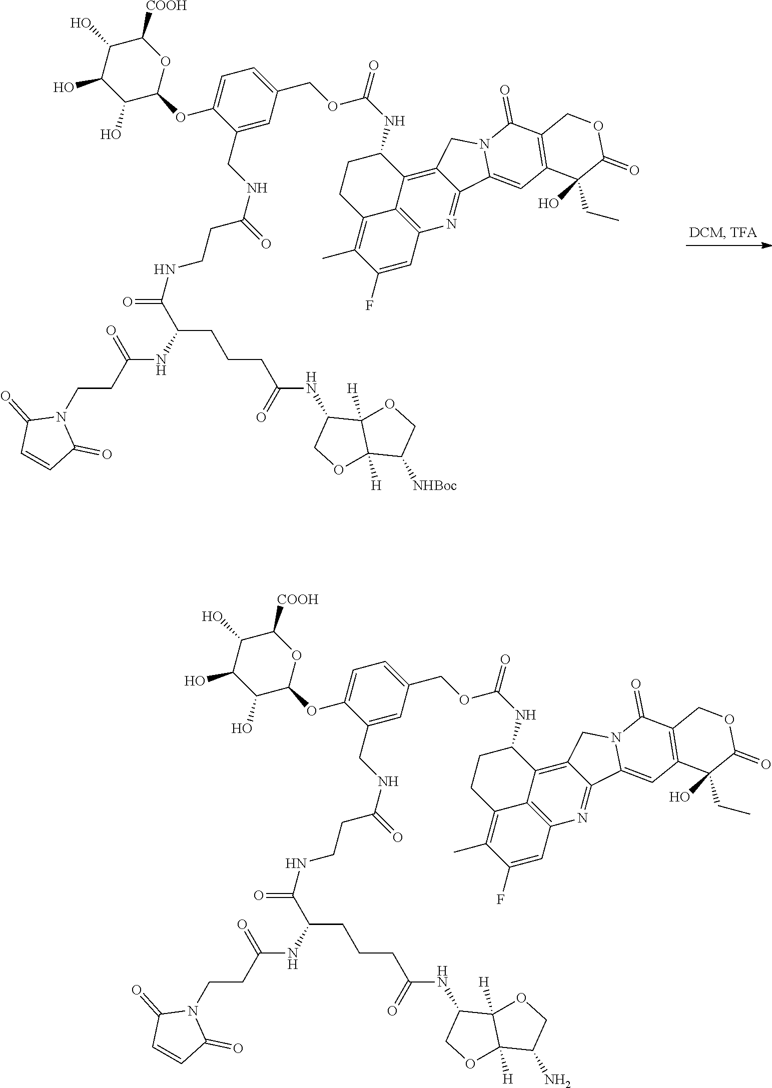 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4NC(=O)OC(C)(C)C)NC(=O)CCN2C(=O)C=CC2=O)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4N)NC(=O)CCN2C(=O)C=CC2=O)c1)CC3]

To a solution of Intermediate I (230 mg, 166 μmol, 1.0 eq) in DCM (2.6 mL) was added TFA (1.3 mL), and the reaction mixture was stirred for 1 hour at 25° C. LC-MS removed under reduced pressure to get crude product. The crude product was purified via silica gel column (5%-50% MeCN in water, 0.1% formic acid) to give (2S,3S,4S,5R,6S)-6-[2-[[3-[[(2S)-6-[[(3S,3aR,6S,6aR)-3-amino-2,3,3a,5,6,6a-hexahydrofuro[3,2-b]furan-6-yl]amino]-2-[3-(2,5-dioxopyrrol-1-yl)propanoylamino]-6-oxo-hexanoyl]amino]propanoylamino]methyl]-4-[[1(1S,23S)-10-ethyl-18-fluoro-10-hydroxy-19-methyl-S,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-trihydroxy-tetrahydropyran-2-carboxylic acid Intermediate J (120 mg, 54.5%). LCMS m/z 641.5 [M+H]⁺

### Linker-Payload LP-1

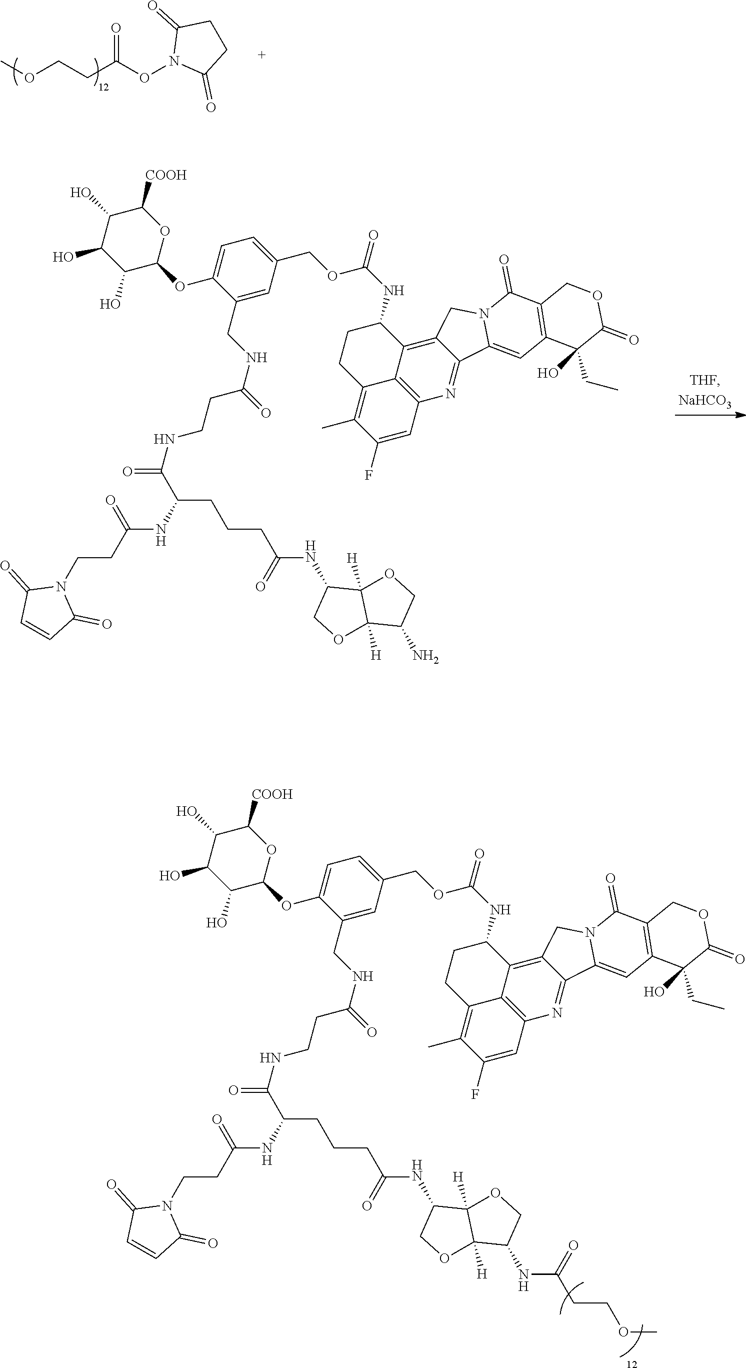

To a solution of Intermediate J (100 mg, 78.0 μmol, 1.0 eq) in THE (1 mL) was added NaHCO₃ (13.1 mg, 156 μmol, 2.0 eq) and 2,5-dioxopyrrolidin-1-yl 2,5,8,11,14,17,20,23,26,29,32,35-dodecaoxaoctatriacontan-38-oate Intermediate 10 (64.2 mg, 93.6 μmol, 1.2 eq). The reaction mixture was stirred at 20-25° C. for 16 h. LC-MS analysis showed formation of the desired product and completion of the reaction. Solvent was removed under reduced pressure to get crude product. Solvent was removed under reduced pressure to get crude product. The crude product was purified via silica gel column (5%-50% MeCN in water, 0.1% formic acid) to give give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid LP-1 (60 mg, 40%). LCMS m/z 927.2 [M+2H]²⁺

### Antibody-Drug Conjugation

### ADC-1: Herceptin-WT-LP-1 (DAR 8)

LP-1 was added as a DMSO solution (16 molar equivalent/antibody, 0.64 μmole, in 0.128 mL DMSO) to 1.82 mL of the Herceptin-wt antibody solution in PBS, 1 mM EDTA, pH 7.4 (6.0 mg, 40.0 nanomoles) for a 10% (v/v) final DMSO concentration. The solution left to react at room temperature for 1 hours with gentle shaking. Then the conjugation was quenched by addition of N-acetyl cysteine (3.2 micromoles, 32.03 μL at 100 mM), then purified in and formulated in PBS pH 7.4 by spin filtration using a 15 mL AMICON ULTRACELL 30 kDa MWCO spin filter, sterile-filtered and analysed.

UHPLC analysis on a SHIMADZU PROMINENCE system using a THERMO SCIENTIFIC MAbPac 50 mm×2.1 mm column eluting with a gradient of water and acetonitrile on a reduced sample of ADC at 214 nm and 330 nm shows a mixture of light chain conjugated to 1 molecule of LP-1, and heavy chain conjugated to 3.0 molecules of LP-1, consistent with a drug-per-antibody ratio (DAR) of 7.9 molecules of LP-1 per antibody.

UHPLC analysis on a SHIMADZU PROMINENCE system using A TOSOH BIOSCIENCE TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ADC at 280 nm shows a monomer purity of 97.9%. UHPLC SEC analysis gives a concentration of final ADC at 2.86 mg/mL in 1.6 mL, obtained mass of is 4.5 mg (75% yield).

LC-MS analysis on a EXACTIVE PLUS EMR mass spectrometer connected to DIONEX 3000 HPLC equipment using a THERMO SCIENTIFIC MAbPac 50 mm×2.1 mm column eluting with a gradient of water and acetonitrile on a de-glycosylated and reduced sample of ADC at 214 nm shows a mixture of light chain conjugated to 1 molecule LP-1, and heavy chain conjugated to 3.0 molecules of LP-1, consistent with a drug-per-antibody ratio (DAR) of 7.9 molecules of LP-1 per antibody.

UHPLC analysis on a SHIMADZU PROMINENCE system using A PROTEOMIX HIC Butyl-NP5, 5 um, non-porous, 4.6×35 mm (SEPAX) column eluting with a gradient of 1.5M ammonium sulphate, 25 mM sodium acetate, pH 7.4 and 25 mM sodium acetate, pH 7.4 with 20% acetonitrile (v/v) on a neat sample of ADC at 214 nm shows singly conjugated LP-1, consistent with a drug-per-antibody ratio (DAR) of 8 molecules of LP-1 per antibody.

### Sequences

| SEQ ID NO: | Identifier | Sequence |
|---|---|---|
| SEQ ID NO: 1 | 40A3GL-LO14 VH CDR 1 | RNSAVWN |
| SEQ ID NO: 2 | 40A3GL-LO14 VH CDR 2 | RTYYRSKWYNDYAPSVKS |
| SEQ ID NO: 3 | 40A3GL-LO14 VH CDR 3 | GLRQNQFYYYMDV |
| SEQ ID NO: 4 | 40A3GL-LO14 VL CDR 1 | RASQSVASNLA |
| SEQ ID NO: 5 | 40A3GL-LO14 VL CDR 2 | GASTRAT |
| SEQ ID NO: 6 | 40A3GL-LO14 VL CDR 3 | QQYNNWPFT |
| SEQ ID NO: 7 | 40A3GL-LO11 VH CDR 1 | RNSAVWN |
| SEQ ID NO: 8 | 40A3GL-LO11 VH CDR 2 | RTYYRSKWYNDYAPSVKS |
| SEQ ID NO: 9 | 40A3GL-LO11 VH CDR 3 | GLLQNQFYYYMDV |
| SEQ ID NO: 10 | 40A3GL-LO11 VL CDR 1 | RASQSVASNLA |
| SEQ ID NO: 11 | 40A3GL-LO11 VL CDR 2 | GASTRAT |
| SEQ ID NO: 12 | 40A3GL-LO11 VL CDR 3 | QQYNNWPFT |
| SEQ ID NO: 13 | STEAP2 VH CDR1 | RNSAVWN |
| SEQ ID NO: 14 | STEAP2 VH CDR2 | RTYYRSKWYNDYAVSVKS |
| SEQ ID NO: 15 | STEAP2 VH CDR3 | GLLQNNFYYYMDV |
| SEQ ID NO: 16 | STEAP2 VL CDR1 (40A3) | RASQSVNSNLA |
| SEQ ID NO: 17 | STEAP2 VL CDR2 | GASTRAT |
| SEQ ID NO: 18 | STEAP2 VL CDR3 | QQYNNWPFT |
| SEQ ID NO: 19 | STEAP2-3 VH CDR1 | RNSAVWN |
| SEQ ID NO: 20 | STEAP2-3 VH CDR2 | RTYYRSKWYNDYAVSVKS |
| SEQ ID NO: 21 | STEAP2-3 VH CDR3 | GLLQNQFYYYMDV |
| SEQ ID NO: 22 | STEAP2-3 VL | RASQSVSSNLA |
| | CDR1(40A3GL-LO7) | |
| SEQ ID NO: 23 | STEAP2-3 VL CDR2 | GASTRAT |
| SEQ ID NO: 24 | STEAP2-3 VL CDR3 | QQYNNWPFT |
| SEQ ID NO: 25 | Humanized STEAP2-2 VH | SYGMS |
| | CDR1 | |
| SEQ ID NO: 26 | Humanized STEAP2-2 VH | TISSGGSYTFYPDIMKG |
| | CDR2 | |
| SEQ ID NO: 27 | Humanized STEAP2-2 VH | RGYGTIYTFSFDA |
| | CDR3 | |
| SEQ ID NO: 28 | Humanized STEAP2-2 VL | RSSQSVVHSNANTYLE |
| | CDR1 | |
| SEQ ID NO: 29 | Humanized STEAP2-2 VL | KVSNRFS |
| | CDR2 | |
| SEQ ID NO: 30 | Humanized STEAP2-2 VL | FQGSHVPYT |
| | CDR3 | |
| SEQ ID NO: 31 | 40A3GL-LO14 VH | QVQLQQSGPGLVKPSQTLSLTCAISGD |
| | | SVSRNSAVWNWIRQSPSRGLEWLGRT |
| | | YYRSKWYNDYAPSVKSRITINPDTSK |
| | | NQFSLQLNSVTPEDTAVYYCARGLRQ |
| | | NQFYYYMDVWGKGTTVTVSS |
| SEQ ID NO: 32 | 40A3GL-LO14 VL | EIVMTQSPATLSVSPGERATLSCRASQ |
| | | SVASNLAWYQQKPGQAPRLLIYGAST |
| | | RATGIPARFSGSGSGTEFTLTISSLQSE |
| | | DFAVYYCQQYNNWPFTFGPGTKVDI |
| | | K |
| SEQ ID NO: 33 | 40A3GL-LO11 VH | QVQLQQSGPGLVKPSQTLSLTCAISGD |
| | | SVSRNSAVWNWIRQSPSRGLEWLGRT |
| | | YYRSKWYNDYAPSVKSRITINPDTSK |
| | | NQFSLQLNSVTPEDTAVYYCARGLLQ |
| | | NQFYYYMDVWGKGTTVTVSS |
| SEQ ID NO: 34 | 40A3GL-LO11 VL | EIVMTQSPATLSVSPGERATLSCRASQ |
| | | SVASNLAWYQQKPGQAPRLLIYGAST |
| | | RATGIPARFSGSGSGTEFTLTISSLQSE |
| | | DFAVYYCQQYNNWPFTFGPGTKVDI |
| | | K |
| SEQ ID NO: 35 | STEAP2 VH | QVQLQQSGPGLVKPSQTLSLTCAISGD |
| | | SVSRNSAVWNWIRQSPSRGLEWLGRT |
| | | YYRSKWYNDYAVSVKSRITINPDTSK |
| | | NQFSLQVNSVTPEDTAVYYCARGLLQ |
| | | NNFYYYMDVWGKGTTVTVSS |
| SEQ ID NO: 36 | STEAP2 VL | EIVMTQSPATLSVSPGERATLSCRASQ |
| | | SVNSNLAWYQQKPGQAPRLLIYGAST |
| | | RATGIPARFSGSGSGTEFTLTISSLQSE |
| | | DFAVYYCQQYNNWPFTFGPGTKVDI |
| | | K |
| SEQ ID NO: 37 | STEAP2-3 VH | QVQLQQSGPGLVKPSQTLSLTCAISGD |
| | | SVSRNSAVWNWIRQSPSRGLEWLGRT |
| | | YYRSKWYNDYAVSVKSRITINPDTSK |
| | | NQFSLQLNSVTPEDTAVYYCARGLLQ |
| | | NQFYYYMDVWGKGTTVTVSS |
| SEQ ID NO: 38 | STEAP2-3 VL | EIVMTQSPATLSVSPGERATLSCRASQ |
| | | SVSSNLAWYQQKPGQAPRLLIYGAST |
| | | RATGIPARFSGSGSGTEFTLTISSLQSE |
| | | DFAVYYCQQYNNWPFTFGPGTKVDI |
| | | K |
| SEQ ID NO: 39 | Humanized STEAP2-2 VH | EVQLVESGGGLVKPGGSLRLSCAASG |
| | | FTFSSYGMSWVRQAPGKRLEWVATIS |
| | | SGGSYTFYPDIMKGRFTISRDNSKNTL |
| | | YLQMNSLRAEDTAVYYCARRGYGTI |
| | | YTFSFDAWGQGTTLTVSS |
| SEQ ID NO: 40 | Humanized STEAP2-2 VL | DVVMTQSPLSLPVTLGQPASISCRSSQ |
| | | SVVHSNANTYLEWYLQKPGQSPQLLI |
| | | YKVSNRFSGVPDRFSGSGSGTDFTLKI |
| | | SRVEAEDVGVYYCFQGSHVPYTFGQ |
| | | GTKLEIK |
| SEQ ID NO: 41 | 40A3GL-LO14 Heavy chain | QVQLQQSGPGLVKPSQTLSLTCAISGD |
| | (L234F/L235E/P331S) | SVSRNSAVWNWIRQSPSRGLEWLGRT |
| | | YYRSKWYNDYAPSVKSRITINPDTSK |
| | | NQFSLQLNSVTPEDTAVYYCARGLRQ |
| | | NQFYYYMDVWGKGTTVTVSSASTKG |
| | | PSVFPLAPSSKSTSGGTAALGCLVKDY |
| | | FPEPVTVSWNSGALTSGVHTFPAVLQ |
| | | SSGLYSLSSVVTVPSSSLGTQTYICNV |
| | | NHKPSNTKVDKRVEPKSCDKTHTCPP |
| | | CPAPEFEGGPSVFLFPPKPKDTLMISRT |
| | | PEVTCVVVDVSHEDPEVKFNWYVDG |
| | | VEVHNAKTKPREEQYNSTYRVVSVLT |
| | | VLHQDWLNGKEYKCKVSNKALPASI |
| | | EKTISKAKGQPREPQVYTLPPSREEMT |
| | | KNQVSLTCLVKGFYPSDIAVEWESNG |
| | | QPENNYKTTPPVLDSDGSFFLYSKLTV |
| | | DKSRWQQGNVFSCSVMHEALHNHYT |
| | | QKSLSLSPGK |
| SEQ ID NO: 42 | 40A3GL-LO14 Light chain | EIVMTQSPATLSVSPGERATLSCRASQ |
| | | SVASNLAWYQQKPGQAPRLLIYGAST |
| | | RATGIPARFSGSGSGTEFTLTISSLQSE |
| | | DFAVYYCQQYNNWPFTFGPGTKVDI |
| | | KRTVAAPSVFIFPPSDEQLKSGTASVV |
| | | CLLNNFYPREAKVQWKVDNALQSGN |
| | | SQESVTEQDSKDSTYSLSSTLTLSKAD |
| | | YEKHKVYACEVTHQGLSSPVTKSFNR |
| | | GEC |
| SEQ ID NO: 43 | 40A3GL-LO11 Heavy chain | QVQLQQSGPGLVKPSQTLSLTCAISGD |
| | (L234F/L235E/P331S) | SVSRNSAVWNWIRQSPSRGLEWLGRT |
| | | YYRSKWYNDYAPSVKSRITINPDTSK |
| | | NQFSLQLNSVTPEDTAVYYCARGLLQ |
| | | NQFYYYMDVWGKGTTVTVSSASTKG |
| | | PSVFPLAPSSKSTSGGTAALGCLVKDY |
| | | FPEPVTVSWNSGALTSGVHTFPAVLQ |
| | | SSGLYSLSSVVTVPSSSLGTQTYICNV |
| | | NHKPSNTKVDKRVEPKSCDKTHTCPP |
| | | CPAPEFEGGPSVFLFPPKPKDTLMISRT |
| | | PEVTCVVVDVSHEDPEVKFNWYVDG |
| | | VEVHNAKTKPREEQYNSTYRVVSVLT |
| | | VLHQDWLNGKEYKCKVSNKALPASI |
| | | EKTISKAKGQPREPQVYTLPPSREEMT |
| | | KNQVSLTCLVKGFYPSDIAVEWESNG |
| | | QPENNYKTTPPVLDSDGSFFLYSKLTV |
| | | DKSRWQQGNVFSCSVMHEALHNHYT |
| | | QKSLSLSPGK |
| SEQ ID NO: 44 | 40A3GL-LO11 Light chain | EIVMTQSPATLSVSPGERATLSCRASQ |
| | | SVASNLAWYQQKPGQAPRLLIYGAST |
| | | RATGIPARFSGSGSGTEFTLTISSLQSE |
| | | DFAVYYCQQYNNWPFTFGPGTKVDI |
| | | KRTVAAPSVFIFPPSDEQLKSGTASVV |
| | | CLLNNFYPREAKVQWKVDNALQSGN |
| | | SQESVTEQDSKDSTYSLSSTLTLSKAD |
| | | YEKHKVYACEVTHQGLSSPVTKSFNR |
| | | GEC |
| SEQ ID NO: 45 | STEAP2-4 VH | EVQLVESGGGLVQPGGSLRLSCAASG |
| | | FTFSSFAMTWVRQAPGKGLEWVSVIT |
| | | YSGGRTYYADSVKGRFTISRDNSKNT |
| | | LYLQMNSLRAEDTAVYFCAKDRIAA |
| | | VGPFDYWGQGTLVTVSS |
| SEQ ID NO: 46 | STEAP2-4 VL | DIQLTQSPSFLSASVGDRVTITCRASQ |
| | | GISVYLAWYQQEPGKAPKLLIYAAST |
| | | LQSGVPSRFSGSGSGTEFTLTISSLQPE |
| | | DFATYYCQQLNSYPRTFGQGTKVEIK |
| SEQ ID NO: 47 | \|STEAP2-5 VH | QVQLVQSGAEVKKPGASVKVSCKAS |
| | | GYTFTSYGISWVRQAPGQGLEWMGW |
| | | ISGYTGNTNYAQKLQGRVTMTADTST |
| | | STAYMELRSLRSDDTAVYYCARGGS |
| | | YFDYWGQGTLVTVSS |
| SEQ ID NO: 48 | STEAP2-5 VL | DIQMTQSPSTLSASVGDRVTITCRASQ |
| | | SISRWLAWYQQKPGKAPKLLIYKASS |
| | | LESGVPSRFSGSGSGTEFTLTISSLQPD |
| | | DFATYYCQQFNSFSPITFGQGTRLEIK |
| SEQ ID NO: 49 | STEAP2-6 VH | QVQLQQPGAELVKPGASVKLSCKASG |
| | | YTFTSYWMEWVKQRPGQGLEWIGMI |
| | | HPNSGITNYNERFKNKATLTVDKSSST |
| | | AYMQLSSLTSEDSAVYYCARDHYYIL |
| | | AYWGQGTLVTVSA |
| SEQ ID NO: 50 | STEAP2-6 VL | DVLMTQTPLSLPVSLGDQASISCRSSQ |
| | | SVVHSNGNTYLEWYLQKPGQSPKLLI |
| | | YKVSNRFSGVPDRFSGSGSGTDFTLKI |
| | | SRVEAEDLGVYYCFQGSHVPYTFGGG |
| | | TKLEIK |
| SEQ ID NO: 51 | STEAP2-7 VH | QVQLQQPGADLVKPGASVKMSCKAS |
| | | GHTFTNYWVTWVKQRPGQGLEWIGN |
| | | FYPGSGIIKYNENFRSKATLTVDISSST |
| | | AYMQLSSLTSEDSAVYYCARSKLGDS |
| | | FYFDYWGQGTTLTVSS |
| SEQ ID NO: 52 | STEAP2-7 VL | DVVMTQTPLSLPVSLGNQASISCRSSQ |
| | | SLVHSNGNTYLHWYLQKPGQSPKLLI |
| | | YKVSNRFSGVPDRFSGSGSGTDFTLKI |
| | | SRVEAEDLGVYFCSQSTHVPLTFGAG |
| | | TKLEIK |
| SEQ ID NO: 53 | Wild type Constant Region | ASTKGPSVFPLAPSSKSTSGGTAALGC |
| | | LVKDYFPEPVTVSWNSGALTSGVHTF |
| | | PAVLQSSGLYSLSSVVTVPSSSLGTQT |
| | | YICNVNHKPSNTKVDKRVEPKSCDKT |
| | | HTCPPCPAPELLGGPSVFLFPPKPKDT |
| | | LMISRTPEVTCVVVDVSHEDPEVKFN |
| | | WYVDGVEVHNAKTKPREEQYNSTYR |
| | | VVSVLTVLHQDWLNGKEYKCKVSNK |
| | | ALPAPIEKTISKAKGQPREPQVYTLPPS |
| | | REEMTKNQVSLTCLVKGFYPSDIAVE |
| | | WESNGQPENNYKTTPPVLDSDGSFFL |
| | | YSKLTVDKSRWQQGNVFSCSVMHEA |
| | | LHNHYTQKSLSLSPGK |
| SEQ ID NO: 54 | Triple Mutation (TM)Variant | ASTKGPSVFPLAPSSKSTSGGTAALGC |
| | IgG1 Constant Region | LVKDYFPEPVTVSWNSGALTSGVHTF |
| | ((L234F/L235E/P331S) | PAVLQSSGLYSLSSVVTVPSSSLGTQT |
| | | YICNVNHKPSNTKVDKRVEPKSCDKT |
| | | HTCPPCPAPEFEGGPSVFLFPPKPKDTL |
| | | MISRTPEVTCVVVDVSHEDPEVKFNW |
| | | YVDGVEVHNAKTKPREEQYNSTYRV |
| | | VSVLTVLHQDWLNGKEYKCKVSNKA |
| | | LPASIEKTISKAKGQPREPQVYTLPPSR |
| | | EEMTKNQVSLTCLVKGFYPSDIAVEW |
| | | ESNGQPENNYKTTPPVLDSDGSFFLYS |
| | | KLTVDKSRWQQGNVFSCSVMHEALH |
| | | NHYTQKSLSLSPGK |
| SEQ ID NO: 55 | 40A3GL-LO14 VH with Wild | QVQLQQSGPGLVKPSQTLSLTCAISGD |
| | Type IgG1 Fc | SVSRNSAVWNWIRQSPSRGLEWLGRT |
| | | YYRSKWYNDYAPSVKSRITINPDTSK |
| | | NQFSLQLNSVTPEDTAVYYCARGLRQ |
| | | NQFYYYMDVWGKGTTVTVSSASTKG |
| | | PSVFPLAPSSKSTSGGTAALGCLVKDY |
| | | FPEPVTVSWNSGALTSGVHTFPAVLQ |
| | | SSGLYSLSSVVTVPSSSLGTQTYICNV |
| | | NHKPSNTKVDKRVEPKSCDKTHTCPP |
| | | CPAPELLGGPSVFLFPPKPKDTLMISRT |
| | | PEVTCVVVDVSHEDPEVKFNWYVDG |
| | | VEVHNAKTKPREEQYNSTYRVVSVLT |
| | | VLHQDWLNGKEYKCKVSNKALPAPI |
| | | EKTISKAKGQPREPQVYTLPPSREEMT |
| | | KNQVSLTCLVKGFYPSDIAVEWESNG |
| | | QPENNYKTTPPVLDSDGSFFLYSKLTV |
| | | DKSRWQQGNVFSCSVMHEALHNHYT |
| | | QKSLSLSPGK |
| SEQ ID NO: 56 | STEAP2 Nucleotide Sequence | AGTCAGCGTTCCTCGGGCCCTCGGC |
| | | GCCACGAGCTGTCCGGGCACGCAGC |
| | | CCCTAGCGGCGCGTCGCTGCCAAGC |
| | | CGGCCTCCGCGCGCCTCCCTCCTTCC |
| | | TTCTCCCCTGGCTGTTCGCGATCCAG |
| | | CTTGGGTAGGCGGGGAAGCAGCTGG |
| | | AGTGCGACCGCCGCGGCAGCCACCC |
| | | TGCAACCGCCAGTCGGAGAGCTAAG |
| | | GGCAAGTCCTGAGGTTGGGCCCAGG |
| | | AGAAAGAAGGCAAGGAGACATTGT |
| | | CCCAGGATATTCTTGGTGATCTTGG |
| | | AAGTGTCCGTATCATGGAATCAATC |
| | | TCTATGATGGGAAGCCCTAAGAGCC |
| | | TTAGTGAAACTTTTTTACCTAATGGC |
| | | ATAAATGGTATCAAAGATGCAAGGA |
| | | AGGTCACTGTAGGTGTGATTGGAAG |
| | | TGGAGATTTTGCCAAATCCTTGACC |
| | | ATTCGACTTATTAGATGCGGCTATC |
| | | ATGTGGTCATAGGAAGTAGAAATCC |
| | | TAAGTTTGCTTCTGAATTTTTTCCTC |
| | | ATGTGGTAGATGTCACTCATCATGA |
| | | AGATGCTCTCACAAAAACAAATATA |
| | | ATATTTGTTGCTATACACAGAGAAC |
| | | ATTATACCTCCCTGTGGGACCTGAG |
| | | ACATCTGCTTGTGGGTAAAATCCTG |
| | | ATTGATGTGAGCAATAACATGAGGA |
| | | TAAACCAGTACCCAGAATCCAATGC |
| | | TGAATATTTGGCTTCATTATTCCCAG |
| | | ATTCTTTGATTGTCAAAGGATTTAAT |
| | | GTTGTCTCAGCTTGGGCACTTCAGTT |
| | | AGGACCTAAGGATGCCAGCCGGCAG |
| | | GTTTATATATGCAGCAACAATATTC |
| | | AAGCGCGACAACAGGTTATTGAACT |
| | | TGCCCGCCAGTTGAATTTCATTCCCA |
| | | TTGACTTGGGATCCTTATCATCAGCC |
| | | AGAGAGATTGAAAATTTACCCCTAC |
| | | GACTCTTTACTCTCTGGAGAGGGCC |
| | | AGTGGTGGTAGCTATAAGCTTGGCC |
| | | ACATTTTTTTTCCTTTATTCCTTTGTC |
| | | AGAGATGTGATTCATCCATATGCTA |
| | | GAAACCAACAGAGTGACTTTTACAA |
| | | AATTCCTATAGAGATTGTGAATAAA |
| | | ACCTTACCTATAGTTGCCATTACTTT |
| | | GCTCTCCCTAGTATACCTCGCAGGTC |
| | | TTCTGGCAGCTGCTTATCAACTTTAT |
| | | TACGGCACCAAGTATAGGAGATTTC |
| | | CACCTTGGTTGGAAACCTGGTTACA |
| | | GTGTAGAAAACAGCTTGGATTACTA |
| | | AGTTTTTTCTTCGCTATGGTCCATGT |
| | | TGCCTACAGCCTCTGCTTACCGATG |
| | | AGAAGGTCAGAGAGATATTTGTTTC |
| | | TCAACATGGCTTATCAGCAGGTTCA |
| | | TGCAAATATTGAAAACTCTTGGAAT |
| | | GAGGAAGAAGTTTGGAGAATTGAA |
| | | ATGTATATCTCCTTTGGCATAATGAG |
| | | CCTTGGCTTACTTTCCCTCCTGGCAG |
| | | TCACTTCTATCCCTTCAGTGAGCAAT |
| | | GCTTTAAACTGGAGAGAATTCAGTT |
| | | TTATTCAGTCTACACTTGGATATGTC |
| | | GCTCTGCTCATAAGTACTTTCCATGT |
| | | TTTAATTTATGGATGGAAACGAGCT |
| | | TTTGAGGAAGAGTACTACAGATTTT |
| | | ATACACCACCAAACTTTGTTCTTGCT |
| | | CTTGTTTTGCCCTCAATTGTAATTCT |
| | | GGGTAAGATTATTTTATTCCTTCCAT |
| | | GTATAAGCCGAAAGCTAAAACGAAT |
| | | TAAAAAAGGCTGGGAAAAGAGCCA |
| | | ATTTCTGGAAGAAGGTATGGGAGGA |
| | | ACAATTCCTCATGTCTCCCCGGAGA |
| | | GGGTCACAGTAATGTGATGACAAAT |
| | | GGTGTTCACAGCTGCCATATAAAGT |
| | | TCTACTCATGCCATTATTTTTATGAC |
| | | TTCTACGTTCAGTTACAAGTATGCTG |
| | | TCAAATTATCGTGGGTTGAAACTTG |
| | | TTAAATGAGATTTCAACTGACTTAG |
| | | TGATAGAGTTTTCTTCAAGTTAA |
| | | TTTTCACAAATGTCATGTTTGCCAAT |
| | | ATGAATTTTTCTAGTCAACATATTAT |
| | | TGTAATTTAGGTATGTTTTGTTTTGT |
| | | TTTGCACAACTGTAACCCTGTTGTTA |
| | | CTTTATATTTCATAATCAGGCAAAA |
| | | ATACTTACAGTTAATAATATAGATA |
| | | TAATGTTAAAAACAATTTGCAAACC |
| | | AGCAGAATTTTAAGCTTTTAAAATA |
| | | ATTCAATGGATATACATTTTTTTCTG |
| | | AAGATTAAGATTTTAATTATTCAACT |
| | | TAAAAAGTAGAAATGCATTATTATA |
| | | CATTTTTTTAAGAAAGGACACGTTA |
| | | TGTTAGCATCTAGGTAAGGCTGCAT |
| | | GATAGCATTCCTATATTTCTCTCATA |
| | | AAATAGGATTTGAAGGATGAAATTA |
| | | ATTGTATGAAGCAATGTGATTATAT |
| | | GAAGAGACACAAATTAAAAAGACA |
| | | AATTAAACCTGAAATTATATTTAAA |
| | | ATATATTTGAGACATGAAATACAT |
| | | ACTGATAATACATACCTCATGAAAG |
| | | ATTTTATTCTTTATTGTGTTACAGAG |
| | | CAGTTTCATTTTCATATTAATATACT |
| | | GATCAGGAAGAGGATTCAGTAACAT |
| | | TTGGCTTCCAAAACTGCTATCTCTAA |
| | | TACGGTACCAATCCTAGGAACTGTA |
| | | TACTAGTTCCTACTTAGAACAAAAG |
| | | TATCAAGTTTGCACACAAGTAATCT |
| | | GCCAGCTGACCTTTGTCGCACCTTA |
| | | ACCAGTCACCACTTGCTATGGTATA |
| | | GGATTATACTGATGTTCTTTGAGGG |
| | | ATTCTGATGTGCTAGGCATGGT |
| | | TCTAAGTACTTTACTTGTATTATCCC |
| | | ATTTAATACTTAGAACAACCCCGTG |
| | | AGATAAGTAGTTATTATCCTCATTTT |
| | | ACACATGAGGGACCGAAGGATAGA |
| | | AAAGTTATTTTTCAAAGGTCTTGCA |
| | | GTTAATAAATGGCAGAGTGAGCATT |
| | | CAAGTCCAGGTAGTCATATTCCAGA |
| | | GGCCACGGTTTTAACCACTAGGCTC |
| | | TAGAGCTCCCGCCGCGCCCCTATGC |
| | | ATTATGTTCACAATGCCAATCTAGA |
| | | TGCTTCCTCTTTTGTATAAAGTCACT |
| | | GACATTCTTTAGAGTGGGTTGGG |
| | | TGCATCCAAAAATGTATAAAAATAT |
| | | TATTATAATAAACTTATTACTGCTTG |
| | | TAGGGTAATTCACAGTTACTTACCCT |
| | | ATTCTTGCTTGGAACATGAGCCTGG |
| | | AGACCCATGGCAGTCCATATGCCTC |
| | | CCTATGCAGTGAAGGGCCCTAGCAG |
| | | TGTTAACAAATTGCTGAGATCCCAC |
| | | GGAGTCTTTCAAAAATCTCTGTAGA |
| | | GTTAGTCTTCTCCTTTTCTCTTCCTG |
| | | AGAAGTTCTCCTGCCTGCATAACCA |
| | | TTCATTAGGGAGTACTTTACAAGCA |
| | | TGAAGGATATTAGGGTAAGTGG |
| | | CTAATTATAAATCTACTCTAGAGAC |
| | | ATATAATCATACAGATTATTCATAA |
| | | AATTTTTCAGTGCTGTCCTTCCACAT |
| | | TTAATTGCATTTTGCTCAAACTGTAG |
| | | AATGCCCTACATTCCCCCCACCCCA |
| | | ATTTGCTATTTCCTTATTAAAATAGA |
| | | AAATTATAGGCAAGATACAATTATA |
| | | TGCGTTCCTCTTCCTGAAATTATAAC |
| | | ATTTCTAAACTTACCCACGTAGGTA |
| | | CTACTGAATCCAACTGCCAACAATA |
| | | AAAAGACTTTTATTTAGTAGAGGCT |
| | | ACCTTTCCCACCAGTGACTCT |
| | | TTTTCTACAACTGCCTTGTCAGTTTG |
| | | GTAATTCACTTATGATTTTCTAATGT |
| | | TCTCTTGGTGAATTTTATTATCTTGT |
| | | ACCCTCTTTTTTTTTTTTTTTTTTTTT |
| | | AAAGACAGAGTCTTGCTCTGTCACC |
| | | CAGGCTGGAGTGCAGTGGCACGATC |
| | | TCGGCTCACTGCAAGCTCTGCCTCCC |
| | | GGGTTCACGCCATTCTCCTGCCTCAG |
| | | CCTCCCGAGTAGCTGGGACTACAGG |
| | | TGCCCGCCACCATGCCCGGCTGATT |
| | | TCTTTTTGTATTTTTAGTAGAGACGG |
| | | AGTTTCACCGTGTTAGCCAGGATGG |
| | | TCTCGATCTCCTGACCTCGTGATCCG |
| | | CCCGCCTTGGCCTCCAAAGTGCTGG |
| | | GATTACAGGTGTGAGCTACCGCGCC |
| | | CGGCCTATTATCTTGTACTTTCTAAC |
| | | TGAGCCCTCTATTTTCTTTATTTTAA |
| | | TAATATTTCTCCCCACTTGAGAATCA |
| | | CTTGTTAGTTCTTGGTAGGAATTCAG |
| | | TTGGGCAATGATAACTTTTATGGGC |
| | | AAAAACATTCTATTATAGTGAACTA |
| | | ATGAAAATAACAGCGTATTTTCAAT |
| | | ATTTTCTTATTCCTTAAATTCCACTC |
| | | TTTTAACACTATGCTTAACCACTTAA |
| | | TGTGATGAAATATTCCTAAAAGTTA |
| | | AATGACTATTAAAGCATATA |
| | | TTGTTGCATGTATATATTAAGTAGCC |
| | | GATACTCTAAATAAAAATACCACTG |
| | | TTACAGATAAATGGGGCCTTTAAAA |
| | | ATATGAAAAACAAACTTGTGAAAAT |
| | | GTATAAAAGATGCATCTGTTGTTTC |
| | | AAATGGCACTATCTTCTTTTCAGTAC |
| | | TACAAAAACAGAATAATTTTGAAGT |
| | | TTTAGAATAAATGTAATATATTTACT |
| | | ATAATTCTAAATGTTTAAATGCTTTT |
| | | CTAAAAATGCAAAACTATGATGTTT |
| | | AGTTGCTTTATTTTACCTCTATGTGA |
| | | TTATTTTTCTTAATTGTTATTTTTTAT |
| | | AATCATTATTTTTCTGAACCATTCTT |
| | | CTGGCCTCAGAAGTAGGACTGAATT |
| | | CTACTATTGCTAGGTGTGAGAAAGT |
| | | GGTGGTGAGAACCTTAGAGCAGTGG |
| | | AGATTTGCTACCTGGTCTGTGTTTTG |
| | | AGAAGTGCCCCTTAGAAAGTTAAAA |
| | | GAATGTAGAAAAGATACTCAG |
| | | TCTTAATCCTATGCAAAAAAAAAAA |
| | | TCAAGTAATTGTTTTCCTATGAGGA |
| | | AAATAACCATGAGCTGTATCATGCT |
| | | ACTTAGCTTTTATGTAAATATTTCTT |
| | | ATGTCTCCTCTATTAAGAG |
| | | TATTTAAAATCATATTTAAATATGA |
| | | ATCTATTCATGCTAACATTATTTTTC |
| | | AAAACATACATGGAAATTTAGCCCA |
| | | GATTGTCTACATATAAGGTTTTTATT |
| | | TGAATTGTAAAATATTTAAAAGTAT |
| | | GAATAAAATATATTTATAGGTATTT |
| | | ATCAGAGATGATTATTTTGTGCTAC |
| | | ATACAGGTTGGCTAATGAGCTCTAG |
| | | TGTTAAACTACCTGATTAATTTCTTA |
| | | TAAAGCAGCATAACCTTGGCTTGAT |
| | | TAAGGAATTCTACTTTCAAAAATTA |
| | | ATCTGATAATAGTAACAAGGTA |
| | | TATTATACTTTCATTACAATCAAATT |
| | | ATAGAAATTACTTGTGTAAAAGGGC |
| | | TTCAAGAATATATCCAATTTTTAAAT |
| | | ATTTTAATATATCTCCTATCTGATAA |
| | | CTTAATTCTTCTAAATTACCACTTGC |
| | | CATTAAGCTATTTCATAATAAATTCT |
| | | GTACAGTTTCCCCCCAAAAAAGAGA |
| | | TTTATTTATGAAATATTTAAAGTTTC |
| | | TAATGTGGTATTTTAAATAAAGTAT |
| | | CATAAATGTAATAAGTAAATATTTA |
| | | TTTAGGAATACTGTGAACACTGAAC |
| | | TAATTATTCCTGTGTCAGTCTATGAA |
| | | ATCCCTGTTTTGAAATACGTAAACA |
| | | GCCTAAAATGTGTTGAAATTATTTTG |
| | | TAAATCCATGACTTAAAACAAGATA |
| | | CATACATAGTATAACACACCTCACA |
| | | GTGTTAAGATTTATATTGTGAAATG |
| | | AGACACCCTACCTTCAATTGTTCATC |
| | | AGTGGGTAAAACAAATTCTGA |
| | | TGTACATTCAGGACAAATGATTAGC |
| | | CCTAAATGAAACTGTAATAATTTCA |
| | | GTGGAAACTCAATCTGTTTTTACCTT |
| | | TAAACAGTGAATTTTACATGAATGA |
| | | ATGGGTTCTTCACTTTTTTTTTAGTA |
| | | TGAGAAAATTATACAGTGCTTAATT |
| | | TTCAGAGATTCTTTCCATATGTTACT |
| | | AAAAAATGTTTTGTTCAGCCTAACA |
| | | TACTGAGTTTTTTTTAACTTTCTAAA |
| | | TTATTGAATTTCCATCATGCATTCAT |
| | | CCAAAATTAAGGCAGACTGTTTGGA |
| | | TTCTTCCAGTGGCCAGATGA |
| | | GCTAAATTAAATCACAAAAGCAGAT |
| | | GCTTTTGTATGATCTCCAAATTGCCA |
| | | ACTTTAAGGAAATATTCTCTTGAAA |
| | | TTGTCTTTAAAGATCTTTTGCAGCTT |
| | | TGCAGATACCCAGACTGAGCTGGAA |
| | | CTGGAATTTGTCTTCCTATTGACTCT |
| | | ACTTCTTTAAAAGCGGCTGCCCATT |
| | | ACATTCCTCAGCTGTCCTTGCAGTTA |
| | | GGTGTACATGTGACTGAGTGTTGGC |
| | | CAGTGAGATGAAGTCTCCTCAAAGG |
| | | AAGGCAGCATGTGTCCTTTTTCATCC |
| | | CTTCATCTTGCTGCTGGGATTGTGGA |
| | | TATAACAGGAGCCCTGGCAGCTGTC |
| | | TCCAGAGGATCAAAGCCACACCCAA |
| | | AGAGTAAGGCAGATTAGAGACCAG |
| | | AAAGACCTTGACTACTTCCCTACTTC |
| | | CACTGCTTTTTCCTGCATTTAAGCCA |
| | | TTGTAAATCTGGGTGTGTTACATGA |
| | | AGTGAAAATTAATTCTTTCTGCCCTT |
| | | CAGTTCTTTATCCTGATACCATTTAA |
| | | CACTGTCTGAATTAACTAGACTGCA |
| | | ATAATTCTTTCTTTTGAAAGCTTTTA |
| | | AAGGATAATGTGCAATTCACATTAA |
| | | AATTGATTTTCCATTGTCAATTAGTT |
| | | ATACTCATTTTCCTGCCTTGATCTTT |
| | | CATTAGATATTTTGTATCTGCTT |
| | | GGAATATATTATCTTCTTTTTAACTG |
| | | TGTAATTGGTAATTACTAAAACTCT |
| | | GTAATCTCCAAAATATTGCTATCAA |
| | | ATTACACACCATGTTTTCTATCATTC |
| | | TCATAGATCTGCCTTATAAACATTTA |
| | | AATAAAAAGTACTATTTAATGATTT |
| | | AACTTCTGTTTTGAAATGTTGTATAC |
| | | ACGTGGATTTTTTTCTCATTAAATAA |
| | | TAATTCTAGTATTTGA |
| SEQ ID NO: | STEAP2 Sequence | MESISMMGSPKSLSETFLPNGINGIKD |
| 57 | | ARKVTVGVIGSGDFAKSLTIRLIRCGY |
| | | HVVIGSRNPKFASEFFPHVVDVTHHE |
| | | DALTKTNIIFVAIHREHYTSLWDLRHL |
| | | LVGKILIDVSNNMRINQYPESNAEYLA |
| | | SLFPDSLIVKGFNVVSAWALQLGPKD |
| | | ASRQVYICSNNIQARQQVIELARQLNF |
| | | IPIDLGSLSSAREIENLPLRLFTLWRGP |
| | | VVVAISLATFFFLYSFVRDVIHPYARN |
| | | QQSDFYKIPIEIVNKTLPIVAITLLSLVY |
| | | LAGLLAAAYQLYYGTKYRRFPPWLE |
| | | TWLQCRKQLGLLSFFFAMVHVAYSL |
| | | CLPMRRSERYLFLNMAYQQVHANIEN |
| | | SWNEEEVWRIEMYISFGIMSLGLLSLL |
| | | AVTSIPSVSNALNWREFSFIQSTLGYV |
| | | ALLISTFHVLIYGWKRAFEEEYYRFYT |
| | | PPNFVLALVLPSIVILGKIILFLPCISRK |
| | | LKRIKKGWEKSQFLEEGMGGTIPHVS |
| | | PERVTVM |
| SEQ ID NO: | Kappa Light Chain Constant | RTVAAPSVFIFPPSDEQLKSGTASVVC |
| 58 | Region | LLNNFYPREAKVQWKVDNALQSGNS |
| | | QESVTEQDSKDSTYSLSSTLTLSKADY |
| | | EKHKVYACEVTHQGLSSPVTKSFNRG |
| | | EC |
| SEQ ID NO: | FC comprising a | DKTHTCPPCPAPEFEGGPSVFLFPPKP |
| 59 | L234F/L235E/P331S triple | KDTLMISRTPEVTCVVVDVSHEDPEV |
| | mutation (TM) | KFNWYVDGVEVHNAKTKPREEQYNS |
| | | TYRVVSVLTVLHQDWLNGKEYKCKV |
| | | SNKALPASIEKTISKAKGQPREPQVYT |
| | | LPPSREEMTKNQVSLTCLVKGFYPSDI |
| | | AVEWESNGQPENNYKTTPPVLDSDGS |
| | | FFLYSKLTVDKSRWQQGNVFSCSVM |
| | | HEALHNHYTQKSLSLSPGK |
| SEQ ID NO: | Wild Type Fc of IgG1 | DKTHTCPPCPAPELLGGPSVFLFPPKP |
| 60 | | KDTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQVYT |
| | | LPPSREEMTKNQVSLTCLVKGFYPSDI |
| | | AVEWESNGQPENNYKTTPPVLDSDGS |
| | | FFLYSKLTVDKSRWQQGNVFSCSVM |
| | | HEALHNHYTQKSLSLSPGK |

1. An antibody or antigen binding fragment thereof which binds to STEAP2, comprising:
i. a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively;
ii. a HCDR1, a HCDR2, a HCDR3, a LCDR1, a LCDR2, and a LCDR3 comprising the amino acid sequence of SEQ ID NO: 7, SEQ ID NO: 8, SEQ ID NO: 9, SEQ ID NO: 10, SEQ ID NO: 11, and SEQ ID NO: 12, respectively;
iii. a HCDR1, a HCDR2, a HCDR3, a LCDR1, a LCDR2, and a LCDR3 comprising the amino acid sequence of SEQ ID NO: 13, SEQ ID NO: 14, SEQ ID NO: 15, SEQ ID NO: 16, SEQ ID NO: 17, and SEQ ID NO: 18, respectively;
iv. a HCDR1, a HCDR2, a HCDR3, a LCDR1, a LCDR2, and a LCDR3 comprising the amino acid sequence of SEQ ID NO: 19, SEQ ID NO: 20, SEQ ID NO: 21, SEQ ID NO: 22, SEQ ID NO: 23, and SEQ ID NO: 24, respectively; or
v. a HCDR1, a HCDR2, a HCDR3, a LCDR1, a LCDR2, and a LCDR3 comprising the amino acid sequence of SEQ ID NO: 25, SEQ ID NO: 26, SEQ ID NO: 27, SEQ ID NO: 28, SEQ ID NO: 29, and SEQ ID NO: 30, respectively.

2. (canceled)

3. The antibody or antigen binding fragment thereof according to claim 1, wherein the antibody or antigen binding fragment thereof comprises:
i. the VH chain of SEQ ID NO: 31 and the VL chain of SEQ ID NO: 32;
ii. the VH chain of SEQ ID NO: 33 and the VL chain of SEQ ID NO: 32;
iii. the VH chain of SEQ ID NO: 35 and the VL chain of SEQ ID NO: 32;
iv. the VH chain of SEQ ID NO: 37 and the VL chain of SEQ ID NO: 32;
v. the VH chain of SEQ ID NO: 39 and the VL chain of SEQ ID NO: 32;
vi. the VH chain of SEQ ID NO: 45 and the VL chain of SEQ ID NO: 32;
vii. the VH chain of SEQ ID NO: 47 and the VL chain of SEQ ID NO: 32;
viii. the VH chain of SEQ ID NO: 49 and the VL chain of SEQ ID NO: 32;
ix. the VH chain of SEQ ID NO: 51 and the VL chain of SEQ ID NO: 32;
x. the VH chain of SEQ ID NO: 31 and the VL chain of SEQ ID NO: 36;
xi. the VH chain of SEQ ID NO: 33 and the VL chain of SEQ ID NO: 36;
xii. the VH chain of SEQ ID NO: 35 and the VL chain of SEQ ID NO: 36;
xiii. the VH chain of SEQ ID NO: 37 and the VL chain of SEQ ID NO: 38;
xiv. the VH chain of SEQ ID NO: 39 and the VL chain of SEQ ID NO: 40;
xv. the VH chain of SEQ ID NO: 45 and the VL chain of SEQ ID NO: 46;
xvi. the VH chain of SEQ ID NO: 47 and the VL chain of SEQ ID NO: 48;
xvii. the VH chain of SEQ ID NO: 49 and the VL chain of SEQ ID NO: 50; or
xviii. the VH chain of SEQ ID NO: 51 and the VL chain of SEQ ID NO: 52.

4.-12. (canceled)

13. The antibody or antigen binding fragment thereof according to claim 1, wherein the antibody or antigen binding fragment thereof comprises a heavy chain comprising the amino acid sequence of SEQ ID NO: 41; and a light chain comprising the amino acid sequence of SEQ ID NO: 42.

14. (canceled)

15. The antibody or antigen binding fragment thereof according to claim 1, wherein the antibody or antigen binding fragment thereof is conjugated to a heterologous agent.

16. The antibody or antigen binding fragment thereof according to claim 1, wherein the antibody or antigen binding fragment thereof is conjugated to one or more heterologous agent selected from the group consisting of a topoisomerase I inhibitor, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, or a combination thereof.

17. (canceled)

18. (canceled)

19. The antibody or antigen binding fragment thereof according to claim 1, wherein the antibody or antigen binding fragment thereof is conjugated to a heterologous agent selected from the group consisting of tubulysin AZ1508, SG3932, or a combination thereof.

20.-29. (canceled)

30. A pharmaceutical composition comprising an antibody or antigen binding fragment thereof according to claim 1.

31. A polynucleotide encoding the antibody or antigen binding fragment thereof according to claim 1.

32. A host cell comprising the polynucleotide of claim 31.

33. A method for producing an antibody or antigen binding fragment thereof that binds to STEAP2, comprising expressing a polynucleotide according to claim 32 in a host cell.

34. (canceled)

35. A method of treating a cancer comprising a cancer cell which expresses STEAP2, the method comprising administering to a subject the antibody or antigen binding fragment of claim 1.

36.-39. (canceled)

40. A method for detecting the presence or absence of a STEAP2 polypeptide in a sample, comprising:
i. contacting a sample with an antibody or antigen binding fragment thereof according to claim 1, to provide an antibody-antigen complex;
ii. detecting the presence or absence of said antibody-antigen complex;
iii. wherein the presence of the antibody-antigen complex confirms the presence of a STEAP2 polypeptide;
iv. wherein the absence of the antibody-antigen complex confirms the absence of STEAP2 polypeptide.

41.-43. (canceled)

44. An antibody-drug conjugate (ADC) comprising:
(i) (a) an antibody or antigen binding fragment thereof which binds to a STEAP2 polypeptide comprising: a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; and a LCDR1 comprising the amino acid sequence of SEQ ID NO: 4; a LCDR2 comprising the amino acid sequence of SEQ ID NO: 5; and a LCDR3 comprising the amino acid sequence of SEQ ID NO: 6, or
(b) an antibody or antigen binding fragment thereof which binds to a STEAP2 polypeptide comprising: a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; and a LCDR1 comprising the amino acid sequence of SEQ ID NO: 10; a LCDR2 comprising the amino acid sequence of SEQ ID NO: 11; and a LCDR3 comprising the amino acid sequence of SEQ ID NO: 12; or
(c) an antibody or antigen binding fragment thereof which binds to a STEAP2 polypeptide comprising: a HCDR1 comprising the amino acid sequence of SEQ ID NO: 19; a HCDR2 comprising the amino acid sequence of SEQ ID NO: 20; a HCDR3 comprising the amino acid sequence of SEQ ID NO: 21; and a LCDR1 comprising the amino acid sequence of SEQ ID NO: 22; a LCDR2 comprising the amino acid sequence of SEQ ID NO: 23; and a LCDR3 comprising the amino acid sequence of SEQ ID NO: 24; and
(ii) a cytotoxic agent wherein the cytotoxic agent is SG3932; and
(iii) wherein the ADC has a drug to antibody ratio (DAR) ranging from about 4 to about 8.

45.-50. (canceled)

51. The ADC of claim 44 comprising a heavy chain (HC) comprising the amino acid sequence of SEQ ID NO: 41, and light chain (LC) comprising the amino acid sequence of SEQ ID NO: 42; or a heavy chain (HC) comprising the amino acid sequence of SEQ ID NO: 43, and light chain (LC) comprising the amino acid sequence of SEQ ID NO: 44.

52.-57. (canceled)

58. A pharmaceutical composition comprising the ADC of claim 44.

59. A method of treating a cancer expressing STEAP2, comprising administering to a subject the ADC of claim 44.

60. (canceled)

61. (canceled)

62. A method for reducing the volume of a tumor expressing STEAP2, comprising administering to a subject claim 44.

63.-65. (canceled)

66. An antibody-drug conjugate as in Formula (IC):
Ab-(G^{A}-J^{A}-D^{C})ₖ (IC)
or a pharmaceutically acceptable salt thereof, wherein
Ab is an antibody or antigen-binding fragment thereof according to a claim 1,
k is an integer from 1 to 10, about 4 to 8, about 4, or about 8,
each G^{A} is independently a conjugation group conjugated to the antibody or antigen-binding fragment thereof,
each D^{C} is 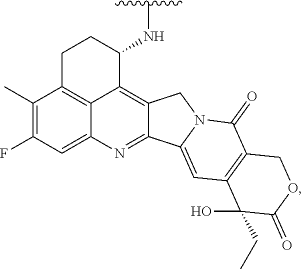 [SMILES: *N[C@H]1CCc2c(C)c(F)cc3nc4c(c1c23)Cn1c-4cc2c(c1=O)COC(=O)[C@]2(O)CC]
each J^{A} is independently a group of Formula (ICA) 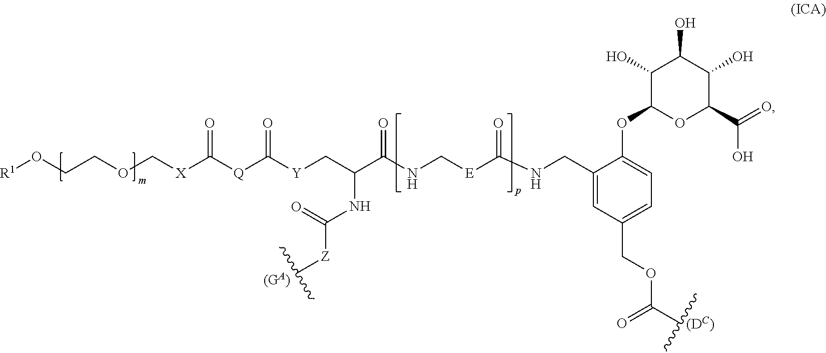
E is (CH₂)ₙ₁, wherein n is 0, 1, 2 or 3,
Q is 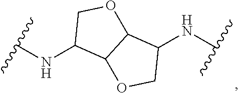 [SMILES: *NC1COC2C(N*)COC12]
R¹ is C₁₋₄ alkyl,
X is (CH₂)ₙ₂, wherein n2 is 0, 1, 2 or 3,
Y is (CH₂)ₙ₃, wherein n3 is 0, 1, 2, 3 or 4,
Z is (CH₂)ₙ₄, wherein n4 is 1, 2, 3, 4 or 5,
m is an integer from 5 to 17,
p is 1 or 0,
(G^{A}) indicates the point of attachment to G^{A}, and
(D^{C}) indicates the point of attachment to D^{C}.

67.-78. (canceled)

79. The antibody-drug conjugate according to claim 66 wherein G^{A}-J^{A}-D^{C} is: 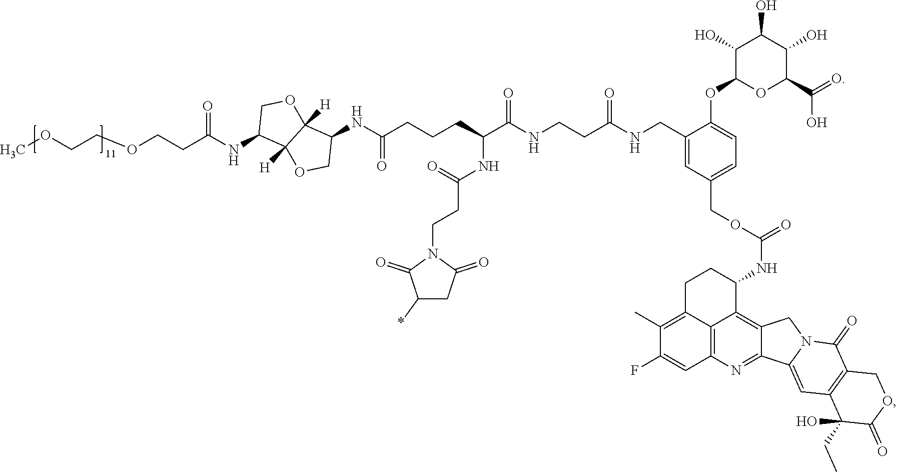
wherein [symbol] indicates the point of attachment to the antibody or antigen-binding fragment thereof.

80.-86. (canceled)

87. A pharmaceutical composition comprising an antibody-drug conjugate according to claim 66.

88. A method of treating a cancer comprising a cancer cell which expresses STEAP2, the method comprising administering to a subject the antibody-drug conjugate of claim 66.

89.-92. (canceled)

93. An antibody-drug conjugate (ADC) comprising:
(i) (a) an antibody or antigen binding fragment thereof which binds to a STEAP2 polypeptide comprising: a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; and a LCDR1 comprising the amino acid sequence of SEQ ID NO: 4; a LCDR2 comprising the amino acid sequence of SEQ ID NO: 5; and a LCDR3 comprising the amino acid sequence of SEQ ID NO: 6, or
(b) an antibody or antigen binding fragment thereof which binds to a STEAP2 polypeptide comprising: a HCDR1 comprising the amino acid sequence of SEQ ID NO: 7; a HCDR2 comprising the amino acid sequence of SEQ ID NO: 8; a HCDR3 comprising the amino acid sequence of SEQ ID NO: 9; and a LCDR1 comprising the amino acid sequence of SEQ ID NO: 10; a LCDR2 comprising the amino acid sequence of SEQ ID NO: 11; and a LCDR3 comprising the amino acid sequence of SEQ ID NO: 12; or
(c) an antibody or antigen binding fragment thereof which binds to a STEAP2 polypeptide comprising: a HCDR1 comprising the amino acid sequence of SEQ ID NO: 19; a HCDR2 comprising the amino acid sequence of SEQ ID NO: 20; a HCDR3 comprising the amino acid sequence of SEQ ID NO: 21; and a LCDR1 comprising the amino acid sequence of SEQ ID NO: 22; a LCDR2 comprising the amino acid sequence of SEQ ID NO: 23; and a LCDR3 comprising the amino acid sequence of SEQ ID NO: 24; and
(ii) a cytotoxic agent wherein the cytotoxic agent is LP-1; and
(iii) wherein the ADC has a drug to antibody ratio (DAR) ranging from about 4 to about 8.

94.-114. (canceled)
